(19) United States
(12) Patent Application Publication
(10) Pub. No.: US 2005/0031651 A1
(43) Pub. Date: Feb. 10, 2005

(54) Therapeutic formulations for the treatment of beta-amyloid related diseases

(76) Inventors: Francine Gervais, Ile Bizard (CA); Francesco Bellini, Mount Royal (CA)

Correspondence Address: LAHIVE & COCKFIELD, LLP., 28 STATE STREET, BOSTON, MA, 02109, US

(21) Appl. No.: 10/871,537

(22) Filed: Jun. 18, 2004

Related U.S. Application Data
(63) Continuation-in-part of application No. 10/746,138, filed on Dec. 24, 2003.
(60) Provisional application No. 60/436,379, filed on Dec. 24, 2002.
(60) Provisional application No. 60/482,214, filed on Jun. 23, 2003.
(60) Provisional application No. 60/480,984, filed on Jun. 23, 2003.
(60) Provisional application No. 60/512,116, filed on Oct. 17, 2003.
(60) Provisional application No. 60/482,058, filed on Jun. 23, 2003.
(60) Provisional application No. 60/512,135, filed on Oct. 17, 2003.
(60) Provisional application No. 60/480,918, filed on Jun. 23, 2003.
(60) Provisional application No. 60/512,017, filed on Oct. 17, 2003.
(60) Provisional application No. 60/480,906, filed on Jun. 23, 2003.
(60) Provisional application No. 60/512,047, filed on Oct. 17, 2003.

(30) Foreign Application Priority Data
Dec. 24, 2003 (WO) PCT/CA03/02011

(51) Int. Cl. A61K 9/00

(52) U.S. Cl. 424/400

(57) ABSTRACT

This invention relates to methods and pharmaceutical compositions for treating amyloid-β related diseases, including Alzheimer's disease. The invention, for example, includes a method of concomitant therapeutic treatment of a subject, comprising administering an effective amount of a first agent and a second agent, wherein said first agent treats an amyloid-β disease, neurodegeneration, or cellular toxicity; and said second agent is a therapeutic drug or nutritive supplement.

### RELATED APPLICATIONS

This application claims priority to U.S. provisional application No. 60/436,379, filed Dec. 24, 2002, identified by Attorney Docket No. NBI-154-1, entitled Combination Therapy for the Treatment of Alzheimer's Disease, U.S. provisional application 60/482,214, filed Jun. 23, 2003, identified by Attorney Docket No. NBI-154-2, U.S. utility patent application Ser. No. 10/746,138, filed Dec. 24, 2003, identified by Attorney Docket No. NBI-154, and International patent application no. PCT/CA2003/0020 11, identified by NBI-154PC entitled Therapeutic Formulations for the Treatment of Beta-Amyloid Related Diseases.

This application is related to U.S. provisional patent application No. 60/480,984, filed Jun. 23, 2003, identified by Attorney Docket No. NBI-152-1, U.S. provisional patent application No. 60/512,116, filed Oct. 17, 2003, identified by Attorney Docket No. NBI-152-2, both entitled, and U.S. application 10/______, filed Jun. 18, 2004, identified by Attorney Docket No. NBI-152, entitled Pharmaceutical Formulations of Amyloid-Inhibiting Compounds. This application is related to U.S. provisional patent application No. 60/482,058, filed Jun. 23, 2003, identified by Attorney Docket No. NBI-156-1, U.S. provisional patent application No. 60/512,135, filed Oct. 17, 2003, identified by Attorney Docket No. NBI-156-2, both entitled Synthetic Process for Preparing Compounds for Treating Amyloidosis, and U.S. application Ser. No. 10/______, filed Jun. 18, 2004, identified by Attorney Docket No. NBI-156, entitled Improved Pharmaceutical Drug Candidates and Method for Preparation Thereof. This application is also related to U.S. provisional patent application No. 60/480,918, filed Jun. 23, 2003, identified by Attorney Docket No. NBI-149-1, U.S. provisional application 60/512,017, filed Oct. 17, 2003, identified by Attorney Docket No. NBI-149-2, and U.S. patent application Ser. No. 10/______, filed Jun. 18, 2004, identified by Attorney Docket No. NBI-149 entitled Methods for Treating Protein Aggregation Disorders. This application is also related to U.S. provisional patent application No. 60/480,906, filed Jun. 23, 2003, identified by Attorney Docket No. NBI-162-1, U.S. provisional patent application No. 60/512,047, filed Oct. 17, 2003, identified by Attorney Docket No. NBI-162-2, U.S. application Ser. No. 10/______, filed Jun. 18, 2004, identified by Attorney Docket No. NBI-162A and U.S. application Ser. No. 10/______, filed Jun. 18, 2004, identified by Attorney Docket No. NBI-162B, all entitled Methods and Compositions for Treating Amyloid-Related Diseases; and U.S. provisional patent application No. 60/480,928, also filed 23 Jun. 2003, identified by Attorney Docket No. NBI-163-1, U.S. provisional patent application No. 60/512,018, filed Oct. 17, 2003, identified by Attorney Docket No. NBI-163-2 and U.S. application 10/______, filed Jun. 18, 2004, identified by Attorney Docket No. NBI-163, all entitled Methods and Compositions for the Treatment of Amyloid- and Epileptogenesis-Associated Diseases; This application is also related to Method for Treating Amyloidosis, U.S. patent application Ser. No. 08/463,548, now U.S. Pat. No. 5,972,328, identified by Attorney Docket No. NCI-003CP4.

The entire contents of each of the foregoing patent applications and patents are expressly incorporated by reference in their entirety including, without limitation, the specification, claims, and abstract, as well as any figures, tables, or drawings thereof.

### BACKGROUND

Alzheimer's disease is a devastating disease of the brain that results in progressive memory loss leading to dementia, physical disability, and death over a relatively long period of time. With the aging populations in developed countries, the number of Alzheimer's patients is reaching epidemic proportions.

People suffering from Alzheimer's disease develop a progressive dementia in adulthood, accompanied by three main structural changes in the brain: diffuse loss of neurons in multiple parts of the brain; accumulation of intracellular protein deposits termed neurofibrillary tangles; and accumulation of extracellular protein deposits termed amyloid or senile plaques, surrounded by misshapen nerve terminals (dystrophic neurites). A main constituent of these amyloid plaques is the amyloid-β peptide (Aβ), a 39-43 amino-acid protein that is produced through cleavage of the β-amyloid precursor protein (APP). Extensive research has been conducted on the relevance of Aβ deposits in Alzheimer's disease, see, e.g., Selkoe, Trends in Cell Biology 8, 447-453 (1998). Aβ naturally arises from the metabolic processing of the amyloid precursor protein (“APP”) in the endoplasmic reticulum (“ER”), the Golgi apparatus, or the endosomal-lysosomal pathway, and most is normally secreted as a 40 (“Aβ1-40”) or 42 (“Aβ1-42”) amino acid peptide (Selkoe, Annu. Rev. Cell Biol. 10, 373-403 (1994)). A role for Aβ as a primary cause for Alzheimer's disease is supported by the presence of extracellular amyloid β peptide (“Aβ”) deposits in senile plaques of Alzheimer's disease (“Alzheimer's disease”), the increased production of Aβ in cells harboring mutant Alzheimer's disease associated genes, e.g., amyloid precursor protein, presenilin I and presenilin II; and the toxicity of extracellular soluble (oligomeric) or fibrillar Aβ to cells in culture. See, e.g., Gervais, Eur. Biopharm. Review, 40-42 (Autumn 2001); May, DDT 6, 459-62 (2001). Although symptomatic treatments exist for Alzheimer's disease, this disease cannot be prevented or cured at this time.

Alzheimer's disease is characterized by diffuse and neuritic plaques, cerebral angiopathy, and neurofibrillary tangles. Plaque and blood vessel amyloid is believed to be formed by the deposition of insoluble Aβ amyloid protein, which may be described as diffuse or fibrillary. Both soluble oligomeric Aβand fibrillar Aβare also believed to be neurotoxic and inflammatory. Amyloid fibrils, once deposited, can become toxic to the surrounding cells. For example, the Aβfibrils organized as senile plaques have been shown to be associated with dead neuronal cells and microgliosis in patients with Alzheimer's disease. When tested in vitro, Aβpeptide was shown to be capable of triggering an activation process of microglia (brain macrophages), which would explain the presence of microgliosis and brain inflammation found in the brain of patients with Alzheimer's disease. Once these amyloids have formed, there is no known, widely accepted therapy or treatment that significantly dissolves amyloid deposits or prevents the formation of deposits in situ. Presently available pharmaceutical technology for treatment of β-amyloid diseases is almost entirely symptomatic, providing only temporary or partial clinical benefit. Although some pharmaceutical agents have been described that offer partial symptomatic relief, no comprehensive pharmacological therapy is currently available for the treatment of Alzheimer's disease.

### SUMMARY OF THE INVENTION

The present invention provides a method of concomitant therapeutic treatment of a subject. The method generally includes administering to a subject in need thereof an effective amount of a pharmaceutical composition for treating or preventing an amyloid-β disease such that activities of daily living otherwise impaired by said amyloid-β disease are improved or stabilized. In one embodiment, the pharmaceutical composition includes a first agent and a second agent in a pharmaceutically acceptable carrier, wherein said first agent prevents or treats amyloid-β related disease, and said second agent is a therapeutic drug or nutritive supplement.

In another embodiment, the present invention provides a method of preventing or treating Alzheimer's disease that includes concomitantly administering to a subject in need thereof an effective amount of a first agent that is efficacious in preventing or treating Alzheimer's disease in said subject and a second agent, wherein said first agent comprises 3-amino-1-propanesulfonic acid or a pharmaceutically acceptable salt thereof.

In another embodiment, the present invention provides a method of preventing or treating Mild Cognitive Impairment that includes concomitantly administering to a subject in need thereof an effective amount of a first agent that is efficacious in preventing or treating Mild Cognitive Impairment in said subject and a second agent, wherein said first agent comprises 3-amino-1-propanesulfonic acid or a pharmaceutically acceptable salt thereof.

In yet another embodiment, the present invention provides a method of preventing or treating comprising concomitantly administering to a subject in need thereof an effective amount of a first agent that is efficacious in preventing or treating Mild Cognitive Impairment in said subject and a second agent, wherein said first agent comprises 3-amino-1-propanesulfonic acid or a pharmaceutically acceptable salt thereof.

In yet another embodiment, the present invention provides a method of preventing or treating Mild Cognitive Impairment that includes concomitantly administering to a subject in need thereof an effective amount of a first agent that is efficacious in preventing or treating Mild Cognitive Impairment in said subject and a second agent, wherein said first agent is 3-amino-1-propanesulfonic acid.

In yet another embodiment, the present invention provides a method of preventing or treating comprising concomitantly administering to a subject in need thereof an effective amount of a first agent that is efficacious in preventing or treating Mild Cognitive Impairment in said subject and a second agent, wherein said first agent is 3-amino-1-propanesulfonic acid. The second agent can be a cholinesterase inhibitor, a statin, or memantine.

### BRIEF DESCRIPTION OF THE DRAWINGS

FIGS. 1-13 and 5-68 depict formulae and compounds of the invention; and

FIG. 14 is a graph illustrating that a test drug given concomitantly with ACHE inhibitors is able to maintain and even improve the cognitive function of patients.

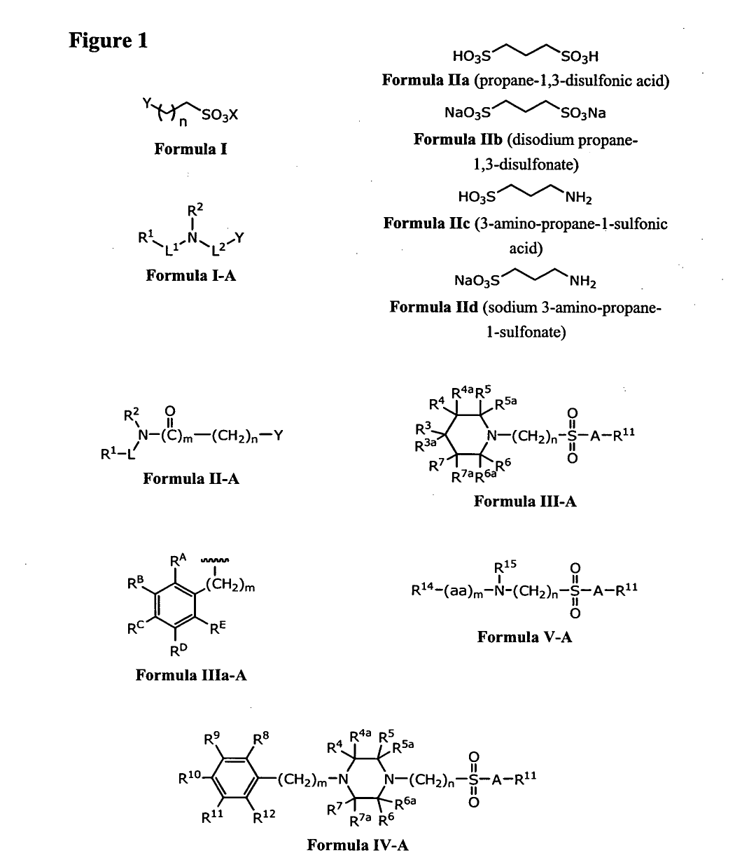

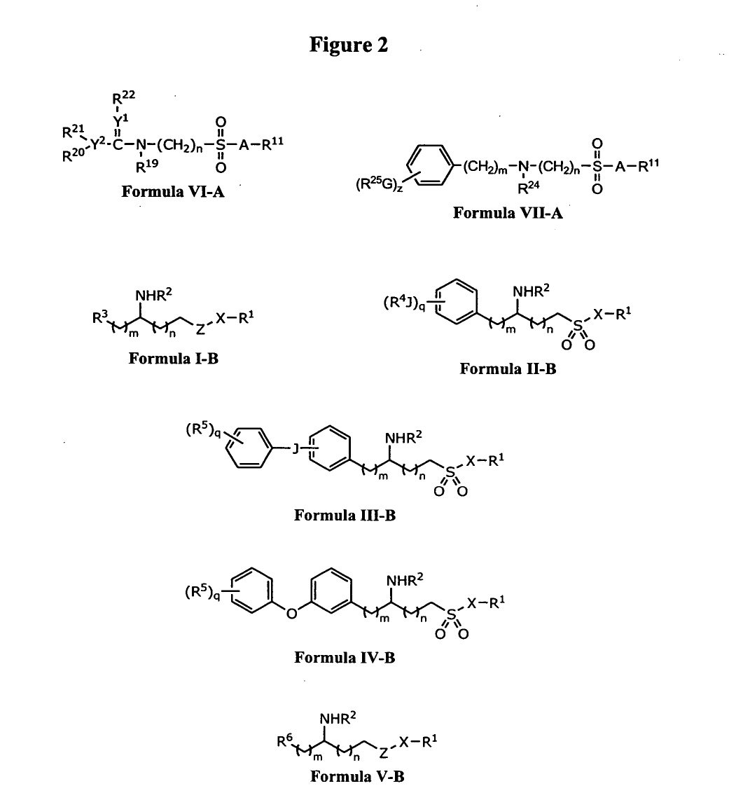

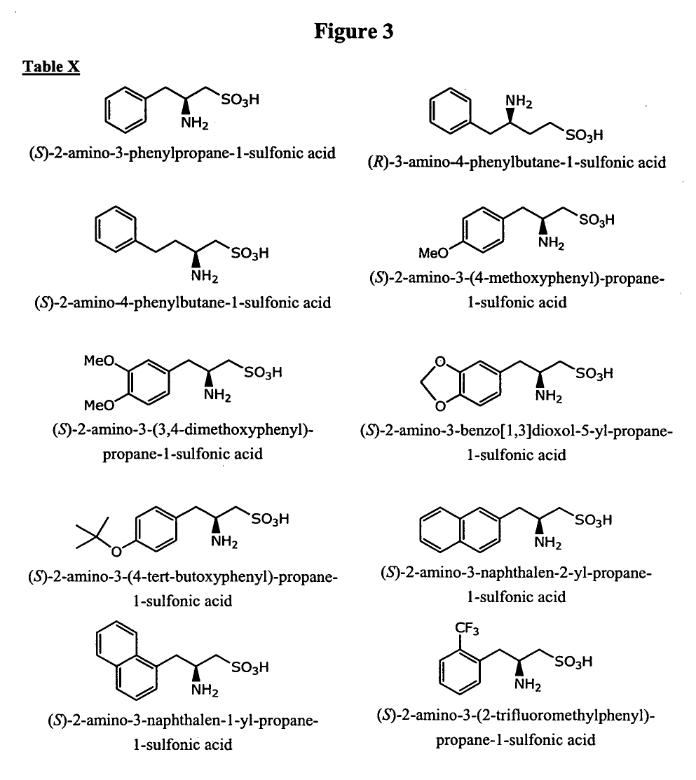

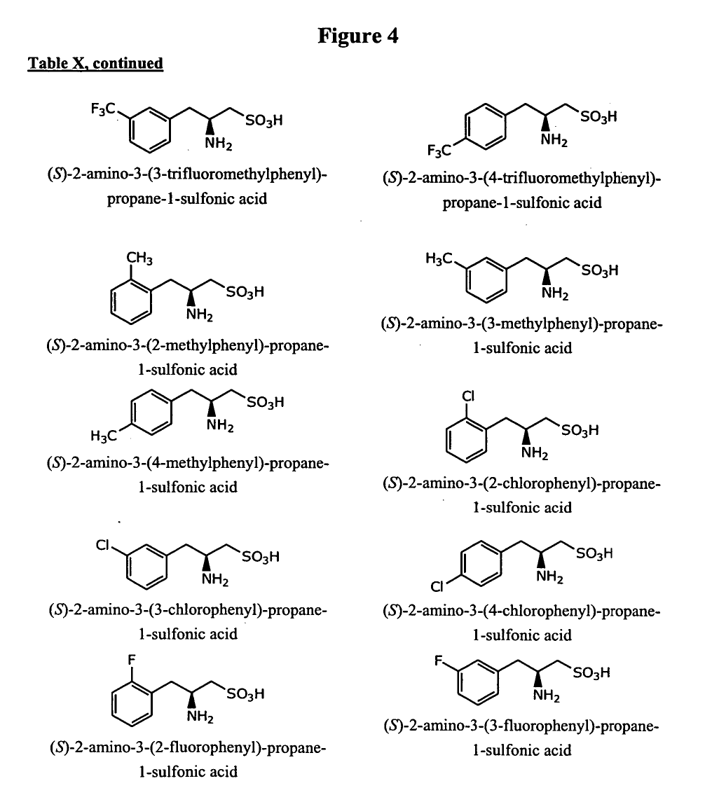

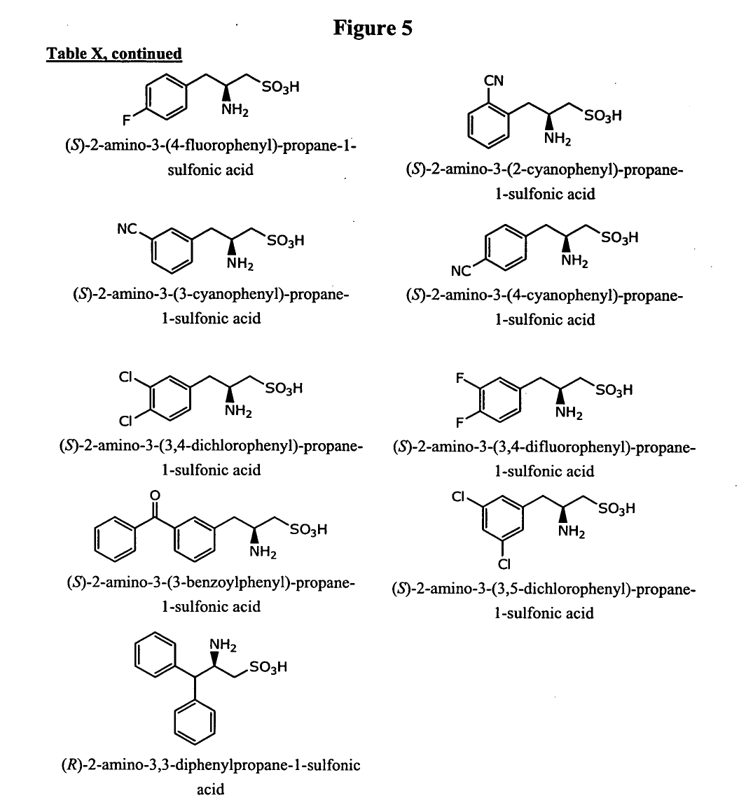

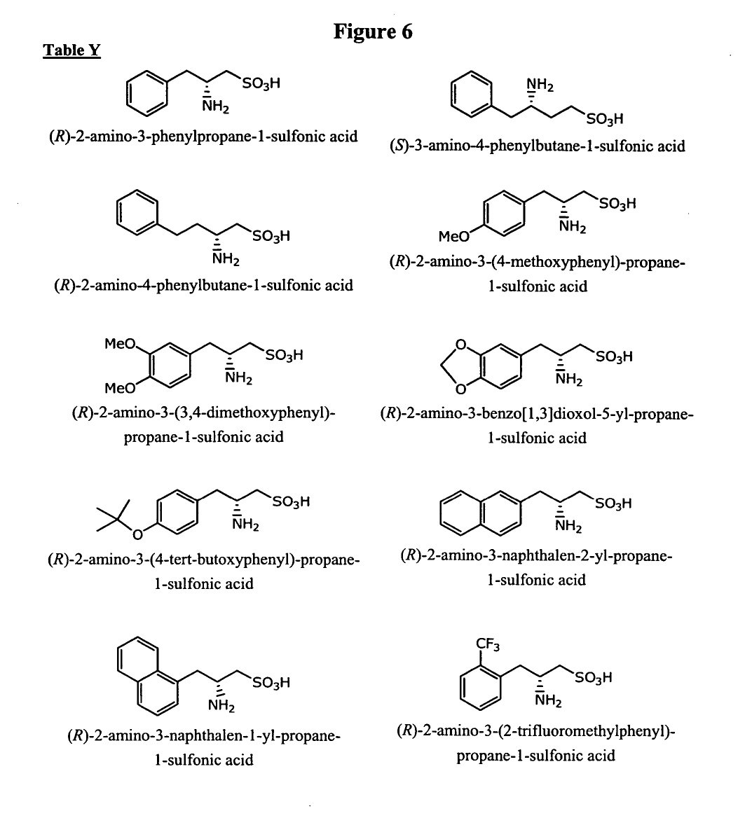

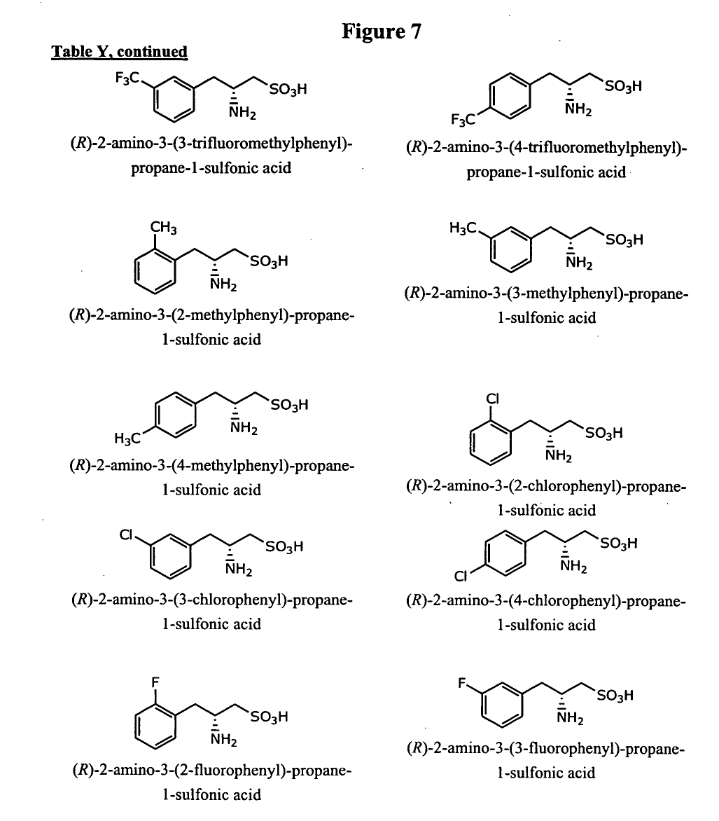

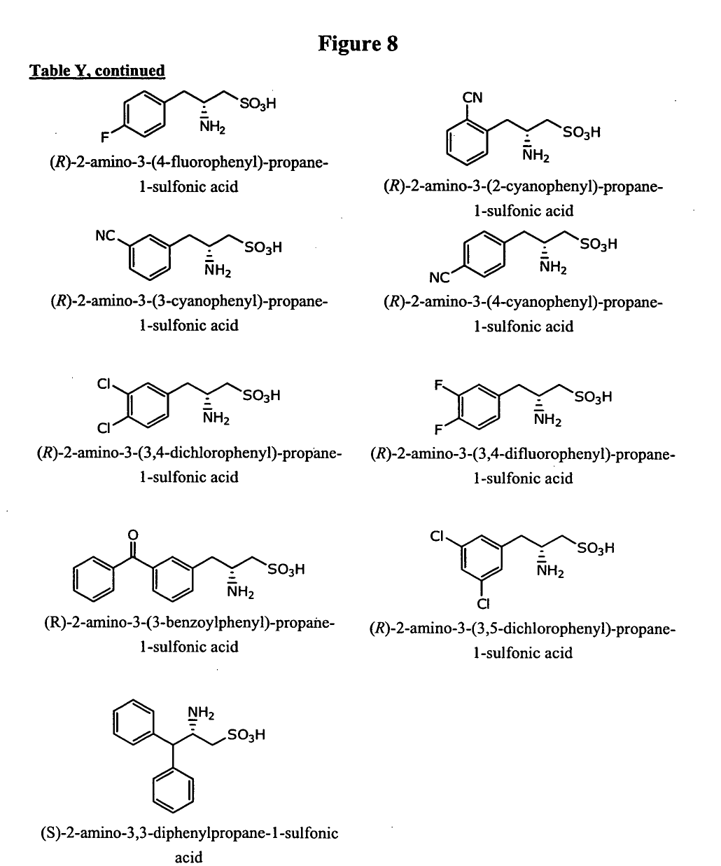

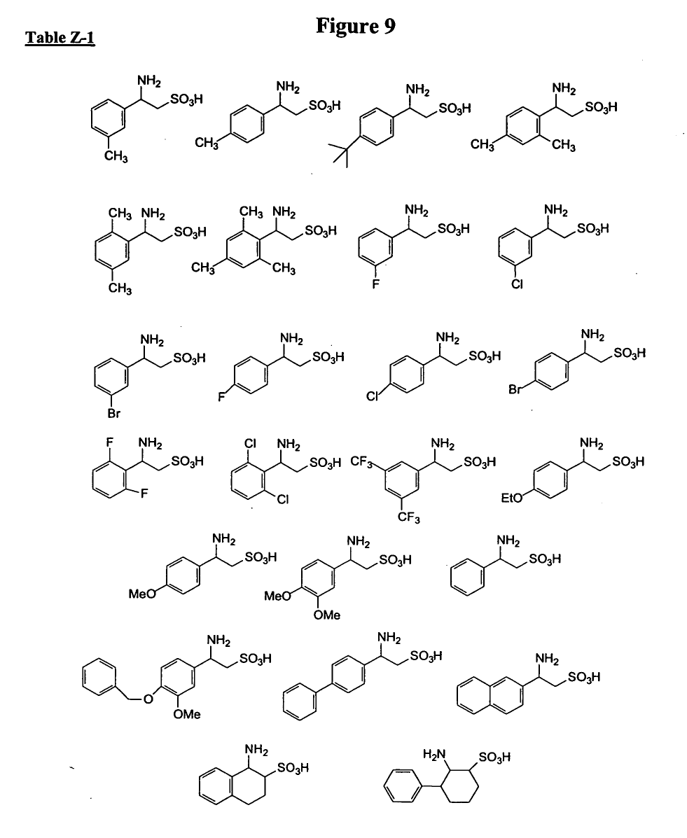

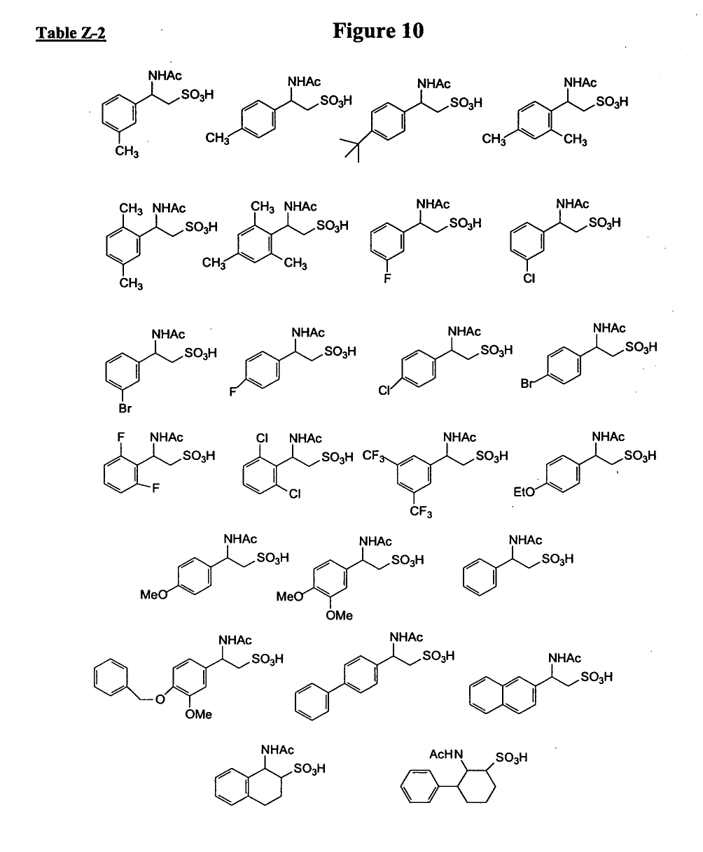

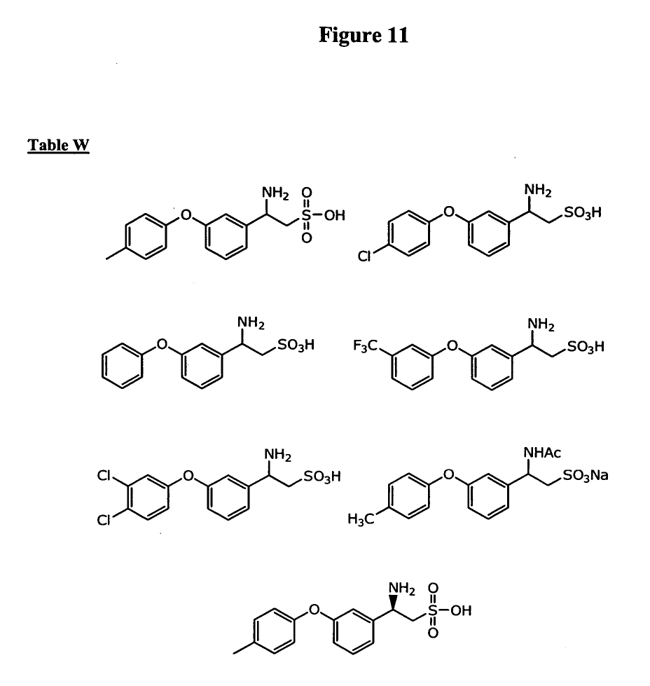

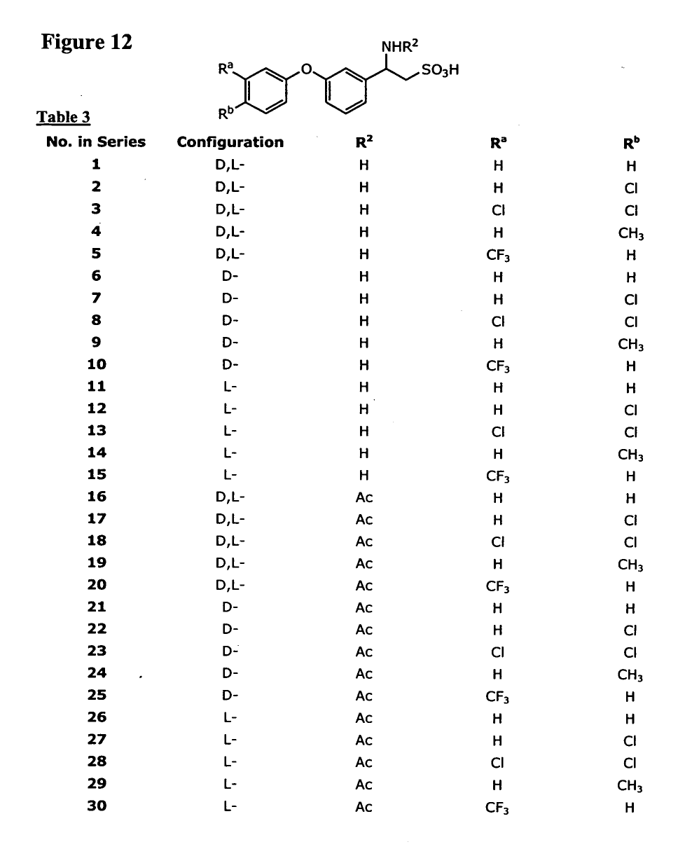

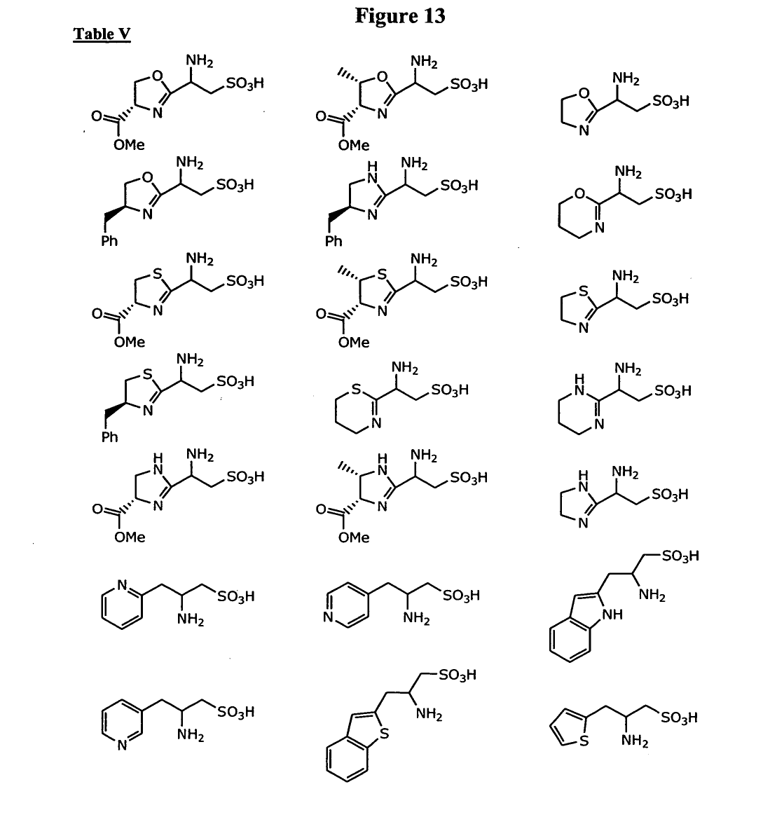

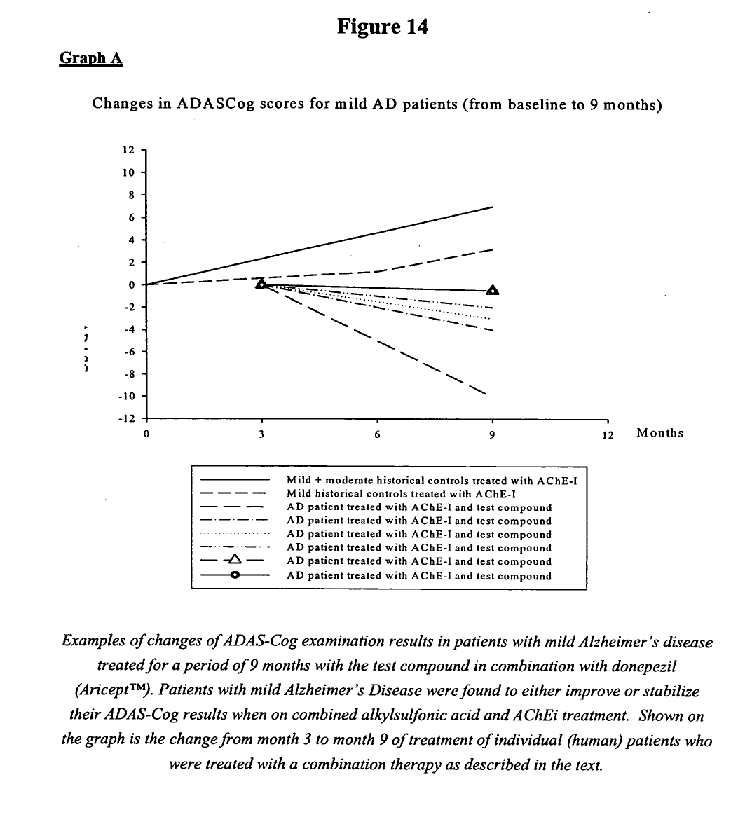

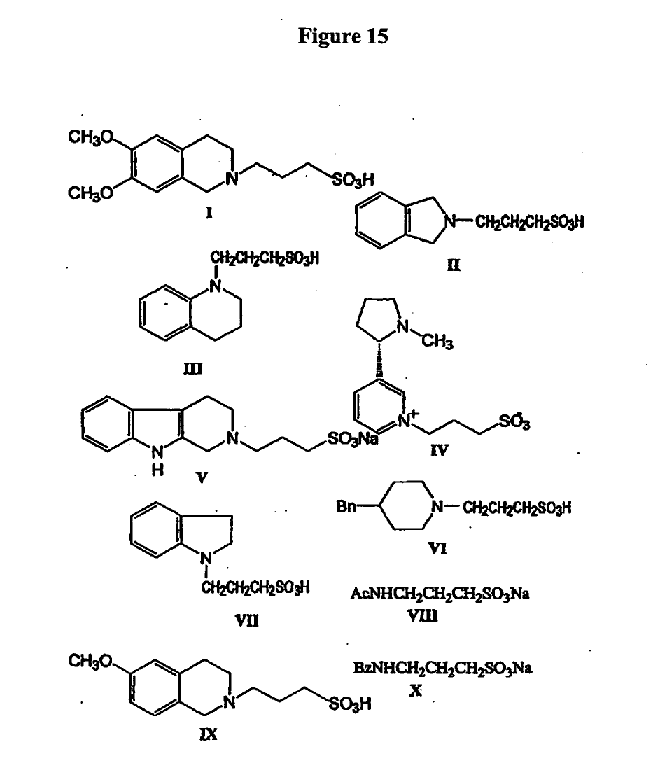

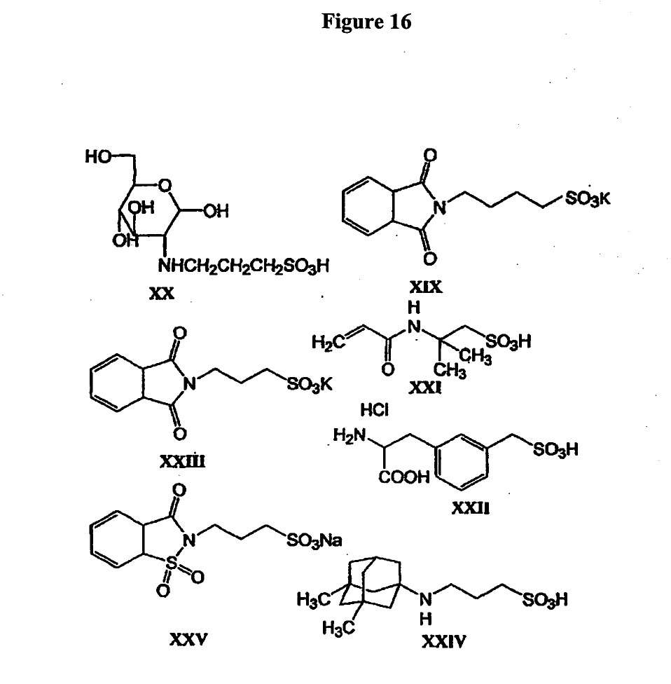

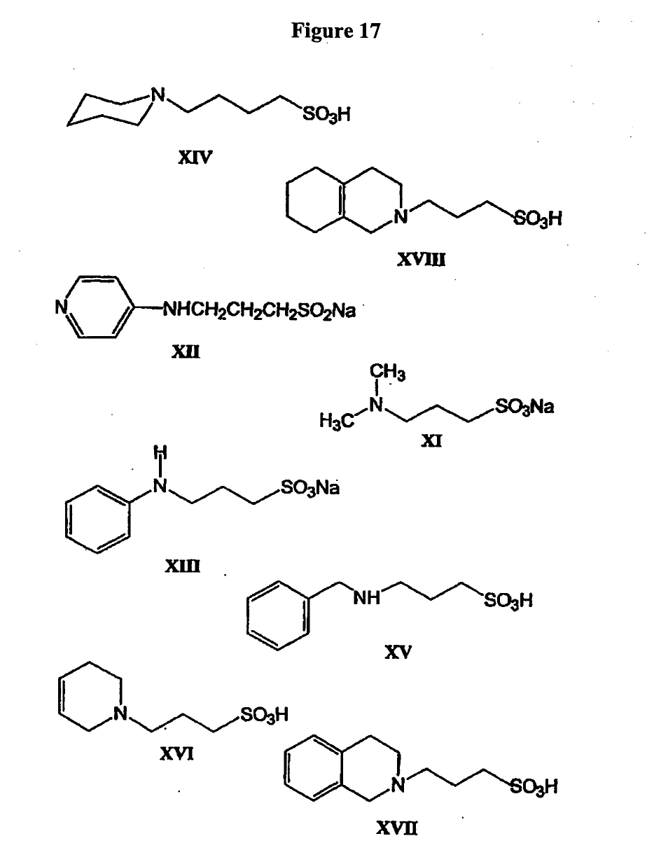

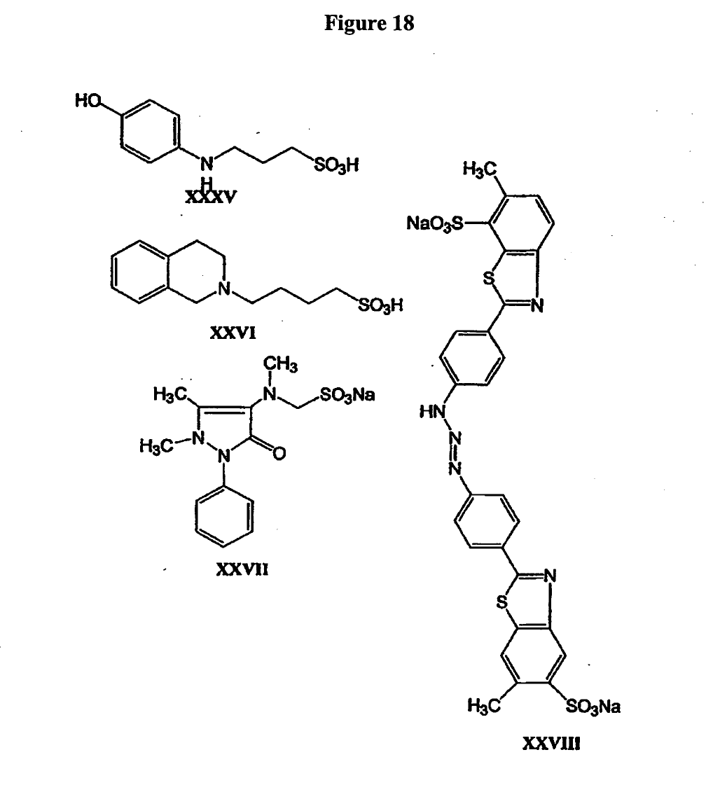

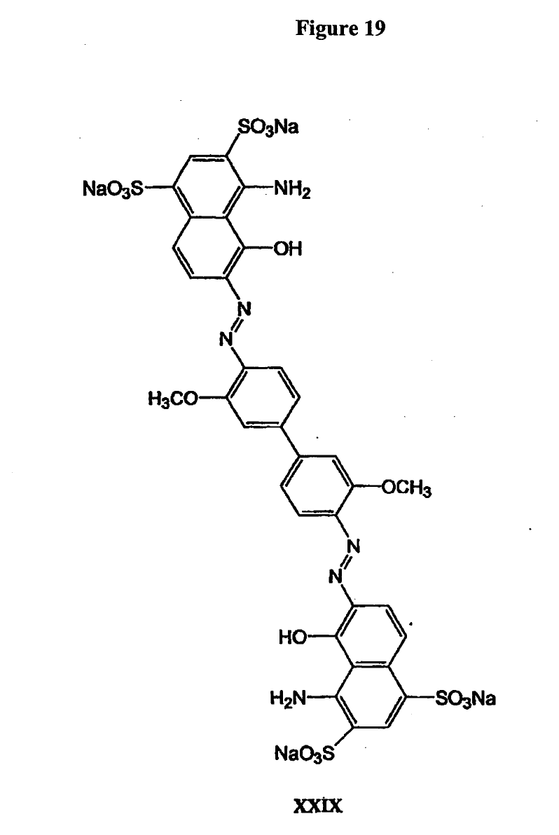

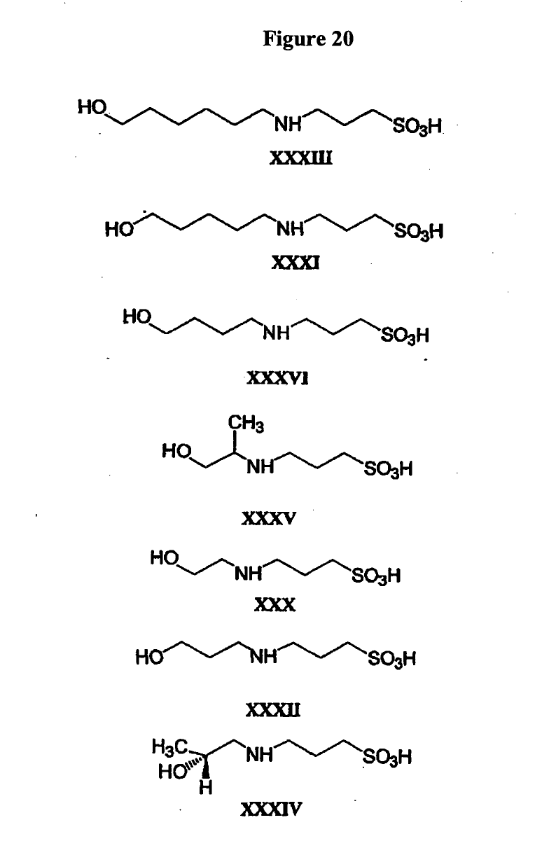

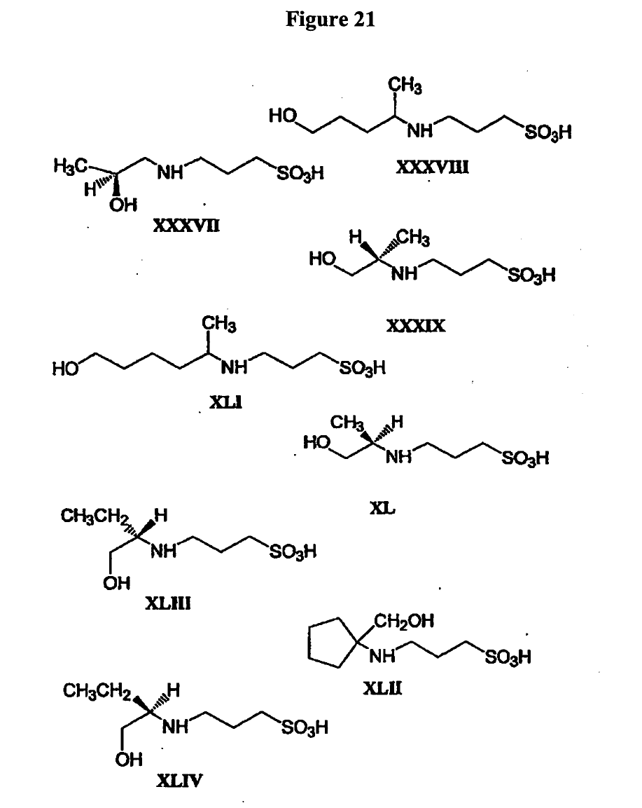

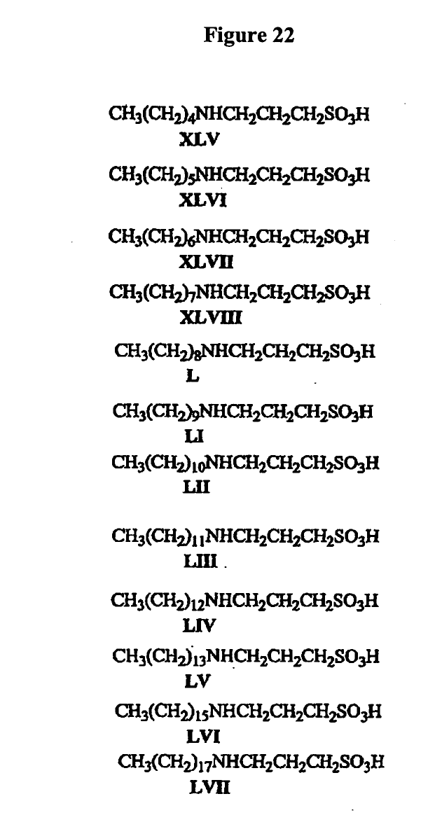

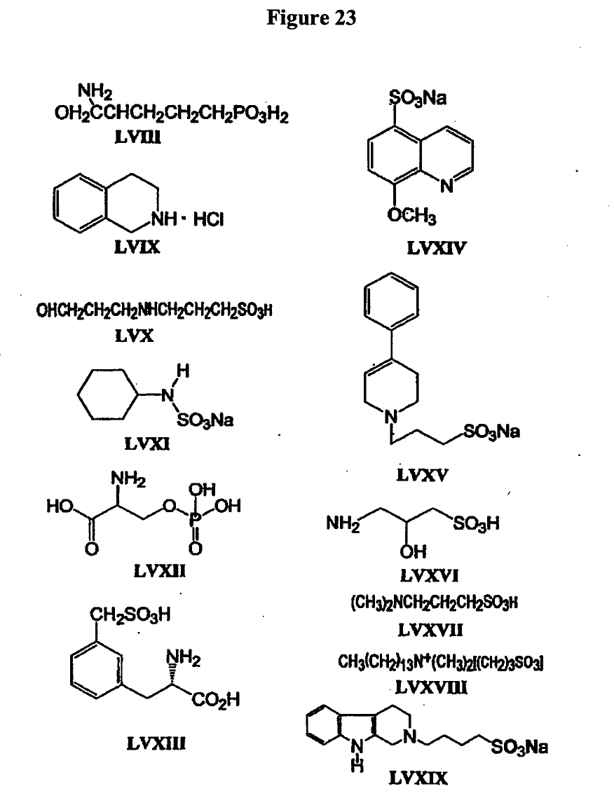

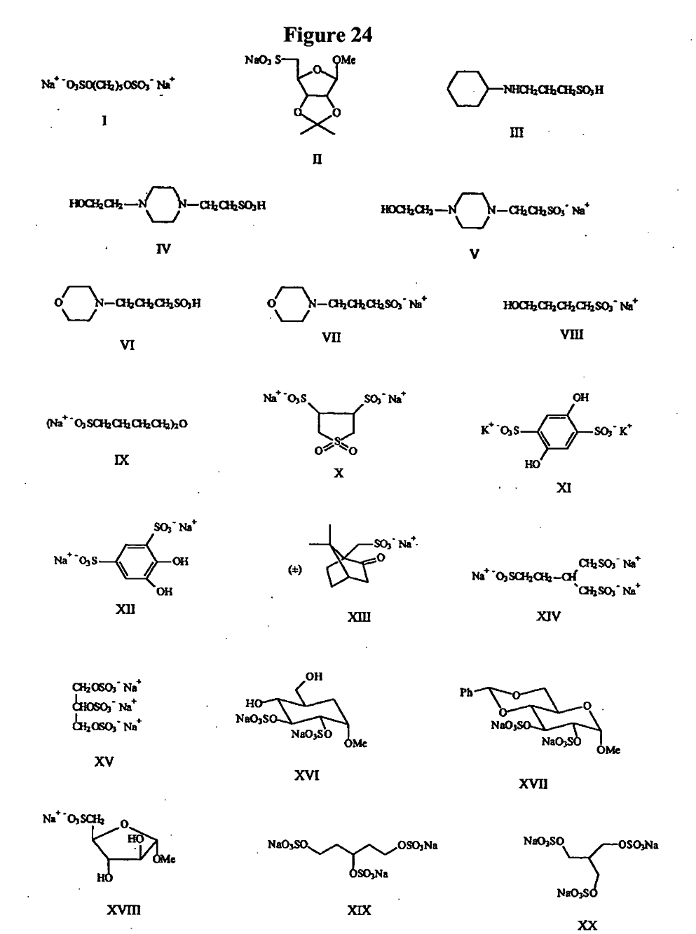

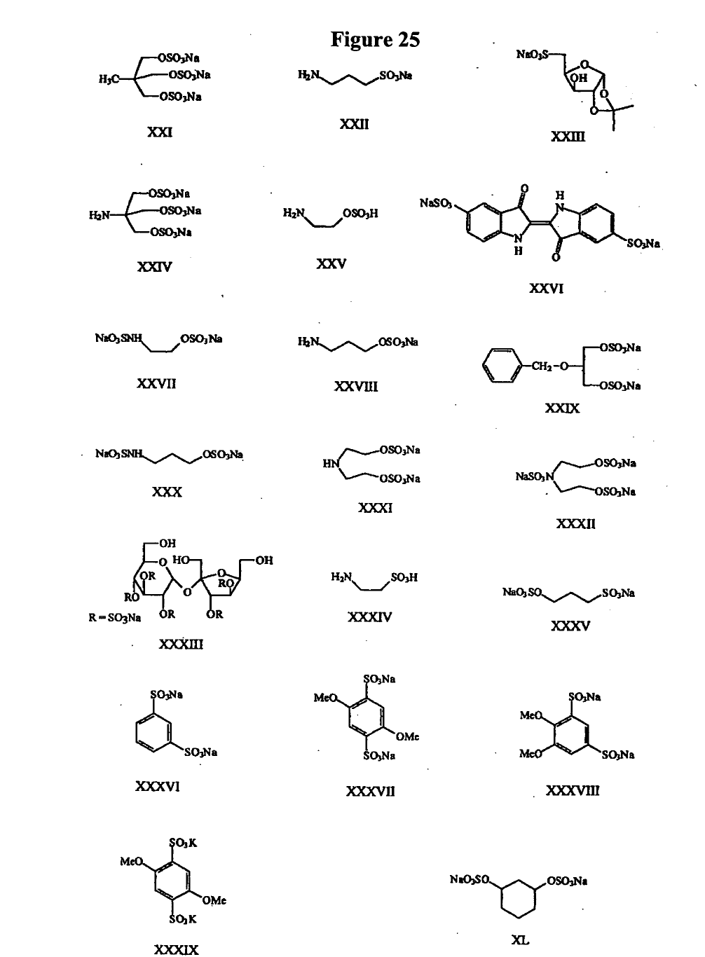

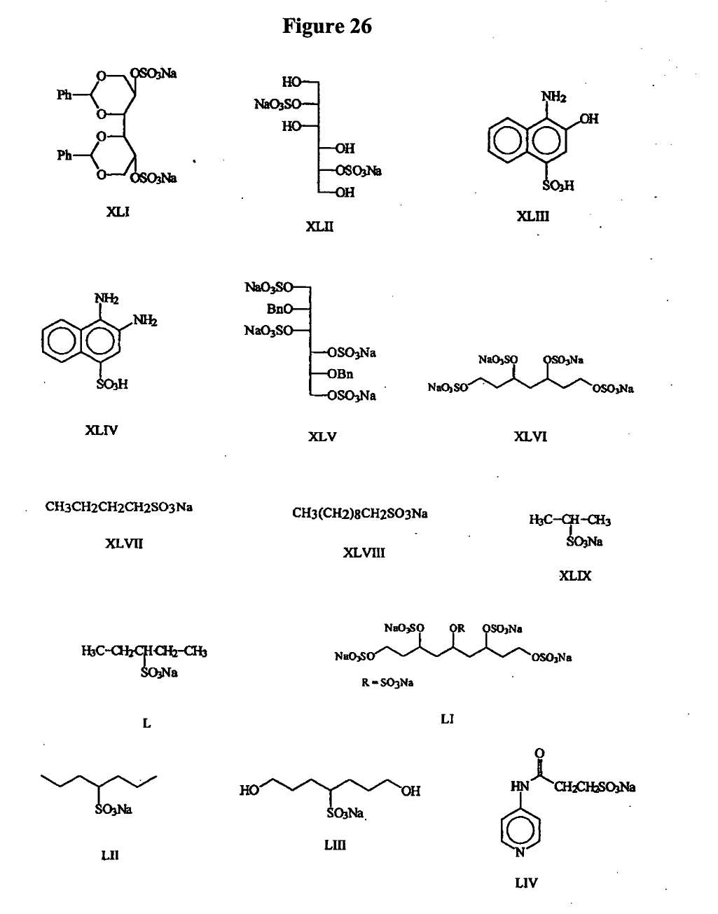

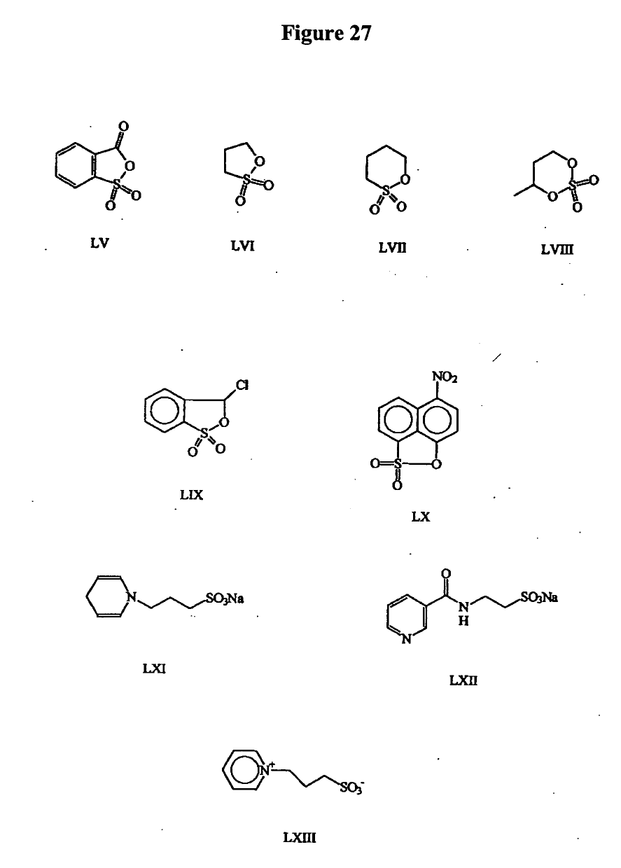

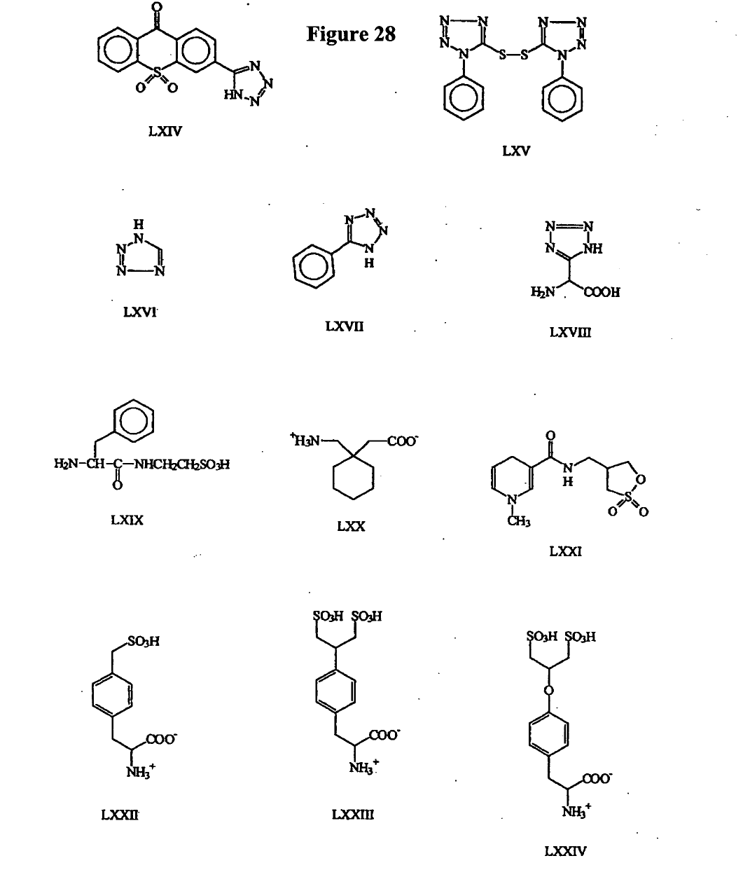

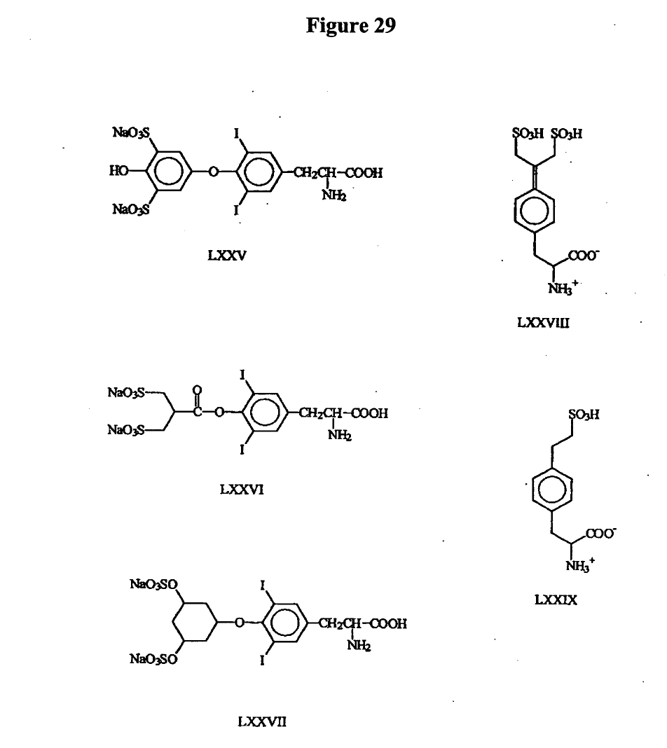

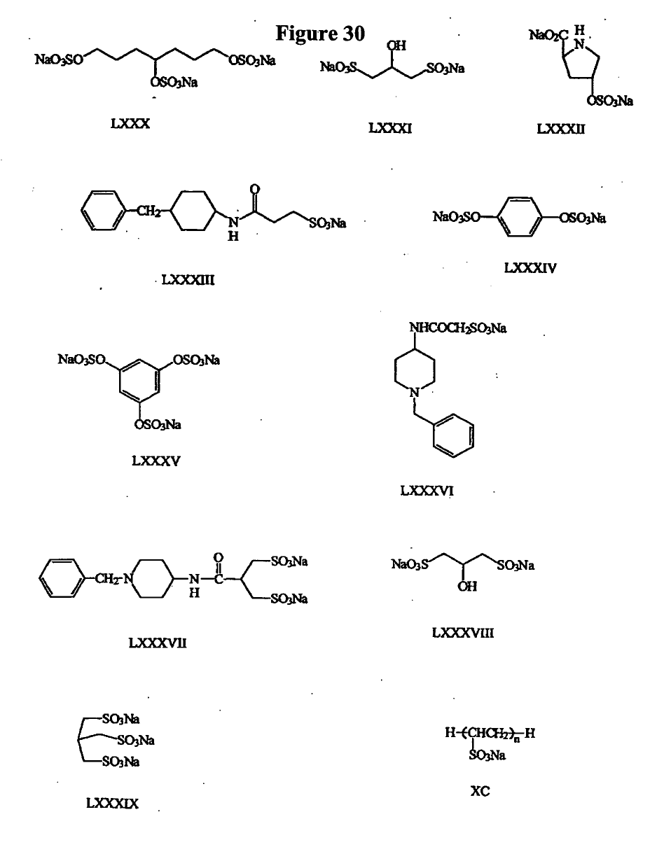

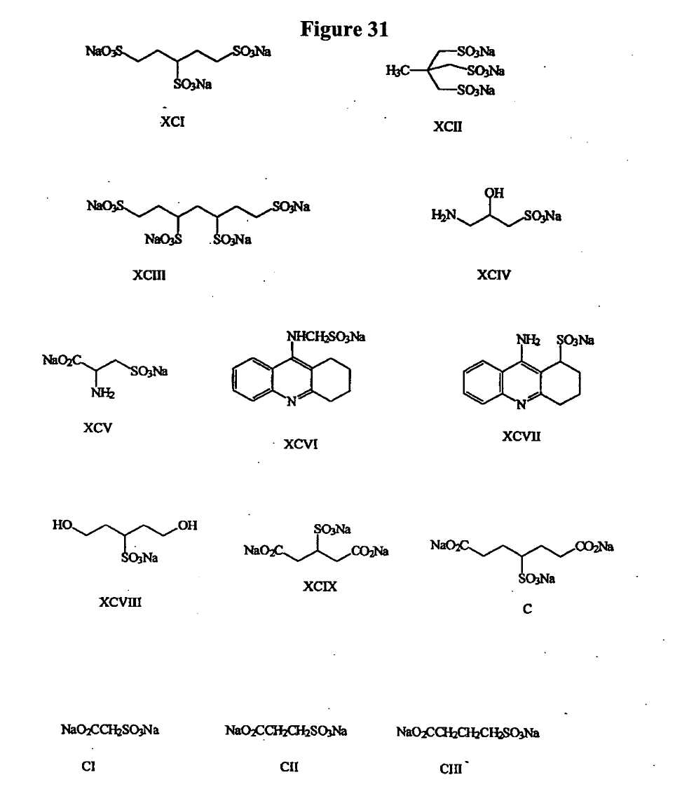

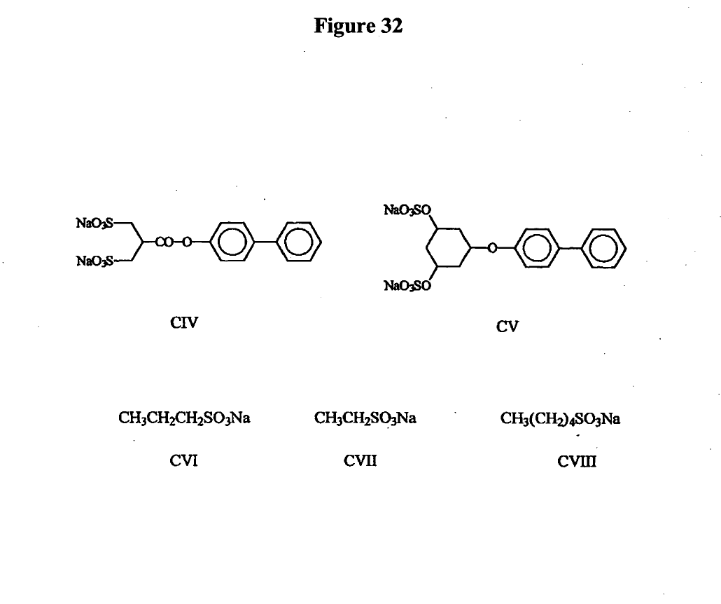

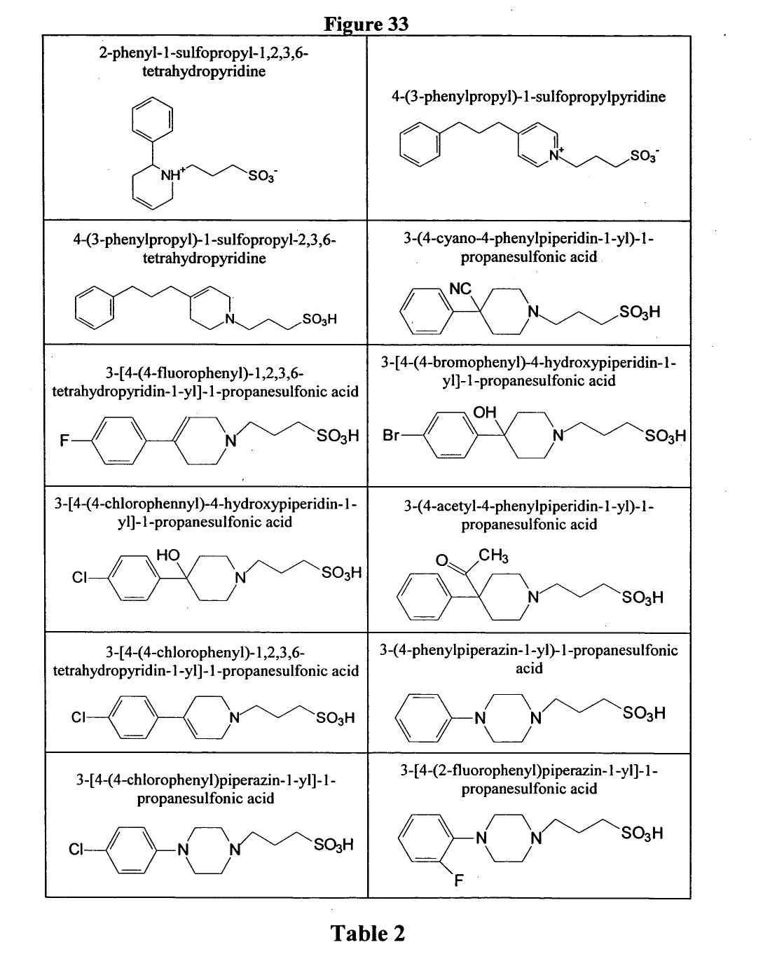

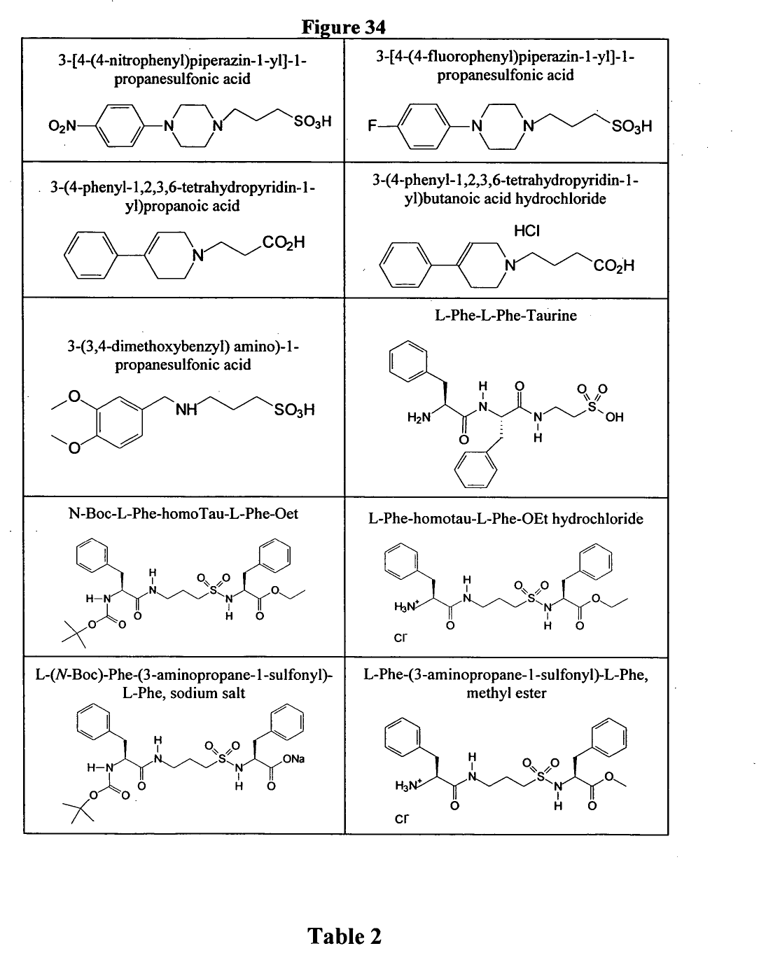

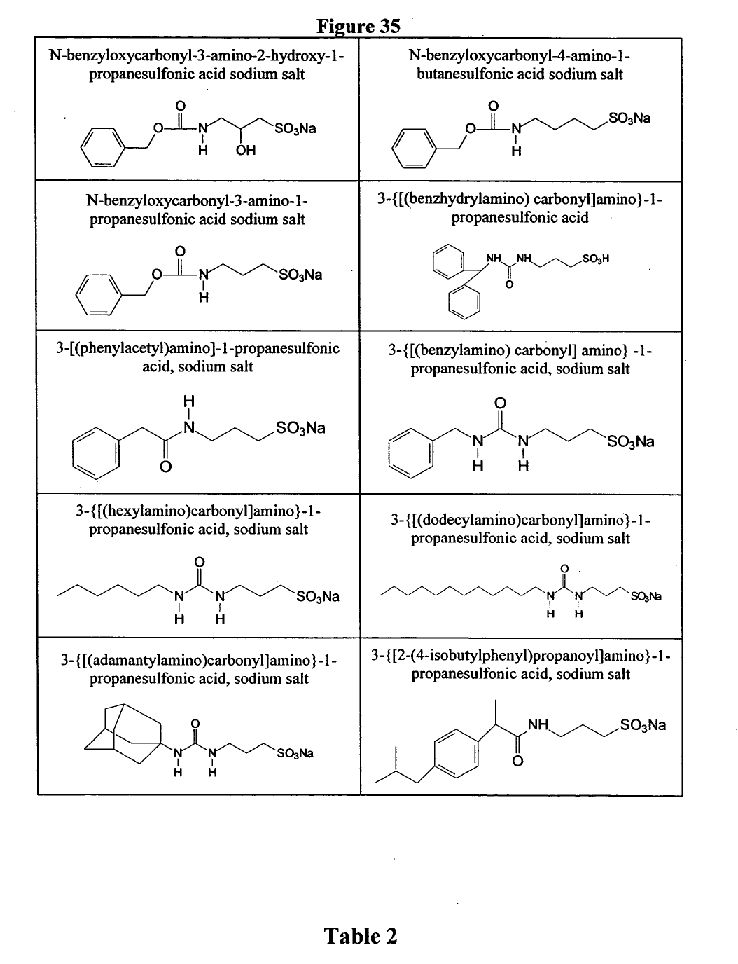

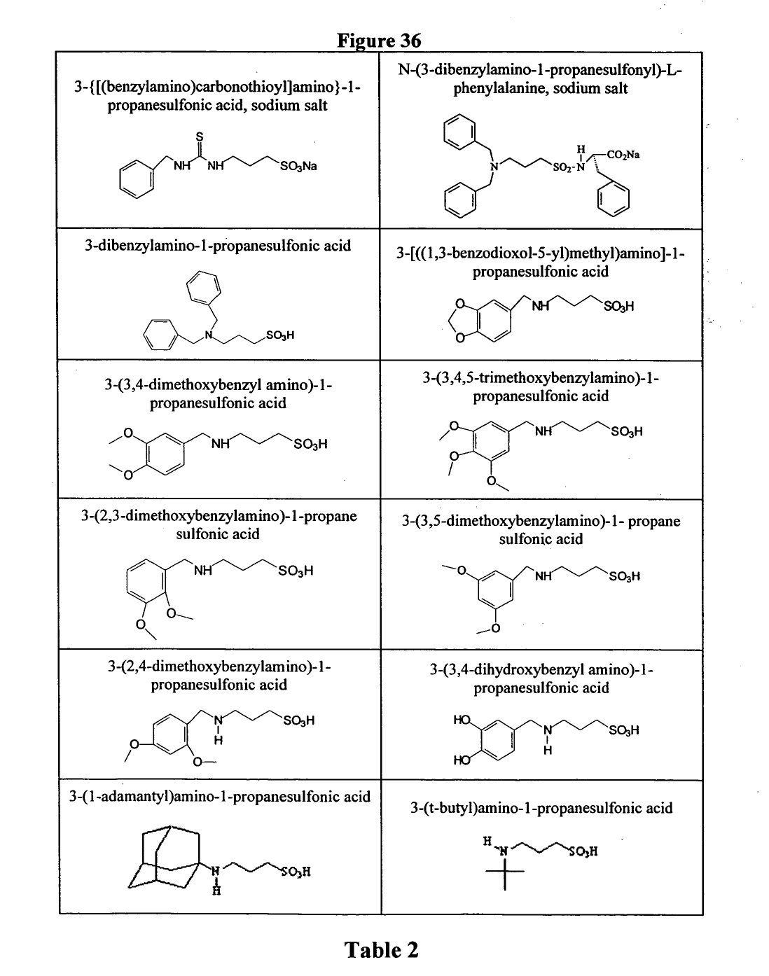

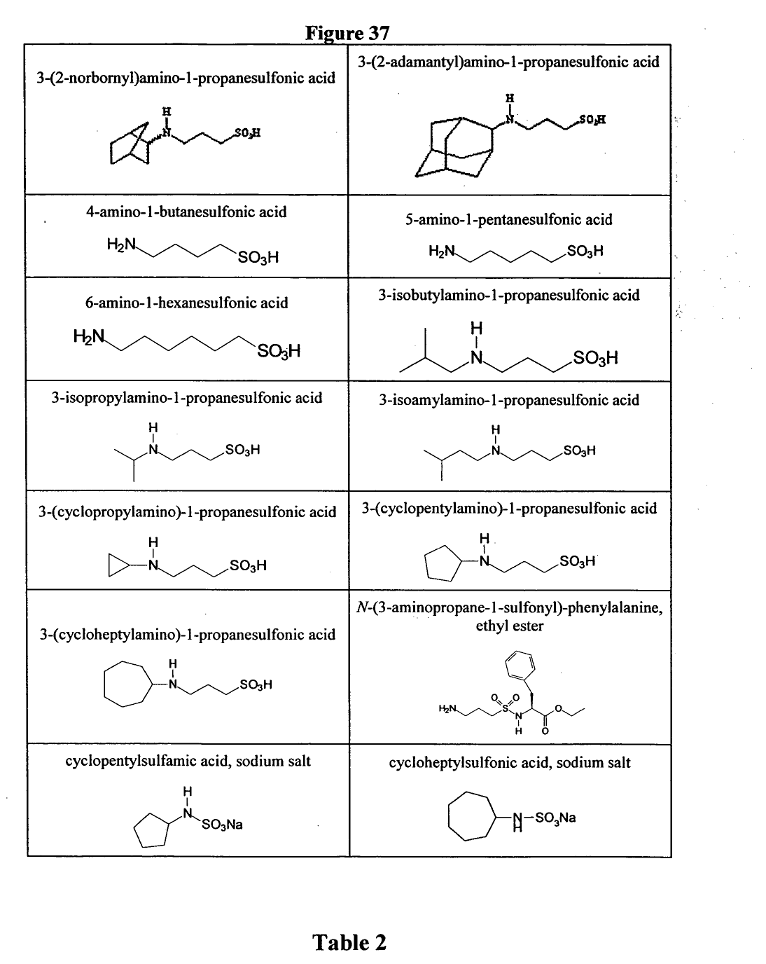

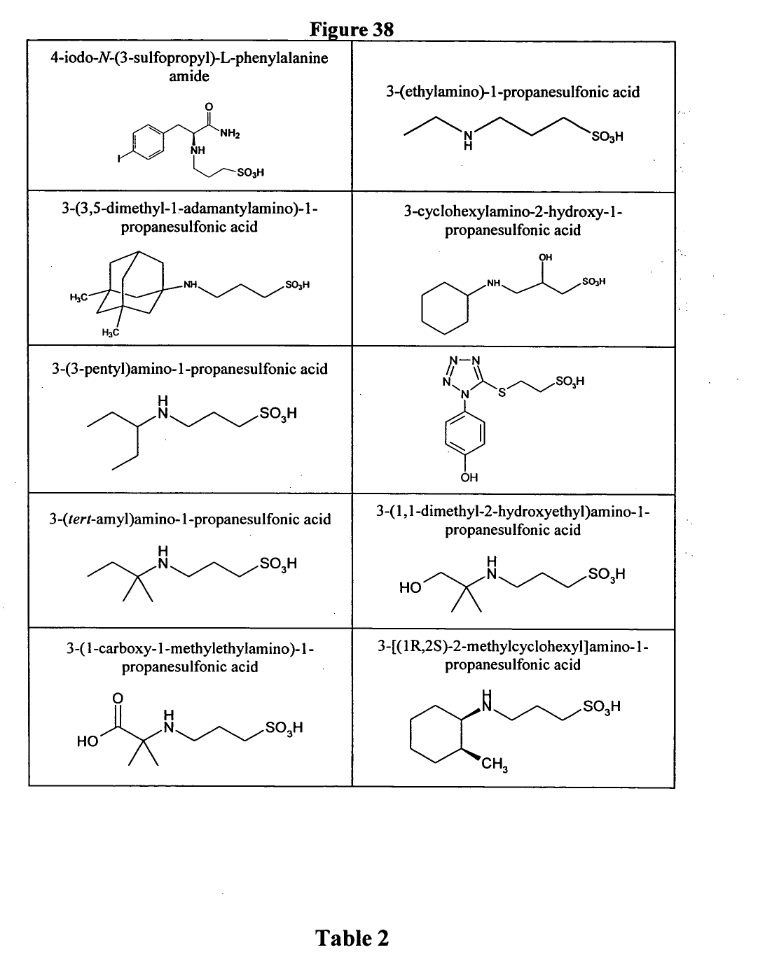

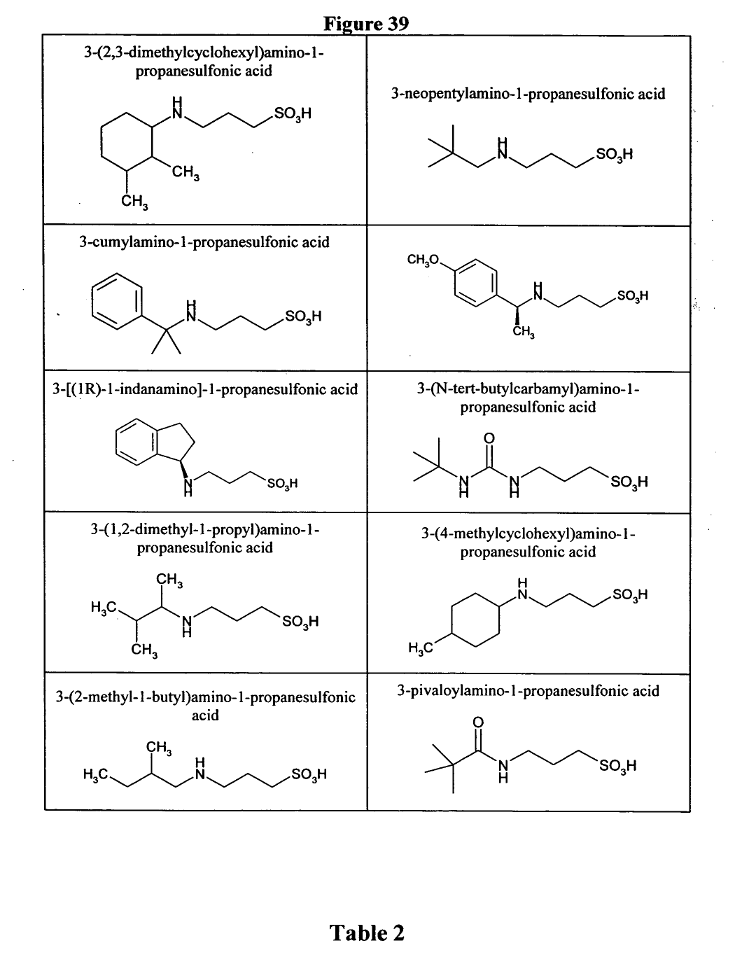

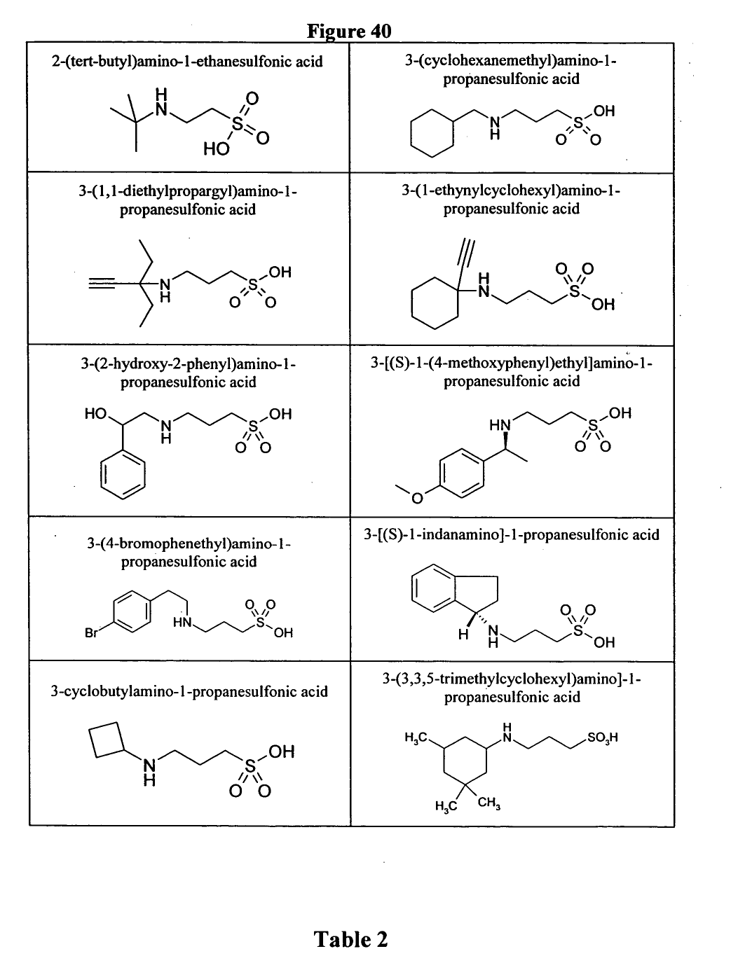

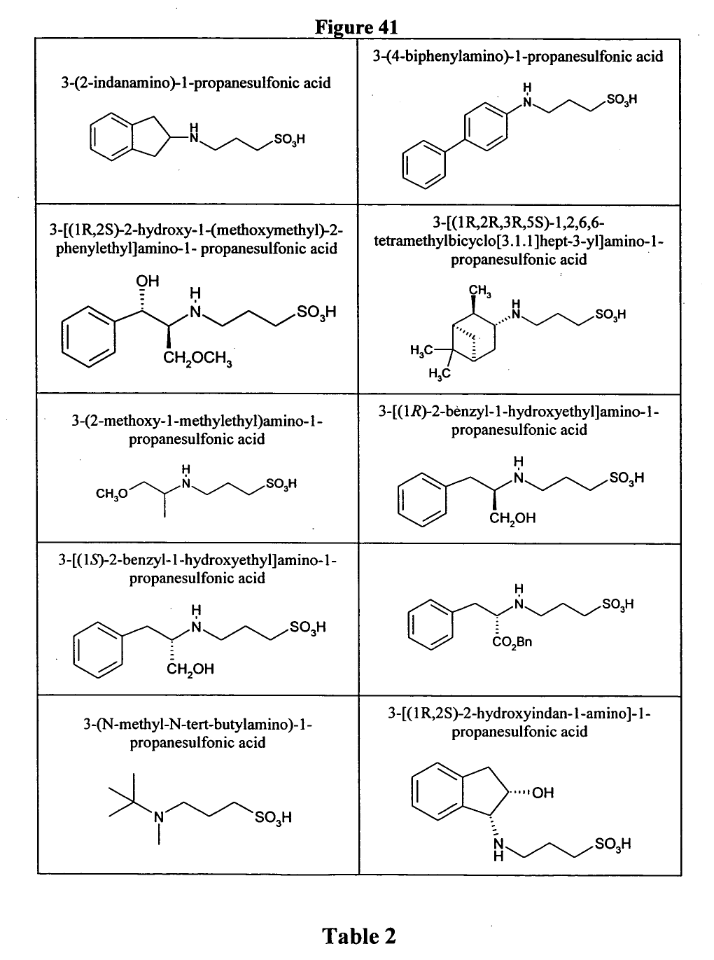

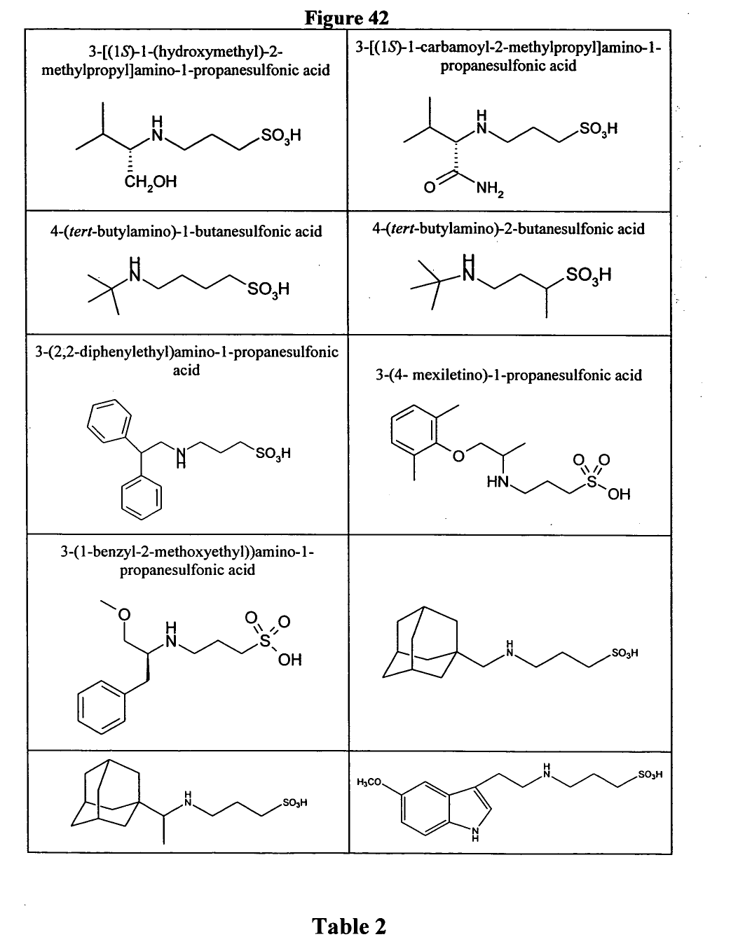

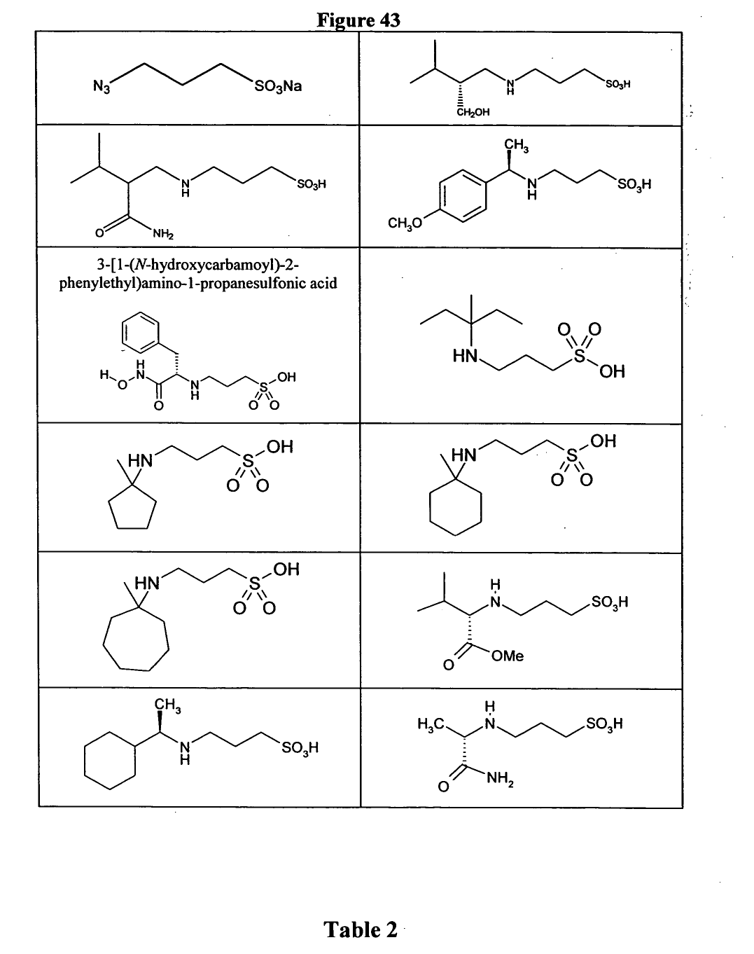

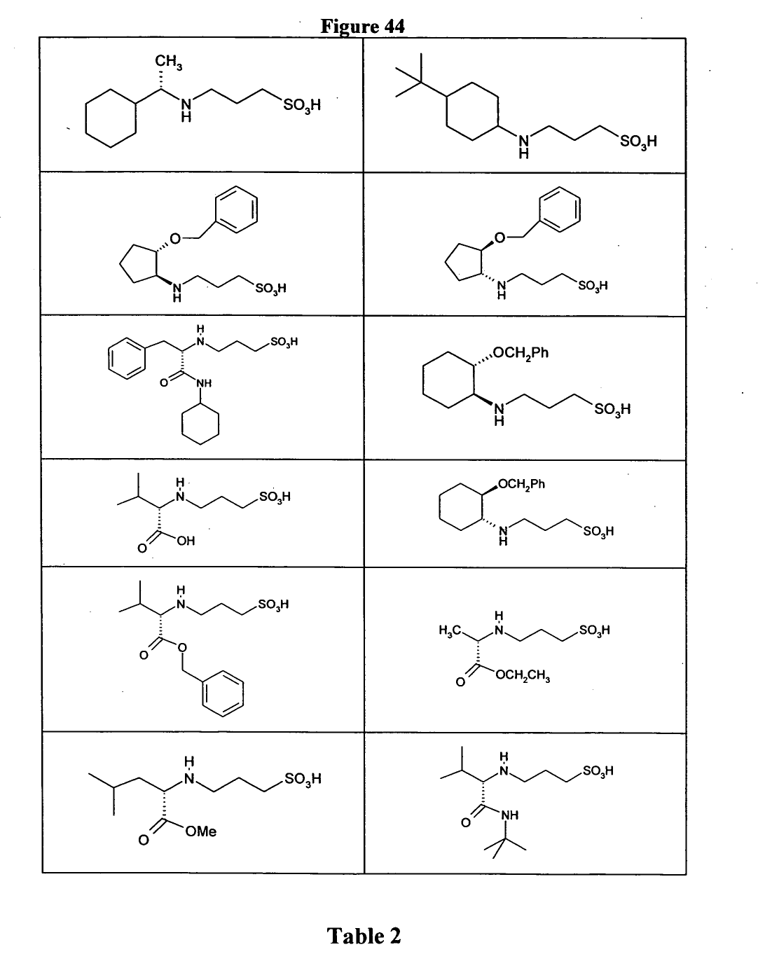

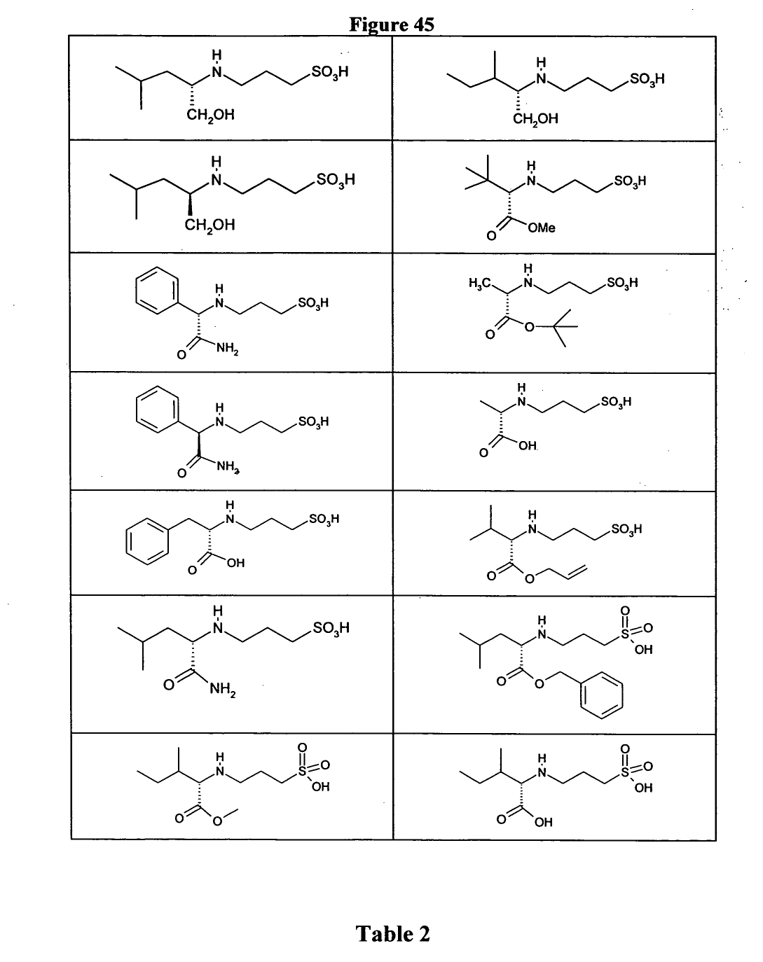

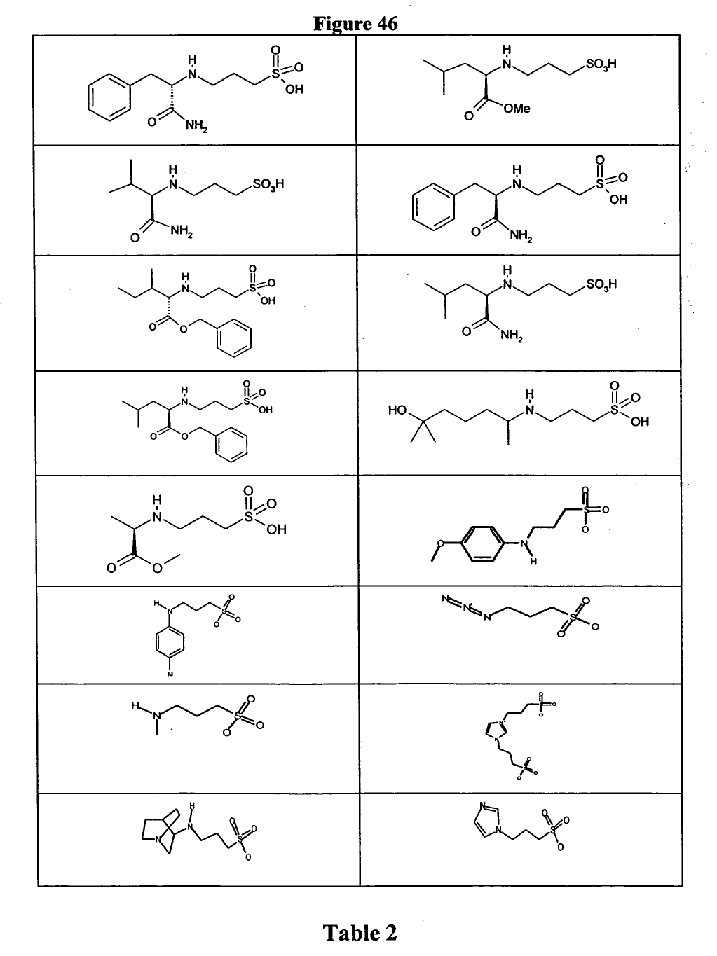

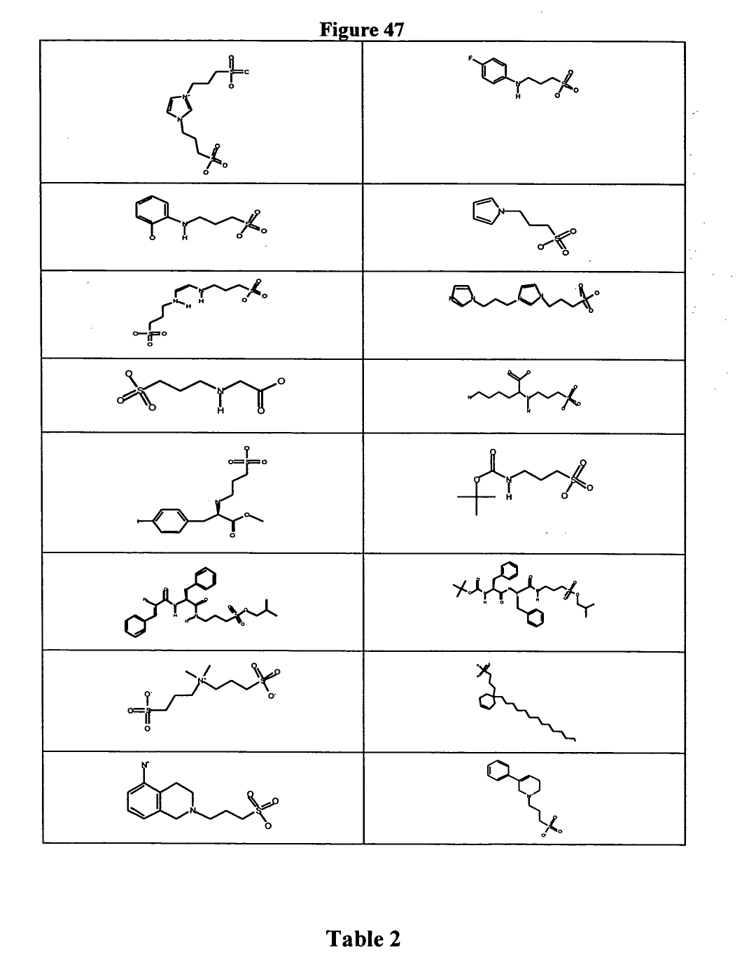

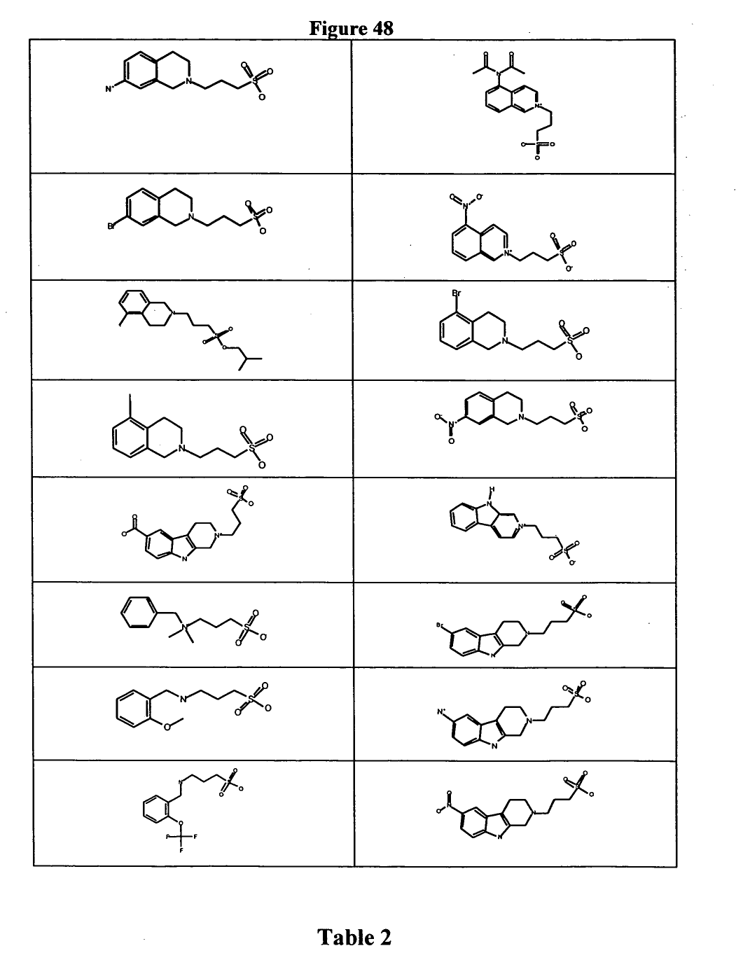

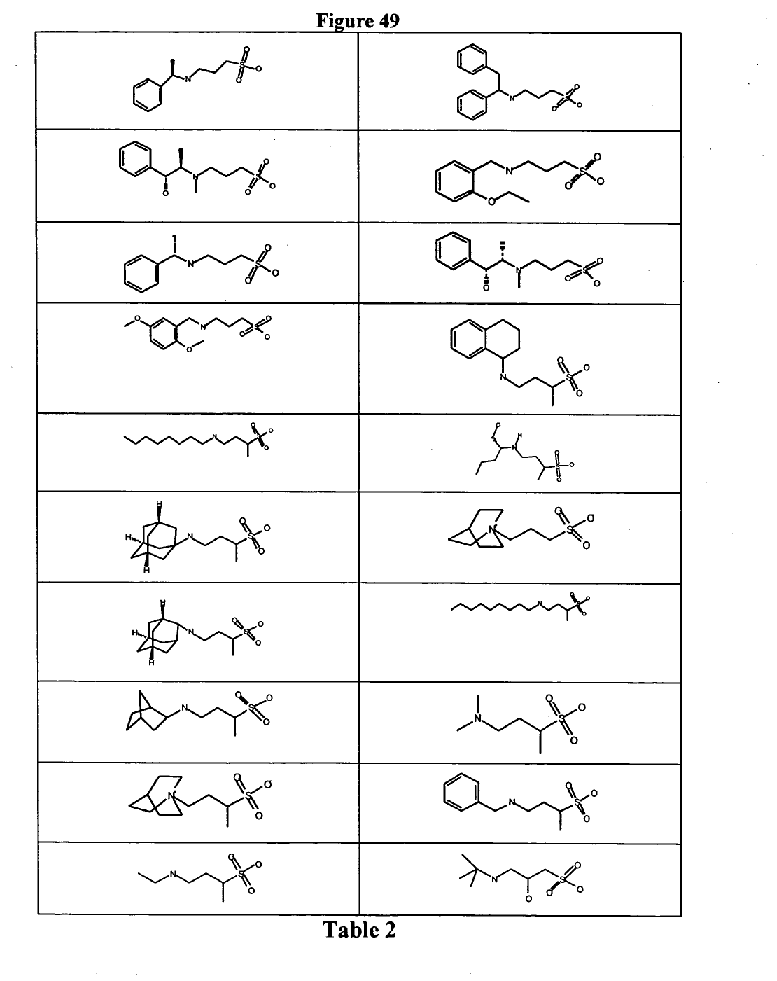

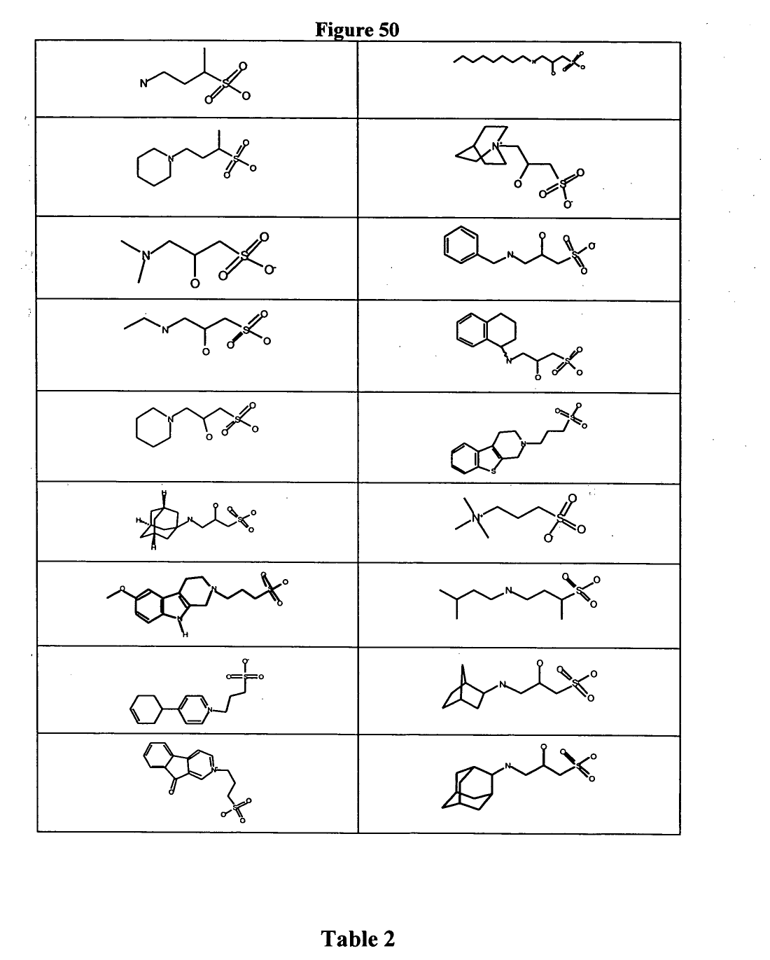

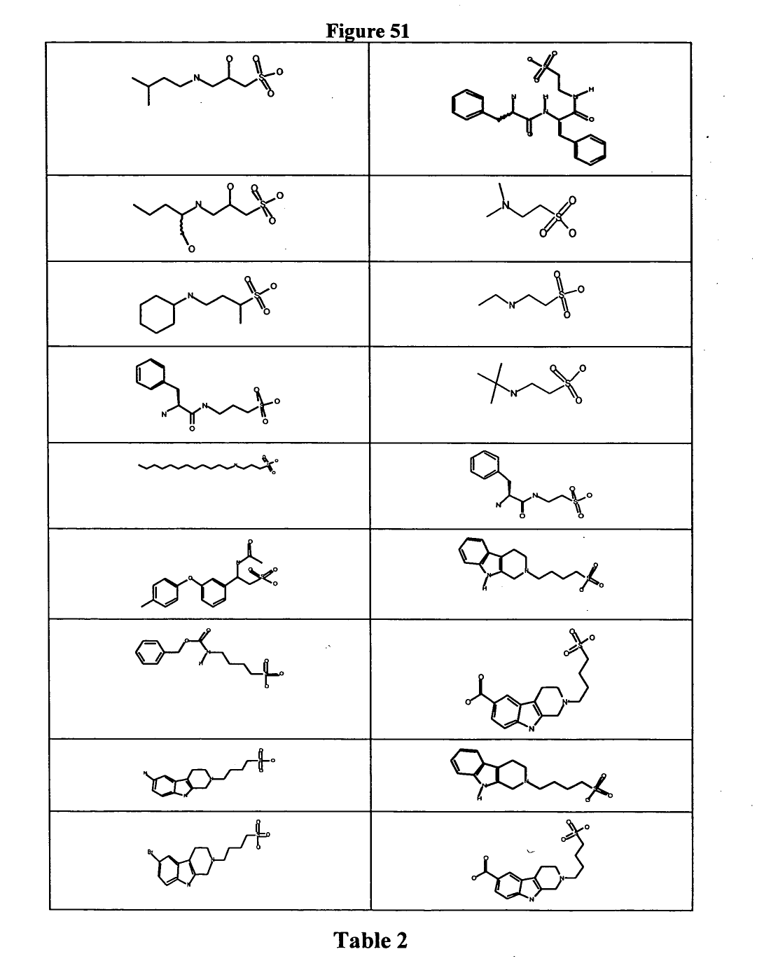

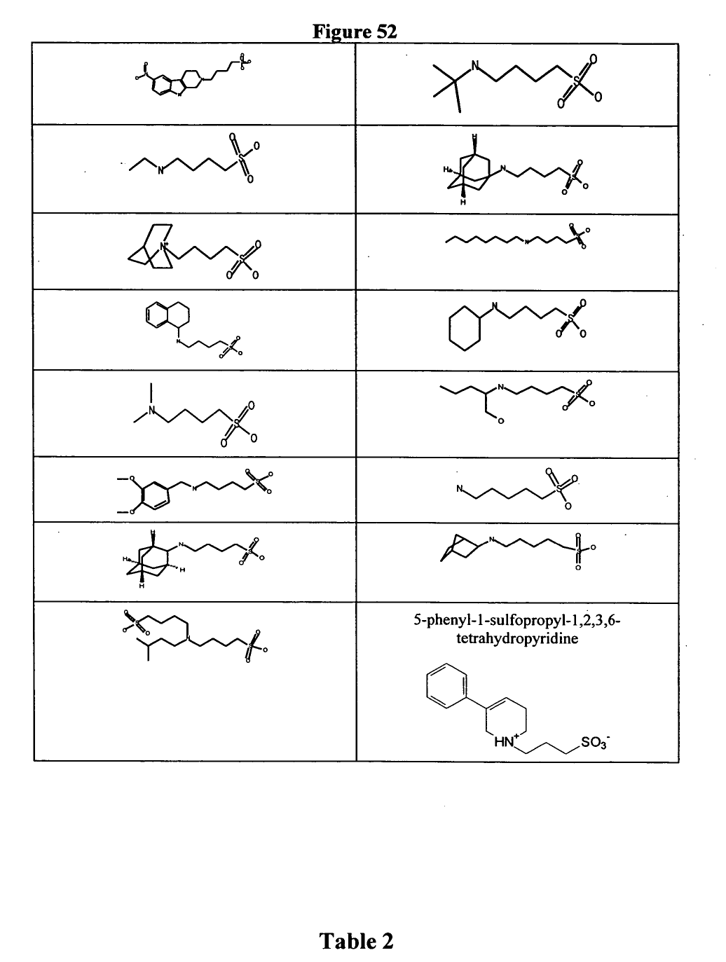

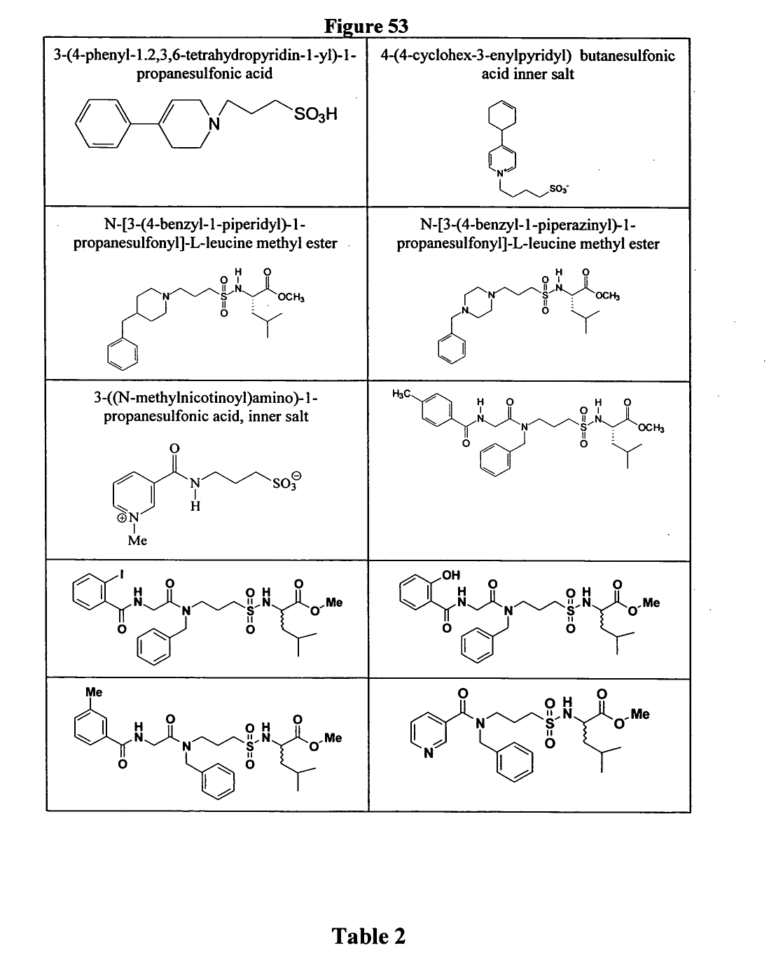

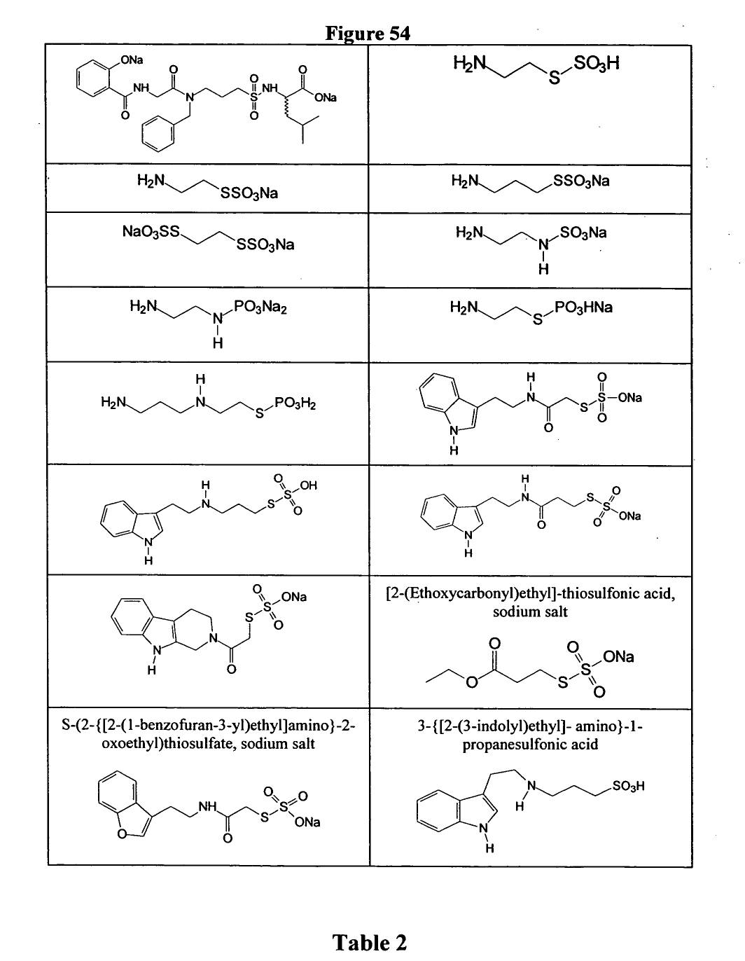

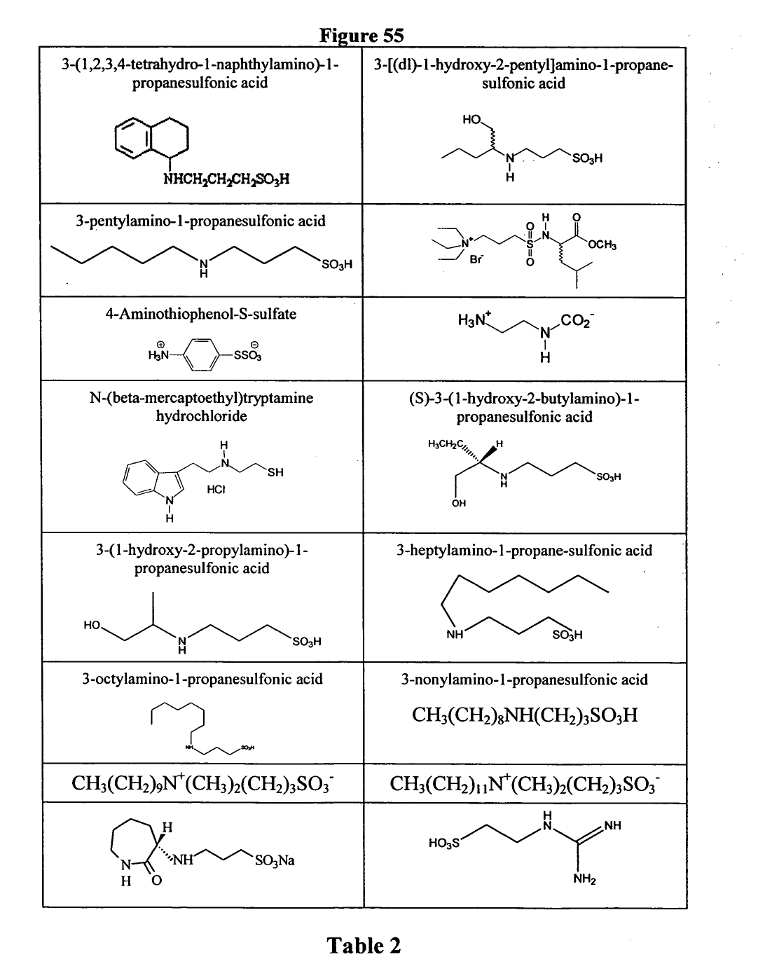

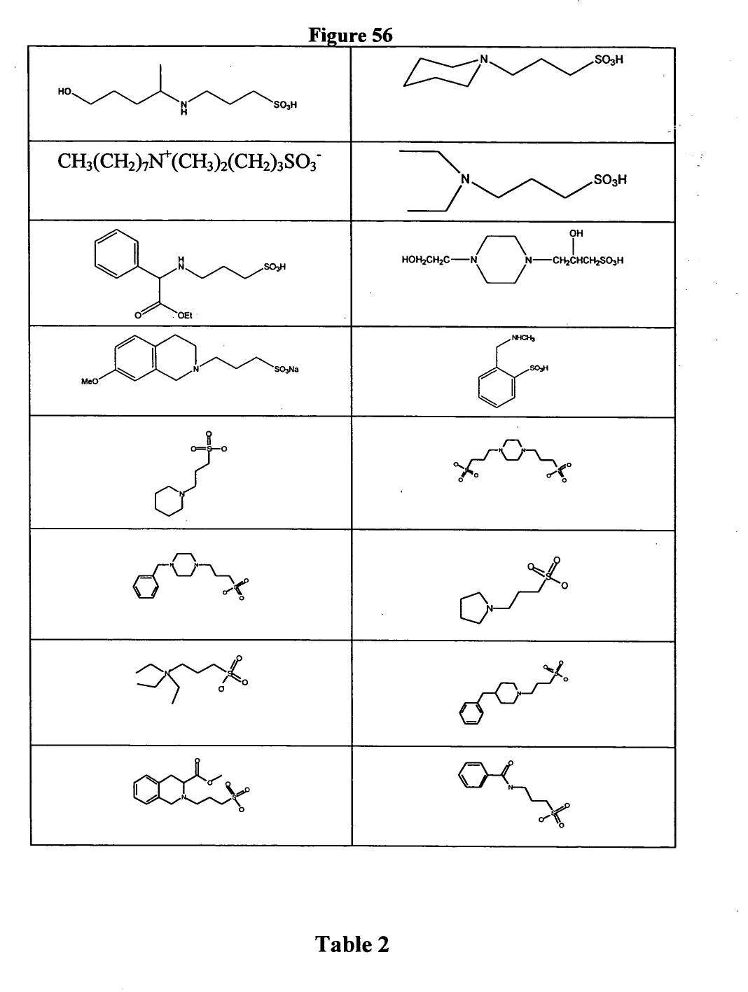

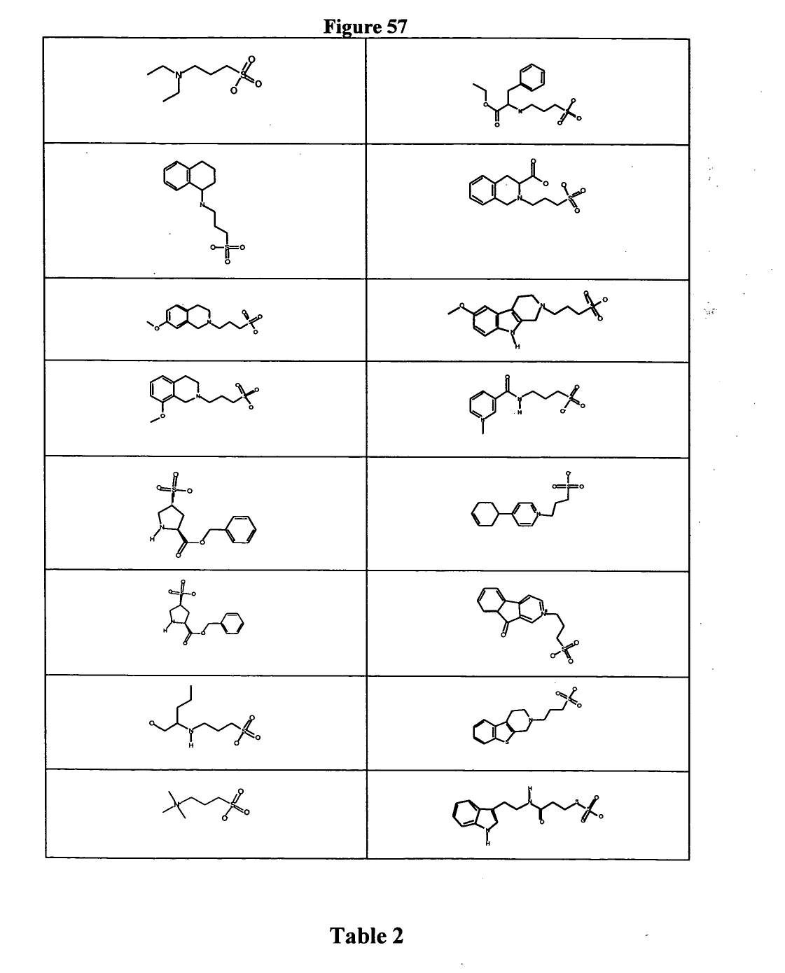

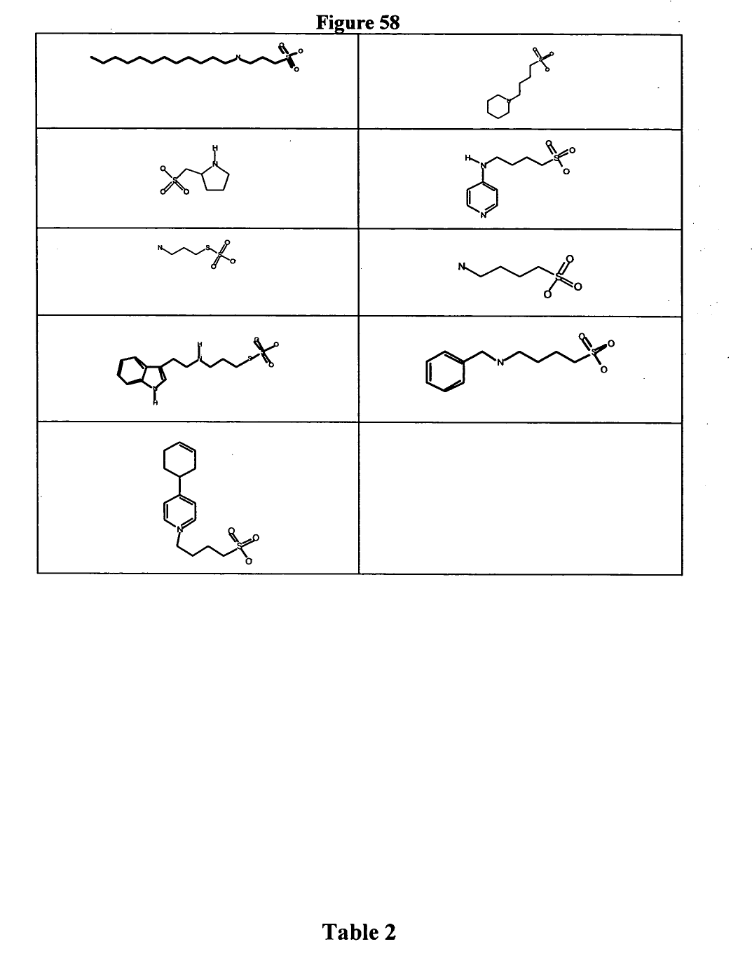

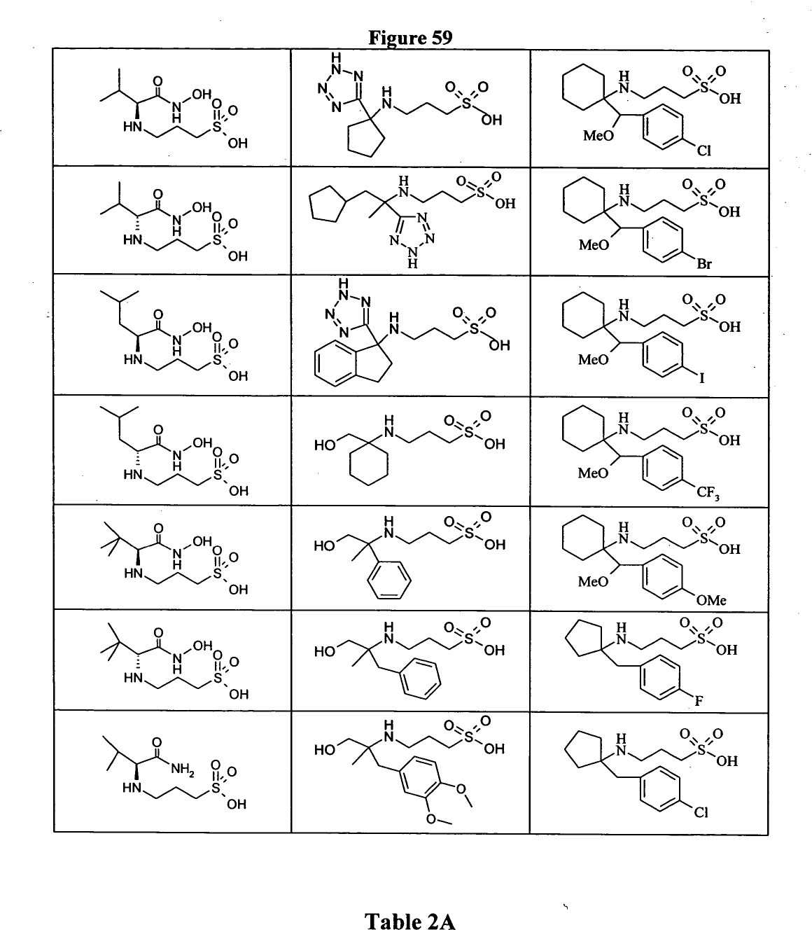

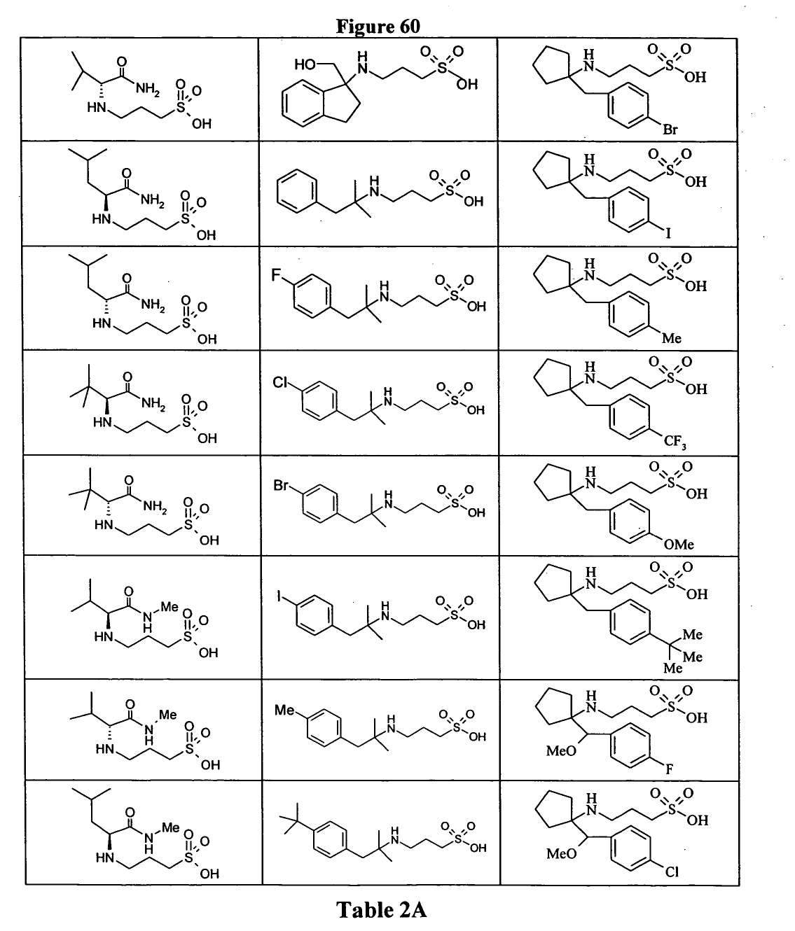

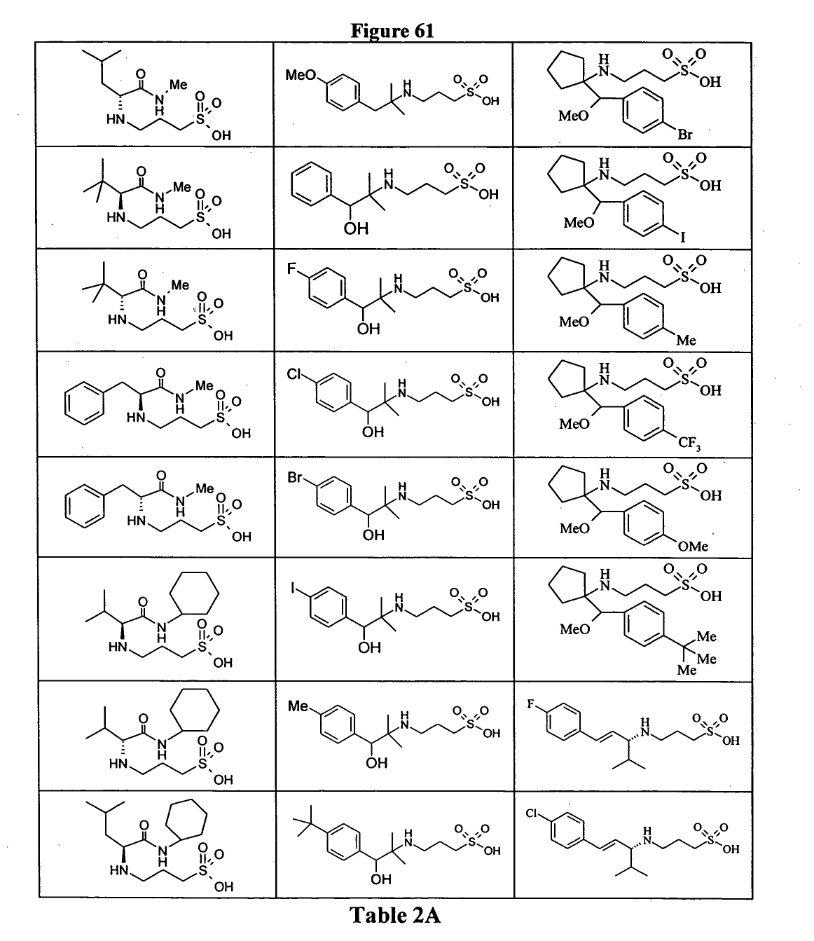

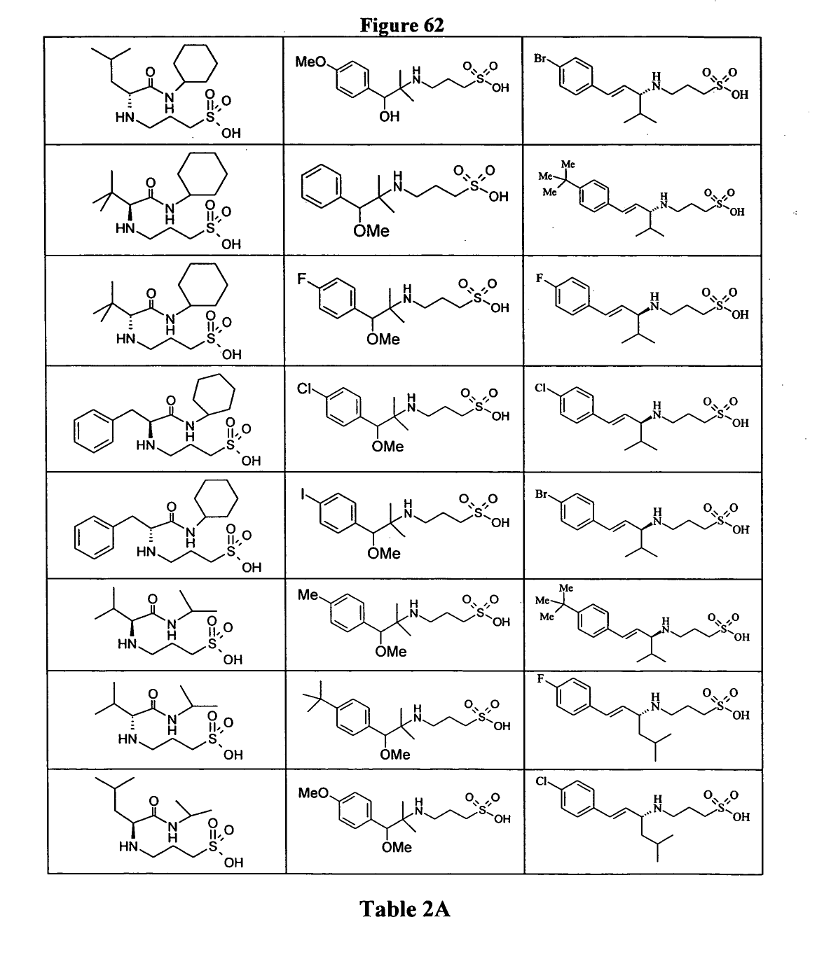

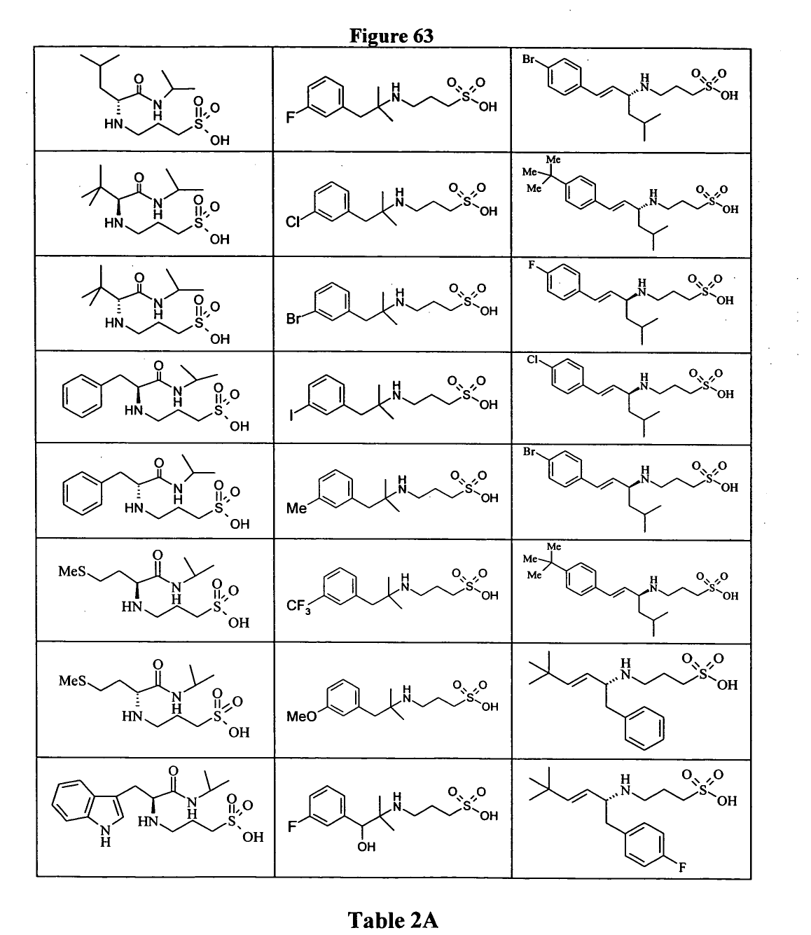

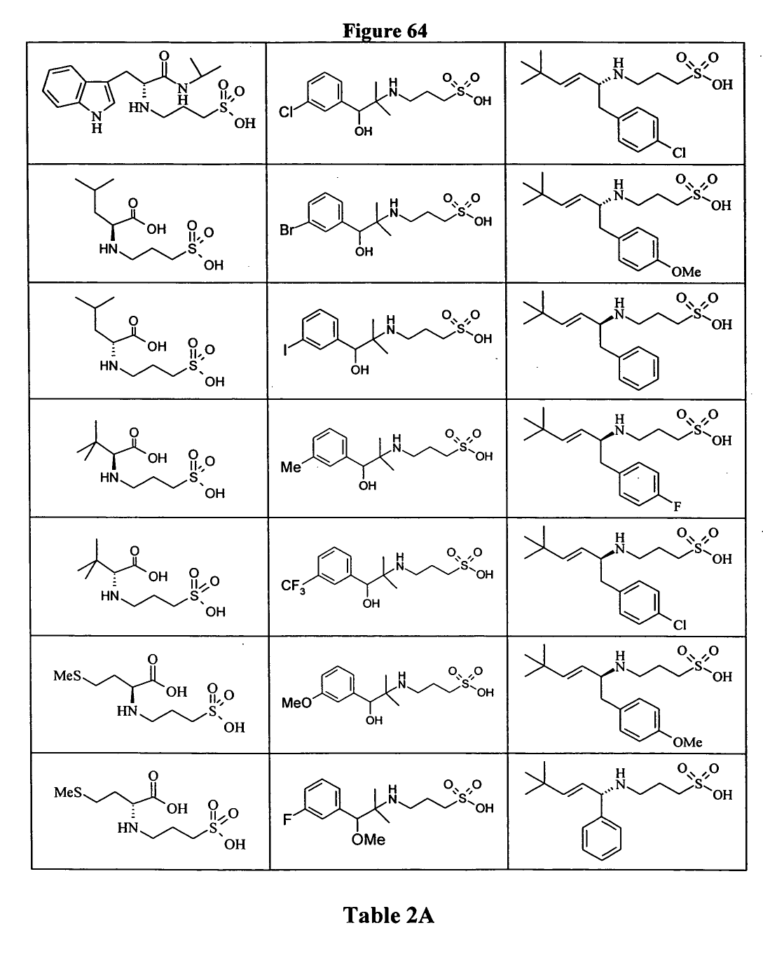

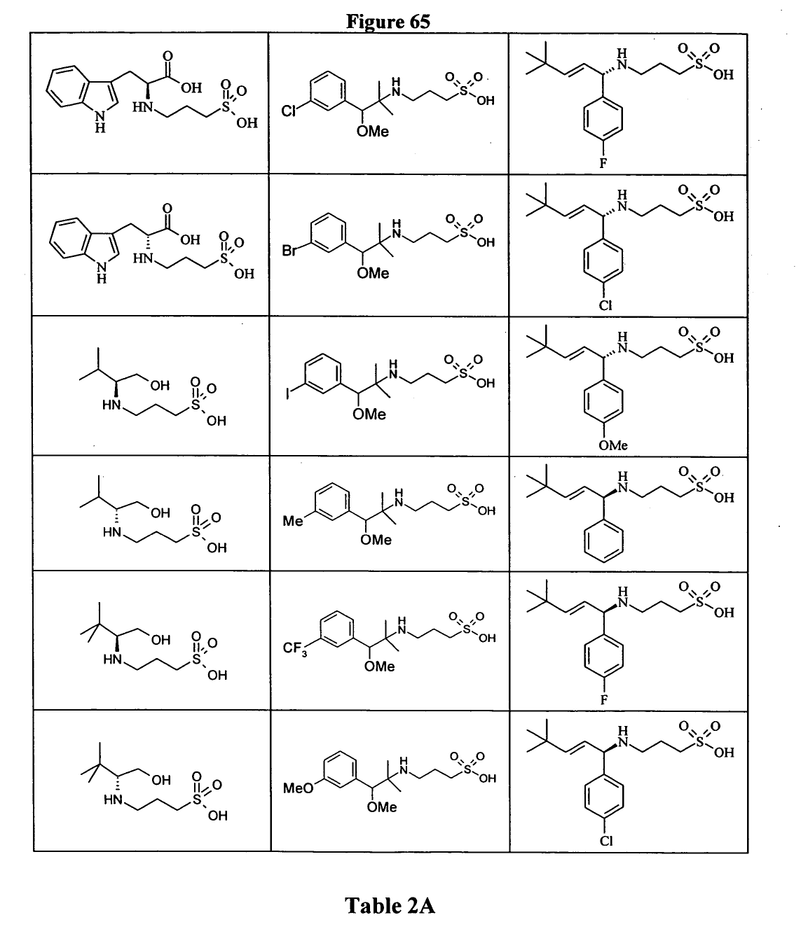

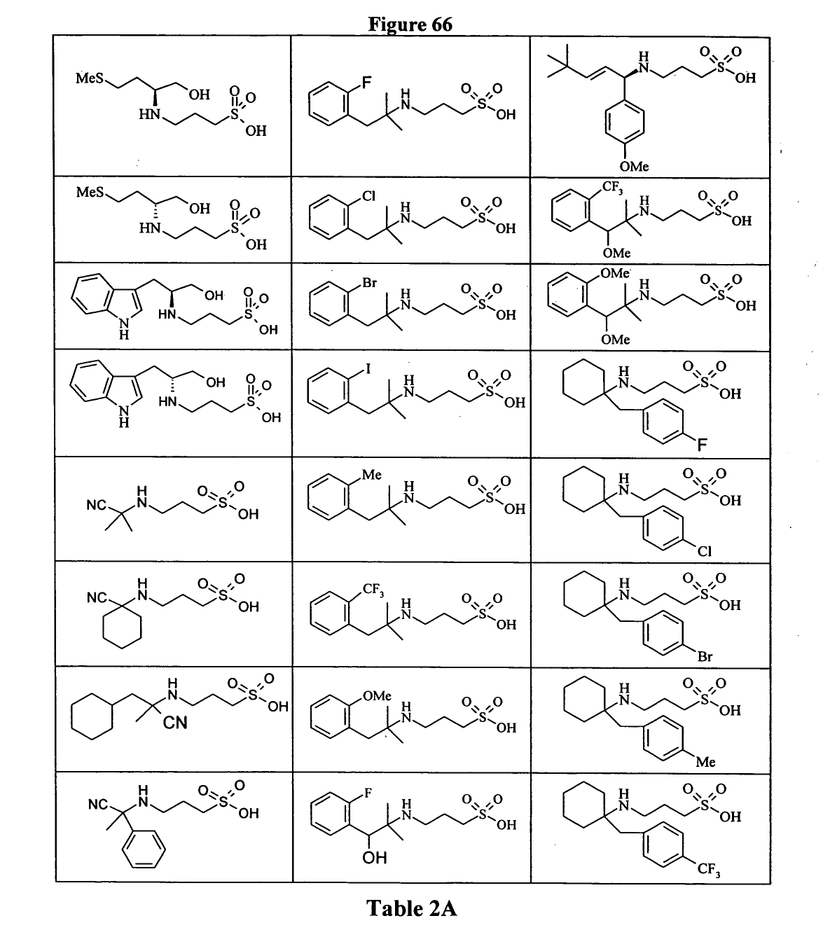

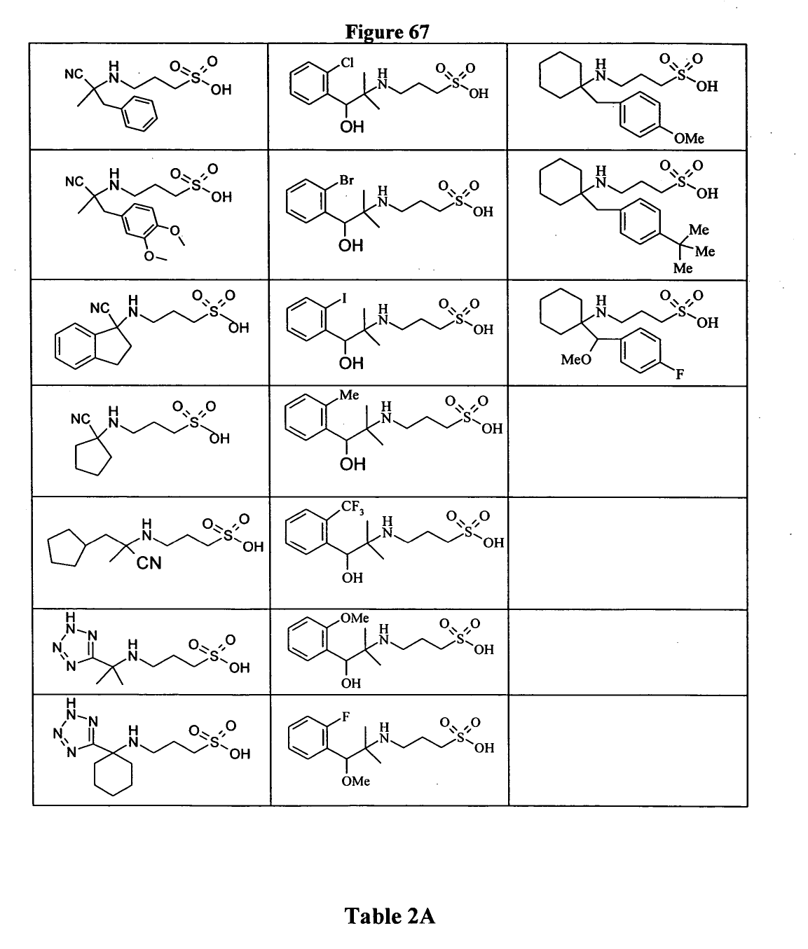

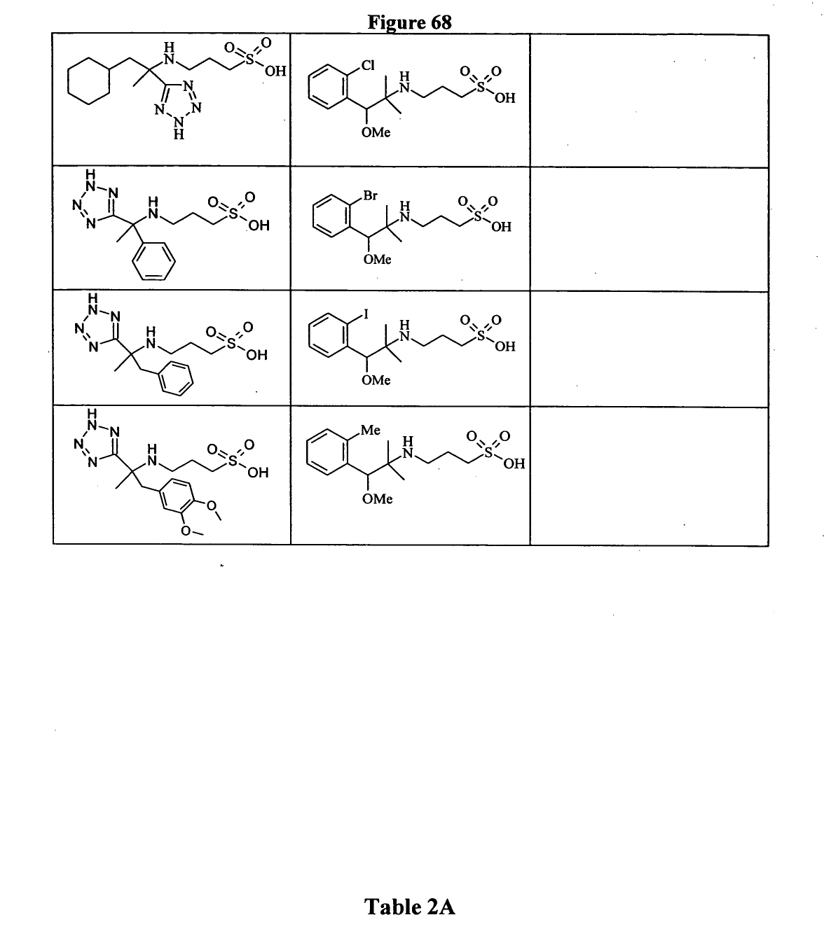

### DETAILED DESCRIPTION OF THE INVENTION

“Amyloidosis” or “amyloid disease” refers to a pathological condition characterized by the presence of amyloid fibers. Amyloid is a generic term referring to a group of diverse but specific protein deposits (intracellular or extracellular) which are seen in a number of different diseases. Though diverse in their occurrence, all amyloid deposits have common morphologic properties, stain with specific dyes (e.g., Congo red), and have a characteristic red-green birefringent appearance in polarized light after staining. They also share common ultrastructural features and common X-ray diffraction and infrared spectra. The term “amyloid-β diseases” includes those diseases, conditions, pathologies, and other abnormalities of the structure or function of the brain, including components thereof, in which the causative agent is amyloid. The area of the brain affected in an amyloid-β disease may be the stroma including the vasculature or the parenchyma including functional or anatomical regions, or neurons themselves. A subject need not have received a definitive diagnosis of a specifically recognized amyloid-β disease.

Local deposition of amyloid is common in the brain, particularly in elderly individuals. The most frequent type of amyloid in the brain is composed primarily of Aβ peptide fibrils, resulting in dementia associated with sporadic (non-hereditary) Alzheimer's disease. In fact, the incidence of sporadic Alzheimer's disease greatly exceeds forms shown to be hereditary. Nevertheless, fibril peptides forming plaques are very similar in both types.

APP is expressed and constitutively catabolized in most cells. The dominant catabolic pathway appears to be cleavage of APP within the Aβ sequence by an enzyme provisionally termed α-secretase, leading to release of a soluble ectodomain fragment known as APPsα. In contrast to this non-amyloidogenic pathway, APP can also be cleaved by enzymes known as and γ-secretase at the N- and C-termini of the Aβ, respectively, followed by release of Aβ into the extracellular space. To date, BACE has been identified as β-secretase (Vasser, et al., Science 286:735-741, 1999) and presenilins have been implicated in γ-secretase activity (De Strooper, et al, Nature 391, 387-90 (1998)).

The 39-43 amino acid Aβ peptide is produced by sequential proteolytic cleavage of the amyloid precursor protein (APP) by the β and γ secretase enzymes. Although Aβ40 is the predominant form produced, 5-7% of total Aβ exists as Aβ42 (Cappai et al, Int. J. Biochem. Cell Biol. 31. 885-89 (1999)). The length of the Aβ peptide appears to dramatically alter its biochemical/biophysical properties. Specifically, the additional two amino acids at the C-terminus of Aβ42 are very hydrophobic, presumably increasing the propensity of Aβ42 to aggregate. For example, Jarrett, et al demonstrated that Aβ42 aggregates very rapidly in vitro compared to Aβ40, suggesting that the longer form of Aβmay be the important pathological proteins that are involved in the initial seeding of the neuritic plaques in Alzheimer's disease (Jarrett, et al, Biochemistry 32, 4693-97 (1993); Jarrett, et al, Ann. N.Y. Acad. Sci. 695, 144-48 (1993)).

This hypothesis has been further substantiated by the recent analysis of the contributions of specific forms of Aβin cases of genetic familial forms of Alzheimer'is disease (“FAD”). For example, the “London” mutant form of APP (APPV717I) linked to FAD selectively increases the production of Aβ42/43 forms versus Aβ 40 (Suzuki, et al, Science 264, 1336-40 (1994)) while the “Swedish” mutant form of APP (APPK670N/M671L) increases levels of both Aβ40 and Aβ42/43 (Citron, et al, Nature 360, 672-674 (1992); Cai, et al., Science 259, 514-16, (1993)). Also, it has been observed that FAD-linked mutations in the Presenilin-1 (“PS1”) or Presenilin-2 (“PS2”) genes will lead to a selective increase in Aβ42/43 production but not Aβ40 (Borchelt, et al., Neuron 17, 1005-13 (1996)). This finding was corroborated in transgenic mouse models expressing PS mutants that demonstrate a selective increase in brain Aβ42 (Borchelt, op cit.; Duff, et al., Neurodegeneration 5(4), 293-98 (1996)). Thus the leading hypothesis regarding the etiology of Alzheimer's disease is that an increase in Aβ42 production or release is a causative event in the disease pathology.

Epidemiological studies show that subjects with elevated cholesterol levels have an increased risk of Alzheimer's disease (Notkola, et al., Neuroepidemiology 17(1), 14-20 (1998); Jarvik, et al., Neurology 45(6), 1092-96 (1995)). In addition to the data which suggests that elevated levels of Aβ are associated with Alzheimer's disease, other environmental and genetic risk factors have been identified. For example, a relationship exists between serum cholesterol levels and the incidence and the pathophysiology of Alzheimer's disease. The best studied of these is polymorphism of the apolipoprotein E (“ApoE”) gene: subjects homozygous for the ε4 isoform of ApoE (apoE4) have consistently been shown to have an increased risk for Alzheimer's disease (Strittmatter, et al., Proc. Nat'l Acad. Sci. USA 90:1977-81 (1993). Because ApoE is a cholesterol transport protein, several groups have observed a correlation between the risk of developing Alzheimer's disease and circulating levels of cholesterol (Mahley, Science 240, 622-30 (1998); Saunders, et al., Neurology 43, 1467-72 (1993); Corder, et al., Science 261, 921-23 (1993); Jarvik, et al., Ann. N.Y. Acad. Sci. 826, 128-46 (1997)). Moreover, cholesterol loading increases the production of Aβprotein (Simons, et al., Proc. Nat'l Acad. Sci. USA 95, 6460-64 (1998)), while pharmacological reduction of cholesterol with the HMG CoA reductase inhibitor simvastatin decreases levels of both Aβ40 and Aβ-42 (Fassbender, et al., Proc. Nat'l Acad. Sci USA 98, 5856-61 (2001)) in vitro. Consistent with these data are the results of epidemiological studies which have shown that treatment with certain HMG CoA reductase inhibitors, commonly used to normalize cholesterol levels in humans, reduces the prevalence of Alzheimer's disease (Wolozin, et al., Arch. Neurol. 57, 1439-43 (2000); Jick, et al., Lancet 356, 1627-31 (2000). Taken together, these data suggest a link between regulation of cholesterol levels and Alzheimer's disease. In addition, a relationship with coronary disease has been demonstrated (discussed further below).

Amyloid-β peptide (Aβ) is a 39-43 amino acid peptide derived by proteolysis from a large protein known as Beta Amyloid Precursor Protein (“βAPP”). Mutations in βAPP result in familial forms of Alzheimer's disease, Down's syndrome, cerebral amyloid angiopathy, and senile dementia, characterized by cerebral deposition of plaques composed of Aβ fibrils and other components, which are described in further detail below. Known mutations in APP associated with Alzheimer's disease occur proximate to the cleavage sites of β or γ-secretase, or within Aβ. For example, position 717 is proximate to the site of gamma-secretase cleavage of APP in its processing to Aβ, and positions 670/671 are proximate to the site of β-secretase cleavage. Mutations at any of these residues may result in Alzheimer's disease, presumably by causing an increase in the amount of the 42/43 amino acid form of Aβ generated from APP. The familial form of Alzheimer's disease represents only 10% of the subject population. Most occurrences of Alzheimer's disease are sporadic cases where APP and Aβ do not possess any mutation.

The structure and sequence of Aβ peptides of various lengths are well known in the art. Such peptides can be made according to methods known in the art, or extracted from the brain according to known methods (e.g., Glenner and Wong, Biochem. Biophys. Res. Comm. 129, 885-90 (1984); Glenner and Wong, Biochem. Biophys. Res. Comm. 122, 1131-35 (1984)). In addition, various forms of the peptides are commercially available.

As used herein, the terms “β amyloid,” “amyloid-β,” and the like refer to amyloid β proteins or peptides, amyloid β precursor proteins or peptides, intermediates, and modifications and fragments thereof, unless otherwise specifically indicated. In particular, “Aβ” refers to any peptide produced by proteolytic processing of the APP gene product, especially peptides which are associated with amyloid pathologies, including Aβ1-39, Aβ1-40, Aβ1-41, Aβ1-42, and Aβ1-43. For convenience of nomenclature, “Aβ1-42” may be referred to herein as “Aβ(1-42)” or simply as “Aβ42” or “Aβ₄₂” (and likewise for any other amyloid peptides discussed herein). As used herein, the terms “β amyloid,” “amyloid-β,” and “Aβ” are synonymous. Unless otherwise specified, the term “amyloid” refers to amyloidogenic proteins, peptides, or fragments thereof which can be soluble (e.g., monomeric or oligomeric) or insoluble (e.g., having fibrillary structure or in amyloid plaque). See, e.g., MP Lambert, et al., Proc. Nat'l Acad. Sci. USA 95, 6448-53 (1998).

According to certain aspects of the invention, amyloid-β is a peptide having 39-43 amino-acids, or amyloid-β is an amyloidogenic peptide produced from βAPP. The amyloid-β diseases that are the subject of the present invention include age-related cognitive decline, early Alzheimer's disease as seen in Mild Cognitive Impairment (“MCI”), vascular dementia, or Alzheimer's disease (“AD”), which may be sporadic (non-hereditary) Alzheimer's disease or familial (hereditary) Alzheimer's disease. The amyloid-β disease may also be cerebral amyloid angiopathy (“CAA”) or hereditary cerebral hemorrhage. The amyloid-β disease may be senile dementia, Down's syndrome, inclusion body myositis (“IBM”), or age-related macular degeneration (“ARMD”).

The present invention relates to the use of certain compounds, denoted a “first agent,” representative examples of which include substituted and unsubstituted alkanesulfonic acids, in combination with a second agent that is biologically active for the treatment or prevention of amyloid-β diseases, including Alzheimer's disease and cerebral amyloid angiopathy. The invention also relates to pharmaceutical compositions for the prevention or treatment of such diseases and methods of preparing and using these compositions.

The invention pertains to pharmaceutical compositions and methods of use thereof for the treatment of amyloid-β diseases. The pharmaceutical compositions comprise a first agent that treats or prevents an amyloid-β disease, e.g., by preventing or inhibiting amyloid-β fibril formation, neurodegeneration, or cellular toxicity. The pharmaceutical composition also comprises a second agent that is an active pharmaceutical ingredient; that is, the second agent is therapeutic and its function is beyond that of an inactive ingredient, such as a pharmaceutical carrier (or vehicle), preservative, diluent, or buffer. The second agent may be useful in treating or preventing an amyloid-βdisease or another neurological disease. The first and second agents may exert their biological effects by similar or unrelated mechanisms of action; or either one or both of the first and second agents may exert their biological effects by a multiplicity of mechanisms of action. A pharmaceutical composition may also comprise a third compound, or even more yet, wherein the third (and fourth, etc.) compound has the same characteristics of a second agent.

It should be understood that the pharmaceutical compositions described herein may have the first and second, third, or additional agents in the same pharmaceutically acceptable carrier or in a different pharmaceutically acceptable carrier for each described embodiment. It further should be understood that the first, second, third and additional agent may be administered simultaneously or sequentially within described embodiments. Alternatively, a first and second agent may be administered simultaneously, and a third or additional agent may be administered before or after the first two agents.

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, relates to methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

The combination of agents used within the methods and pharmaceutical compositions described herein may have a therapeutic additive or synergistic effect on the condition(s) or disease(s) targeted for treatment. The combination of agents used within the methods or pharmaceutical compositions described herein also may reduce a detrimental effect associated with at least one of the agents when administered alone or without the other agent(s) of the particular pharmaceutical composition. For example, the toxicity of side effects of one agent may be attenuated by another agent of the composition, thus allowing a higher dosage, improving patient compliance, and improving therapeutic outcome. Physicians may achieve the clinical benefits of previously recognized drugs while using lower dosage levels, thus minimizing adverse side effects. The additive or synergistic effects, benefits, and advantages of the compositions apply to classes of therapeutic agents, either structural or functional classes, or to individual compounds themselves.

The present methods and compositions relate to the treatment of amyloid-β diseases and conditions. As explained elsewhere herein, the various diseases and conditions involve several biological processes that produce the clinically recognized disease or condition. The inventors believe that targeting more than one of these biological processes simultaneously by the concomitant methods described herein enhances the therapeutic benefits of the individual agents. For example, potentiating the activity of the acetylcholine secreted by the remaining cholinergic neurons by administering cholinesterase inhibitors, while at the same time preventing further neuronal loss by enhancing clearance of Aβ from the brain, is clearly desirable compared to the use of only one individual treatment. Because the therapeutic targets outlined herein are independent, yet interconnected, it is desirable to act on more than one target at the same time. Two agents administered simultaneously and acting on different targets may act synergistically to modify or ameliorate disease progression or symptoms. Accordingly, one embodiment of the invention is concomitant therapy with a pharmaceutical composition described herein. In another embodiment, the combination of the first agent of the invention with a second (therapeutic) agent produces an enhanced therapeutic profile, for example, a profile that is greater than the sum of the benefits of the treatment with each agent independently.

In addition, Alzheimer's disease patients often suffer from secondary conditions such as depression, delusions and psychosis, or sleep disturbance. From the point of view of ease of manufacture, patient compliance, and ease of administration, it is advantageous to combine multiple medicines that the Alzheimer's patient self-administers into one combined medicament. Because of cognitive impairment, patient compliance among Alzheimer's disease patients is very low, and therefore the methods and pharmaceutical compositions of the present invention are especially advantageously applied to the treatment of this subject population because this combination of medicines is less likely to result in forgotten doses and may produce greater compliance. Combination of the compounds of the invention (i.e., the first agents of the compositions discussed below) with other palliative medications, which may be for diseases other than Alzheimer's, is another beneficial application of the present invention.

In one embodiment, the pharmaceutical compositions disclosed herein prevent or inhibit amyloid protein assembly into insoluble fibrils which, in vivo, are deposited in various organs, or it reverses or favors deposition in subjects already having deposits. In another embodiment, the compound may also prevent the amyloid protein, in its soluble, oligomeric form or in its fibrillar form, from binding or adhering to a cell surface and causing cell damage or toxicity. In yet another embodiment, the composition may block amyloid-induced cellular toxicity or microglial activation. In another embodiment, the compound may block amyloid-induced neurotoxicity. The pharmaceutical compositions of the invention may be administered therapeutically or prophylactically to treat diseases associated with amyloid-β fibril formation, aggregation or deposition. The pharmaceutical compositions of the invention may act to ameliorate the course of an amyloid-β related disease using any of the following mechanisms (this list is meant to be illustrative and not limiting): slowing the rate of amyloid-β fibril formation or deposition; lessening the degree of amyloid-β deposition; inhibiting, reducing, or preventing amyloid-β fibril formation; inhibiting neurodegeneration or cellular toxicity induced by amyloid-β; inhibiting amyloid-β induced inflammation; or enhancing the clearance of amyloid-β from the brain.

The invention pertains to a pharmaceutical composition for the treatment of an amyloid-β disease comprising a first agent and a second agent in a pharmaceutically acceptable carrier, where the first agent prevents or inhibits amyloid-β fibril formation, neurodegeneration, or cellular toxicity; and the second agent is a therapeutic drug or nutritive supplement.

Similarly, the invention includes a pharmaceutical composition for the treatment of an amyloid-β disease comprising a first agent and a second agent in a pharmaceutically acceptable carrier, wherein the first agent prevents or inhibits amyloid-β fibril formation, neurodegeneration, or cellular toxicity; and the second agent is a therapeutic drug or nutritive supplement, such that cognitive function is stabilized or further deterioration in cognitive function is prevented, slowed, or stopped.

In another embodiment, the invention is a pharmaceutical composition for the treatment of an amyloid-β disease comprising a first agent and a second agent in a pharmaceutically acceptable carrier, wherein the first agent prevents or inhibits amyloid-β fibril formation, neurodegeneration, or cellular toxicity; and the second agent is a therapeutic drug or nutritive supplement, such that activities of daily living otherwise impaired by an amyloid-β disease are improved or stabilized.

In yet another embodiment, the invention is a pharmaceutical composition for the treatment of an amyloid-β disease comprising a first agent and a second agent in a pharmaceutically acceptable carrier, wherein the first agent prevents or inhibits amyloid-β fibril formation, neurodegeneration, or cellular toxicity; and the second agent is a therapeutic drug or nutritive supplement, such that the pharmaceutical composition inhibits an interaction between an amyloidogenic protein and a glycoprotein or proteoglycan constituent of a basement membrane to thereby prevent or inhibit amyloid deposition.

Further aspects of the invention include a pharmaceutical composition for the treatment of an amyloid-β disease comprising a first agent and a second agent in a pharmaceutically acceptable carrier, wherein the first agent prevents or inhibits amyloid-β fibril formation, neurodegeneration, or cellular toxicity; and the second agent is a therapeutic drug or nutritive supplement, such that the concentration of amyloid-β or tau in the CSF of the subject changes versus an untreated subject.

A pharmaceutical composition for the treatment of an amyloid-β disease is also within the scope of the invention in which the composition has a first agent and at least two second agents in a pharmaceutically acceptable carrier, wherein the first agent prevents or inhibits amyloid-β fibril formation, neurodegeneration, or cellular toxicity; and each of the second agent is a therapeutic drug or nutritive supplement.

Also included is a pharmaceutical composition for the treatment of an amyloid-β disease comprising a first agent and a second agent in a pharmaceutically acceptable carrier, wherein the first agent binds amyloid-β; and the second agent is a therapeutic drug or nutritive supplement; such that amyloid-β fibril formation, neurodegeneration, or cellular toxicity in the subject is prevented or inhibited.

Another example of the invention is a pharmaceutical composition for the treatment of an amyloid-β disease comprising a first agent and a second agent in a pharmaceutically acceptable carrier, wherein the first agent binds amyloid-β; and the second agent is a therapeutic drug or nutritive supplement; such that cognitive function is stabilized or further deterioration in cognitive function is prevented, slowed, or stopped in the subject.

In another aspect, the invention pertains to a pharmaceutical composition for the treatment of an amyloid-β disease comprising a first agent and a second agent in a pharmaceutically acceptable carrier, wherein the first agent binds amyloid-β; and the second agent is a therapeutic drug or nutritive supplement; such that activities of daily living otherwise impaired by the amyloid-β disease are improved or stabilized in the subject.

The invention also includes a pharmaceutical composition for the treatment of an amyloid-β disease comprising a first agent and a second agent in a pharmaceutically acceptable carrier, wherein the first agent binds amyloid-β; and the second agent is a therapeutic drug or nutritive supplement; such that the pharmaceutical composition inhibits an interaction between an amyloidogenic protein and a glycoprotein or proteoglycan constituent of a basement membrane to thereby prevent or inhibit amyloid deposition in the subject.

Furthermore, the invention may be a pharmaceutical composition for the treatment of an amyloid-β disease comprising a first agent and a second agent in a pharmaceutically acceptable carrier, wherein the first agent binds amyloid-β; and the second agent is a therapeutic drug or nutritive supplement; such that the concentration of amyloid-β or tau in the CSF of the subject changes versus an untreated subject.

In another representation, the invention is a pharmaceutical composition for the treatment of an amyloid-β disease comprising a first agent and at least two second agents in a pharmaceutically acceptable carrier, wherein the first agent binds amyloid-β; and the second agent is a therapeutic drug or nutritive supplement; such that amyloid-β fibril formation, neurodegeneration, or cellular toxicity in the subject is prevented or inhibited.

The invention also relates to methods of making pharmaceutical compositions for use in the therapeutic and prophylactic methods described herein. The first agent and the second agent are supplied as a pharmaceutical product, and they may be packaged in separate containers for sale or delivery to the consumer. The first agent and the second agent may be dissolved in a liquid pharmaceutically acceptable carrier, or they may be provided in a solid formulation, for example, as a homogenous mixture in a capsule or pill. The pharmaceutical compositions may further comprise a pharmaceutically acceptable acid, base, buffering agent, inorganic salt, solvent, or preservative. Furthermore, the pharmaceutical compositions of the invention may also include a compound that increases the cerebral bioavailability of either the first agent or the second agent. The invention also relates to the use of a first agent and a second agent in the preparation of a pharmaceutical composition for the treatment or prevention of an amyloid-β disease comprising a first agent and a second agent in a pharmaceutically acceptable carrier, wherein the first agent prevents or inhibits amyloid-β fibril formation, neurodegeneration, or cellular toxicity; and the second agent is a therapeutic drug or nutritive supplement.

The term “container” includes any receptacle for holding the therapeutic formulation. For example, in one embodiment, the container is the packaging that contains the formulation. In other embodiments, the container is not the packaging that contains the formulation, i.e., the container is a receptacle, such as a box or vial that contains the packaged formulation or unpackaged formulation and the instructions for use of the formulation. Moreover, packaging techniques are well known in the art. It should be understood that the instructions for use of the therapeutic formulation may be contained on the packaging containing the therapeutic formulation, and as such the instructions form an increased functional relationship to the packaged product.

Pharmaceutical compositions of the invention may be effective in controlling amyloid-β deposition either following their entry into the brain (following penetration of the blood brain barrier) or from the periphery. When acting from the periphery, a compound of a pharmaceutical composition may alter the equilibrium of Aβ between the brain and the plasma so as to favor the exit of Aβ from the brain. An increase in the exit of Aβ from the brain would result in a decrease in Aβ brain concentration and therefore favor a decrease in Aβ deposition. Alternatively, compounds that penetrate the brain could control deposition by acting directly on brain Aβ e.g., by maintaining it in a non-fibrillar form or favoring its clearance from the brain, or protecting brain cells from the detrimental effect of Aβ. These compounds could also prevent Aβ in the brain from interacting with a cell surface and therefore prevent neurotoxicity or inflammation.

In some aspects the pharmaceutical compositions of the invention contain a first agent that prevents or inhibits β-amyloid fibril formation, either in the brain or other organ of interest (acting locally) or throughout the entire body (acting systemically). Without wishing to be bound by theory, the inventors believe that the first agent as described herein may inhibit or reduce an interaction between amyloid-β and a cell surface constituent, for example, a glycosaminoglycan or proteoglycan constituent of a basement membrane, and that inhibiting or reducing this interaction is primarily responsible for the observed neuroprotective effects. For example, the first agent may also prevent an amyloid-β peptide from binding or adhering to a cell surface, a process which is known to cause cell damage or toxicity. Similarly, the first agent may block amyloid-induced cellular toxicity or microglial activation or amyloid-induced neurotoxicity, or inhibit amyloid-β induced inflammation. The first agent may also reduce the rate or amount of β-amyloid aggregation, fibril formation, or deposition, or the first agent lessens the degree of amyloid-β deposition. The first agent may also inhibit, reduce, or prevent amyloid-β fibril formation.

Additionally, the first agent may enhance the clearance of amyloid-β from the brain; or the first agent may favorably alter the equilibrium of amyloid-β between the brain and the plasma to decrease the amount of amyloid-β in the brain. The first agent may lower the levels of amyloid β peptides, e.g., both Aβ40 and Aβ42 in the CSF and the plasma, or the first agent may lower the levels of amyloid β peptides, e.g., Aβ40 and Aβ42 in the CSF and increase it in the plasma.

Regardless of the particular mechanism by which the first agent exerts its biological effects, the first agent prevents or treats amyloid-β diseases, such as for example Alzheimer's disease. The first agent may reverse or favor deposition of amyloid in a subject having amyloid deposits, or the first agent may favor plaque clearance or slow deposition in a subject having amyloid deposits. For example, the first agent decreases the amyloid-β concentration in the brain of a subject versus an untreated subject, and the first agent penetrates into the brain, that is, it crosses the blood-brain barrier (“BBB”) where is exerts it biological effect. Therefore, the first agent may maintain soluble amyloid in a non-fibrillar form. Accordingly, the first agent may increase the rate of clearance of soluble amyloid from the brain of a subject versus an untreated subject.

The invention also includes a method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or inhibits amyloid-β fibril formation, neurodegeneration, or cellular toxicity; and said second agent is a therapeutic drug or nutritive supplement.

Also within the purview of the invention is a method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or inhibits amyloid-β fibril formation, neurodegeneration, or cellular toxicity; and said second agent is a therapeutic drug or nutritive supplement, such that cognitive function is stabilized or further deterioration in cognitive function is prevented, slowed, or stopped.

Similarly, the invention is a method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or inhibits amyloid-β fibril formation, neurodegeneration, or cellular toxicity; and said second agent is a therapeutic drug or nutritive supplement, such that activities of daily living otherwise impaired by said amyloid-β disease are improved or stabilized.

In another embodiment, the invention is a method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or inhibits amyloid-β fibril formation, neurodegeneration, or cellular toxicity; and said second agent is a therapeutic drug or nutritive supplement, such that said pharmaceutical composition inhibits an interaction between an amyloidogenic protein and a glycoprotein or proteoglycan constituent of a basement membrane to thereby prevent or inhibit amyloid deposition.

In yet another embodiment, the invention may be a method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or inhibits amyloid-β fibril formation, neurodegeneration, or cellular toxicity; and said second agent is a therapeutic drug or nutritive supplement, such that the concentration of amyloid-β or tau in the CSF of said subject changes versus an untreated subject.

The invention also pertains to a method of concomitant therapeutic treatment of a subject, comprising administering to a subject in need thereof an effective amount of a pharmaceutical composition for treating or preventing an amyloid-β disease, said pharmaceutical composition comprising a first agent and at least two second agents in a pharmaceutically acceptable carrier, wherein said first agent prevents or inhibits amyloid-β fibril formation, neurodegeneration, or cellular toxicity; and each of said second agent is a therapeutic drug or nutritive supplement.

In other aspects, the invention is a method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent binds amyloid-β; and said second agent is a therapeutic drug or nutritive supplement; such that amyloid-β fibril formation, neurodegeneration, or cellular toxicity in said subject is prevented or inhibited.

In further aspects, the invention is a method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent binds amyloid-β; and said second agent is a therapeutic drug or nutritive supplement; such that cognitive function is stabilized or further deterioration in cognitive function is prevented, slowed, or stopped in said subject.

Another method of concomitant therapeutic treatment of a subject of the invention comprises administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent binds amyloid-β; and said second agent is a therapeutic drug or nutritive supplement; such that activities of daily living otherwise impaired by said amyloid-β disease are improved or stabilized in said subject.

Additionally, the invention pertains to a method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent binds amyloid-β; and said second agent is a therapeutic drug or nutritive supplement; such that said pharmaceutical composition inhibits an interaction between an amyloidogenic protein and a glycoprotein or proteoglycan constituent of a basement membrane to thereby prevent or inhibit amyloid deposition in said subject.

A further example of the invention is a method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent binds amyloid-β; and said second agent is a therapeutic drug or nutritive supplement; such that the concentration of amyloid-β or tau in the CSF of said subject changes versus an untreated subject.

In another embodiment, the invention is a method of concomitant therapeutic treatment of a subject, comprising administering to a subject in need thereof an effective amount of a pharmaceutical composition for treating or preventing an amyloid-β disease, said pharmaceutical composition comprising a first agent and at least two second agents in a pharmaceutically acceptable carrier, wherein said first agent binds amyloid-β; and said second agent is a therapeutic drug or nutritive supplement; such that amyloid-β fibril formation, neurodegeneration, or cellular toxicity in said subject is prevented or inhibited.

As used herein, “combination therapy” or “therapeutic combination” means the administration of two or more “first agents,” e.g., compounds represented by Formulae (I-X), or administration of one or more first agents, such as compounds represented by Formulae (I-X) with other Alzheimer's disease treatments different from the first agent, as discussed below, e.g., cholesterol biosynthesis inhibitors or lipid-lowering agents, to prevent or treat Alzheimer's Disease, by for example reducing levels of one or more amyloid β peptides; regulating production of amyloid β peptides or regulating levels of ApoE isoform 4 in the bloodstream or the brain. Such administration includes coadministration of these therapeutic agents in a substantially simultaneous manner, such as in a single tablet or capsule having a fixed ratio of active ingredients or in multiple, separate capsules for each therapeutic agent. Also, such administration includes use of each type of therapeutic agent in a sequential manner. In either case, the treatment using the combination therapy will provide beneficial effects in treating the condition. A potential advantage of the combination therapy disclosed herein may be a reduction in the required amount of an individual therapeutic compound or the overall total amount of therapeutic compounds that are effective in treating the condition. By using a combination of therapeutic agents, the side effects of the individual compounds can be reduced as compared to a monotherapy, which can improve subject compliance. Also, therapeutic agents can be selected to provide a broader range of complimentary effects or complimentary modes of action.

According to the invention, “combination therapy” also includes simultaneous co-administration of a first agent (e.g., an alkanesulfonic acid) and a second agent; and the term also includes methods comprising the steps of administration of the first agent, followed by the second agent, or treatment and administration of the second agent, followed by administration of the first agent.

In one embodiment of the present invention, the therapeutic value of the first agent is enhanced or maximized by co-administration with a second agent. The second agent may facilitate and/or enhance the activity or efficacy of the first agent by the same and/or complementary mechanisms of action. For example, the first agent may prevent or inhibit amyloid-B fibril formation and the second agent may have a complementary effect of altering the biodistribution and/or the equilibrium amount of the aggregation forms for amyloid-B. Additionally or alternatively, the second agent may alleviate one or more symptoms of the diseases addressed herein, e.g., it may enhance cognitive function or memory, or treat depression.

Some general examples of compounds that may be used as a second agent according to the invention include neuro-transmission enhancers; psychotherapeutic drugs; acetylcholine-esterase inhibitors; calcium channel blockers; biogenic amines; benzodiazepine tranquilizers; acetylcholine synthesis, storage, or release enhancers; acetylcholine postsynaptic receptor agonists; monoamine oxidase-A or -B inhibitors; N-methyl-D-aspartate glutamate receptor antagonists; nonsteroidal anti-inflammatory drugs; antioxidants; and serotonergic receptor antagonists.

Additional examples of compounds that may be used as a second agent according to the invention include agents that enhance acetylcholine synthesis, storage, or release, such as phosphatidylcholine, 4-aminopyridine, bifemelane, 3,4-diaminopyridine, choline, vesamicol, secoverine, bifemelane, tetraphenylurea, and nicotinamide; postsynaptic receptor agonists, such as arecoline, oxotremorine, bethanechol, ethyl nipecotate, and levacecamine; N-methyl-D-aspartate glutamate receptor antagonists, such as milacemide and memantine; specific monoamine oxidase A inhibitors, such as moclobemide; monoamine oxidase B inhibitors, such as selegiline; thiamine and sulbutiamine; D-cycloserine; anfacine; linopirdine; deferoxamine and nonsteroidal anti-inflammatory drugs; serotoneregic receptor antagonists, such as ketanserin and mianserin; vasodilator or other nootropic direct brain metabolic enhancer drugs such as idebenone, bromvincamine, propentophylline, pentoxifylline, citicoline, piracetam, oxiracetam, aniracetam, pramiracetam, pyroglutamic acid, tenilsetam, rolziracetam, etiracetam, dupracetam, vinpocetine (Cavinton™, Chemical Works of Gedeon Richter, Ltd., Budapest, Hungary), ebiratide, β-carbolines (e.g., ethyl-5-isopropyloxy-4-methyl-β-carboline-3-carboxylate and N′-methyl-β-carboline-3-carboximide, methyl-6,7-dimethoxy-4-ethyl-β-carboline-3-carboxylate, and ethyl 5-methoxy-4-ethyl-p-carboline-3-carboxylate), naloxone, ergoloid mesylates (e.g., Hydergine), cyclandelate, isoxsuprene, nafronyl, papaverine, suloctidil, vinbumine, vincamine, vindebumol, flunarizine, nimodipine, nicergoline, razobazam, exifone, rolipram, sabeluzole, phosphatidylserine, and ifenprodil; neurotransmission enhancers, such as amantadine, calcium hopantenate, lisuride, bifemelane, and indeloxazine; tiapride, a selective D₂ antagonist; psychotherapeutic drugs, such as haloperidol, bromperidol, thioridazine, thiothixene, fluphenazine, perphenazine, and molindone; antioxidants, such as tocopherols, ascorbic acid, and deferoxamine; acetylcholinesterase inhibitors, such as physostigmine (optionally with lecithin), heptylphysostigmine, tetrahydroaminoacridine (tacrine) and a the related compound 9-amino-1,2,3,4-tetrahydroacridin-1-ol, metrifonate, velnacrine maleate, sulfonyl fluorides (e.g., methanesulfonyl fluoride and phenylmethanesulfonyl fluoride), huperzines A and B, galanthamine, edrophonium and miotine and derivatives therof; calcium channel blocker agents, such as diltiazem, verapamil, nifedipine, nicardipine, isradipine, amlodipine and felodipine; biogenic amines and related compounds, such as clonidine (a noradrenergic α₂-receptor agonist), guanfacine (an adrenergic agonist), alaproclate, fipexide, zimeldine, and citalopram; anti-rage drugs, such as propranolol, carbamazepine, and fluoxetine; minor tranquilizers such as benzodiazepine agents; and angiotensin-converting enzyme inhibitors, such as captopril (Capoten™ and Capozide™ (Bristol-Myers Squibb Co., New York, N.Y.). See, e.g., R. Anand, et al., Adv. Neurol. 51, 261-68 (1990); W. G. Bradley, Muscle & Nerve 13, 833-42 (1990); V. Chan-Palay, Psychopharmacology 106, S137-S139 (1992); J. K. Cooper, et al., Arch. Intern. Med. 151, 245-49 (1991); N. R. Cutler, et al., Ann. Pharmacother. 26, 1118-22 (1992); P. Davies, Clin. Neuropharmacol. 14(Suppl. 1), S24-S33 (1991); M. W. Dysken, et al., J. Am. Geriatr. Soc. 40, 503-06 (1992); S. H. Ferris, Acta Neurol. Scand Suppl. 129, 23-26 (1990); P. T. Francis, et al., Ann. N.Y. Acad. Sci. 640, 184-88 (1991); D. Groo, et al., Drug Dev. Res. 11, 29-36 (1987); A. L. Harvey, Adv. Neurol. 51, 227-33 (1990); P. L. McGeer, et al., Neurology 42, 447-49 (1992); L. Parnetti, et al., Eur. J. Clin. Pharmacol. 42, 89-93 (1992); M. Shimizu, et al., Alzheimer's Dis. Assoc. Disord. 5(Suppl. 1), S13-S24 (1991); J. E. Sweeney, et al., Psychopharmacology 102, 191-200 (1990); P. J. Whitehouse, Alzheimer Dis. Assoc. Disord. 5(Suppl. 1), S32-S36 (1991); and R. J. Wurtman, et al., Adv. Neurol. 51, 117-25 (1990).

In another aspect of the invention, the amyloid-β peptide is a peptide having 39-43 amino-acids, which is an amyloidogenic peptide produced from βAPP.

The “amyloid-β disease” (or “amyloid-β related disease,” which terms as used herein are synonymous) may be Mild Cognitive Impairment; vascular dementia; Alzheimer's disease, including sporadic (non-hereditary) Alzheimer's disease and familial (hereditary) Alzheimer's disease; cerebral amyloid angiopathy or hereditary cerebral hemorrhage; senile dementia; Down's syndrome; inclusion body myositis; or age-related macular degeneration.

In another embodiment, the method is used to treat Alzheimer's disease (e.g., sporadic or familial Alzheimer's disease). The method can also be used prophylactically or therapeutically to treat other clinical occurrences of amyloid-β deposition, such as in Down's syndrome individuals and in subjects with cerebral amyloid angiopathy (“CAA”) or hereditary cerebral hemorrhage.

Cerebral amyloid angiopathy (“CAA”) refers to the specific deposition of amyloid fibrils in the walls of leptomingeal and cortical arteries, arterioles and in capillaries and veins. It is commonly associated with Alzheimer's disease, Down's syndrome and normal aging, as well as with a variety of familial conditions related to stroke or dementia (see Frangione, et al., Amyloid: J. Protein Folding Disord. 8, Suppl. 1, 36-42 (2001)). CAA can occur sporadically or be hereditary. Multiple mutation sites in either Aβ or the APP gene have been identified and are clinically associated with either dementia or cerebral hemorrhage. Exemplary CAA disorders include, but are not limited to, hereditary cerebral hemorrhage with amyloidosis of Icelandic type (HCHWA-I); the Dutch variant of HCHWA (HCHWA-D; a mutation in Aβ); the Flemish mutation of Aβ; the Arctic mutation of Aβ; the Italian mutation of Aβ; the Iowa mutation of Aβ; familial British dementia; and familial Danish dementia.

Additionally, abnormal accumulation of APP and of amyloid-β protein in muscle fibers has been implicated in the pathology of sporadic inclusion body myositis (“IBM”) (Askanas, et al., Proc. Natl. Acad. Sci. USA 93, 1314-19 (1996); Askanas, et al., Current Opinion in Rheumatology 7, 486-96 (1995)). Accordingly, the compounds of the invention can be used prophylactically or therapeutically in the treatment of disorders in which amyloid-β protein is abnormally deposited at non-neurological locations, such as treatment of IBM by delivery of the compounds to muscle fibers.

Additionally, it has been shown that Aβ is associated with abnormal extracellular deposits, known as drusen, that accumulate along the basal surface of the retinal pigmented epithelium in individuals with age-related macular degeneration (ARMD). ARMD is a cause of irreversible vision loss in older individuals. It is believed that Aβ deposition could be an important component of the local inflammatory events that contribute to atrophy of the retinal pigmented epithelium, drusen biogenesis, and the pathogenesis of ARMD (Johnson, et al., Proc. Natl. Acad. Sci. USA 99(18), 11830-5 (2002)). Therefore, the invention also relates to the treatment or prevention of age-related macular degeneration.

The invention pertains to pharmaceutical compositions and methods of use thereof for the treatment of amyloid-β diseases. The pharmaceutical compositions comprise a first agent that, e.g., prevents or inhibits amyloid-β fibril formation, neurodegeneration, or cellular toxicity. The pharmaceutical composition also comprises a second agent that is an active pharmaceutical ingredient; that is, the second agent is therapeutic and its function is beyond that of an inactive ingredient, such as a pharmaceutical carrier, preservative, diluent, or buffer. The second agent may be useful in treating or preventing an amyloid-β disease or another neurological disease. The first and second agents may exert their biological effects by similar or unrelated mechanisms of action; or either one or both of the first and second agents may exert their biological effects by a multiplicity of mechanisms of action.

Accordingly, in particular embodiments, the invention pertains to pharmaceutical compositions and methods of use thereof for the treatment of amyloid diseases, amyloid-β diseases, or Alzheimer's Disease. The pharmaceutical composition comprises a first agent that modulates a first biological process and/or exerts a first biological effect, and a second agent that modulates a second biological process and/or exerts a second biological effect. The first agent and the second agent may modulate different biological processes in the pathogenesis of the disease and/or exert different biological effects and/or act on different targets. For example, the first agent may be therapeutically useful in the treatment of Alzheimer's disease, and the second agent may be therapeutically useful in the treatment of CAA, dementia, ALS, or Parkinson's Disease. The first agent and the second agent may have different binding affinities or specificities for peptides, proteins, or enzymes involved in the pathogenesis of the amyloid disease (e.g., Alzheimer's disease). In preferred embodiments, the first agent and the second agent, when simultaneously present in a subject, act synergistically to reduce, inhibit, or ameliorate the symptoms or pathogenesis of Alzheimer's disease.

A pharmaceutical composition may also comprise a third compound, or even more yet, wherein the third (and fourth, etc.) compound has the same characteristics of a second agent. The “second agent” is selected in accord with the following therapeutic principles.

Pharmacologic Treatment of Alzheimer's Disease and Other Amyloid-β Diseases

The pathology of Alzheimer's disease includes a number of characteristic components, including but not limited to β-amyloid deposits, such as diffuse plaques and senile plaques; cytoskeletal pathology, such as hyperphosphorylated tau and paired helical filaments; cholinergic degeneration, such as basal cholinergic neuronal loss and reduced ChAT in cortex and septum; inflammation, such as gliosis; and cognitive and behavioral dysfunction, such as cognitive loss, apathy and aggression. To reflect the varied characteristics of the disease, a variety of therapeutic approaches to the disease have been taken. To date, clinically validated treatments for Alzheimer's disease remain confined to symptomatic interventions such as treatment with enhancers of cognitive function, e.g., acetylcholinesterase, acetyl/butyrylcholinesterase inhibitors, or NMDA receptor antagonists.

Several broad therapeutic strategies for disease-modifying agents are currently being approached. These include, for example, the following: inhibiting the β and gamma-secretase enzymes that generate Aβ from APP; preventing oligomerization or fibrillogenesis of Aβ or enhancing its clearance from the brain, e.g. by active or passive immunization with Aβ, by administering anti-fibrillogenic small molecule compounds or peptides, or by metal chelation; blocking or inhibiting inflammation and neurodegeneration induced by Aβ; reducing the formation of phosphorylated Tau protein in the neurofibrillary tangles; and modulating cholesterol homeostasis. A range of compounds with antioxidant, neuroprotective or neurotrophic properties are contemplated for treating Alzheimer's disease; numerous approaches such as these and others are intended to be within the scope of the invention. See, e.g., J. Hardy, et al, Science 297, 353-56 (2002).

There are many different mechanisms by which these therapeutic approaches could treat Alzheimer's disease. For example, vaccination therapy could stimulate an immune response against Aβ peptides, leading to clearance of the peptides from the body. P and gamma-secretase inhibitors could lead to decreased production of Aβ peptides. Copper/zinc chelators such as clioquinol could decrease the interaction of copper and zinc with Aβ peptides, leading to the clearance of amyloid plaques. Activating α-secretase, which cleaves within Aβ, would be expected to decrease the production of Aβ and is thus another target.

Pathways involved in neurodegeneration or apoptosis are also targets for therapeutic intervention. For example, the phosphorylation of poly Q ataxin by the Akt kinase is required for neurodegeneration, suggesting that the Akt kinase could be a target. Orr, et al., Neuron 38(3), 375-87 (2003); Zoghbi, et al, Cell 113(4), 457-68 (2003). Tau is found in neurofibrillary tangles hyperphosphorylated and inhibition of the kinases involved in its phosphorylation, e.g., GSK-3, is also a target. See, e.g., WO 96/35,126.

Many strategies for targeting amyloid include, inter alia, preventing oxidative damage with anti-oxidants (e.g., melatonin, curcumin); inhibiting amyloid formation or deposition with anti-aggregation agents (e.g., peptides, metal chelators, glycosaminoglycan mimetics); altering APP metabolism (e.g., with wortmannin or secretase inhibitors); shifting the equilibrium between levels of amyloidogenic peptides in the periphery and the central nervous system (e.g., with antibodies, vaccines, gelsolin, GM1, IGF-1) and decreasing microglial activation leading to inflammation (e.g., Fc, TGFβ1).

Therapeutic treatment strategies may employ anti-fibrillogenic agents. For example, a therapeutic agent may bind to Aβ to prevent or inhibit its fibril formation. For example, the 16-21 region of the Aβ peptide, KLVFFA, is responsible for the β-sheet formation and the intermolecular interactions of Aβ during fibrillogenesis. Peptides from this region have been extensively tested for their antifibrillogenic activity (Tjemberg LO, et al., J. Biol. Chem. 272, 12601-05 (1997); Findeis, et al., Biochemistry 38, 6791-6800 (1999); Findeis, et al., Amyloid, 231-41 (December 2001)). Agents, including non-peptidic agents, of the invention may be used as an anti-fibrillogenic agent in this way. The non-amyloidogenic pathway may be regulated through phosphorylation processes. Alteration of PKC levels and activity is one of the most consistent findings in Alzheimer's disease brain tissue. In addition, altered signal transduction mechanisms, particularly PKC, are found consistently in peripheral tissues from Alzheimer's disease subjects suggesting that these changes are not secondary to neuronal loss and may be directly involved in Alzheimer's disease pathogenesis. Altered APP metabolism is a key event in the amyloid cascade hypothesis. The studies on the role of PKC in the regulated APP processing have established that the Aβ forming amyloidogenic pathway and the α-secretase non-amyloidogenic pathway appear to be balanced. The target of PKC phosphorylation is not the APP molecule itself, yet the possibility that PKC targets directly the α-secretase or other key cellular factors possibly related to the vesicular trafficking of APP or the α-secretase, has not been resolved. M. Racchi, et al., Experimental Gerontology 38, 145-57 (2003). Neurofibrillary tangles are composed of hyperphosphorylated tau proteins. One or more kinases are principally responsible for initiating the hyperphosphorylation of tau in vivo that leads to its apparent dissociation from microtubules and aggregation into insoluble paired helical filaments. Hyperphosphorylation of tau may underlie tangle formation in Alzheimer's disease. Calpain is responsible for cleavage of p35 and treating cells with Aβ aggregates can trigger p35 activation and the subsequent cdk5-mediated phosphorylation of tau and perhaps other cytoplasmic substrates. D. Selkoe, Physiol. Rev. 81(2), 741-66 (2001). Suitable agents for use in the invention may target any of these biological processes.

In some cases, one drug may target more than one therapeutic approach. For example, studies suggest that butyryl cholinesterase inhibitors which inhibit the activity of the cholinesterase enzyme are also associated with Aβ (Darvesh, et al., Cell. Mol. NeurobioL 21, 285-96 (2001)). Phenserine, an acetylcholinesterase inhibitor, may inhibit both the activity of the acetylcholinesterase enzyme and the processing or translation of the APP mRNA. Cholesterol-lowering drugs such as statins, e.g. avorstatin, could increase processing of amyloid precursor protein by alpha-secretase, leading to the decreased production of Aβ peptides. Nonsteroidal anti-inflammatory drugs such as ibuprofen, flurbiprofen, indomethacin, and sulindac sulphide, could selectively inhibit the production of the Aβ42 peptide, in addition to inhibiting the inflammation induced by Aβ. The decrease in Aβ42 peptide that occurs in transgenic mice when administered fluribuprofin, marketed as Ansaid™ (Upjohn, now Pfizer, New York, N.Y.), which is in Phase II human clinical trials Myriad Genetics, Inc. (Salt Lake City, Utah), has been correlated with improved memory and special learning.

The method relates to a method for treating or preventing an amyloid-β related disease by administering at least two agents, each of which is a compound that exerts a therapeutic effect when so administered and is useful in treating or preventing a neurological or psychological condition or disease. The first compound of the invention, as described further below, may be an alkanesulfonic acid that is useful for treating or preventing an amyloid-β related disease. The second compound is therapeutic, i.e., its function is beyond that of an inactive ingredient, such as a pharmaceutical carrier, preservative, diluent, or buffer. The second compound may be useful in treating or preventing an amyloid-β related disease or another neurological disease. The second compound may also be useful in diminishing specific symptoms which are characteristic of Alzheimer's disease (e.g. memory loss, anxiety, etc.) The first and second compounds may exert their biological effects by similar or unrelated mechanisms of action; or either one or both of the first and second compounds may exert their biological effects by a multiplicity of mechanisms of action. A third compound, or even more yet, may likewise be used in a method of the invention, wherein the third (and fourth, etc.) compound has the same characteristics of a second compound.

In one embodiment, pharmaceutical compositions of the invention are formulated to be orally administered to a subject. The first agent and said second agent may be simultaneously administered. The first agent and the second agent may modulate different biological processes in the pathogenesis of Alzheimer's disease. The first agent and the second agent may act on different targets. For example, the first agent may be therapeutically useful in the treatment of Alzheimer's disease, and the second agent may be therapeutically useful in the treatment of CAA. The first agent and the second agent may have different binding affinities or specificities for peptides, proteins, or enzymes involved in the pathogenesis of Alzheimer's disease. The first agent and the second agent, when simultaneously present in a subject, act synergistically to reduce, inhibit, or ameliorate the symptoms or pathogenesis of Alzheimer's disease.

The term “subject” includes living organisms in which amyloidosis can occur, or which are susceptible to amyloid diseases, e.g., Alzheimer's disease. Examples of subjects include humans, monkeys, cows, sheep, goats, dogs, cats, mice, rats, and transgenic species thereof. Administration of the compositions of the present invention to a subject to be treated can be carried out using known procedures, at dosages and for periods of time effective to modulate amyloid aggregation or amyloid-induced neurotoxicity in the subject as further described herein. An effective amount of the therapeutic compound necessary to achieve a therapeutic effect may vary according to factors such as the amount of amyloid already deposited at the clinical site in the subject, the age, sex, and weight of the subject, and the ability of the therapeutic compound to modulate amyloid aggregation in the subject. Dosage regimens can be adjusted to provide the optimum therapeutic response. For example, several divided doses may be administered daily or the dose may be proportionally reduced as indicated by the exigencies of the therapeutic situation.

In an exemplary aspect of the invention, the subject is a human. For example, the subject may be a human over 40 years old, or a human over 50 years old, or a human over 60 years old, or even a human over 70 years old. The subject may be a female human, including a postmenopausal female human, who may be on hormone (estrogen) replacement therapy. The subject may also be a male human.

A subject may be a human at risk for Alzheimer's disease, e.g., being over the age of 40 or having a predisposition for Alzheimer's disease. Alzheimer's disease predisposing factors identified or proposed in the scientific literature include, among others, a genotype predisposing a subject to Alzheimer's disease; environmental factors predisposing a subject to Alzheimer's disease; past history of infection by viral and bacterial agents predisposing a subject to Alzheimer's disease; and vascular factors predisposing a subject to Alzheimer's disease. A subject may also have one or more risk factors for cardiovascular disease (e.g., atherosclerosis of the coronary arteries, angina pectoris, and myocardial infarction) or cerebrovascular disease (e.g., atherosclerosis of the intracranial or extracranial arteries, stroke, syncope, and transient ischemic attacks), such as hypercholesterolemia, hypertension, diabetes, cigarette smoking, familial or previous history of coronary artery disease, cerebrovascular disease, and cardiovascular disease. Hypercholesterolemia typically is defined as a serum total cholesterol concentration of greater than about 5.2 mmol/L (about 200 mg/dL).

Several genotypes are believed to predispose a subject to Alzheimer's disease. These include the genotypes such as presenilin-1, presenilin-2, and amyloid precursor protein (APP) missense mutations associated with familial Alzheimer's disease, and α-2-macroglobulin and LRP-1 genotypes, which are thought to increase the risk of acquiring sporadic (late-onset) Alzheimer's disease. E van Uden, et al., J. Neurosci. 22(21), 9298-304 (2002); J. J. Goto, et al., J. Mol. Neurosci. 19(1-2), 37-41 (2002). Another genetic risk factor for the development of Alzheimer's disease are variants of ApoE, the gene that encodes apolipoprotein E (particularly the apoE4 genotype), a constituent of the low-density lipoprotein particle. W J Strittmatter, et al., Annu. Rev. Neurosci. 19, 53-77 (1996). The molecular mechanisms by which the various ApoE alleles alter the likelihood of developing Alzheimer's disease are unknown, however the role of ApoE in cholesterol metabolism is consistent with the growing body of evidence linking cholesterol metabolism to Alzheimer's disease. For example, chronic use of cholesterol-lowering drugs such as statins has recently been associated with a lower incidence of Alzheimer's disease, and cholesterol-lowering drugs have been shown to reduce pathology in APP transgenic mice. These and other studies suggest that cholesterol may affect APP processing. Environmental factors have been proposed as predisposing a subject to Alzheimer's disease, including exposure to aluminum, although the epidemiological evidence is ambiguous. In addition, prior infection by certain viral or bacterial agents may predispose a subject to Alzheimer's disease, including the herpes simplex virus and chlamydia pneumoniae. Finally, other predisposing factors for Alzheimer's disease can include risk factors for cardiovascular or cerebrovascular disease, including cigarette smoking, hypertension and diabetes. “At risk for Alzheimer's disease” also encompasses any other predisposing factors not listed above or as yet identified and includes an increased risk for Alzheimer's disease caused by head injury, medications, diet, or lifestyle.

In certain embodiments of the invention, the subject is in need of treatment by the methods of the invention, and is selected for treatment based on this need. A subject in need of treatment is art-recognized, and includes subjects that have been identified as having a disease or disorder related to amyloid-deposition or amyloidosis, has a symptom of such a disease or disorder, or is at risk of such a disease or disorder, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or reducing the risk of the disease or disorder).

The methods of the present invention can be used for one or more of the following: to prevent, to treat Alzheimer's disease, or ameliorate symptoms of Alzheimer's disease, to regulate production of or levels of amyloid β (Aβ) peptides or regulate the amount of ApoE isoform 4 in the bloodstream or brain of a subject. In one alternative embodiment, the human carries one or more mutations in the genes that encode β-amyloid precursor protein, presenilin-1 or presenilin-2. In another alternative embodiment, the human carries the Apolipoprotein ε4 gene. In another alternative embodiment, the human has a family history of Alzheimer's Disease or dementia illness. In another alternative embodiment, the human has trisomy 21 (Down's Syndrome). In another alternative embodiment, the subject has a normal or low serum total blood cholesterol level. In another embodiment, the serum total blood cholesterol level is less than about 200 mg/dL, or less than about 180, and it can range from about 150 to about 200 mg/dL. In another embodiment, the total LDL cholesterol level is less than about 100 mg/dL, or less than about 90 mg/dL and can range from about 30 to about 100 mg/dL. Methods of measuring serum total blood cholesterol and total LDL cholesterol are well known to those skilled in the art and for example include those disclosed in WO 99/38498 at p.11, incorporated by reference herein. Methods of determining levels of other sterols in serum are disclosed in H. Gylling, et al., “Serum Sterols During Stanol Ester Feeding in a Mildly Hypercholesterolemic Population”, J. Lipid Res. 40: 593-600 (1999).

In another alternative embodiment, the subject has an elevated serum total blood cholesterol level. In another embodiment, the serum total cholesterol level is at least about 200 mg/dL, or at least about 220 mg/dL and can range from about 200 to about 1000 mg/dL. In another alternative embodiment, the subject has an elevated total LDL cholesterol level. In another embodiment, the total LDL cholesterol level is greater than about 100 mg/dL, or even greater than about 110 mg/dL and can range from about 100 to about 1000 mg/dL.

In another alternative embodiment, the human is at least about 40 years of age. In another alternative embodiment, the human is at least about 60 years of age. In another embodiment, the human is at least about 70 years of age. In one embodiment, the human is between about 60 and 100 years of age.

In still a further embodiment, the subject is shown to be at risk by a diagnostic brain imaging technique, for example, that measures brain activity, plaque deposition, or brain atrophy.

In another embodiment, the subject exhibits no symptoms of Alzheimer's Disease. In another embodiment, the subject is a human who is at least 40 years of age and exhibits no symptoms of Alzheimer's Disease. In another embodiment, the subject is a human who is at least 40 years of age and exhibits one or more symptoms of Alzheimer's Disease.

By using the methods of the present invention, the levels of amyloid β peptides in a subject's brain or blood can be reduced from levels prior to treatment from about 10 to about 100 percent, or even about 50 to about 100 percent.

In an alternative embodiment, the subject can have an elevated level of amyloid Aβ₄₀ and Aβ₄₂ peptide in the blood and CSF prior to treatment, according to the present methods, of greater than about 10 pg/mL, or greater than about 20 pg/mL, or greater than about 35 pg/mL, or even greater than about 40 pg/mL. In another embodiment, the elevated level of amyloid Aβ₄₂ peptide can range from about 30 pg/mL to about 200 pg/mL, or even to about 500 pg/mL. One skilled in the art would understand that as Alzheimer's disease progresses, the measurable levels of amyloid β peptide in the CSF may decrease slightly from elevated levels present before onset of the disease. This effect is attributed to increased deposition, i.e., trapping of Aβ peptide in the brain instead of normal clearance from the brain into the CSF.

In an alternative embodiment, the subject can have an elevated level of amyloid Aβ₄₀ peptide in the blood and CSF prior to treatment, according to the present methods, of greater than about 5 pg Aβ₄₂/mL or greater than about 50 pg Aβ₄₀/mL, or greater than about 400 pg/mL. In another embodiment, the elevated level of amyloid Aβ₄₀ peptide can range from about 200 pg/mL to about 800 pg/mL, to even about 1000 pg/mL.

In another embodiment, the subject can have an elevated level of amyloid Aβ₄₂ peptide in the CSF prior to treatment, according to the present methods, of greater than about 5 pg/mL, or greater than about 10 pg/mL, or greater than about 200 pg/mL, or greater than about 500 pg/nL. In another embodiment, the level of amyloid β peptide can range from about 10 pg/mL to about 1,000 pg/mL, or even about 100 pg/mL to about 1,000 pg/mL.

In another embodiment, the subject can have an elevated level of amyloid Aβ₄₀ peptide in the CSF prior to treatment according to the present methods of greater than about 10 pg/mL, or greater than about 50 pg/mL, or even greater than about 100 pg/mL. In another embodiment, the level of amyloid β peptide can range from about 10 pg/mL to about 1,000 pg/mL.

The amount of amyloid β peptide in the brain or blood of a subject can be evaluated by enzyme-linked immunosorbent assay (“ELISA”) or quantitative immunoblotting test methods or by quantitative SELDI-TOF which are well known to those skilled in the art, such as is disclosed by Zhang, et al., J. Biol. Chem. 274, 8966-72 (1999) and Zhang, et al., Biochemistry 40, 5049-55 (2001). See also, A. K. Vehmas, et al., DNA Cell Biol. 20(11), 713-21 (2001), P.Lewczuk, et al., Rapid Commun. Mass Spectrom. 17(12), 1291-96 (2003); B. M. Austen, et al., J. Peptide Sci. 6, 459-69 (2000); and H. Davies, et al., BioTechniques 27, 1258-62 (1999). These tests are performed on samples of the brain or blood which have been prepared in a manner well known to one skilled in the art. Another example of a useful method for measuring levels of amyloid β peptides is by Europium immunoassay (EIA). See, e.g., WO 99/38498 at p. 11.

In another embodiment, the amount of total ApoE in the bloodstream or brain of a subject can be reduced from levels prior to treatment by about 5 to about 75 percent, or, in another embodiment, by about 5 to about 50 percent. The amount of total ApoE can be measured in a manner well known to one skilled in the art, for example using an ELISA test kit such as Apo-Tek ApoE test kit that is available from Organon Teknica.

The methods of the invention may be applied as a therapy for a subject having Alzheimer's disease or a dementia, or the methods of the invention may be applied as a prophylaxis against Alzheimer's disease or dementia for subject with such a predisposition, as in a subject, e.g., with a genomic mutation in the APP gene, the ApoE gene, or a presenilin gene. The subject may have (or may be predisposed to developing or may be suspected of having) vascular dementia, or senile dementia, or Mild Cognitive Impairment. In addition to Alzheimer's disease, the subject may have another amyloid-β related disease such as cerebral amyloid angiopathy, or the subject may have amyloid deposits, especially amyloid-β amyloid deposits in the subject's brain.

Definition of Dementia

The essential features of a dementia are multiple cognitive deficits that include memory impairment and at least one of the following: aphasia, apraxia, agnosia, or a disturbance in executive functioning (the ability to think abstractly and to plan, initiate, sequence, monitor, and stop complex behavior). The order of onset and relative prominence of the cognitive disturbances and associated symptoms vary with the specific type of dementia, as discussed in the following.

Memory impairment is generally a prominent early symptom. Individuals with dementia have difficulty learning new material and may lose valuables, such as wallets and keys, or forget food cooking on the stove. In more severe dementia, individuals also forget previously learned material, including the names of loved ones. Individuals with dementia may have difficulty with spatial tasks, such as navigating around the house or in the immediate neighborhood (where difficulties with memory are unlikely to play a role). Poor judgment and poor insight are common as well. Individuals may exhibit little or no awareness of memory loss or other cognitive abnormalities. They may make unrealistic assessments of their abilities and make plans that are not congruent with their deficits and prognosis (e.g., planning to start a new business). They may underestimate the risks involved in activities (e.g., driving).

In order to make a diagnosis of dementia, the cognitive deficits must be sufficiently severe to cause impairment in occupational or social functioning and must represent a decline from a previous level of functioning. The nature and degree of impairment are variable and often depend on the particular social setting of the individual. For example, Mild Cognitive Impairment may significantly impair an-individual's ability to perform a complex job but not a less demanding one.

Cognitive or degenerative brain disorders are characterized clinically by progressive loss of memory, cognition, reasoning, judgment and emotional stability that gradually leads to profound mental deterioration and ultimately death. It is generally believed that the disease begins a number of years before it manifests itself in the mild cognitive changes that are the early signs of Alzheimer's disease. “Dementia of the Alzheimer's Type” begins gradually, and is usually diagnosed after other specific causes have been ruled out. Diagnostic criteria for Dementia of the Alzheimer's Type include the development of multiple cognitive deficits manifested by both memory impairment (anterograde or retrograde, i.e., impaired ability to learn new information or to recall previously learned information); and one or more of the following cognitive disturbances: aphasia (language disturbance), apraxia (impaired ability to carry out motor activities despite intact motor function), agnosia (failure to recognize or identify objects despite intact sensory function), disturbance in executive functioning (i.e., planning, organizing, sequencing, and abstracting); where these cognitive deficits each cause significant impairment in social or occupational functioning and represent a significant decline from a previous level of functioning. The course is characterized by gradual onset and continuing cognitive decline, and the cognitive deficits are not due to another condition that causes progressive deficits in memory and cognition (e.g., cerebrovascular disease, brain tumor, hypothyroidism, vitamin B or folic acid deficiency, niacin deficiency, hypercalcemia, neurosyphilis, HIV infection, or chemical exposure). The cognitive disturbance may be accompanied by a behavioral disturbance, such as wandering, aggression, or agitation, or a psychological disturbance, such as depression or psychosis. See “Diagnostic and Statistical Manual of Mental Disorders,” 4th Ed., Text Revision, by American Psychiatric Association (2000). For example, the National Institute of Neurological and Communicative Disorders and Stroke-Alzheimer's Disease and the Alzheimer's Disease and Related Disorders Association (NINCDS-ADRDA) criteria can be used to diagnose Alzheimer's Disease (McKhann et al., 1984, Neurology 34:939-944). The patient's cognitive function can be assessed by the Alzheimer's Disease Assessment Scale-cognitive subscale (ADAS-cog; Rosen, et al., 1984, Am. J. Psychiatry 141:1356-1364).

Alzheimer's Disease is the prototype of a cortical degenerative disease. A major component of the presenting symptoms is usually subjective complaints of memory difficulty, language impairment, dyspraxia, at which point diagnosis is primarily based on exclusion of other possible etiologies for dementia. No features of the physical examination or laboratory evaluation are pathognomonic for dementia of the Alzheimer's type. Some studies have apparently discriminated patients with dementia of the Alzheimer's type from patients with dementia of other etiologies and from normal controls by using techniques such as EEG, MRI, and SPECT, but these studies have been difficult to replicate consistently, and at present, brain-imaging studies are best used to exclude other identifiable causes.

A variety of diagnostic tests have been developed for Alzheimer's disease. Clinical criteria have been verified prospectively in autopsy studies and have been found to be highly specific although only moderately sensitive. Implementation of the criteria requires extensive evaluation, including an informant-based history, neurological examination, neuropsychological testing, and laboratory, and neuroimaging data. Alzheimer's disease is characterized pathologically by generalized atrophy of the cerebral cortex and by neurofibrillary tangles, neuritic (amyloid) plaques, and granulovacuolar degeneration. Although plaques and tangles may be detected in the brains of the elderly without Alzheimer's disease, they are more numerous in patients with dementia. Controversy remains whether brains with plaques from individuals without Alzheimer's diseasewere “normal variations” or early pathological signs of incipient disease. A definitive diagnosis ultimately requires both the characteristic dementia in life and the characteristic pathology after death.

The natural course of Alzheimer's disease is exacerbation and progression of clinical symptomatology. Brain degeneration as measured by in vivo imaging techniques such as MRI has not been found to correlate closely with the state of clinical disease. The final common clinical picture is of a bedridden patient, wholly dependent on others for all basic functions, even for turning in bed. Nutrition can often be provided only by nasogastric or gastrointestinal tubes.

The study of the pathobiology of Alzheimer's disease has identified at least four chromosomal loci associated with familial cases; the degeneration of central neurochemical systems, especially basal forebrain structures related to acetylcholine-mediated neurotransmission; factors associated with the formation of plaques and tangles; and exogenous (e.g., infectious and toxic) processes that may contribute to the development of sporadic cases. Although amyloid itself is a normal brain product, excessive amounts of oligomeric or fibrillar forms of Aβ may be neurotoxic.

For Alzheimer's disease, advanced age and a family history of the disease are the greatest important risk factor. A family history of Down's syndrome or of hematological malignancies, such as leukemia, myelolymphoma, or Hodgkin's disease, is also associated with an increased risk for Alzheimer's disease. For Alzheimer's disease, other risk factors identified tentatively in recent years include female sex, a past history of head trauma, and lower education. Vascular dementias are highly associated with the risk factors for cerebrovascular disease. Those factors include hypertension (especially with systolic pressures greater than 160 mmHg), cardiac disease, transient ischemic attacks, diabetes mellitus, carotid bruits, and sickle cell disease. Obesity, a sedentary lifestyle, tobacco use, alcohol consumption, and elevated serum cholesterol and lipid levels may also be risk factors for cerebrovascular disease.

A general physical examination is a routine component of the workup for Alzheimer's disease. It may reveal evidence of systemic disease causing brain dysfunction, such as an enlarged liver and hepatic encephalopathy, or it may demonstrate systemic disease related to particular CNS processes. Focal neurological findings, such as asymmetrical hyperreflexia or weakness, are seen more often in vascular than in degenerative diseases. Frontal release signs and primitive reflexes, while suggesting pathology in the frontal lobe, are present in many disorders and often point to a greater extent of progression. The first step in the diagnosis of Alzheimer's disease is to exclude delirium, which may be distinguished from dementia by its cardinal feature: disturbance of consciousness. Level of consciousness or arousal should be determined to be stable before a diagnosis of Alzheimer's diseasecan be made with confidence. It should also be distinguished from focal or specific cognitive impairments, such as those seen in aphasic or amnestic patients. Mood disorders can present with cognitive symptoms, particularly in the dementia of depression or pseudodementia. A history of a mood disorder or a current disturbance in neurovegetative function indicates the possibility of a major depressive disorder.

The course and prognosis of a dementia syndrome vary with its cause. Alzheimer's disease does not necessarily equal progressive deterioration, although many of the pathobiological processes underlying dementia are degenerative. The rate of progression may vary within families or from individual to individual. Age at onset is an important feature of Alzheimer's disease, the most common cause of dementia in the United States. Onset usually occurs after age 60 years and the prevalence increases exponentially with each successive decade, although cases have been reported in patients as young as 30 years. Familial forms of dementia of the Alzheimer's type appear to have an earlier age at onset. Cerebrovascular disease, the second most common cause of Alzheimer's disease, is associated with an earlier age at onset overall.

As a class, the dementias can be distinguished to some extent by their course, especially earlier in the disease process. Degenerative dementias are insidious in onset and gradually progressive. Despite the clinical rule of a steadily progressive course in dementia of the Alzheimer's type, some individuals may reach a plateau for several years in the overall functional impairment before progression resumes and continues on to death. Vascular dementias may follow a stepwise pattern, in which new deficits appear abruptly and are associated with new vascular events, but the vascular dementias also often have an insidious onset and a slow but steadily progressive course. The first step in the treatment of dementia is verification of the diagnosis. Preventive pharmacological agents include antihypertensive, anticoagulant, or antiplatelet agents. Blood pressure control has been demonstrated to improve cognitive function in patients with vascular dementia, but it should be noted that antihypertensive beta-blocking agents have been associated with exaggeration of cognitive impairment. Angiotensin-converting enzyme (“ACE”) inhibitors and diuretics have not been linked to the exaggeration of cognitive impairment and are thought to lower blood pressure without affecting cerebral blood flow (cerebral blood flow is presumed to correlate with cognitive function). Surgical removal of carotid plaques may prevent subsequent vascular events in carefully selected patients.

Numerous neurotransmitters, including acetylcholine, dopamine, norepinephrine, GABA, and serotonin, and several neuropeptides, including somatostatin and substance P, are decreased in dementia. Multiple neuropharmacological strategies have been devised in the hope of replenishing the deficient neurotransmitters. Replacement therapy for acetylcholine has been the most common and widely publicized strategy. Efforts at replenishment have included the use of acetylcholine precursors, e.g., choline salycilate (Arthropan™, Purdue Pharma, L. P., Stamford, Conn.) and lecithin from polyenylphosphatidylcholine (Phoschol™, Nutrasal LLC, Oxford, Conn.); cholinergic agonists, e.g., arecoline (methyl N-methyltetrahydronicotinate, and cholinesterase inhibitors, such as described herein. Instead of targeting a neurotransmitter defect, other strategies are directed toward neuronal protection and regeneration. Selegiline (Eldepryl™, Somerset, Tampa, Fla.), a monoamine oxidase (“MAO”) type B (“MAO-B”) inhibitor, slows the progression of Parkinson's disease, presumably by limiting endogenous generation of destructive oxidative products, and the same effect may be used therapeutically in the treatment of Alzheimer's patients. Similar antioxidant treatments are being used experimentally with other dementias, including Huntington's disease and vascular dementia. Naloxone (Narcan), an opiate antagonist, is thought to have possible application in vascular dementia based on animal studies in which it was demonstrated to decrease the sequelae of cerebral ischemia. See, e.g., C. Stowe, et al., Ann. Pharmacother. 27, 447-48 (1993). Nerve growth factor is being studied as a means of promoting neural regeneration or sprouting.

As used herein, “treatment” of a subject includes the application or administration of a composition of the invention to a subject, or application or administration of a composition of the invention to a cell or tissue from a subject, who has a amyloid-β related disease or condition, has a symptom of such a disease or condition, or is at risk of (or susceptible to) such a disease or condition, with the purpose of curing, healing, alleviating, relieving, altering, remedying, ameliorating, improving, or affecting the disease or condition, the symptom of the disease or condition, or the risk of (or susceptibility to) the disease or condition. The term “treating” refers to any indicia of success in the treatment or amelioration of an injury, pathology or condition, including any objective or subjective parameter such as abatement; remission; diminishing of symptoms or making the injury, pathology or condition more tolerable to the subject; slowing in the rate of degeneration or decline; making the final point of degeneration less debilitating; improving a subject's physical or mental well-being; or, in some situations, preventing the onset of dementia. The treatment or amelioration of symptoms can be based on objective or subjective parameters; including the results of a physical examination or a psychiatric evaluation. For example, the methods of the invention successfully treat a subject's dementia by slowing the rate of or extent of cognitive decline.

Also, the invention relates to a method for preventing or inhibiting amyloid production in a subject. For example, such a method comprises administering to a subject a therapeutically effective amount of a pharmaceutical composition of the invention capable of reducing the concentration of Aβ, such that amyloid production or accumulation is prevented or inhibited.

In another aspect, the invention relates to a method where at least the first compound is for preventing, reducing, or inhibiting amyloid production in a subject. For example, such a method comprises administering to a subject a therapeutically effective amount of a pharmaceutical composition capable of inhibiting Aβ accumulation, such that Aβ amyloidosis is prevented, reduced, or inhibited.

“Inhibition” of amyloid deposition includes preventing or stopping of amyloid formation, e.g., fibrillogenesis, clearance of soluble Aβ from brain, inhibiting or slowing down of further amyloid deposition in a subject with amyloidosis, e.g., already having amyloid deposits, and reducing or reversing amyloid fibrillogenesis or deposits in a subject with ongoing amyloidosis. Inhibition of amyloid deposition is determined relative to an untreated subject, or relative to the treated subject prior to treatment, or, e.g., determined by clinically measurable improvement, e.g., or in the case of a subject with brain amyloidosis, e.g., an Alzheimer's or cerebral amyloid angiopathy subject, stabilization of cognitive function or prevention of a further decrease in cognitive function (i.e., preventing, slowing, or stopping disease progression), or improvement of parameters such as the concentration of Aβ or tau in the CSF.

“Modulation” of amyloid deposition includes both inhibition, as defined above, and enhancement of amyloid deposition or fibril formation. The term “modulating” is intended, therefore, to encompass prevention or stopping of amyloid formation or accumulation, inhibition or slowing down of further amyloid aggregation in a subject with ongoing amyloidosis, e.g., already having amyloid aggregates, and reducing or reversing of amyloid aggregates in a subject with ongoing amyloidosis; and enhancing amyloid deposition, e.g., increasing the rate or amount of amyloid deposition in vivo or in vitro. Amyloid-enhancing compounds may be useful in animal models of amyloidosis, for example, to make possible the development of amyloid deposits in animals in a shorter period of time or to increase amyloid deposits over a selected period of time. Amyloid-enhancing compounds may be useful in screening assays for compounds which inhibit amyloidosis in vivo, for example, in animal models, cellular assays and in vitro assays for amyloidosis. Such compounds may be used, for example, to provide faster or more sensitive assays for compounds. In some cases, amyloid enhancing compounds may also be administered for therapeutic purposes, e.g., to enhance the deposition of amyloid in the lumen rather than the wall of cerebral blood vessels to prevent CAA. Modulation of amyloid aggregation is determined relative to an untreated subject or relative to the treated subject prior to treatment.

In an embodiment, the method is used to treat Alzheimer's disease (e.g., sporadic or familial Alzheimer's disease). The method may also be used prophylactically or therapeutically to treat other clinical occurrences of amyloid-β deposition, such as in Down's syndrome individuals and in subjects with hereditary or sporadic cerebral amyloid angiopathy (“CAA”), which lead to cerebral hemorrhage (or hemorrhagic stroke).

Additionally, abnormal accumulation of APP and of amyloid-β protein in muscle fibers has been implicated in the pathology of sporadic inclusion body myositis (“IBM”) (Askanas, et al., Proc. Natl. Acad. Sci. USA 93, 1314-19 (1996); Askanas, et al., Current Opinion in Rheumatology 7, 486-96 (1995)). Accordingly, the compounds of the invention can be used prophylactically or therapeutically in the treatment of disorders in which amyloid-β protein is abnormally deposited at non-neurological locations, such as treatment of IBM by delivery of the compounds to muscle fibers.

Additionally, it has been shown that Aβ is associated with abnormal extracellular deposits, known as drusen, that accumulate along the basal surface of the retinal pigmented epithelium in individuals with age-related macular degeneration (“ARMD”). ARMD is a cause of irreversible vision loss in older individuals. It is believed that Aβ deposition could be an important component of the local inflammatory events that contribute to atrophy of the retinal pigmented epithelium, drusen biogenesis, and the pathogenesis of ARMD (Johnson, et al., Proc. Natl. Acad. Sci. USA 99(18), 11830-5 (2002)).

The present invention therefore relates to the use of a first agent, e.g., an alkanesulfonic acid compound, in the prevention or treatment of amyloid-β related diseases, including, inter alia, Alzheimer's disease, cerebral amyloid angiopathy, inclusion body myositis, Down's syndrome, Mild Cognitive Impairment, and macular degeneration, in combination with a second therapeutic agent.

Accordingly, the invention relates to methods employing and compositions including substituted or unsubstituted alkylsulfonic acids that are substituted or unsubstituted straight-chain alkylsulfonic acids, substituted or unsubstituted cycloalkylsulfonic acids, and substituted or unsubstituted branched-chain alkylsulfonic acids. Also, it is noted that the term “alkylsulfonic acid” as used herein is to be interpreted as being synonymous with the term “alkanesulfonic acid.”

In another embodiment, the subject has mild cognitive impairment (MCI), which is a condition characterized by a state of mild but measurable impairment in thinking skills, but is not necessarily associated with the presence of dementia. MCI frequently, but not necessarily, precedes Alzheimer's disease. It is a diagnosis that has most often been associated with mild memory problems, but it can also be characterized by mild impairments in other thinking skills, such as language or planning skills. However, in general, an individual with MCI will have more significant memory lapses than would be expected for someone of their age or educational background. As the condition progresses, a physician may change the diagnosis to Mild-to-Moderate Cognition Impairment, as is well understood in this art.

In one embodiment, the pharmaceutical compositions disclosed herein prevent or inhibit amyloid protein assembly into insoluble fibrils which, in vivo, are deposited in various organs, or it reverses or favors deposition in subjects already having deposits. In another embodiment, the agent may also prevent the amyloid protein, in its soluble, oligomeric form or in its fibrillar form, from binding or adhering to a cell surface and causing cell damage or toxicity. In yet another embodiment, the agent may block amyloid-induced cellular toxicity or microglial activation. In another embodiment, the agent may block amyloid-induced neurotoxicity.

The pharmaceutical compositions of the invention may be administered therapeutically or prophylactically to treat diseases associated with amyloid-β fibril formation, aggregation or deposition. The pharmaceutical compositions of the invention may act to ameliorate the course of an amyloid-β related disease using any of the following mechanisms (this list is meant to be illustrative and not limiting): slowing the rate of amyloid-β fibril formation or deposition; lessening the degree of amyloid-β deposition; inhibiting, reducing, or preventing amyloid-β fibril formation; inhibiting neurodegeneration or cellular toxicity induced by amyloid-β; inhibiting amyloid-β induced inflammation; or enhancing the clearance of amyloid-β from the brain; or enhancing the catabolism or degradation of amyloid-β; or lowering the levels of amyloid-β in the CSF; or modulating the levels of amyloid-β in the plasma. Another way of decreasing Aβ could be that these compounds act on secretases so that Aβ levels are reduced (as seen with proteoglycans).

Pharmaceutical compositions of the invention may be effective in controlling amyloid-β deposition either following their entry into the brain (following penetration of the blood brain barrier) or from the periphery. When acting from the periphery, an agent of a pharmaceutical composition may alter the equilibrium of Aβbetween the brain and the plasma so as to favor the exit of Aβfrom the brain. An increase in the exit of Aβfrom the brain would result in a decrease in Aβbrain concentration and therefore favor a decrease in Aβdeposition. Alternatively, agents that penetrate the brain could control deposition by acting directly on brain Aβ, e.g., by maintaining it in a non-fibrillar form or favoring its clearance from the brain or enhancing its degradation rate in the brain or in the peripheral organs. These agents may also prevent Aβin the brain from interacting with a cell surface and therefore prevent neurotoxicity or inflammation.

The compositions of the invention may be administered therapeutically or prophylactically to treat diseases associated with amyloid-β fibril formation, aggregation, or deposition. The compositions of the invention may act to ameliorate the course of an amyloid-β related disease by a variety of mechanisms. In one embodiment, the pharmaceutical compositions disclosed herein prevent or inhibit amyloid protein assembly into insoluble fibrils which, in vivo, are deposited in various organs, or it favors plaque clearance or slows deposition in subjects already having deposits. In another embodiment, the pharmaceutical compositions may also prevent the amyloid protein, in its soluble, oligomeric form or in its fibrillar form, from binding or adhering to a cell surface and causing cell damage or toxicity. In yet another embodiment, the pharmaceutical compositions may block amyloid toxicity. In other embodiments, the agent may act be slowing the rate of amyloid-β fibril formation or deposition. In yet another embodiment, the agent may lessen the degree of amyloid-β deposition. Still other examples include inhibiting, reducing, or preventing amyloid-β fibril formation; inhibiting neurodegeneration or cellular toxicity induced by amyloid-β; inhibiting amyloid-β induced inflammation; or enhancing the clearance of amyloid-β from the brain brain or enhancing its degradation rate in the brain or in the peripheral organs.

At least one of the therapeutic agents of the invention may be effective in controlling amyloid-β deposition either following their entry into the brain (following penetration of the blood brain barrier) or from the periphery. When acting from the periphery, an agent may alter the equilibrium of Aβ between the brain and the plasma so as to favor the exit of Aβ from the brain. An increase in the exit of Aβ from the brain would result in a decrease in Aβ brain concentration and therefore favor a decrease in Aβ deposition. Alternatively, agents that penetrate the brain could control deposition by acting directly on brain Aβ, e.g., by maintaining it in a non-fibrillar form or favoring its clearance from the brain, or by favoring catabolism or acting on secretase so that Aβ production is reduced.

In one aspect, the invention relates to pharmaceutical compositions comprising two agents, each of which exerts a therapeutic effect when administered to a subject in need thereof, and is useful in treating or preventing a neurological disease. The first agent of a pharmaceutical composition of the invention is selected from substituted and unsubstituted alkanesulfonic acids and alkanesulfuric acids that are useful for treating or preventing an amyloid-β related disease. The second agent is therapeutic, i.e., its function is beyond that of an inactive ingredient, such as a pharmaceutical carrier, preservative, diluent, or buffer. The second agent may be useful in treating or preventing an amyloid-β related disease or another neurological disease. The first and second agents may exert their biological effects by similar or unrelated mechanisms of action; or either one or both of the first and second agents may exert their biological effects by a multiplicity of mechanisms of action. A pharmaceutical composition may also comprise a third agent, or even more yet, wherein the third (and fourth, etc.) agent has the same characteristics of a second agent.

The invention also relates to packaged pharmaceutical products containing two agents, each of which exerts a therapeutic effect when administered to a subject in need thereof, and is useful in treating or preventing a neurological disease. The first agent of a pharmaceutical composition of the invention may be selected from substituted and unsubstituted alkanesulfonic acids and alkanesulfuric acids that are useful for treating or preventing an amyloid-β related disease. The second agent is therapeutic, i.e., its function is beyond that of an inactive ingredient, such as a pharmaceutical carrier, preservative, diluent, or buffer. The second agent may be useful in treating or preventing an amyloid-β related disease or another neurological disease. The agents may exert their biological effects by similar or unrelated mechanisms of action; or either one or more than one of the agents may exert their biological effects by a multiplicity of mechanisms. A pharmaceutical composition may also comprise a third agent, or even more agents yet, wherein the third (and fourth, etc.) agent has the same characteristics of a second agent. In some cases, the individual agents may be packaged in separate containers for sale or delivery to the consumer. The agents of the invention may be supplied in a solution with an appropriate solvent or in a solvent-free form (e.g., lyophilized). Additional components may include acids, bases, buffering agents, inorganic salts, solvents, antioxidants, preservatives, or metal chelators. The additional kit components are present as pure compositions, or as aqueous or organic solutions that incorporate one or more additional kit components. Any or all of the kit components optionally further comprise buffers.

The present invention also includes packaged pharmaceutical products containing a first agent in combination with (e.g., intermixed with) a second agent. The invention also includes a pharmaceutical product comprising a first agent packaged with instructions for using the first agent in the presence of a second agent or instructions for use of the first agent in a method of the invention. The invention also includes a pharmaceutical product comprising a second or additional agents packaged with instructions for using the second or additional agents in the presence of a first agent or instructions for use of the second or additional agents in a method of the invention. Alternatively, the packaged pharmaceutical product may contain at least one of the agents and the product may be promoted for use with a second agent.

This invention also relates to a pharmaceutical composition for the treatment of a condition selected from mental retardation, developmental disorders, disruptive behavioral disorders, organic mental disorders (including dementia and psychoactive substance induced organic mental disorders), psychoactive substance abuse disorders, mood disorders, anxiety disorders, somatoform disorders, dissociative disorders, attention deficit disorder, schizophrenia and personality disorders in a human, comprising an acetylcholine esterase inhibitor. The second agent may also be a neurotransmitter-release enhancer, i.e., they possess the ability to enhance or stimulate the release of neurotransmitters such as acetylcholine, dopamine, and serotonin in humans. They are therefore able to function as therapeutic agents in the treatment of a variety of conditions in humans, the treatment or prevention of which can be effected or facilitated by the enhancement or stimulation of acetylcholine, dopamine, or serotonin release. Such conditions include Alzheimer's disease, age associated memory impairment and Mild Cognitive Impairment, and Parkinson's disease. They also include mental retardation, developmental disorders, disruptive behavioral disorders, organic mental disorders (including dementia and psychoactive substance induced organic mental disorders), psychoactive substance abuse disorders, mood disorders, anxiety disorders, somatoform disorders, dissociative disorders, attention deficit disorder, schizophrenia, and personality disorders.

The invention is also ideally suited for the treatment of familiar or hereditary forms of Alzheimer's disease because, for example, therapeutic prophylactic pharmaceutical treatment could be initiated earlier in a patient's life. Presently, all therapeutic regimens that are commercially available treat only the symptoms of Alzheimer's disease, as explained elsewhere herein. The present invention provides methods and compositions, however, that treat the underlying etiology of the disease itself, and therefore may be used in a prophylactic manner. The biological processes that give rise to Alzheimer's disease may occur in a person for some time before clinically observable symptoms arise. Ordinarily, according to current medical science, such a period in a person's life would be undetectable and treatment with current medicines would be useless. For people with an identified predisposition for developing Alzheimer's disease compositions of the present invention may delay the onset of symptoms.

Furthermore, the invention pertains to any novel chemical agent described herein. That is, the invention relates to novel agents, and novel methods of their use as described herein, including those coumpounds that may be within the scope of the Formulae disclosed herein, and which are not disclosed in the cited Patents and Patent Applications.

Example Therapeutic Drug Targets for the Treatment of Alzheimer's Disease

In the pharmaceutical compositions of the invention, an alkanesulfonic acid compound may be combined with a second agent that is also useful in the treatment of Alzheimer's disease. In general, the second agent may be any therapeutic drug. A “therapeutic drug” is a drug or medicine administered for legitimate or medically-approved, therapeutic or diagnostic, purpose. Therapeutic drugs may be available over-the-counter or by prescription. Examples of therapeutic drugs include an adrenergic, anti-adrenergic, anti-androgen, anti-anginal, anti-anxiety, anticonvulsant, antidepressant, anti-epileptic, antihyperlipidemic, antihyperlipoproteinemic, antihypertensive, anti-inflammatory, antiobessional, antiparkinsonian, antipsychotic, adrenocortical steroids, adrenocortical suppressant, aldosterone antagonists, amino acids, anabolic steroids, analeptic agents; androgens; blood glucose regulators, cardioprotectants; cardiovascular agents; cholinergic agonist and antagonists, cholinesterase deactivators or inhibitors, cognition adjuvants and enhancers, dopaminergic agents, enzyme inhibitors, estrogen and related steroid hormones, free oxygen radical scavengers, GABA agonists, glutamate antagonists, hormones, antihypocholesterolemic agents, hypolipidemic agents, hypotensive agents, immunizing agents, immunostimulants, monoamine oxidase inhibitors, neuroprotective agents, NMDA antagonists, AMPA antagonists, competitive and non-competitive NMDA antagonists, opioid antagonists, potassium channel openers, non-hormonal sterol derivatives, post-stroke and post-head trauma treatments, prostaglandins, psychotropics, relaxants, sedatives, sedative-hypnotics, selective adenosine antagonists, serotonin antagonists, serotonin inhibitors, selective serotonin uptake inhibitor, serotonin receptor antagonists, sodium and calcium channel blockers, steroids, stimulants, thyroid hormones and inhibitors, etc.

In one aspect, the invention pertains to a pharmaceutical composition comprising an alkanesulfonic acid and a second agent that is useful in the treatment or prevention of Alzheimer's disease. The second agent may be curative, ie., modulate the causative agents of Alzheimer's disease, or it may be palliative, i.e., alleviate the symptoms of the disease, e.g., by enhancing memory or improving cognitive function. The second agent may be a drug that is useful in the treatment of Alzheimer's disease itself, or it may be used to treat a condition associated with Alzheimer's disease, e.g. a secondary condition, or it may be a drug commonly prescribed to Alzheimer's disease subjects.

According to current understanding of the natural history of Alzheimer's disease, several different drug targets have been identified. A pharmaceutical composition of the invention may comprise a second agent that is specific for any one of the biological processes giving rise to the clinical presentation of Alzheimer's disease. The invention is not to be considered bound by any particular theory of the etiology of Alzheimer's disease, and so the second agent according to the pharmaceutical compositions of the invention may be any agent that either by itself or in combination with an alkanesulfonic acid is empirically observed to be efficacious in the treatment or prevention of Alzheimer's disease. Nevertheless, a summary of the biological processes believed to give rise to Alzheimer's disease itself or the symptoms thereof is useful inasmuch as any one of these biological processes may be modulated by a second agent in a pharmaceutical composition of the invention.

The language “in combination with” a second agent or treatment includes co-administration of an alkanesulfonic acid, administration of an alkanesulfonic acid first, followed by the second, or treatment and administration of the second agent first, followed by an alkanesulfonic acid.

The condition associated with Alzheimer's disease may be a symptom characteristic of Alzheimer's disease, for example, hypothyroidism, cerebrovascular or cardiovascular disease, memory loss, anxiety, or a behavioral dysfunction (e.g., apathy, aggression, or incontinence); a psychological condition or a neurological condition. The neurological condition may be Huntington's disease, amyotrophic lateral sclerosis, acquired immunodeficiency, Parkinson's disease, aphasia, apraxia, agnosia, Pick disease, dementia with Lewy bodies, altered muscle tone, seizures, sensory loss, visual field deficits, incoordination, gait disturbance, transient ischemic attack or stroke, transient alertness, attention deficit, frequent falls, syncope, neuroleptic sensitivity, normal pressure hydrocephalus, subdural hematoma, brain tumor, posttraumatic brain injury, or posthypoxic damage. The psychological condition is depression, delusions, illusions, hallucinations, sexual disorders, weight loss, psychosis, a sleep disturbance such as insomnia, behavioral disinhibition, poor insight, suicidal ideation, depressed mood or irritability, anhedonia, social withdrawal, or excessive guilt.

The second agent, i.e., a therapeutic drug may be a psychotropic medication, antidepressant (a selective serotonin reuptake inhibitor, atypical antidepressant), antipsychotic, appetite stimulants, or another drug used to treat a condition associated with Alzheimer's disease, or a a nutritive supplement that is a precursor of acetylcholine (lecithin or choline), Ginkgo biloba, acetyl-L-carnitine, idebenone, propentofylline, or a xanthine derivative.

Antidepressants include selective serotonin reuptake inhibitors such as citalopram (Celexa); escitalopram (Lexapro); fluoxetine (Prozac™); alprazolam (Xanax™), triptolene, fluvoxamine (Luvox™); paroxetine (Paxil™); sertraline (Zoloft™); mixed norepinephrine/dopamine reuptake inhibitors such as bupropion (Wellbutrimm); drugs with mixed serotonin effects such as nefazodone (Serzone™) and trazodone (Desyrel™); mixed serotonin/norepinephrine reuptake inhibitors venlafaxine (Effexomm); monoamine oxidase inhibitors including phenelzine (Nardil™) and tranylcypromine (Parnate™); and tetracyclic antidepressants such as maprotiline, mirtazapine (Remeron™), amitriptyline (Elavil™), amoxapine, clomipramine (Anafranil™), desipramine (Norpramin™), doxepin (Sinequan™), imipramine (Tofranil™), nortriptyline (Aventyl™, Pamelor™), protriptyline (Vivactil™), and trimipramine (Surmontil™). Antidepressants: tricyclic and selective serotonin reuptake inhibitors; fluoxetine (Prozac™); sertraline (Zoloft™); paroxetine (Paxil™); citalopram (Celexa™); nortriptyline; moclobemide; miratazepine; Nardil™; Pamate™; Manerix™; Tofranil™; Elavil™; Sinequanm; Surmontil™; Anafranil™; Norpramine™; Aventyl™; Effexorm; Serzone™; Welbutrin™; Desyrel™; and Remeromm.

Antipyschotics include aripiprazole (Abilify™), clozapine (Clozaril™), olanzapine (Zyprexa™), quetiapine (Seroquel™), risperidone (Risperdal™), and ziprasidone (Geodomm). Antipsychotics: conventional and atypical; olanzapine (Zyprexa™); quetiapine (Seroquel™); haloperidol (Haldol™); risperidone (Risperidal™); zuclopenhixol (Clopixol™); ziprazidone; thioridazine (Mellaril™, Sandoz Pharmaceutical Corp., now Novertis, Basel, Switzerland); clozapine (Clozaril™); olanzapine; and lithium.

Still other examples of drugs that may be a second agent include: cholinesterase inhibitors: huperzine A; antidepressants: venlafaxine, desipramine, nefazodone, trazodone, citalopram, escitalopram, nortriptyline, paroxetine; anti-agitation/mood-stabilizing agents, or anti-epileptics for convulsions: carbamazepine, gabapentin, primidone, phenyloin, clonazepam, valproic acid; neuroleptics: ziprasidone, haloperidol, risperidone, olanzapine, quetiapine; anti-inflammatory/immunomodulating drugs: colchicine, dapsone, meloxicam, nimesulide, flurbiprofen, cyclophosphamide, methotrexate, β-interferon, gamma-interferon, etanercept, infliximab; chelators: penicillamine; hormonal therapies: leuprolide; homocysteine reducing vitamins: metafolin; antioxidants: lipoic acid, selegeline; anti-thrombotics: aspirin; others: carbidopa, levodopa (Sinemet™), folic acid; anxiolytics hypnotics, and sedatives (anti-anxiety drugs): clonazepan, lorazaparn, oxazepam, bupropion., benzodiazepines including diazepam (Valium™, Roche Products, Hoffmann-La Roche Inc. (Roche), Nutley, N.J.), chlordiazepoxide (Librium™ or Libritabs™, F. Hoffman-LaRoche Ltd., Basel, Switzerland), lorazepam (Ativanm, Wyeth, Madison, N.J.), oxazepam (Seraxm, Wyeth, Madison, N.J.), buspirone (Buspar™), zolpidem (Ambien™, bromazepam (Lectopam™), alprazolam (Xanax™), clonazepam (Rivotril™), flurazepam (Dalmane™), temazepam (Restoril™), triazolam (Halcion™), nitrazepam (Mogadon™); and anti-Parkinson including benztopine (Cogentin™) and procyclidine (Kernadrin™); ACE inhibitors such as captopril (Capoten™), captopril in combination with hydrochlorothiazide (Capozide™), enalapril maleate (Vasotec™), enalaprilat, enalapril maleate/hydrochlorothiazide combination (Vaseretic™), fosinopril (Monopril™), lisinopril (Zestril™), ramipril (Altace™), epi-captopril, alacepril, quinapril, perindopril, delapril, cilazapril, pivalopril, rentiapril, zofenopril, zofenoprilat; analgesics; anesthetics; antienemic and mineral or vitamin dietary supplements; antibiotics; antidiarrheal; antiepileptics; antigout; antihistamines; antihypertensives; antiinflammatory and antirheumatoid; antipruritics; antithrombotics; beta-blockers; calcium channel blockers; cardioactive glycosides; corticosteroids such as prednisone (Deltasone™) and dexamethasone (Decadromm); antitussives; diuretics; antidiabetes; antiseptics; antiinfectives; laxatives; psychoanaleptics and psycholeptics; serum lipid-reducing drugs; sex hormones; thyroid hormones; and urological drugs.

Suitable second agents further include therapeutic drugs that are useful in the treatment of Parkinson's Disease or its symptoms, which second agents include: (a) cabidopa and levodopa compositions; (b) dopamine agonists such as bromocriptine mesylate (Parlodel™), pergolide mesylate (Permaxm™), (+)-4-propyl-9-hydroxynaphthoxazine, and apomorphine; (c) anticholinergic medications such as benztropine mesylate (Cogentin™), and biperiden; (d) antihistamines such as orphenadrine citrate (Norflex™, Norgesic™); (e) tricyclic antidepressants such as amitriptyline HCl (Elavil™), amitriptyline HCl/perphenazine combinations (Etrafon™), arnitriptyline and chlordiazepoxide combinations (Limbitrol™), nortriptyline HCl (Pamelor™), imipramine, and doxepin; (f) serotonin reuptake inhibitor antidepressants such as fluoxetine HCl (Prozac™), and sertraline (Zoloftm); (g) beta blocker agents such as propranolol HCl (Inderal™), pindolol (Visken™), metoprolol tartrate (Lopressor™), metoprolol succinate (Toprol XL™), and atenolol (Tenormimm); (h) selegiline (Eldepryl™) or selegiline in combination with tocopherol; (i) D-cycloserine with or without a cholinesterase inhibitor co-agent; (j) neurotransmission enhancer drugs such as lisuride; (k) peripheral decarboxylase inhibitors such as benserazide used in combination with levodopa; (1) N-methyl-D-aspartate glutamate receptor antagonists such as trihexyphenidyl (Artane™), ethopropazine (Paridol™), procyclidine (Kemadrin™), diphenhydramine (Benadryl™), dizocilpine (Neurogard™), amantadine (Symmetrel™), memantine, and milacemide; (m) tacrine (Cognex™), optionally with phosphatidylcholine co-agent; (n) (+/−)-9-amino-1,2,3,4-tetrahydroacridin-1-ol; (O) lazabemide; (p) tiapride; and (q) antioxidant agents which may be used in combination such as ascorbic acid, N-acetylcysteine, penicillamine, and cysteamine.

Suitable second agents further include therapeutic drugs that are useful in the treatment of Alzheimer's Disease or its symptoms, which second agents include: (a) vasodilators or other nootropic direct brain metabolic enhancer drugs such as idebenone, propentophylline, pentoxifylline, citicoline, ebiratide, vinpocetine (Cavinton™), bromvincamine, cyclandelate, isoxsuprene, nafronyl, papaverine, suloctidil, vinburnine, vincamine, vindeburnol, naloxone, ethyl 5-isopropyloxy-4-methyl-β-carboline-3-carboxylate, N′-methyl-α-carboline-3-carboxamide, methyl 6,7-dimethoxy-4-ethyl-p-carboline-3-carboxylate, ethyl 5-methoxy-4-ethyl-β-carboline-3-carboxylate, ifenprodil tartrate, piracetam, aniracetam, pyroglutamic acid, tenilsetam, pramiracetam, oxiracetam, rolziracetam, razobazam, exifone, rolipram, sabeluzole, nimodipine (Nimotop™), flunarizine, nicergoline (Sermion™), phosphatidylserine, etiracetam, dupracetam, and ergoloid mesylates (Hydergine™); (b) neurotransmission enhancer drugs such as amantadine (Symmetrel™), calcium hopantenate, lisuride, and indeloxazine; (c) tiapride; (d) psychotherapeutic drugs such as haloperidol (Haldol™), bromperidol, thioridazine (Mellaril™), thiothixene (Navane™), fluphenazine (Prolixin™), perphenazine in amitriptyline/perphenazine combinations (Etrafon™), and molindone (Moban™); (e) acetylcholinesterase inhibitors such as physostigmine (Antilirium™), optionally with phosphatidylcholine co-agent, heptylphysostigmine, donepezil (Aricept™), tacrine (Coanex™), optionally with phosphatidylcholine co-agent, (+/−)-9-amino-1,2,3,4-tetrahydroacridin-1-ol, metrifonate, velnacrine (Mentane™), phenylmethylsulfonyl fluoride, methanesulfonyl fluoride, huperzine A, huperzine B, edrophonium chloride, galanthamine, and miotine; (f) calcium channel blocker agents such as diltiazem (Cardizemm), verapamil (Calan™), nifedipine (Procardia™), nifedipine (Procardia, nicardipine (Cardene™), isradipine (DynaCire™), amlodipine (Norvasc™), felodipine (Plendil™); (g) biogenic amines and agents related thereto such as clonidine (Catapres™), guanfacine (Tenex™), alaproclate, fipexide, zimeldine, and citalopram; (h) antirage drugs such as propranolol (Inderal™), carbamazepine (Tegretol™), and fluoxetine; (i) minor tranquilizers such as benzodiazepine agents including diazepam (Valium™), lorazepam (Ativan™), prazepam (Centrax™), chlordiazepoxide (Libritabs™), chlordiazepoxide/clidinium combination (Librax™), chlordiazepoxide/amitriptyline combination (Limbitrol™), chlordiazepoxide/esterified estrogen combination (Menrium™), oxazepam (Serax™), and clorazepate dipotassium (Tranxene™); (j) angiotensin-converting enzyme inhibitors such as captopril (Capoten™), captopril in combination with hydrochlorothiazide (Capozide™), enalapril maleate (Vasotec™), enalaprilat, enalapril maleate/hydrochlorothiazide combination (Vaseretic™), fosinopril (Monopril™), lisinopril (Zestril™), ramipril (Altace™), epi-captopril, alacepril, quinapril, perindopril, delapril, cilazapril, pivalopril, rentiapril, zofenopril, zofenoprilat; (k) agents which may enhance acetylcholine synthesis, storage or release such as phosphatidylcholine, 4-aminopyridine, 3,4-diaminopyridine, choline chloride, choline bitartrate, bifemelane, vesamicol, secoverine, tetraphenylurea, nicotinamide; (1) postsynaptic receptor agonists such as arecoline, oxotremorine, ethyl nipecotate, bethanechol (Urecholine™), and levacecamine; (m) ganglioside GM₁; (n) specific monoamine oxidase-A inhibitors such as moclobemide (Aurorix™); (o) N-methyl-D-aspartate glutamate receptor antagonists such as milacemide, trihexyphenidyl (Artane™), ethopropazine (Paridol™), procyclidine (Kemadrin™), diphenhydramine (Benadryl™), dizocilpine (NeuroQard™), amantadine (Symmetrel™), and memantine; (p) nonsteroidal anti-inflammatory agents such as those recognized for treatment of rheumatoid arthritis, including flurbiprofen (Ansaid™), aspirin, mesalamine (Asacol™), phenylbutazone (Butazolidimm), sulindac (Clinoril™), penicillamine (Cuprimine™), oxaprozin (Daypro™), salsalate (Disalcid™), diflunisal (Dolobid™), piroxicam (Feldene™), indomethacin (Indocin™), etodolac (Lodine™), meclofenamate sodium (Meclomen™), ibuprofen (Motrin™, Advil™), fenoprofen calcium (Nalfomm), naproxen sodium (Anaprox™), naproxen, ketoprofen (Orudis™), mefenamic acid (Ponstel™), nabumetone (Relafemm), auranofin (Ridaura™), tolmetin sodium (Tolectin™), ketorolac tromethamine (Toradol™), diclofenac sodium (Voltaren™), and deferoxamine mesylate (Desferal™); (q) selegiline (Eldepryl™); (r) thiamine; (s) anfacine; (t) sulbutiamine (Arcalion™); (u) antioxidant agents which may be used in combination such as ascorbic acid, N-acetylcysteine, penicillamine, cysteamine, and deferoxamine mesylate (Desferal); (v) specific monoamine oxidase-B inhibitors such as lazabemide; (w) linopirdine (Aviva™); (x) D-cycloserine; and (y) serotonergic receptor antagonists such as ketanserin, and mianserin (Mian™).

Suitable second agents further include therapeutic drugs that are useful in the treatment of aging or its symptoms, which second agents include: (a) monoamine oxidase B inhibitors such as selegiline; (b) acetylcholinesterase inhibitors such as physostigrnine (Antilirium Injectable™), heptylphysostigmine, tacrine (Cognex™), optionally with phosphatidylcholine coagent; (+/−)-9-amino-1,2,3,4-tetrahydroacridin-1-ol, velnacrine maleate (Mentane™), methanesulfonyl fluoride, phenylmethylsulfonyl fluoride, huperzine A, huperzine B, edrophonium chloride, galanthamine, and miotine; (c) angiotensin-converting enzyme inhibitors such as captopril (Capoten™), captopril in combination with hydrochlorothiazide (Capozide™), enalapril maleate (Vasotec™), enalaprilat, enalapril maleate/hydrochlorothiazide combination (Vaseretic™), fosinopril (Monopril™), lisinopril (Zestril™), ramipril (Altace™), epi-captopril, alacepril, quinapril, perindopril, delapril, cilazapril, pivalopril, rentiapril, zofenopril, and zofenoprilat; (d) N-methyl-D-aspartate glutamate receptor antagonists such as milacemide, trihexyphenidyl (Artane™), ethopropazine (Paridol™), procyclidine (Kemadrin™), diphenhydramine (Benadryl™), dizocilpine (Neurogard™), amantadine (Symmetrel™), and memantine (Namenda™); (e) the antioxidant co-agent ascorbic acid; (f) vasodilator and other nootropic direct brain metabolic enhancer drugs such as flunarizine, nimodipine (Nimotop™), idebenone, ebiratide, vinpocetine (Cavinton™), pentoxifylline, citicoline, bromvincamine, cyclandelate, isoxsuprene, nafronyl, papaverine, suloctidil, vinburnine, vincamine, vindeburnol, nicergoline (Sermion™), razobazam, exifone, rolipram, naloxone, ethyl 5-isopropyloxy-4-methyl-β-carboline-3-carboxylate, N′-methyl-α-carboline-3-carboxamide, methyl 6,7-dimethoxy-4-ethyl-β-carboline-3-carboxylate, ethyl 5-methoxy-4-ethyl-β-carboline-3-carboxylate, sabeluzole, phosphatidylserine, piracetam, aniracetam, pyroglutamic acid, tenilsetam, pramiracetam, oxiracetam, rolziracetam, etiracetam, propentophylline, dupracetam, and ergoloid mesylates (Hydergine™); (g) postsynaptic receptor agonists such as arecoline, oxotremorine, bethanechol (Urecholine™), levacecamine (acetyl-L-carnitine or Alcar™), and ethyl nipecotate; (h) biogenic amines and co-agents related thereto such as clonidine (Catapres™), guanfacine (Tenex™), alaproclate, fipexide, zimeldine, and citalopram; (i) anfacine; (j) agents which may enhance acetylcholine synthesis, storage or release such as phosphatidylcholine, 4-aminopyridine, 3,4-diaminopyridine, choline chloride, choline bitartrate, bifemelane, vesamicol, secoverine, tetraphenylurea, and nicotinamide; (k) acetylhomocysteine thiolactone; (l) ganglioside GM₁; (m) sulbutiamine; and (n) serotonergic receptor antagonists such as ketanserin (Ketan™), and mianserin (Mian™).

Suitable second agents further include therapeutic drugs that are useful in the treatment of tinnitus (nerve deafness) or its symptoms, which second agents include: antidepressants or antianxiety medications such as amitriptyline (Elavil™), amitriptyline/perphenazine combinations (Etrafon™), alprazolam (Xanax™), and triptolene; anticonvulsants such as primidone (Mysoline™), phenyloin (Dilantin™), and carbamazepine (TegQretol™); lidocaine (Xylocalne™); tocainide; flecinide; nicotinamide; aminooxyacetic acid; praxilene; aniracetam; piracetam; 13-cis-retinoic acid; and 13-trans-retinoic acid.

Suitable second agents further include therapeutic drugs that are useful in the treatment of multiple sclerosis or its symptoms, which second agents include: (a) azathioprine (Imuran™); (b) copolymer-1 (random polymer of L-alanine, L-glutamic acid, L-lysine and L-tyrosine, ratio of 6.0:1.9:4.7:1.0, of molecular weight between 14,000 and 23,000 Daltons); (c) cyclosporine (Sandimmune™); (d) interferons such as alfa-2a interferon (Roferon-A™), alfa-2b interferon (Intron-A™), alfa-N3 interferon (Alferon N Injection™), beta interferon (Betaseromm), and gamma-lb interferon (Actimmune™); (e) corticosteroids such as prednisone (Deltasone™), and dexamethasone (Decadron™); (f) cyclophosphamide (Cytoxan™); (g) 4-aminopyridine; (h) baclofen (Atrofen™); and (i) 3,4-diaminopyridine.

Suitable second agents further include therapeutic drugs that are useful in the treatment of amyotrophic lateral sclerosis or its symptoms, which second agents include: thyrotropin releasing factor (Relefact TRH); serine; L-threonine; N-methyl-D-aspartate glutamate receptor antagonists such as milacemide, trihexyphenidyl (Artane™), ethopropazine (Paridol™), procyclidine (Kemadrin™), diphenhydramine (Benadryl™), dizocilpine (Neurogard™), amantadine (Symmetrel™), and memantine (Namenda™).

Suitable second agents further include therapeutic drugs that are useful in the treatment of Huntington's disease or its symptoms, which second agents include: (a) N-methyl-D-aspartate glutamate receptor antagonists such as milacemide, trihexyphenidyl (Artane™), ethopropazine (Paridol™), procyclidine (Kemadrin™), diphenhydramine (Benadryl™), dizocilpine (Neurogard™), amantadine (Symmetrel™), and memantine (Namenda™); (b) agents which may enhance acetylcholine synthesis, storage or release such as phosphatidylcholine, 3,4-diaminopyridine, choline chloride, and choline bitartrate; and (c) postsynaptic receptor agonists such as arecoline.

Suitable second agents further include therapeutic drugs that are useful in the treatment of olivopontocerebellar atrophy or its symptoms, which second agents include: N-methyl-D-aspartate glutamate receptor antagonists such as milacemide, trihexyphenidyl (Artane™), ethopropazine (Paridol™), procyclidine (Kemadrin™, diphenhydramine (Benadryl™), dizocilpine (Neurogard™), amantadine (Symmetrel™), and memantine (Namenda™).

Suitable second agents further include therapeutic drugs that are useful in the treatment of alcoholic polyneuropathy or its symptoms, which second agents include: tiapride, physostigmine, optionally with phosphatidylcholine co-agent, piracetam, and cyclandelate.

Suitable second agents further include therapeutic drugs that are useful in the treatment of hereditary motor and sensory neuropathies or its symptoms, which second agents include 3,4-diaminopyridine.

Suitable second agents further include therapeutic drugs that are useful in the treatment of urinary incontinence resulting from Alzheimer's senile dementia, demyelinating diseases such as multiple sclerosis, peripheral nerve lesions, diabetes mellitus and alcoholic polyneuropathy or its symptoms, which second agents include: (a) cholinergics such as bethanechol (Urecholine™), alone or in combination with prazosin; (b) anti-cholinergics such as hyoscyamine sulfate, atropine sulfate, propantheline (Pro-Banthine™), oxybutynin (Ditropamm), and dicyclomine (Bentyl™); (c) α-adrenergics such as ephedrine and phenylpropanolamine; (d) tricyclic agents such as imipramine (Tofranil™) and doxepin (Adapin™); (e) flavoxate (Uripas™); (f) β-adrenergic blockers such as propranolol (Inderal™), pindolol (Visken™), metoprolol tartrate (Lopressor™), metoprolol succinate (Toprol XL™), and atenolol (Tenormin™); and (g) vasopressin analogues such as desmopressin (DDAVP Nasal Spray™).

Suitable second agents further include therapeutic drugs that are useful in the treatment of gastroesophageal reflux disease, hypoperistalsis and/or delayed gastric emptying or its symptoms, which second agents include: metoclopramide (Reglan™), cisapride (Prepulsid™), famotidine (Pepcid™), cimetidine (Tagamet™), ranitidine (Zantac™), omeprazole (Prilosec™), and galanthamine.

Suitable second agents further include therapeutic drugs that are useful in the treatment of symptomology related to onset and development of atherosclerosis or its symptoms, which second agents include: (a) angiotensin-converting enzyme inhibitor free radical scavenging agents possessing sulfhydryl groups such as captopril (Capoten™), captopril in combination with hydrochlorothiazide (Capozide™), epi-captopril, alacepril, pivalopril, and rentiapril; (b) fibric acid derivative anti-hyperlipidemia agents such as gemfibrozil (Lopid™), clofibrate (Atromid-S™), bezafibrate, and fenofibrate; (c) metformin; (d) nicotinic acid (Nicolar™); (e) natural hydroscopic non-digestible edible plant carbohydrate polymers such as guar gum; (f) 3-hydroxy-3-methylglutaryl-CoA reductase inhibitors such as lovastatin (Mevacor™), pravastatin (Pravachol™), and simvastatin (Zocor™); (g) acipimox; (h) bile acid sequestrants such as cholestyramine resin (Questran Light™), and colestipol (Colestid™); (i) anti-hypertensive agents including oral diuretics such as bendroflumethiazide (Naturetin™), benzthiazide (Exna™), chlorothiazide (Diuril™), chlorthalidone (Hvqroton™), cyclothiazide (Anhydron™), hydrochlorothiazide (Hydro-Diuril™), hydroflumethiazide (Saluron™), indapamide (Lozol™), methylclothiazide (Enduron™), metolazone (Zaroxolyn™), polythiazide (Renese™), quinethazone (Hydromox™), trichlormethiazide (Nagua™) and idebenone; loop diuretics such as bumetanide (Bumex™), ethacrynic acid (Edecrin™), furosemide (Lasix™) and torsemide (Presaril™); potassium-sparing diuretics such as amiloride (Midamomm), spironolactone (Aldactone™), and triamterene (Dyrenium™); β-adrenergic antagonists such as acebutolol (Sectral™), atenolol (Tenormin™), betaxolol (Kerlone™), carteolol (Cartrol™), labetalol (Normodyne™), metoprolol (Lopressor™), nadolol (Corgard™), penbutolol (Levatol™), pindolol (Visken™), propranolol (Inderal™ or Inderal LA™), timolol (Blocadren™) and bisoprolol (Zebeta™); calcium antagonists such as diltiazem (Cardizem™ or Cardizem SR™), verapamil (Calan™ or Calan SR™), nifedipine (Procardia™), nifedipine (Procardia XL™), nicardipine (Cardene™), isradipine (DynaCirc™), amlodipine (Norvasc™), felodipine (Plendil™), nimodipine (Nimotop™) and flunarizine; angiotensin-converting enzyme inhibitors such as captopril (Capoten™), enalapril (Vasotec™), fosinopril (Monopril™), lisinopril (Zestril™), ramipril (Altace™), quinapril (Accupril™), quinapril/hydrochlorothiazide combinations (Accuretic™) and benazepril (Lotensin™); peptide-based renin inhibitors such as [(2S)-3-(4-methylpiperazin-11-yl)sulfonyl-2-(phenylmethyl)-propionyl]-N-[(1S,2R,3 S)-1-(cyclohexylmethyl)-2,3-dihydroxy-5-methylhexyl]-L-[3-(thiazol-4-yl)alaninamide]; centrally acting α-adrenergic agonists such as clonidine (Catapres™), clonidine TTS (Catapres™), guanabenz (Wytensin™), guanfacine (Tenex™) and methyldopa (Aldomet™); peripherally acting α-adrenergic antagonists such as guanadrel (Hylorel™), guanethidine (Ismelin™), whole root Rauwolfia alkaloids (Raudixin™) and reserpine (Serpasil™); α-adrenergic antagonists such as prazosin (Minipress™), prazosin/polythiazide combination (Minizide™), terazosin (Hytrin™) and doxazosin (Cardura™); direct-acting vasodilators such as hydralazine (Apresoline™) and minoxidil (Loniten™); and (j) drugs for use in treatment of ischemic heart disease including nitrates such as oral isosorbide dinitrate and sustained-release trinitroglycerin; β-adrenergic antagonists such as acebutolol (Sectral™), atenolol (Tenormin™), betaxolol (Kerlone™), carteolol (Cartrol™), labetalol (Nommodyne™), metoprolol (Lopressor™), nadolol (Corgard™), penbutolol (Levatol™), pindolol (Visken™), propranolol (Inderal™ or Inderal LA™), timolol (Blocadren™) and bisoprolol (Zebeta™); and calcium channel antagonists such as diltiazem (Cardizem™ or Cardizem SR™), verapamil (Calan™ or Calan SR™), nifedipine (Procardia™), nifedipine (Procardia XL™), nicardipine (Cardene™), isradipine (DynaCire™), amlodipine (Norvasc™) and felodipine (Plendil™); and (k) ventricular antiarrhythmic drugs such as sotalol (Betapace™), mexilitene (Mexitil™), propafenone (Rythmol™), quinidine (Quinaglute Dura-Tabs™), procainamide (Procan SR™), and pirmenol (Pimavar™).

The First Agent

Compositions of certain alkanesulfur-oxides, including alkanesulfonic acids and alkanesulfuric acids, and more particularly including, for example, 3-amino-1-propanesulfonic acid and certain salts thereof have been shown to be useful in the treatment of amyloid-β related diseases, including Alzheimer's disease and cerebral amyloid angiopathy. See WO 96/28187, WO 01/85093, and U.S. Pat. No. 5,840,294. The anionic group of the composition is believed to inhibit an interaction between an amyloidogenic protein and a glycosaminoglycan (GAG) or proteoglycan constituent of a basement membrane to thus inhibit amyloid deposition.

The ability of a therapeutic compound of the invention to inhibit an interaction between an amyloidogenic protein and a glycoprotein or proteoglycan constituent of a basement membrane can be assessed by an in vitro binding assay, such as that described herein or in U.S. Pat. No. 5,164,295. Briefly, a solid support such as a polystyrene microtiter plate is coated with an amyloidogenic protein (e.g., serum amyloid A protein or β-amyloid precursor protein (β-APP)) and any residual hydrophobic surfaces are blocked. The coated solid support is incubated with various concentrations of a constituent of basement membrane, for example HSPG, either in the presence or absence of a compound to be tested. The solid support is washed extensively to remove unbound material. The binding of the basement membrane constituent (e.g., HSPG) to the amyloidogenic protein (e.g., β-APP) is then measured using an antibody directed against the basement membrane constituent that is conjugated to a detectable substance (e.g., an enzyme, such as alkaline phosphatase) by detecting the detectable substance. A compound which inhibits an interaction between an amyloidogenic protein and a glycoprotein or proteoglycan constituent of a basement membrane will reduce the amount of substance detected (e.g., will inhibit the amount of enzyme activity detected). A therapeutic compound of the invention may interact with a binding site for a basement membrane glycoprotein or proteoglycan in an amyloidogenic protein and thereby inhibit the binding of the amyloidogenic protein to the basement membrane constituent. Basement membrane glycoproteins and proteoglycans include laminin, collagen type IV, fibronectin and heparan sulfate proteoglycan (HSPG), perlecan, and agrin. In a similar embodiment, the therapeutic compound inhibits an interaction between an amyloidogenic protein and HSPG. Consensus binding site motifs for HSPG in amyloidogenic proteins have been described (see, e.g., Cardin and Weintraub, Arteriosclerosis 9, 21-32 (1989)).

The method also relates to a method for treating or preventing an amyloid-β related disease by administering at least two agents, each of which exerts a therapeutic effect when so administered and is useful in treating or preventing a neurological disease. The first agent of the invention is selected from alkanesulfonic acids that are useful for treating or preventing an amyloid-β related disease. The second agent is therapeutic, i.e., its function is beyond that of an inactive ingredient, such as a pharmaceutical carrier, preservative, diluent, or buffer. The second agent may be useful in treating or preventing an amyloid-β related disease or another neurological disease. The second agent may also be useful in diminishing specific symptoms which are characteristic of Alzheimer's disease (e.g., memory loss, anxiety, etc.). The first and second agents may exert their biological effects by similar or unrelated mechanisms of action; or either one or both of the first and second agents may exert their biological effects by a multiplicity of mechanisms of action. A third agent, or even more yet, may likewise be used in a method of the invention, wherein the third (and fourth, etc.) agent has the same characteristics of a second agent. The invention relates to a method of treating or preventing an amyloid-β related disease in a subject (for example, a human) comprising administering to the subject a therapeutic amount of a alkanesulfonic acid, such that amyloid fibril formation or deposition, neurodegeneration, or cellular toxicity is reduced or inhibited. In another embodiment, the invention relates to a method of treating or preventing an amyloid-β related in a subject (for example, a human) comprising administering to the subject a therapeutic amount of an alkanesulfonic acid, such that cognitive function is stabilized or further deterioration in cognitive function is prevented, slowed, or stopped in subjects with brain amyloidosis, e.g., Alzheimer's disease or cerebral amyloid angiopathy. In another embodiment, the invention relates to a method of treating or preventing an amyloid-β disease in a subject (for example, human) comprising administering to the subject a therapeutic amount of a alkanesulfonic acid, such that activities of daily living are improved or stabilized in subjects with brain amyloidosis, e.g., Alzheimer's disease.

The “first agent” according to the invention may be an alkanesulfonic acid or an alkanolsulfuric acid. The term “alkanesulfonic acid” includes substituted or unsubstituted alkanesulfonic acids, and substituted or unsubstituted lower alkanesulfonic acids. Amino-substituted compounds are especially noteworthy and the invention pertains to substituted- or unsubstituted-amino-substituted alkanesulfonic acids, and substituted- or unsubstituted-amino-substituted lower alkanesulfonic acids, and example of which is 3-amino-1-propanesulfonic acid.

The methods and pharmaceutical compositions of the invention are therefore directed to a first agent that is a substituted or unsubstituted alkanesulfonic acid, substituted or unsubstituted alkanesulfuric acid (also known as an alkanol sulfuric acid), substituted or unsubstituted alkylthiosulfonic acid, substituted or unsubstituted alkylthiosulfuric acid, or an ester or amide thereof, including pharmaceutically acceptable salts thereof. For example, the invention relates to a first agent that is a substituted or unsubstituted alkanesulfonic acid, or an ester or amide thereof, including pharmaceutically acceptable salts thereof. In another embodiment, the invention pertains to a first agent that is a substituted or unsubstituted lower alkanesulfonic acid, or an ester or amide thereof, including pharmaceutically acceptable salts thereof. Similarly, the invention includes a first agent that is a (substituted- or unsubstituted-amino)-substituted alkanesulfonic acid, or an ester or amide thereof, including pharmaceutically acceptable salts thereof. In yet another embodiment, the first agent is a (substituted- or unsubstituted-amino)-substituted lower alkanesulfonic acid, or an ester or amide thereof, including pharmaceutically acceptable salts thereof.

As used herein, “alkyl” groups include saturated hydrocarbons having one or more carbon atoms, including straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (e.g., cyclopropyl, cyclopentyl, cyclohexyl, cycloheptyl, cyclooctyl, etc.), branched-chain alkyl groups (isopropyl, tert-butyl, sec-butyl, isobutyl, etc.), and alkyl-substituted alkyl groups (e.g., alkyl-substituted cycloalkyl groups and cycloalkyl-substituted alkyl groups). The term “aliphatic group” includes organic moieties characterized by straight or branched-chains, typically having between 1 and 22 carbon atoms. In complex structures, the chains may be branched, bridged, or cross-linked. Aliphatic groups include alkyl groups, alkenyl groups, and alkynyl groups.

Accordingly, the invention relates to substituted or unsubstituted alkanesulfonic acids that are substituted or unsubstituted straight-chain alkanesulfonic acids, substituted or unsubstituted cycloalkanesulfonic acids, and substituted or unsubstituted branched-chain alkanesulfonic acids.

The structures of some of the compounds of this invention include stereogenic carbon atoms. It is to be understood that isomers arising from such asymmetry (e.g., all enantiomers and diastereomers) are included within the scope of this invention unless indicated otherwise. That is, unless otherwise stipulated, any chiral carbon center may be of either (R)— or (S)-stereochemistry. Such isomers can be obtained in substantially pure form by classical separation techniques and by stereochemically-controlled synthesis. In addition, the compounds of the present invention may exist in unsolvated as well as solvated forms with acceptable solvents such as water, THF, ethanol, and the like. In general, the solvated forms are considered equivalent to the unsolvated forms for the purposes of the present invention. The term “solvate” represents an aggregate that comprises one or more molecules of a compound, with one or more molecules of a pharmaceutical solvent, such as water, ethanol, and the like.

In certain embodiments, a straight-chain or branched-chain alkyl group may have 30 or fewer carbon atoms in its backbone, e.g., C₁-C₃₀ for straight-chain or C₃-C₃₀ for branched-chain. In certain embodiments, a straight-chain or branched-chain alkyl group may have 20 or fewer carbon atoms in its backbone, e.g., C₁-C₂₀ for straight-chain or C₃-C₂₀ for branched-chain, and more, for example, 18 or fewer. Likewise, example cycloalkyl groups have from 4-10 carbon atoms in their ring structure, or 4-7 carbon atoms in the ring structure.

The term “lower alkyl” refers to alkyl groups having from 1 to 6 carbons in the chain, and to cycloalkyl groups having from 3 to 6 carbons in the ring structure. Unless the number of carbons is otherwise specified, “lower” as in “lower alkyl,” means that the moiety has at least one and less than about 8 carbon atoms. In certain embodiments, a straight-chain or branched-chain lower alkyl group has 6 or fewer carbon atoms in its backbone (e.g., C₁-C₆ for straight-chain, C₃-C₆ for branched-chain), and more preferably 4 or fewer, for example, methyl, ethyl, propyl, isopropyl, butyl, isobutyl, sec-butyl, and tert-butyl. Likewise, cycloalkyl groups may have from 3-8 carbon atoms in their ring structure, for example, 5 or 6 carbons in the ring structure. The term “C₁-C₆” as in “C₁-C₆ alkyl” means alkyl groups containing 1 to 6 carbon atoms.

Moreover, unless otherwise specified the term alkyl includes both “unsubstituted alkyls” and “substituted alkyls,” the latter of which refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkanesulfinyl, sulfonato, sulfamoyl, sulfonamido, nitro, trifluoromethyl, cyano, azido, heterocyclyl, alkylaryl, or aromatic (including heteroaromatic) groups.

An “arylalkyl” group is an alkyl group substituted with an aryl group (e.g., phenylmethyl (i.e., benzyl)). An “alkylaryl” moiety is an aryl group substituted with an alkyl group (e.g., p-methylphenyl (i.e., p-tolyl)). The term “n-alkyl” means a straight-chain (ie., unbranched) unsubstituted alkyl group. An “alkylene” group is a divalent analog of the corresponding alkyl group. The terms “alkenyl” and “alkynyl” refer to unsaturated aliphatic groups analogous to alkyls, but which contain at least one double or triple carbon-carbon bond respectively. Suitable alkenyl and alkynyl groups include groups having 2 to about 12 carbon atoms, preferably from 2 to about 6 carbon atoms.

The term “aromatic group” or “aryl group” includes unsaturated and aromatic cyclic hydrocarbons as well as unsaturated and aromatic heterocycles containing one or more rings. Aryl groups may also be fused or bridged with alicyclic or heterocyclic rings that are not aromatic so as to form a polycycle (e.g., tetralin). An “arylene” group is a divalent analog of an aryl group. Aryl groups can also be fused or bridged with alicyclic or heterocyclic rings which are not aromatic so as to form a polycycle (e.g., tetralin).

The term “heterocyclic group” includes closed ring structures analogous to carbocyclic groups in which one or more of the carbon atoms in the ring is an element other than carbon, for example, nitrogen, sulfur, or oxygen. Heterocyclic groups may be saturated or unsaturated. Additionally, heterocyclic groups (such as pyrrolyl, pyridyl, isoquinolyl, quinolyl, purinyl, and furyl) may have aromatic character, in which case they may be referred to as “heteroaryl” or “heteroaromatic” groups.

Unless otherwise stipulated, aryl and heterocyclic (including heteroaryl) groups may also be substituted at one or more constituent atoms. Examples of heteroaromatic and heteroalicyclic groups may have 1 to 3 separate or fused rings with 3 to about 8 members per ring and one or more N, O, or S heteroatoms. In general, the term “heteroatom” includes atoms of any element other than carbon or hydrogen, preferred examples of which include nitrogen, oxygen, sulfur, and phosphorus. Heterocyclic groups may be saturated or unsaturated or aromatic.

Examples of heterocycles include, but are not limited to, acridinyl; azocinyl; benzimidazolyl; benzofuranyl; benzothiofuranyl; benzothiophenyl; benzoxazolyl; benzthiazolyl; benztriazolyl; benztetrazolyl; benzisoxazolyl; benzisothiazolyl; benzimidazolinyl; carbazolyl; 4aH-carbazolyl; carbolinyl; chromanyl; chromenyl; cinnolinyl; decahydroquinolinyl; 2H,6H-1,5,2-dithiazinyl; dihydrofuro[2,3-b]tetrahydrofuran; furanyl; furazanyl; imidazolidinyl; imidazolinyl; imidazolyl; 1H-indazolyl; indolenyl; indolinyl; indolizinyl; indolyl; 3H-indolyl; isobenzofuranyl; isochromanyl; isoindazolyl; isoindolinyl; isoindolyl; isoquinolinyl; isothiazolyl; isoxazolyl; methylenedioxyphenyl; morpholinyl; naphthyridinyl; octahydroisoquinolinyl; “oxadiazolyl; 1,2,3-oxadiazolyl; 1,2,4-oxadiazolyl; 1,2,5-oxadiazolyl; 1,3,4-oxadiazolyl; oxazolidinyl; oxazolyl; oxazolidinyl; pyrimidinyl; phenanthridinyl; phenanthrolinyl; phenazinyl; phenothiazinyl; phenoxathiinyl; phenoxazinyl; phthalazinyl; piperazinyl; piperidinyl; piperidonyl; 4-piperidonyl; piperonyl; pteridinyl; purinyl; pyranyl; pyrazinyl; pyrazolidinyl; pyrazolinyl; pyrazolyl; pyridazinyl; pyridooxazole; pyridoimidazole; pyridothiazole; pyridinyl; pyridyl; pyrimidinyl; pyrrolidinyl; pyrrolinyl; 2H-pyrrolyl; pyrrolyl; quinazolinyl; quinolinyl; 4H-quinolizinyl; quinoxalinyl; quinuclidinyl; tetrahydrofuranyl; tetrahydroisoquinolinyl; tetrahydroquinolinyl; tetrazolyl; 6H-1,2,5-thiadiazinyl; 1,2,3-thiadiazolyl; 1,2,4-thiadiazolyl; 1,2,5-thiadiazolyl; 1,3,4-thiadiazolyl; thianthrenyl; thiazolyl; thienyl; thienothiazolyl; thienooxazolyl; thienoimidazolyl; thiophenyl; triazinyl; 1,2,3-triazolyl; 1,2,4-triazolyl; 1,2,5-triazolyl; 1,3,4-triazolyl; and xanthenyl. Preferred heterocycles include, but are not limited to, pyridinyl; furanyl; thienyl; pyrrolyl; pyrazolyl; pyrrolidinyl; imidazolyl; indolyl; benzimidazolyl; 1H-indazolyl; oxazolidinyl; benzotriazolyl; benzisoxazolyl; oxindolyl; benzoxazolinyl; and isatinoyl groups. Also included are fused ring and spiro compounds containing, for example, the above heterocycles.

A common hydrocarbon aryl group is a phenyl group having one ring. Two-ring hydrocarbon aryl groups include naphthyl, indenyl, benzocyclooctenyl, benzocycloheptenyl, pentalenyl, and azulenyl groups, as well as the partially hydrogenated analogs thereof such as indanyl and tetrahydronaphthyl. Exemplary three-ring hydrocarbon aryl groups include acephthylenyl, fluorenyl, phenalenyl, phenanthrenyl, and anthracenyl groups.

Aryl groups also include heteromonocyclic aryl groups, i.e., single-ring heteroaryl groups, such as thienyl, furyl, pyranyl, pyrrolyl, imidazolyl, pyrazolyl, pyridinyl, pyrazinyl, pyrimidinyl, and pyridazinyl groups; and oxidized analogs thereof such as pyridonyl, oxazolonyl, pyrazolonyl, isoxazolonyl, and thiazolonyl groups. The corresponding hydrogenated (i.e., non-aromatic) heteromonocylic groups include pyrrolidinyl, pyrrolinyl, imidazolidinyl, imidazolinyl, pyrazolidinyl, pyrazolinyl, piperidyl and piperidino, piperazinyl, and morpholino and morpholinyl groups.

Aryl groups also include fused two-ring heteroaryls such as indolyl, isoindolyl, indolizinyl, indazolyl, quinolinyl, isoquinolinyl, phthalazinyl, quinoxalinyl, quinazolinyl, cinnolinyl, chromenyl, isochromenyl, benzothienyl, benzimidazolyl, benzothiazolyl, purinyl, quinolizinyl, isoquinolonyl, quinolonyl, naphthyridinyl, and pteridinyl groups, as well as the partially hydrogenated analogs such as chromanyl, isochromanyl, indolinyl, isoindolinyl, and tetrahydroindolyl groups. Aryl groups also include fused three-ring groups such as phenoxathiinyl, carbazolyl, phenanthridinyl, acridinyl, perirnidinyl, phenanthrolinyl, phenazinyl, phenothiazinyl, phenoxazinyl, and dibenzofuranyl groups.

Some typical aryl groups include substituted or unsubstituted 5- and 6-membered single-ring groups. In another aspect, each Ar group may be selected from the group consisting of substituted or unsubstituted phenyl, pyrrolyl, furyl, thienyl, thiazolyl, isothiaozolyl, imidazolyl, triazolyl, tetrazolyl, pyrazolyl, oxazolyl, isooxazolyl, pyridinyl, pyrazinyl, pyridazinyl, and pyrimidinyl groups. Further examples include substituted or unsubstituted phenyl, 1-naphthyl, 2-naphthyl, biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl groups.

The term “amine” or “amino,” as used herein, refers to an unsubstituted or substituted moiety of the formula —NR^{a}R^{b}, in which R^{a} and R^{b} are each independently hydrogen, alkyl, aryl, or heterocyclyl, or R^{a} and R^{b}, taken together with the nitrogen atom to which they are attached, form a cyclic moiety having from 3 to 8 atoms in the ring. Thus, the term amino includes cyclic amino moieties such as piperidinyl or pyrrolidinyl groups, unless otherwise stated. Thus, the term “alkylamino” as used herein means an alkyl group having an amino group attached thereto. Suitable alkylamino groups include groups having 1 to about 12 carbon atoms, for example, 1 to about 6 carbon atoms. The term amino includes compounds or moieties in which a nitrogen atom is covalently bonded to at least one carbon or heteroatom. The term “dialkylamino” includes groups wherein the nitrogen atom is bound to at least two alkyl groups. The term “arylamino” and “diarylamino” include groups wherein the nitrogen is bound to at least one or two aryl groups, respectively. The term “alkylarylamino” refers to an amino group which is bound to at least one alkyl group and at least one aryl group. The term “alkaminoalkyl” refers to an alkyl, alkenyl, or alkynyl group substituted with an alkylamino group. The term “amide” or “aminocarbonyl” includes compounds or moieties which contain a nitrogen atom which is bound to the carbon of a carbonyl or a thiocarbonyl group.

The term “alkylthio” refers to an alkyl group, having a sulfhydryl group attached thereto. Suitable alkylthio groups include groups having 1 to about 12 carbon atoms, preferably from 1 to about 6 carbon atoms.

The term “alkylcarboxyl” as used herein means an alkyl group having a carboxyl group attached thereto.

The term “alkoxy” as used herein means an alkyl group having an oxygen atom attached thereto. Representative alkoxy groups include groups having 1 to about 12 carbon atoms, preferably 1 to about 6 carbon atoms, e.g., methoxy, ethoxy, propoxy, tert-butoxy and the like. Examples of alkoxy groups include methoxy, ethoxy, isopropyloxy, propoxy, butoxy, and pentoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulffiydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc., as well as perhalogenated alkyloxy groups.

The term “acylamino” includes moieties wherein an amino moiety is bonded to an acyl group. For example, the acylamino group includes alkylcarbonylamino, arylcarbonylamino, carbamoyl and ureido groups.

The terms “alkoxyalkyl”, “alkylaminoalkyl” and “thioalkoxyalkyl” include alkyl groups, as described above, which further include oxygen, nitrogen or sulfur atoms replacing one or more carbons of the hydrocarbon backbone.

The term “carbonyl” or “carboxy” includes compounds and moieties which contain a carbon connected with a double bond to an oxygen atom. Examples of moieties which contain a carbonyl include aldehydes, ketones, carboxylic acids, amides, esters, anhydrides, etc.

The term “ether” or “ethereal” includes compounds or moieties which contain an oxygen bonded to two carbon atoms. For example, an ether or ethereal group includes “alkoxyalkyl” which refers to an alkyl, alkenyl, or alkynyl group substituted with an alkoxy group.

The term “nitro” means —NO₂; the term “halogen” or “halogeno” or “halo” designates —F, —Cl, —Br or —I; the term “thiol,” “thio,” or “mercapto” means SH; and the term “hydroxyl” or “hydroxy” means —OH.

The term “acyl” refers to a carbonyl group that is attached through its carbon atom to a hydrogen (i.e., a formyl), an aliphatic group (e.g., acetyl), an aromatic group (e.g., benzoyl), and the like. The term “substituted acyl” includes acyl groups where one or more of the hydrogen atoms on one or more carbon atoms are replaced by, for example, an alkyl group, alkynyl group, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulffydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

A “sulfonic acid” or “sulfonate” group is a —SO₃H or —SO₃⁻X⁺ group bonded to a carbon atom, where X⁺ is a cationic counter ion group. Similarly, a “sulfonic acid” compound has a —SO₃H or —SO₃⁻X⁺ group bonded to a carbon atom, where X⁺ is a cationic group. A “sulfate” as used herein is a —OSO₃H or —OSO₃⁻X⁺ group (which may also be represented as —SO₄H or —SO₄⁻X⁺) bonded to a carbon atom, and a “sulfuric acid” compound has a —SO₃H or —OSO₃⁻X⁺ group bonded to a carbon atom, where X⁺ is a cationic group. According to the invention, a suitable cationic group may be a hydrogen atom. In certain cases, the cationic group may actually be another group on the therapeutic compound that is positively charged at physiological pH, for example an amino group. A “counter ion” is required to maintain electroneutrality, and is pharmaceutically acceptable in the compositions of the invention. Examples of anionic counter ions include halide, triflate, sulfate, nitrate, hydroxide, carbonate, bicarbonate, acetate, phosphate, oxalate, cyanide, alkylcarboxylate, N-hydroxysuccinimide, N-hydroxybenzotriazole, alkoxide, thioalkoxide, alkane sulfonyloxy, halogenated alkane sulfonyloxy, arylsulfonyloxy, bisulfate, oxalate, valerate, oleate, palmitate, stearate, laurate, borate, benzoate, lactate, citrate, maleate, fumarate, succinate, tartrate, naphthylate mesylate, glucoheptonate, or lactobionate. Compounds containing a cationic group covalently bonded to an anionic group may be referred to as an “internal salt.”

Unless otherwise specified, the chemical moieties of the compounds of the invention, including those groups discussed above, may be “substituted or unsubstituted.” In some embodiments, the term “substituted” means that the moiety has substituents placed on the moiety other than hydrogen (i.e., in most cases, replacing a hydrogen), which allow the molecule to perform its intended function. Examples of substituents include moieties selected from straight or branched alkyl (e.g.,C₁-C₅), cycloalkyl (e.g., C₃-C₈), amino groups (including —NH₂), —SO₃H, —OSO₃H, —CN, —NO₂, halogen (e.g., —F, —Cl, —Br, or —I), —CH₂OCH₃, —OCH₃, —SH, —SCH₃, —OH, and —CO₂H, alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, and heteroaryl groups, as well as (CR′R″)₀₋₃NR′R″ (e.g., —NH₂), (CR′R″)₀₋₃CN (e.g., —CN), —NO₂, halogen (e.g., —F, —Cl, —Br, or —I), (CR′R″)₀₋₃C(halogen)₃ (e.g, —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₃R′ (e.g., —SO₃H), (CR′R″)₀₋₃(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃OH(e.g.—OH), (CR′R″)₀₋₃COR′, (CR′R″)₀₋₃(substituted or unsubstituted phenyl), (CR′R″)₀₋₃(C₃-C₈ cycloalkyl), (CR′R″)₀₋₃CO₂R′ (e.g., —CO₂H), and (CR′R″)₀₋₃OR′ groups, wherein R′ and R″ are each independently hydrogen, a C₁-C₅ alkyl, C₂-C₅ alkenyl, C₂-C₅ alkynyl, or aryl group; or the side chain of any naturally occurring amino acid.

In another embodiment, a substituent may be selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₁₀NR′R″ (e.g., —NH₂), (CR′R″)₀₋₁₀CN (e.g., —CN), NO₂, halogen (e.g., F, Cl, Br, or I), (CR′R″)₀₋₁₀C(halogen)₃ (e.g., —CF₃), (CR′R″)₀₋₁₀CH(halogen)₂, (CR′R″)₀₋₁₀CH₂(halogen), (CR′R″)₀₋₁₀CONR′R″, (CR′R″)₀₋₁₀(CNH)NR′R″, (CR′R″)₀₋₁₀S(O)₁₋₂NR′R″, (CR′R″)₀₋₁₀CHO, (CR′R″)₀₋₁₀O(CR′R″)₀₋₁₀H, (CR′R″)₀₋₁₀S(O)₀₋₃R′ (e.g., —SO₃H), (CR′R″)₀₋₁₀O(CR′R″)₀₋₁₀H (e.g., —CH₂OCH₃ and —OCH₃), (CR′R″)₀₋₁₀S(CR′R″)₀₋₃H (e.g., —SH and —SCH₃), (CR′R″)₀₋₁₀OH (e.g., —OH), (CR′R″)₀₋₁₀COR′, (CR′R″)₀₋₁₀(substituted or unsubstituted phenyl), (CR′R″)₀₋₁₀(C₃-C₈ cycloalkyl), (CR′R″)₀₋₁₀CO₂R′ (e.g, —CO₂H), or (CR′R″)₀₋₁₀OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group.

As used herein, the term “substituted” is meant to include all permissible substituents of organic compounds. In a broad aspect, the permissible substituents include acyclic and cyclic, branched and unbranched, carbocyclic and heterocyclic, aromatic and nonaromatic substituents of organic compounds. The permissible substituents can be one or more.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is meant to include all permissible substituents of organic compounds. In a broad aspect, the permissible substituents include acyclic and cyclic, branched and unbranched, carbocyclic and heterocyclic, aromatic and nonaromatic substituents of organic compounds. The permissible substituents can be one or more and the same or different for appropriate organic compounds.

In some embodiments, a “substituent” may be, selected from the group consisting of, for example, halogeno, trifluoromethyl, nitro, cyano, C₁-C₆ alkyl, C₂-C₆ alkenyl, C₂-C₆ alkynyl, C₁-C₆ alkylcarbonyloxy, arylcarbonyloxy, C₁-C₆ alkoxycarbonyloxy, aryloxycarbonyloxy, C₁-C₆ alkylcarbonyl, C₁-C₆ alkoxycarbonyl, C₁-C₆ alkoxy, C₁-C₆ alkylthio, arylthio, heterocyclyl, aralkyl, and aryl (including heteroaryl) groups.

One group of example alkanesulfonic acids have the following structure as depicted in Formula I (see the Drawings, attached hereto) where Y is either an amino group (having the formula —NR^{a}R^{b}) or a sulfonic acid group (having the formula —SO₃⁻X⁺), n is an integer from 1 to 5, and X is hydrogen or a cationic group (e.g., sodium). Some exemplary alkanesulfonic acids include the those depicted within Formula IIa, Formula lIb, Formula IIc, and Formula lId (see Figures).

One embodiment of the invention is the use of 3-amino-1-propanesulfonic acid and pharmaceutically acceptable salts thereof as a first agent of the pharmaceutical compositions described herein and the methods of using them.

An “agent,” as in a “first agent” or a “second agent” is generally intended to describe a chemical compound of suitable purity for use in a pharmaceutical preparation. In some cases, the agent is a “small molecule,” that is, a compound that that is not itself the product of gene transcription or translation (e.g., protein, RNA, or DNA) and has a low molecular weight, e.g., less than about 2500. In other cases, the agent may be a biological product, such as an antibody or an immunogenic peptide.

In general, alkanesulfonic acids may be prepared by the methods illustrated in the general reaction schemes as, for example, described in U.S. Pat. Nos. 5,643,562; 5,972,328; 5,728,375; 5,840,294; 4,657,704; and the U.S. Provisional Patent Application No. 60/482,058, filed 23 Jun. 2003, entitled “Synthetic Processfor Preparing Compoundsfor Treating Amyloidosis”, or by modifications thereof, using readily available starting materials, reagents and conventional synthesis procedures. In these reactions, it is also possible to make use of variants which are in themselves known, but are not mentioned. Functional and structural equivalents of the agents described herein and that have the same general properties, wherein one or more simple variations of substituents are made which do not adversely affect the essential nature or the utility of the agent may be prepared according to a variety of methods known in the art.

In general, the agents of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here. Functional and structural equivalents of the agents described herein and which have the same general properties, wherein one or more simple variations of substituents are made which do not adversely affect the essential nature or the utility of the agent. The agents of the present invention may be readily prepared in accordance with the synthesis schemes and protocols described herein, as illustrated in the specific procedures provided. However, those skilled in the art will recognize that other synthetic pathways for forming the agents of this invention may be used, and that the following is provided merely by way of example, and is not limiting to the present invention. See, e.g., “Comprehensive Organic Transformations,” 2^{nd} Ed., by R.C. Larock, John Wiley & Sons, Ltd. (1999); “March's Advanced Organic Chemistry,” 5^{th} Ed., by M. B. Smith and J. March, John Wiley & Sons, Ltd. (2000); and “Reagents for Organic Synthesis,” Vol. I-XX, by M. Fieser and L. Fieser, John Wiley & Sons (2000). It will be further recognized that various protecting and deprotecting strategies will be employed that are standard in the art (see, e.g., “Protective Groups in Organic Synthesis,” 3rd Ed., by T. W. Greene, John Wiley & Sons, Ltd. (1999)). Those skilled in the relevant arts will recognize that the selection of any particular protecting group (e.g., amine and carboxylprotecting groups) will depend on the stability of the protected moiety with regards to the subsequent reaction conditions and will understand the appropriate selections. Further illustrating the knowledge of those skilled in the art is the following sampling of the extensive chemical literature: “Comprehensive Asymmetric Catalysis”, by E. N. Jacobsen, et al., Springer Verlag (1999) “Chemistry of the Amino Acids” by J. P. Greenstein and M. Winitz, John Wiley & Sons, Inc., New York (1961); T. D. Ocain, et al., J. Med. Chem. 31, 2193-99 (1988); E. M. Gordon, et al., J. Med. Chem. 31, 2199-10 (1988); “Practice of Peptide Synthesis” by M. Bodansky and A. Bodanszky, Springer-Verlag, N. Y. (1984); “Asymmetric Synthesis: Construction of Chiral Molecules Using Amino Acids” by G. M. Coppola and H. F. Schuster, John Wiley & Sons, Inc., New York (1987); “The Chemical Synthesis of Peptides” by J. Jones, Oxford University Press, New York (1991); and “Introduction of Peptide Chemistry” by P. D. Bailey, John Wiley & Sons, Inc., New York (1992).

The chemical structures herein are drawn according to the conventional standards known in the art. Thus, where an atom, such as a carbon atom, as drawn appears to have an unsatisfied valency, then that valency is assumed to be satisfied by a hydrogen atom even though that hydrogen atom is not necessarily explicitly drawn. The structures of some of the compounds of this invention include stereogenic carbon atoms. It is to be understood that isomers arising from such asymmetry (e.g., all enantiomers and diastereomers) are included within the scope of this invention unless indicated otherwise. That is, unless otherwise stipulated, any chiral carbon center may be of either (R)- or (S)-stereochemistry. Such isomers can be obtained in substantially pure form by classical separation techniques and by stereochemically-controlled synthesis. Furthermore, alkenes can include either the E- or Z-geometry, where appropriate. In addition, the compounds of the present invention may exist in unsolvated as well as solvated forms with acceptable solvents such as water, THF, ethanol, and the like. In general, the solvated forms are considered equivalent to the unsolvated forms for the purposes of the present invention.

Further examples of compounds that may be used as a first agent according to the present invention include those described in the U.S. Provisional Patent Application No. 60/480,906, filed 23 Jun. 2003, entitled “Methods and Compositions for Treating Amyloid-Related Diseases” and Application No. 60/480,928, also filed 23 Jun. 2003 “Methods and Compositions for The Treatment of Amyloid-and Epileptogenesis-Associated Diseases”.

In an embodiment, the invention pertains, at least in part to a pharmaceutical composition having a first agent that is a compound of Formula I-A (see the Drawings sheets attached hereto), wherein R¹ is a substituted or unsubstituted cycloalkyl, aryl, arylcycloalkyl, bicyclic or tricyclic ring, a bicyclic or tricyclic fused ring group, or a substituted or unsubstituted C₂-C₁₀ alkyl group; R² is selected from a group consisting hydrogen, alkyl, mercaptoalkyl, alkenyl, alkynyl, cyclo-alkyl, aryl, arylalkyl, thiazolyl, triazolyl, imidazolyl, benzothiazolyl, and benzoimidazolyl; Y is SO₃⁻X⁺, OSO₃⁻X⁺, or SSO₃⁻X⁺; X⁺ is hydrogen, a cationic group, or an ester-forming group (i.e., as in a prodrug, which are described elsewhere herein); and each of L¹ and L² is independently a substituted or unsubstituted C₁-C₅ alkyl group or absent, or a pharmaceutically acceptable salt thereof, provided that when R¹ is alkyl, L¹ is absent.

In another embodiment, the invention pertains, at least in part a pharmaceutical composition having a first agent that is a compound of Formula II-A (see the Drawings sheets attached hereto) wherein R¹ is a substituted or unsubstituted cyclic, bicyclic, tricyclic, or benzoheterocyclic group or a substituted or unsubstituted C₂-C₁₀ alkyl group; R² is hydrogen, alkyl, mercaptoalkyl, alkenyl, alkynyl, cycloalkyl, aryl, arylalkyl, thiazolyl, triazolyl, imidazolyl, benzothiazolyl, benzoimidazolyl, or linked to R¹ to form a heterocycle; Y is SO₃⁻X⁺, OSO₃⁻X⁺, or SSO₃⁻X^{+; X}⁺ is hydrogen, a cationic group, or an ester forming moiety; m is 0 or 1; n is 1, 2, 3, or 4; L is substituted or unsubstituted C₁-C₃ alkyl group or absent, or a pharmaceutically acceptable salt thereof, provided that when R¹ is alkyl, L is absent.

In yet another embodiment, the invention pertains, at least in part to a pharmaceutical composition having a first agent that is a compound of Formula III-A (see the Drawings sheets attached hereto) wherein A is nitrogen or oxygen; R¹¹ is hydrogen, salt-forming cation, ester forming group, —(CH₂)ₓ-Q, or when A is nitrogen, A and R¹¹ taken together may be the residue of a natural or unnatural amino acid or a salt or ester thereof; Q is hydrogen, thiazolyl, triazolyl, imidazolyl, benzothiazolyl, or benzoimidazolyl; x is 0, 1, 2, 3, or 4; n is 0, 1, 2 ,3, 4, 5, 6, 7, 8, 9, or 10; R³, R^{3a}, R⁴, R^{4a}, R⁵, R^{5a}, R⁶, R^{6a}, R⁷ and R^{7a} are each independently hydrogen, alkyl, mercaptoalkyl, alkenyl, alkynyl, cycloalkyl, aryl, alkylcarbonyl, arylcarbonyl, alkoxycarbonyl, cyano, halogen, amino, amidino, tetrazolyl, or two R groups on adjacent ring atoms taken together with the ring atoms form a double bond, provided that one of R³, R^{3a}, R⁴, R^{4a}, R⁵, R^{5a}, R⁶, R^{6a}, R⁷ and R^{7a} is a moiety of Formula IIIa-A, as depicted in the Drawings, wherein m is 0, 1, 2, 3, or 4; R⁸, R⁹, R¹⁰, R¹¹, and R¹² are independently selected from a group of hydrogen, halogen, hydroxyl, alkyl, alkoxyl, halogenated alkyl, mercaptoalkyl, alkenyl, alkynyl, cycloalkyl, aryl, cyano, amidino, thiazolyl, triazolyl, imidazolyl, tetrazolyl, benzothiazolyl, and benzoimidazoly; and pharmaceutically acceptable salts and esters thereof, provided that said compound is not 3-(4-phenyl-1, 2, 3, 6-tetrahydro-1-pyridyl)-1-propanesulfonic acid.

In yet another embodiment, the invention pertains at least in part to a pharmaceutical composition having a first agent that is a compound of Formula IV (see the attached Drawings) wherein: A is nitrogen or oxygen; R¹¹ is hydrogen, salt-forming cation, ester forming group, —(CH₂)ₓ-Q, or when A is nitrogen, A and R¹¹ taken together may be the residue of a natural or unnatural amino acid or a salt or ester thereof; Q is hydrogen, thiazolyl, triazolyl, imidazolyl, benzothiazolyl, or benzoimidazolyl; x is 0, 1, 2, 3, or 4; n is 0, 1, 2 , 3, 4, 5, 6, 7, 8, 9, or 10; R⁴, R^{4a}, R⁵, R^{5a}, R⁶, R^{6a}, R⁷, and R^{7a} are each independently hydrogen, alkyl, mercaptoalkyl, alkenyl, alkynyl, cycloalkyl, aryl, alkylcarbonyl, arylcarbonyl, alkoxycarbonyl, cyano, halogen, amino, amidino, tetrazolyl, R⁴ and R⁵ are taken together, with the ring atoms they are attached to, form a double bond, or R⁶ and R⁷ are taken together, with the ring atoms they are attached to, form a double bond; m is 0, 1, 2, 3, or 4; R⁸, R⁹, R¹⁰, R¹¹, and R¹² are independently selected from a group of hydrogen, halogen, hydroxyl, alkyl, alkoxyl, halogenated alkyl, mercaptoalkyl, alkenyl, alkynyl, cycloalkyl, aryl, cyano, amidino, thiazolyl, triazolyl, imidazolyl, tetrazolyl, benzothiazolyl, and benzoimidazolyl, and pharmaceutically acceptable salts and esters thereof.

In another embodiment, the invention includes a pharmaceutical composition having a first agent that is a compound of Formula V-A (see the attached Drawings) wherein A is nitrogen or oxygen; R¹¹ is hydrogen, salt-forming cation, ester forming group, —(CH₂)ₓ-Q, or when A is nitrogen, A and R′ ¹ taken together may be the residue of a natural or unnatural amino acid or a salt or ester thereof; Q is hydrogen, thiazolyl, triazolyl, imidazolyl, benzothiazolyl, or benzoimidazolyl; x is 0, 1, 2, 3, or 4; n is 0, 1, 2, 3, 4, 5, 6, 7, 8, 9, or 10; aa is a natural or unnatural amino acid residue; m is 0, 1, 2, or 3; R¹⁴ is hydrogen or protecting group; R¹⁵ is hydrogen, alkyl or aryl, and pharmaceutically acceptable salts and prodrugs thereof.

In another embodiment, the invention includes a pharmaceutical composition having a first agent that is a compound of the Formula VI-A (see the attached Drawings) wherein n is 1, 2, 3, 4, 5, 6, 7, 8, 9, or 10; A is oxygen or nitrogen; R¹¹ is hydrogen, salt-forming cation, ester forming group, —(CH₂)ₓ-Q, or when A is nitrogen, A and R¹¹ taken together may be the residue of a natural or unnatural amino acid or a salt or ester thereof; Q is hydrogen, thiazolyl, triazolyl, imidazolyl, benzothiazolyl, or benzoimidazolyl; x is 0, 1, 2, 3, or 4; R¹⁹ is hydrogen, alkyl or aryl; Y¹⁻ is oxygen, sulfur, or nitrogen; y² is carbon, nitrogen, or oxygen; R²⁰ is hydrogen, alkyl, amino, mercaptoalkyl, alkenyl, alkynyl, cycloalkyl, aryl, arylalkyl, thiazolyl, triazolyl, tetrazolyl, imidazolyl, benzothiazolyl, or benzoimidazolyl; R²¹ is hydrogen, alkyl, mercaptoalkyl, alkenyl, alkynyl, cycloalkyl, aryl, arylalkyl, thiazolyl, triazolyl, tetrazolyl, imidazolyl, benzothiazolyl, benzoimidazolyl, or absent if Y² is oxygen; R²² is hydrogen, alkyl, mercaptoalkyl, alkenyl, alkynyl, cycloalkyl, aryl, arylalkyl, thiazolyl, triazolyl, tetrazolyl, imidazolyl, benzothiazolyl, benzoimidazolyl; or R²² is hydrogen, hydroxyl, alkoxy or aryloxy if Y¹ is nitrogen; or R²² is absent if Y¹ is oxygen or sulfur; or R²² and R²¹ may be linked to form a cyclic moiety if Y¹ is nitrogen; or pharmaceutically acceptable salts thereof.

In another embodiment, the invention includes a pharmaceutical composition having a first agent that is a compound of Formula VII-A (see the attached Drawings) wherein: n is 2, 3, or 4; A is oxygen or nitrogen; R¹¹ is hydrogen, salt-forming cation, ester forming group, —(CH₂)ₓ-Q, or when A is nitrogen, A and R¹¹ taken together may be the residue of a natural or unnatural amino acid or a salt or ester thereof; Q is hydrogen, thiazolyl, triazolyl, imidazolyl, benzothiazolyl, or benzoimidazolyl; x is 0, 1, 2, 3, or 4; G is a direct bond or oxygen, nitrogen, or sulfur; z is 0, 1, 2, 3, 4, or 5; m is 0 or 1; R²⁴ is selected from a group consisting hydrogen, alkyl, mercaptoalkyl, alkenyl, alkynyl, aroyl, alkylcarbonyl, aminoalkylcarbonyl, cycloalkyl, aryl, arylalkyl, thiazolyl, triazolyl, imidazolyl, benzothiazolyl, and benzoimidazolyl; each R²⁵ is independently selected from hydrogen, halogen, cyano, amidino, hydroxyl, alkoxy, thiol, amino, nitro, alkyl, aryl, carbocyclic, or heterocyclic, and pharmaceutically acceptable salts thereof.

Such compounds of the invention include, for example, compounds of Formula I-B (see the attached Drawings) wherein X is oxygen or nitrogen; Z is C═O, S(O)₂, or P(O)OR 7; m and n are each independently 0, 1, 2, 3, 4, 5, 6, 7, 8, 9 or 10; R¹ and R⁷ are each independently hydrogen, metal ion, alkyl, mercaptoalkyl, alkenyl, alkynyl, cycloalkyl, aryl, a moiety together with X to form natural or unnatural amino acid residue, or —(CH₂)ₚ—Y; Y is hydrogen or a heterocyclic moiety selected from the group consisting of thiazolyl, triazolyl, tetrazolyl, amidino, imidazolyl, benzothiazolyl, and benzoimidazolyl; p is 0, 1, 2, 3, or 4; R² is hydrogen, alkyl, mercaptoalkyl, alkenyl, alkynyl, cycloalkyl, aryl, alkylcarbonyl, arylcarbonyl, or alkoxycarbonyl; R³ is hydrogen, amino, cyano, alkyl, mercaptoalkyl, alkenyl, alkynyl, cycloalkyl, heterocyclic, sunbstituted or unsubstituted aryl, heteroaryl, thiazolyl, triazolyl, tetrazolyl, amidino, imidazolyl, benzothiazolyl, or benzoimidazolyl, and pharmaceutically acceptable salts, esters, and prodrugs thereof.

In a further embodiment, in the compounds of Formula I-B, m is 0, 1, or 2. In another further embodiment, n is 0, 1, or 2. In another further embodiment, R³ is aryl, e.g., heteroaryl or phenyl. In yet another embodiment, Z is S(O)₂.

In another embodiment, the compound of the invention is of the Formula II-B (see the attached Drawings) wherein: each R⁴ is independently selected from the group consisting of hydrogen, halogen, hydroxyl, thiol, amino, amidino, cyano, nitro, alkyl, aryl, carbocyclic or heterocyclic; J is absent, oxygen, nitrogen, sulfur, or a divalent link-moiety consisting of, without limiting to, lower alkylene, alkylenyloxy, alkylenylamino, alkylenylthio, alkylenyl-oxyalkyl, alkylenylamonialkyl, alkylenylthioalkyl, alkenyl, alkenyloxy, alkenylamino, or alkenylthio; and q is 1, 2, 3, 4, or 5, and pharmaceutically acceptable salts, esters and prodrugs thereof.

In a further embodiment of compounds of Formula II-B, R⁴ is aryl, e.g., substituted or unsubstituted phenyl. In another embodiment, R⁴ is halogen (e.g., chlorine, fluorine, bromine, or iodine). In yet another embodiment, R⁴ is alkyl, e.g., methyl, ethyl, propyl, butyl, pentyl, trifluoromethyl, etc. In another embodiment, J is absent or oxygen. In a further embodiment, m is 1 or n is 1. In another further embodiment, the compound can be R- or S-isomer.

In yet a further embodiment, the compound may be selected from the group consisting of those compounds depicted in either Table X or Table Y (see the attached Drawings) and pharmaceutically acceptable salts, prodrugs, and esters thereof.

In still a further embodiment, the compound is selected from the group consisting of those compounds depicted in either Table Z-1 or Table Z-2 (see the attached Drawings) and pharmaceutically acceptable salts, prodrugs, and esters thereof.

In a further embodiment, the compound of the invention is of the Formula III-B (see the attached Drawings) wherein: X is oxygen or nitrogen; m and n are each independently 0, 1, 2, 3, 4, 5, 6, 7, 8, 9 or 10; q is 1, 2, 3, 4, or 5; R¹ is hydrogen, metal ion, alkyl, mercaptoalkyl, alkenyl, alkynyl, cycloalkyl, aryl, or a moiety together with X to form a natural or unnatural amino acid residue, or —(CH₂)ₚ—Y; Y is hydrogen or a heterocyclic moiety selected from the group consisting of thiazolyl, triazolyl, tetrazolyl, amidino, imidazolyl, benzothiazolyl, and benzoimidazolyl; p is 0, 1, 2, 3, or 4; R² is hydrogen, alkyl, mercaptoalkyl, alkenyl, alkynyl, cycloalkyl, aryl, alkylcarbonyl, arylcarbonyl, or alkoxycarbonyl; R⁵ is selected from the group consisting of hydrogen, halogen, amino, nitro, hydroxy, carbonyl, thiol, carboxy, alkyl, alkoxy, alkoxycarbonyl, acyl, alkylamino, acylamino; q is an integer selected from 1 to 5; J is absent, oxygen, nitrogen, sulfur, or a divalent link-moiety consisting of, without limiting to, lower alkylene, alkylenyloxy, alkylenylamino, alkylenylthio, alkylenyloxyalkyl, alkylenylamonialkyl, alkylenylthioalkyl, alkenyl, alkenyloxy, alkenylamino, or alkenylthio; and pharmaceutically acceptable salts, esters, and prodrugs thereof.

In still yet another embodiment, the compound of the invention may be a compound of Formula IV-B (see the attached Drawings). In a related embodiment, m is 0.

Examples of compounds of the invention include those compounds depicted in Table W (see the attached Drawings) and pharmaceutically acceptable salts, esters, and prodrugs thereof.

In another embodiment, the invention pertains to compounds of Formula V-B (see the attached Drawings) wherein: R⁶ is a substituted or unsubstituted heterocyclic moiety. In a further embodiment, m is 0 or 1. In another embodiment, n is 0 or 1. In another further embodiment, R⁶ is thiazolyl, oxazoylyl, pyrazolyl, indolyl, pyridinyl, thiazinyl, thiophenyl, benzothiophenyl, dihydroimidazolyl, dihydrothiazolyl, oxazolidinyl, thiazolidinyl, tetrahydropyrimidinyl, or oxazinyl. In yet another embodiment, Z is S(O)₂.

In a further embodiment, the invention pertains to the following compounds depicted in Table V (see the attached Drawings) and pharmaceutically acceptable salts, esters, and prodrugs thereof.

In yet another embodiment, the invention pertains to compounds of Formula I-C: 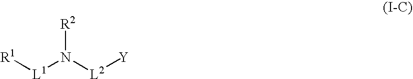
wherein:

R¹ is a substituted or unsubstituted cycloalkyl, heterocyclic, aryl, arylcycloalkyl, bicyclic or tricyclic ring, a bicyclic or tricyclic fused ring group, or a substituted or unsubstituted C₂-C₁₀ alkyl group;
R² is selected from a group consisting of hydrogen, alkyl, mercaptoalkyl, alkenyl, alkynyl, cycloalkyl, aryl, arylalkyl, thiazolyl, triazolyl, imidazolyl, benzothiazolyl, and benzoimidazolyl;
Y is SO₃⁻X⁺, SO₃⁻X⁺, or SSO₃⁻X⁺;
X⁺ is hydrogen, a cationic group, or ester-forming group; and
each of L¹ and L² is independently a substituted or unsubstituted C₁-C₅ alkyl group or absent, or a pharmaceutically acceptable salt thereof, provided that when R₁ is alkyl, L¹ is absent.

In a further embodiment, the invention pertains to compounds of Formula II-C: 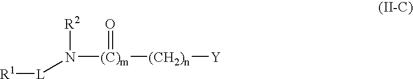
wherein:
R¹ is a substituted or unsubstituted cyclic, bicyclic, tricyclic, or benzoheterocyclic group or a substituted or unsubstituted C₂-C₁₀ alkyl group;
R² is hydrogen, alkyl, mercaptoalkyl, alkenyl, alkynyl, cycloalkyl, aryl, arylalkyl, thiazolyl, triazolyl, imidazolyl, benzothiazolyl, benzoimidazolyl, or linked to R¹ to form a heterocycle;
Y is SO₃⁻X⁺, OSO₃⁻X⁺, or SSO₃⁻X⁺;
X⁺ is hydrogen, a cationic group, or an ester forming moiety;
m is 0 or 1;
n is 1,2,3, or 4;
L is substituted or unsubstituted C₁-C₃ alkyl group or absent, or a pharmaceutically acceptable salt thereof, provided that when R¹ is alkyl, L is absent.

In a further embodiment, R² is hydrogen. In another further embodiment, R¹ is straight chain alkyl, for example, ethyl, n-pentyl, n-heptyl, or n-octyl. In another embodiment, R¹ is t-butyl. In yet another alternate embodiment, R¹ is C₇-C₁₀ bicycloalkyl or tricycloalkyl, such as, for example, tricyclo[3.3.1.0^{3.7}]decyl (or adamantyl), bicyclo[2.1.2]heptyl, or indolyl. In another alternate embodiment, R¹ is tetrahydronaphthyl.

In one embodiment, L² is —(CH₂)₃—. In another further embodiment, L² is —(CH₂)₄— or —(CH₂)₅—. In yet another further embodiment, L₂ is —(CH₂)₂—. In yet another further embodiment, L² is substituted alkyl, e.g., —CH₂—(CHOH)—CH₂—.

In another embodiment, L¹ is CH₂CH₂ or absent.

In a further embodiment, R¹ is branched alkyl, e.g., t-butyl. In another embodiment, R¹ is adamanyl. In another embodiment, R¹ is cyclic alkyl, e.g., cyclopropyl, cyclohexyl, cycloheptyl, cyclo-octyl, etc. The cycloalkyl moieties may be substituted further, e.g., with additional alkyl groups or other groups which allow the molecule to perform its intended function. In another embodiment, R¹ is alkyl substituted with a propargyl moiety (e.g., HC—C—). In another embodiment, R¹ is cyclohexyl substituted with one or more methyl or propargyl groups.

In other embodiments, L¹ is a C₁-C₂ alkyl linker group (e.g., —CH(CH₃)— or —(CH₂)₂-/. In a further embodiment, R¹ is phenyl. In certain embodiments, R¹ is substituted with a methoxy group. In other embodiments, L¹ is C₃, e.g., —(CH₂)₃— or C(CH₃)₂—. In certain embodiments, L¹ is substituted, e.g., with an alkoxy, carboxylate (—COOH), benzyl, amido (—C═O—NH—), or ester (C═O—C—O) group. In certain embodiment, the ester group is a methyl, ethyl, propyl, butyl, cyclohexyl, or benzyl ester. In other embodiments, the ester group may be propenyl. In other embodiments, L¹ is substituted with a carboxylate group. In a further embodiment, R¹ is substituted with a subsituted amido group, wherein the amido group is substituted with an alkyl, e.g., methyl, ethyl, propyl, butyl, pentyl, or hexyl group. In another embodiment, the alkyl R¹ group is a substituted with a —C═O—NH—OH, C═O—NH₂, or an amido group. In certain embodiments, the amido group is substituted with an alkyl (e.g., methyl, ethyl, propyl, butyl, pentyl, hexyl, cyclohexyl, bezyl or aryl group. In another embodiment, the amido group is substituted with a —CH(CH₂)₂ group. R¹ itself may be substituted with a phenyl or may be branched or straight chain alkyl. In certain embodiments, R¹ may also be substituted with a thioether moiety. Examples of thioethers include S-Me, S-Et, etc. In certain embodiments, the alkyl R¹ moiety is substituted with both an aryl or a thioether moiety and an amido moiety. In other embodiments, the alkyl R¹ moiety may be substituted with both a thioether and a carboxylate moiety. In other embodiments, alkyl R¹ groups are substituted with hydroxyl. R¹ groups, e.g., alkyl R¹ groups, may also be substituted with both thioether and hydroxyl groups.

In other embodiments, R¹ groups, e.g., alkyl R¹ groups are substituted with cyano groups. Examples of R¹ groups including —CN moieties include —C CH₃)₂CN, cyclohexyl substituted with one or more cyano groups, etc.

In other embodiments, alkyl R¹ groups are substituted with aryl groups. The aryl groups may be substituted phenyl, for example. The substituted phenyl may be substituted with one or more substituents such as hydroxy, cyano and alkoxy. In other embodiments, alkyl R¹ groups are substituted with tetrazolyl or substituted or unsubstituted benzyl.

In a further embodiment, L¹ is —C(CH₃)₂—(CH₂)—. In another embodiment, L¹ is —(C(CH₃)₂—CHOH—. In yet another embodiment, L¹ is —(C(CH₃)₂CH(OMe)-. In another embodiment, R¹ is substituted or unsubstituted phenyl. In a further embodiment, R¹ is para-substituted phenyl. Examples of substitutuents include but are not limited to fluorine, chlorine, bromine, iodine, methyl, t-butyl, alkoxy, methoxy, etc. In other embodiment, R¹ is substituted at the meta position. Examples of substituents include methoxy, chloro, methyl, t-butyl, fluoro, alkyl, alkoxy, iodo, trifluoroalkyl, methoxy, etc. In another embodiment, R¹ is phenyl substituted in the ortho position, with similar substituents. In another embodiment, L¹ comprises a cycloalkyl moiety, e.g., cyclopentyl. In another embodiment, L¹ comprises an alkyenyl group and, optionally, a substituted aryl group, with substittuents similar to those described about.

In certain embodiments, R¹ is cyclopropyl or cyclohexyl. In certain embodiments, the cyclopropyl or cyclohexyl group is subsituted with an ether group or an alkyl group. In certain further embodiments, the ether group is a benzyl ether group.

In another embodiment, wherein R′ is alkyl, it is substituted with groups such as phenyl, or hydroxy.

In another embodiment, the invention pertains to compounds of Formula III-C: 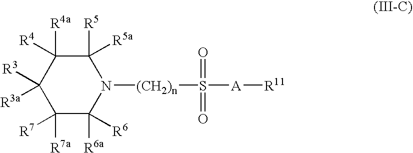
wherein:
A is nitrogen or oxygen;
R¹¹ is hydrogen, salt-forming cation, ester forming group, —CH₂)ₓ—, or when A is nitrogen, A and R¹¹ taken together may be the residue of a natural or unnatural amino acid or a salt or ester thereof;
Q is hydrogen, thiazolyl, triazolyl, imidazolyl, benzothiazolyl, or benzoimidazolyl;
x is 0, 1, 2,3, or 4;
n is 0, 1, 2, 3, 4, 5, 6, 7, 8, 9, or 10;
R³, R^{3a}, R⁴, R^{4a}, R⁵, R^{5a}, R⁶, R^{6a}, R⁷ and R^{7a} are each independently hydrogen, alkyl, mercaptoalkyl, alkenyl, alkynyl, cycloalkyl, aryl, alkylcarbonyl, arylcarbonyl, alkoxycarbonyl, cyano, halogen, amino, tetrazolyl, or two R groups on adjacent ring atoms taken together with the ring atoms form a double bond, provided that one of R³, R^{3a}, R⁴, R^{4a}, R⁵, R^{5a}, R⁶, R^{6a}, R⁷ and R^{7a} is a moiety of the Formula IIIa-C: 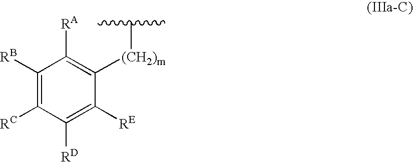
wherein:
m is 0, 1, 2,3, or 4;
R^{A}, R^{B}, R^{C}, R^{D}, and R^{E} are independently selected from a group of hydrogen, halogen, hydroxyl, alkyl, alkoxyl, halogenated alkyl, mercaptoalkyl, alkenyl, alkynyl, cycloalkyl, aryl, cyano, thiazolyl, triazolyl, imidazolyl, tetrazolyl, benzothiazolyl, and benzoimidazolyl; and pharmaceutically acceptable salts and esters thereof, provided that said compound is not 3-(4-phenyl-1,2, 3, 6-tetrahydro-1-pyridyl)-1-propanesulfonic acid. In a further embodiment, n is 2, 3 or 4.

In another embodiment, R¹¹ is a salt-forming cation. Examples of salt forming cations include pharmaceutically acceptable salts described herein as well as lithium, sodium, potassium, magnesium, calcium, barium, zinc, iron, and ammonium. In another embodiment, R¹¹ is an ester-forming group. An ester-forming group include groups when bound form an ester. Examples of such groups include substituted or unsubstituted alkyl, aryl, alkenyl, alkynyl, or cycloalkyl. In another embodiment, A is oxygen.

In another embodiment, R³ and R⁴ are taken together with the carbon atoms to which they are attached to form a double bond. In another embodiment, R^{A}, R^{B}, R^{C}, R^{D}, and R^{E} are each hydrogen. R^{A}, R^{B}, R^{D}, and R^{E} are each hydrogen and R^{C} is a halogen, such as fluorine, chlorine, iodine, or bromine.

In another embodiment, R³ or R^{5a} is a moiety of Formula IIIa-C.

In another embodiment, R⁴, R⁵, R⁶, and R⁷ are each hydrogen. In another further embodiment, R^{4a}, R^{5a}, R^{6a}, and R^{7a} are each hydrogen.

In another, R^{3a} is hydroxyl, cyano, acyl, or hydroxyl.

In another further embodiment, R¹¹ and A taken together are a natural or unnatural amino acid residue or a pharmaceutically acceptable salt or ester thereof. Examples of amino acid residues include esters and salts of phenylalanine and leucine.

In another embodiment, m is 0, 1, or 3.

In another embodiment, the invention pertains to compounds of Formula IV-C: 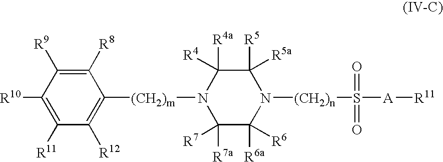
wherein:
A is nitrogen or oxygen;
R¹¹ is hydrogen, salt-forming cation, ester forming group, —(CH₂)ₓ—, or when A is nitrogen, A and R¹¹ taken together may be the residue of a natural or unnatural amino acid or a salt or ester thereof;
Q is hydrogen, thiazolyl, triazolyl, imidazolyl, benzothiazolyl, or benzoimidazolyl;
x is 0, 1, 2, 3, or 4;
n is 0, 1, 2, 3, 4, 5, 6, 7, 8, 9, or 10;
R⁴, R^{4a}, R⁵, R^{5a}, R⁶, R^{6a}, R⁷, and R^{7a} are each independently hydrogen, alkyl, mercaptoalkyl, alkenyl, alkynyl, cycloalkyl, aryl, alkylcarbonyl, arylcarbonyl, alkoxycarbonyl, cyano, halogen, amino, tetrazolyl, R⁴ and R⁵ taken together, with the ring atoms they are attached to, form a double bond, or R⁶ and R⁷ taken together, with the ring atoms they are attached to, form a double bond;
m is 0, 1, 2,3, or 4;
R⁸, R⁹, R¹⁰, R¹¹, and R¹² are independently selected from a group of hydrogen, halogen, hydroxyl, alkyl, alkoxyl, halogenated alkyl, mercaptoalkyl, alkenyl, alkynyl, cycloalkyl, aryl, cyano, thiazolyl, triazolyl, imidazolyl, tetrazolyl, benzothiazolyl, and benzoimidazolyl, and pharmaceutically acceptable salts and esters thereof.

In another embodiment, R¹¹ is a salt-forming cation. Examples of salt forming cations include pharmaceutically acceptable salts described herein as well as lithium, sodium, potassium, magnesium, calcium, barium, zinc, iron, and ammonium. In another embodiment, R¹¹ is an ester-forming group. An ester-forming group include groups when bound form an ester. Examples of such groups include substituted or unsubstituted alkyl, aryl, alkenyl, alkynyl, or cycloalkyl. In another embodiment, A is oxygen.

In another embodiment, m is 0 or 1. In another further embodiment, n is 2, 3, or 4. In another further embodiment, R⁴, R⁵, R⁶ and R⁷ are each hydrogen. R^{4a}, R^{5a}, R^{6a}, and R^{7a} also may be hydrogen. Examples of R⁸, R⁹, R¹⁰, R¹¹, and R¹² include hydrogen. In other embodiment R⁸, R⁹, R¹¹, R¹² are each hydrogen, and R¹⁰ is a halogen, (e.g., fluorine, chlorine, bromine, or iodine), nitro, or alkyl (e.g., methyl, ethyl, butyl).

In another embodiment, A-R¹¹ may be the residue of an amino acid, e.g., a phenyl alanine residue. In another embodiment, R⁹, R¹⁰, R¹¹ and R¹² are each hydrogen, and R⁸ is not hydrogen, e.g., halogen, e.g., fluorine, bromine, chlorine, or iodine.

In another embodiment, the invention pertains to compounds of Formula V-C: 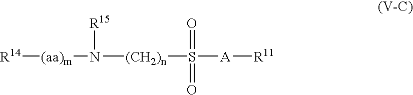
wherein:
A is nitrogen or oxygen;
R¹¹ is hydrogen, salt-forming cation, ester forming group, —(CH₂)ₓ-Q, or when A is nitrogen, A and R¹¹ taken together may be the residue of a natural or unnatural amino acid or a salt or ester thereof;
Q is hydrogen, thiazolyl, triazolyl, imidazolyl, benzothiazolyl, or benzoimidazolyl;
x is 0, 1, 2,3, or 4;
n is 0, 1, 2, 3, 4, 5, 6, 7, 8, 9, or 10;
aa is a natural or unnatural amino acid residue;
m is 0, 1, 2, or 3;
R¹⁴ is hydrogen or protecting group;
R¹⁵ is hydrogen, alkyl or aryl, and pharmaceutically acceptable salts and prodrugs thereof.

In another embodiment, R¹¹ is a salt-forming cation. Examples of salt forming cations include pharmaceutically acceptable salts described herein as well as lithium, sodium, potassium, magnesium, calcium, barium, zinc, iron, and ammonium. In another embodiment, R¹¹ is an ester-forming group. An ester-forming group includes groups when bound form an ester. Examples of such groups include substituted or unsubstituted alkyl, aryl, alkenyl, alkynyl, or. cycloalkyl. In another embodiment, A is oxygen.

In an embodiment, n is 2, 3 or 4. In certain embodiments, m is 0. In certain embodiments, A-R¹¹ is a residue of a natural amino acid, or a salt or ester thereof. Examples of amino acid residues, include, but are not limited, to leucine or phenylalanine residues, and pharmaceutically acceptable salts and esters thereof. Examples of possible esters include methyl, ethyl, and t-butyl.

In another embodiment, m is 1. Examples of aa include natural and unnatural amino acid residues such as phenylalanine, glycine, and leucine.

In another embodiment, (aa)ₘ is a residue of phe-phe; and pharmaceutically acceptable salts or an appropriate protecting group.

In certain embodiments, R¹⁵ is hydrogen or substituted alkyl, e.g., arylalkyl.

The term “unnatural amino acid” refers to any derivative of a natural amino acid including D forms, and α- and β-amino acid derivatives. It is noted that certain amino acids, e.g., hydroxyproline, that are classified as a non-natural amino acid herein, may be found in nature within a certain organism or a particular protein. Amino acids with many different protecting groups appropriate for immediate use in the solid phase synthesis of peptides are commercially available. In addition to the twenty most common naturally occurring amino acids, the following examples of non-natural amino acids and amino acid derivatives may be used according to the invention (common abbreviations in parentheses): β-alanine (β-ALA), γ-aminobutyric acid (GABA), 2-aminobutyric acid (2-Abu), α,β-dehydro-2-aminobutyric acid (8-AU), 1-aminocyclopropane-1-carboxylic acid (ACPC), aminoisobutyric acid (Aib), 2-amino-thiazoline-4-carboxylic acid, 5-aminovaleric acid (5-Ava), 6-aminohexanoic acid (6-Ahx), 8-aminooctanoic acid (8-Aoc), 11-aminoundecanoic acid (11-Aun), 12-aminododecanoic acid (12-Ado), 2-aminobenzoic acid (2-Abz), 3-aminobenzoic acid (3-Abz), 4-aminobenzoic acid(4-Abz), 4-amino-3-hydroxy-6-methylheptanoic acid (Statine, Sta), aminooxyacetic acid (Aoa), 2-aminotetraline-2-carboxylic acid (ATC), 4-amino-5-cyclohexyl-3-hydroxypentanoic acid (ACHPA), para-aminophenylalanine (4-NH₂-Phe), biphenylalanine (Bip), para-bromophenylalanine (4-Br-Phe), ortho-chlorophenylalanine] (2-Cl-Phe), meta-chlorophenylalanine (3-Cl-Phe), para-chlorophenylalanine (4-Cl-Phe), meta-chlorotyrosine (3-Cl-Tyr), para-benzoylphenylalanine (Bpa), tert-butylglycine (TLG), cyclohexylalanine (Cha), cyclohexylglycine (Chg), 2,3-diaminopropionic acid (Dpr), 2,4-diaminobutyric acid (Dbu), 3,4-dichlorophenylalanine (3,4-Cl₂-Phe), 3,4-diflurorphenylalanine (3,4-F₂-Phe), 3,5-diiodotyrosine (3,5-I₂-Tyr), ortho-fluorophenylalanine (2-F-Phe), meta-fluorophenylalanine (3-F-Phe), para-fluorophenylalanine (4-F-Phe), meta-fluorotyrosine (3-F-Tyr), homoserine (Hse), homophenylalanine (Hfe), homotyrosine (Htyr), 5-hydroxytryptophan (5-OH-Trp), hydroxyproline (Hyp), para-iodophenylalanine (4-1-Phe), 3-iodotyrosine (3-1-Tyr), indoline-2-carboxylic acid (Idc), isonipecotic acid (Inp), meta-methyltyrosine (3-Me-Tyr), 1-naphthylalanine (1-Nal), 2-naphthylalanine (2-Nal), para-nitrophenylalanine (4-NO₂-Phe), 3-nitrotyrosine (3-NO₂-Tyr), norleucine (Nle), norvaline (Nva), ornithine (Orn), ortho-phosphotyrosine (H₂PO₃-Tyr), octahydroindole-2-carboxylic acid (Oic), penicillamine (Pen), pentafluorophenylalanine (F₅-Phe), phenylglycine (Phg), pipecolic acid (Pip), propargylglycine (Pra), pyroglutamic acid (PGLU), sarcosine (Sar), tetrahydroisoquinoline-3-carboxylic acid (Tic), and thiazolidine4-carboxylic acid (thioproline, Th). Additionally, N-alkylatd amino acids may be used, as well as amino acids having amine-containing side chains (such as Lys and Orn) in which the amine has been acylated or or alkylated.

In another embodiment, the invention pertains, at least in part, to compounds of Formula VI-C: 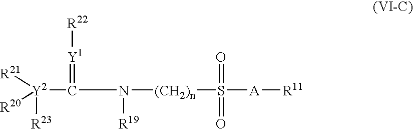
wherein:
n is 1, 2, 3, 4, 5, 6, 7, 8, 9, or 10;
A is oxygen or nitrogen;
R¹¹ is hydrogen, salt-forming cation, ester forming group, —(CH₂)ₓ-Q, or when A is nitrogen, A and R¹¹ taken together may be the residue of a natural or unnatural amino acid or a salt or ester thereof;
Q is hydrogen, thiazolyl, triazolyl, imidazolyl, benzothiazolyl, or benzoimidazolyl;
x is 0, 1, 2,3, or 4;
R¹⁹ is hydrogen, alkyl or aryl;
Y¹ is oxygen, sulfur, or nitrogen;
Y² is carbon, nitrogen, or oxygen;
R²⁰ is hydrogen, alkyl, amino, mercaptoalkyl, alkenyl, alkynyl, cycloalkyl, aryl, arylalkyl, thiazolyl, triazolyl, tetrazolyl, imidazolyl, benzothiazolyl, or benzoimidazolyl;
R²¹ is hydrogen, alkyl, mercaptoalkyl, alkenyl, alkynyl, cycloalkyl, aryl, arylalkyl, thiazolyl, triazolyl, tetrazolyl, imidazolyl, benzothiazolyl, benzoimidazolyl, or absent if Y² is oxygen;
R²² is hydrogen, alkyl, mercaptoalkyl, alkenyl, alkynyl, cycloalkyl, aryl, arylalkyl, thiazolyl, triazolyl, tetrazolyl, imidazolyl, benzothiazolyl, benzoimidazolyl; or R²² is hydrogen, hydroxyl, alkoxy or aryloxy if Y¹ is nitrogen; or R²² is absent if Y¹ is oxygen or sulfur; or R²² and R²¹ may be linked to form a cyclic moiety if Y¹ is nitrogen;
R²³ is hydrogen, alkyl, amino, mercaptoalkyl, alkenyl, alkynyl, cycloalkyl, aryl, arylalkyl, thiazolyl, triazolyl, tetrazolyl, imidazolyl, benzothiazolyl, or benzoimidazolyl, or absent if Y² is nitrogen or oxygen;
or pharmaceutically acceptable salts thereof.

In another embodiment, R¹¹ is a salt-forming cation. Examples of salt forming cations include pharmaceutically acceptable salts described herein as well as lithium, sodium, potassium, magnesium, calcium, barium, zinc, iron, and ammonium. In a further embodiment, the salt is a sodium salt. In a further, embodiment, A is oxygen.

In another embodiment, Y¹ is oxygen or sulfur, and R²² is absent.

In another embodiment, y² is oxygen and R²¹ is absent. Examples of R²⁰ include benzyl, aryl (e.g., phenyl), alkyl, cycloalkyl (e.g., adamantyl), etc. In other embodiment, Y² is nitrogen and R²¹ is hydrogen. In other embodiment, R²¹ is benzyl. In another further embodiment, R²⁰ and R²¹ are linked to form a pyridyl ring. In another embodiment, Y¹ is sulfur.

In another embodiment, the invention pertains to compounds of Formula VII-C: 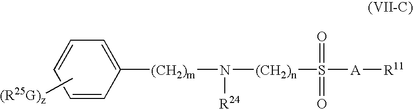
wherein:
n is 2,3, or 4;
A is oxygen or nitrogen;
R¹¹ is hydrogen, salt-forming cation, ester forming group, —(CH₂)ₓ-Q, or when A is nitrogen, A and R¹¹ taken together may be the residue of a natural or unnatural amino acid or a salt or ester thereof;
Q is hydrogen, thiazolyl, triazolyl, imidazolyl, benzothiazolyl, or benzoimidazolyl;
x is 0, 1, 2,3, or 4;
G is a direct bond or oxygen, nitrogen, or sulfur;
z is 0, 1, 2,3,4, or 5;
m is 0 or 1;
R²⁴ is selected from a group consisting of hydrogen, alkyl, mercaptoalkyl, alkenyl, alkynyl, aroyl, alkylcarbonyl, aminoalkylcarbonyl, cycloalkyl, aryl, arylalkyl, thiazolyl, triazolyl, imidazolyl, benzothiazolyl, and benzoimidazolyl;
each R²⁵ is independently selected from hydrogen, halogen, cyano, hydroxyl, alkoxy, thiol, amino, nitro, alkyl, aryl, carbocyclic, or heterocyclic, and pharmaceutically acceptable salts thereof.

In one embodiment, R¹¹ is hydrogen. In another, A is oxygen. For example, n may be 3 and m may be 1. In other embodiments, R¹⁴ is hydrogen or benzyl.

In certain embodiments, z is 0, 2, or 3. In others, R²⁵ is hydroxyl or alkoxy, e.g., methoxy, ethoxy, etc. In certain embodiments, two or more R²⁵ substituents can be linked to form a fused ring (e.g., to form a methylendioxyphenyl moiety).

The invention pertains to both salt forms and acid/base forms of the compounds of the invention. For example, the invention pertains not only to the particular salt forms of compounds shown herein as salts, but also the invention includes other pharmaceutically acceptable salts, and the acid and/or base form of the compound. The invention also pertains to salt forms of compounds shown herein.

Exemplary compounds of the invention are shown in the Figures and Tables attached hereto. Intended to be part of this invention are the exemplary compounds and selected groups and subsets thereof for any of the formulas recited herein, e.g., Formula I-C through VII-C, provided in the two U.S. Patent Applications filed Jun. 18, 2004, both entitled “Methods and Compositions for Treating Amyloid Related Diseases” (Attorney Docket Nos. NBI-162A and NBI-162B), and the U.S. Patent Application filed Jun. 18, 2004, entitled “Methods and Compositions for the Treatment of Amyloid- and Epileptogenesis-Associated Diseases” (Attorney Docket Nos. NBI-163), which are expressly incorporated by reference herein.

In one embodiment, the invention does not pertain to the compounds described in WO 00/64420, WO 97/023458 and WO 96/28187. In one embodiment, the invention does not pertain to methods of using the compounds described in WO 00/64420, WO 97/023458 and WO 96/28187 for the treatment of diseases or disorders described therein. In a further embodiment, the invention pertains to methods of using the compounds described in WO 00/64420, WO 97/023458 and WO 96/28187 for methods described in this application, which are not described in WO 00/64420, WO 97/023458 and WO 96/28187. Moreover WO 00/64420, WO 97/023458 and WO 96/28187 are incorporated by reference herein in their entirety.

In one embodiment the invention relates to the compounds in Table 2 (see attached Drawings). Additionally or alternatively the invention relates to the compounds in Table 2A (see attached Drawings). In one embodiment the invention does not pertain to the compounds in Table 2A and/or FIGS. 15 through 32. In one embodiment, the invention does pertain to the compounds in Table 2A and/or FIGS. 15 through 32.

Blockers of sodium or calcium ion channel activity are well known in the art and can be used as the A moiety in the compounds and methods of the present invention. Similarly, any compound that opens potassium or chloride ion channels can be used as the A moiety in the compounds and methods of the present invention. Antagonists of NMDA receptors and augmenters of endogenous GABA inhibition are also known to one of skill in the art and can be used in the methods and compounds of the invention. For example, 2,3-quinoxalinediones are reported to have NMDA receptor antagonistic activity (see, e.g., U.S. Pat. No. 5,721,234). Exemplary calcium and zinc chelators include moieties known in the art for chelation of divalent cations, including (in addition to those mentioned supra) ethylenediaminetetraacetic acid (EDTA), ethylene glycol bis(beta-aminoethyl ether)-N,N,N′,N′-tetraacetic acid, and the like. Exemplary iron chelators include enterobactin, pyridoxal isonicotinyl hydrazones, N,N′-bis(2-hydroxybenzoyl)-ethylenediamine-N,N′-diacetic acid (HBED), 1-substituted-2-alkyl-3-hydroxy-4-pyridones, including 1-(2′-carboxyethyl)-2-methyl-3-hydroxy-4-pyridone, and other moieties known in the art to chelate iron. Compounds which inhibit NO synthase activity are known in the art and include, e.g., Nγ-substituted arginine analogs (especially of the L configuration), including L-Nγ-nitro-arginine (a specific inhibitor of cerebral NO synthase), L-Nγ-amino-arginine, and L-Nγ-alkyl-arginines; or an ester (for example, the methyl ester) thereof. Exemplary antioxidants include ascorbic acid, tocopherols including alpha-tocopherol, and the like.

In another aspect, the invention relates to pharmaceutical compositions comprising a first agent, e.g., an alkanesulfonic acid, and another drug that targets secondary symptoms of Alzheimer's disease, such as behavioral and emotional difficulties. For example, some approved medications exist that appear to improve memory and cognition, but do not address the underlying pathology, as discussed more fully elsewhere herein.

The discussion below explains in more detail the various therapeutic approaches and classes of medications for Alzheimer's disease.

Cognitive Enhancers—Cholinesterase Inhibitors

Alzheimer's Disease is associated with degeneration of cholinergic neurons in the basal forebrain that play a fundamental role in cognitive functions, including memory. Alzheimer's disease patients exhibit a marked reduction in acetylcholinesterase activity and choline uptake. Becker, et al., Drug Development Research 12, 163-95 (1988). It has been observed that Aβ can inhibit acetylcholine release via its effects on, for example, choline transport, post-synaptic events and nicotinic acetylcholine receptors (α7, α2, β4) (Wang et al., J. Biol. Chem., vol. 275, pp. 5626-32 (2000); Melo et al., Amyloid, vol. 9, pp. 221-8 (2002); Kar et al., J. Neurosci., vol. 16, pp. 1034-1040 (1996)). By binding to Aβ, an alkylsulfonic acid could thus normalize acetylcholine esterase (ACHE) efflux levels (e.g. from the hippocampus) which are usually decreased in the presence of Aβ in the brain. In addition, AChE has been found to be associated with senile plaques and has been shown to interact with Aβ, promote amyloid fibril formation and increase its toxicity (De Ferrari et al., Biochemistry 40: 10447-10457, 2001). These findings suggest that in combination with a cholinesterase inhibitor or with an acetylcholine receptor agonist, an alkylsulfonic acid could act synergistically to treat an amyloid-β disease such as Alzheimer's disease.

Accordingly, in one aspect, the present invention is related to increasing levels of acetylcholine by the administration of an inhibitor of choline esterase (e.g., acetylcholinesterase or butyrylcholinesterase), and a first agent alkyl sulfonic acid which acts synergistically to treat an amyloid, (e.g., amyloid-β) disease. Cholinergic neurons make up a major neuronal system of the central and peripheral nervous systems. Cholinergic neurons produce the neurotransmitter acetylcholine. In the central nervous system, acetylcholine is a neurotransmitter and is released by cholinergic neurons in, among other places, the hippocampus and frontal cortex of the brain. The hippocampal area of the brain, particularly those areas where acetylcholine is released, is believed to have functions associated with cognition, learning, and memory. Degenerative diseases with symptoms such as loss of cognition, learning, and memory, have been linked to a loss in cholinergic neurons. Cholinergic dysfunction, characterized by marked degeneration of cholinergic innervation in the basal forebrain, and reduction of choline acetyltransferase, acetylcholinesterase, and the nicotinic and muscarinic receptors are known to be very early features of Alzheimer's disease. Other neurotransmitter systems, such as glutamatergic, serotonergic, and dopaminergic, are also disrupted in Alzheimer's disease, but at a later stage of the disease.

The present invention also relates to the combination use of a nicotinic acetylcholine receptor agonist or muscarinic agonist that results in cognition enhancement. Nicotinic acetylcholine receptor agonists improve cognitive function in Alzheimer's patients. Wilson, et al., Pharmacol. Biochem. Behavior 51, 509-14 (1995); Arneric, et al., Alzheimer Disease Assoc. Disorders 9(suppl. 2), 50-61 (1995); Buccafusco, et al., Behav. Pharmacol. 10, 681-90 (1999). Muscarinic and nicotinic agonists have been reported to enhance cognitive tasks in animal models and in humans. Schwarz, et al., J. Pharmacol. Experim. Theraput. 291, 812-22 (1999); Veroff, et al., Alzheimer Disease Assoc. Disorders 12, 304-12 (1998); Bodick, et al., Alzheimer Disease Assoc. Disorders 11 (Suppl. 4), S16-22 (1997).

In subjects with Alzheimer's disease, the number of cholinergic neurons innervating the hippocampus typically decreases, and the progressive loss of these cholinergic neurons mirrors the loss in memory and cognitive function in these subjects. Acetylcholine is synthesized by choline acetyltransferase (“ChAT”). Once released by the neuron, it is degraded by cholinesterases, e.g., acetylcholinesterase (“AChE”). Thus either potentiating the activity of ChAT or inhibiting the activity of a cholinesterase, e.g., AchE, may raise levels of the neurotransmitter. These medications alone appear to provide primarily symptomatic improvement.

Another therapeutic strategy for increasing levels of acetylcholine is based on up-regulating ChAT in the neurons. For example, estrogen increases the level of acetylcholine by up-regulating ChAT in the hippocampus of rats. Luine, et al., Brain Res. 191, 273-77 (1980); Luine, Exp. Neurology 89, 484-90 (1985), Singh, et al., Brain Res. 644, 305-12 (1994). Also clinical information implies that post-menopausal women on hormone replacement therapy (estrogen with or without progestins) may be less likely to develop Alzheimer's disease and more likely to have existing symptoms alleviated. See, e.g., WO 93/014085 (indole derivatives as having the ability to enhance the release of acetylcholine); U.S. Pat. No. 5,278,162 (substituted polycyclic compounds that enhance acetylcholine release).

Many choline esterase inhibitors are known. Certain cholinesterase inhibitors are approved for use in treatments for improving memory and learning in Alzheimer's subjects. Tacrine (Cognex™, Warner-Lambert Co., now Pfizer, New York, N.Y.) was the first approved cholinesterase inhibitor but is rarely used because of negative side effects like stomach and liver problems. Donepezil (Aricept™, Eisai Co, Ltd) is more selective for acetyl-cholinesterase and shows fewer side effects than tacrine. Rivastigmine (Exelon™, Novartis Pharma SA) targets a specific subtype of acetyl-cholinesterase that is present at high concentrations in the brains of Alzheimer's subjects. Galanthamine (Reminyl™, Janssen Pharmaceutica Products, LP) has a dual mode of action in the brain; in addition to working as an acetylcholinesterase inhibitor, galanthamine also appears to exert action on the nicotinic acetylcholine receptors in the brain. These cholinesterase inhibitors may be acetyl-cholinesterase or butyryl-cholinesterase inhibitors or both. Another example is phenserine (currently in advanced clinical trials in the United States). In addition to its cholinergic effects, phenserine may inhibit β-APP production by a separate and distinct mechanism of action at the level of the mRNA level. Another example is AIT-082 (also in advanced clinical trials). The degradation pathway of AchE may also be inhibited by inhibitors such as physostigmine (Synaptomm, or (Antilirium Injectable™, Forrest Laboratories, New York, N.Y.)), quilostigmine, tolserine, thiatolserine, cymserine, thiacymserine, neostigmine, eseroline, zifrosilone, mestinon, huperzine A and icopezil.

Phenserine, an acetylcholinesterase inhibitor, is in development (Axonyx, New York, N.Y.) for the treatment of Alzheimer's Disease. Phenserine, which has been shown to increase memory and learning in the laboratory animals, works through two mechanisms: it inhibits the degradation of the neurotransmitter acetylcholine in the brains of animals, and it inhibits the production of a toxic form of the α-amyloid protein in the brain that is thought to be a cause of the death of brain cells in Alzheimer's disease. Unlike other acetylcholinesterase inhibitors that simply suppress the activity of the enzyme, Phenserine's dual mechanism of action suggests that it not only has the potential to improve memory and cognition but also to slow the progression of the disease. Compared to currently marketed drugs for Alzheimer's, Phenserine is more brain-targeted versus the rest of the body and is more rapidly cleared from the blood. In preclinical studies, Phenserine demonstrated a brain-to-blood ratio of 10:1. These properties of Phenserine could potentially maximize the therapeutic effects of the drug in the brain and reduce side effects by clearing the drug from the blood quickly. Since undesirable side effects and drug interactions often arise due to the presence of drugs in the body for an extended period, Phenserine's rapid disappearance from the blood suggests that it will represent a more tolerable treatment option to existing therapies. Even though Phenserine is rapidly cleared from the body, the drug remains bound to the acetylcholinesterase enzyme in the brain allowing it to have a long duration of therapeutic action. Substituted phenserines and phenylcarbamates of eseroline, noreseroline, and benzylnoreseroline are also specific inhibitors of acetylcholinesterase See, e.g., U.S. Pat. Nos. 5,171,750; 5,378,723; 5,409,948; 5,998,460; 5,948,763; 6,410,747; 6,462,171; and 6,495,700; as well as WO 93/06105.

Suitable cholinesterase inhibitors include galanthamine derivatives available from Janssen, metrifonate available from Bayer Corp., ipidacrine available from Nikken Chemicals Co. Ltd., TAK-147, T-82 available from SS Pharmaceutical Co. Ltd., methanesulfonyl fluoride, CHF-2819, phenserine, physostigmine available from Forest Laboratories, Inc., huperzine, cymserine available from Axonyx Inc., tolserine available from National Institutes of Health, ER-127528 available from Eisai Co. Ltd., and combinations thereof.

In addition, the present invention relates to a method for maintaining or preventing a decrease in the levels of acetylcholine in the frontal cortex or hippocampus regions of the brain in mammals comprising administering to a mammal in need thereof, an effective amount of a first agent, e.g., an alkanesulfonic acid or a pharmaceutically acceptable salt thereof, and optionally a choline esterase inhibitor.

Further, the present invention relates to a method for inhibiting conditions or detrimental effects caused by a deficiency of choline acetyltransferase or acetylcholine in the frontal cortex or hippocampus regions of the brain in mammals comprising administering to a mammal in need thereof, an effective amount of a first agent, e.g., an alkanesulfonic acid, or a pharmaceutically acceptable salt thereof, and optionally a choline esterase inhibitor.

Moreover, the present invention relates to a pharmaceutical formulation comprising a first agent, e.g., an alkanesulfonic acid or a pharmaceutically acceptable salt thereof, and optionally a choline esterase inhibitor; and a pharmaceutical carrier, diluent, or excipient.

Another embodiment of the present invention is where the condition caused by a decrease of choline acetyltransferase or acetylcholine in the frontal cortex or hippocampus regions of the brain is Alzheimer's disease.

As used herein, the term “effective amount” means an amount of a first agent, e.g., an alkanesulfonic acid, that is capable of maintaining brain cell ability to produce stable levels of acetylcholine in the brain, such as in the hippocampus and frontal cortex regions, or inhibiting conditions or detrimental effects caused by a decrease of acetylcholine in mammals. When an alkanesulfonic acid or other such first agent is co-administered with an AChE inhibitor the term “effective amount” also means an amount of such an agent capable of inhibiting AChE. An inhibitor of AChE may be represented as “AChEi.”

In this context, the term “inhibiting” in the context of inhibiting conditions or detrimental effects caused by a deficiency of ChAT or acetylcholine in the frontal cortex or hippocampus regions of the brain includes its generally accepted meaning, i.e., prohibiting, restraining, alleviating, ameliorating, slowing, stopping, or reversing the progression or severity of a decrease in ChAT and acetylcholine and the pathological sequelae, i.e., symptoms, resulting from that event.

The term “up-regulating ChAT” refers to increasing the enzymatic activity of CHAT, i.e., promoting the conversion of choline to acetylcholine. This promotion would include an increase in the efficiency or rate of reaction of CHAT and choline or an increase in the amount of CHAT present at the site of action. This increase in the amount of enzyme present may be due to gene regulation or another synthetic step in the enzyme's formation or a decrease in the enzyme's de-activation and metabolism.

It has been shown that Aβ can inhibit the efflux of acetylcholine from neurons upon new stimulation, and in addition that exogenous Aβ may inhibit high affinity choline uptake. Normally acetylcholine efflux levels (e.g. from hippocampus) are decreased in the presence of Aβ in the brain. Aβ may act in several different ways to exert these effects, such as acting at the choline transporter, modulating post synaptic events, or acting on neuronal acetyl cholinesterase receptors (e.g., nAChr (α7, α2, β4). It has been shown that antibodies capable of binding to Aβ can normalize acetylcholine efflux levels, which are usually reduced in the presence of Aβ in the brain (Bales, et al., Cholignergic dysfunction in APP V717F transgenic mice is normalized following anti-Aβ antibody administration. See, Abstract from Neuroscience Meeting, New Orleans, November 2003 program no. 133.9.). A first agent of the invention, e.g., an alkanesulfonic acid, may act similarly to normalize acetylcholine levels by binding to AP. The presence of an alkanesulfonic acid may thus prevent Aβ from inhibiting the efflux of acetylcholine, thereby leading to an increase in the amount of acetylcholine at the synapse. It is likely therefore that an alkanesulfonic acid and an acetylcholinesterase inhibitor will act synergistically to ameliorate cholinergic neurotransmission, as both agents act to potentiate the levels of acetylcholine.

Ester Neurosciences (Herzlia Pituach, Israel) antisense drug (EN101) for the treatment of myasthenia gravis, demonstrated for the first time effective and safe use of an orally-administered antisense therapy for a neurological condition that lessened the severity of symptoms of myasthenia gravis, with no cholinergic symptoms nor significant adverse events based on balancing cholinergic transmission via controlled modulation of the company's novel target, a stress-response variant of acetylcholinesterase. AChE is an enzyme that degrades the neurotransmitter acetylcholine. EN101 selectively inhibits the production of the target at the critical stage of its biosynthesis, thereby allowing an effective treatment, while minimizing side effects and substantially improving upon the short-duration palliative relief currently observed with conventional inhibitors. EN101 is the lead compound in Ester's disease-modifying platform technology for the pre-expression control of a specific variant of the AChE protein, which is applicable to a wide range of neurological disorders.

Useful muscarinic receptor agonists include cevimeline, PD-151832 available from Pfizer Inc., YM-796 available from Yamanouchi Pharmaceutical Inc., and P-58 available from Phytopharm plc. Suitable acetylcholine release stimulators include minaprine, and montirelin available from Grunenthal GmbH, T-588 available from Toyama Chemical Co. Ltd., XE-991. Useful choline uptake stimulators include MKC-231 available from Mitsubishi-Tokyo Pharmaceuticals Inc. Suitable nicotinic cholinergic receptor agonists include altinicline available from SIBIA Neurosciences, Inc., SIB-1553A, ABT-089 (U.S. Pat. No. 5,278,176, Abbott Laboratories), nicotine patch, GRS-21, and TC-2403.

In 1993, tacrine became the first agent approved specifically for the treatment of cognitive symptoms of Alzheimer's disease. Tacrine is a reversible cholinesterase inhibitor and is thought to work by increasing the availability of intrasynaptic acetylcholine in the brains of Alzheimer's disease patients. The medication may also have other actions. Donepezil, another reversible cholinesterase inhibitor, is now available for the treatment of Alzheimer's disease. Another example of a second agent is xanomeline, which is a muscarinic selective m1 and m4 (muscarinic) acetylcholine receptor agonist and shows moderate improvement in cognitive performance, greater efficacy in decreasing psychotic symptoms, and agitation. N. C. Bodick, et al. “Effects of xanomeline, a selective muscarinic receptor agonist, on cognitive function and behavioral symptoms in Alzheimer disease.” Arch. Neurol. 54, 465-73 (1997). The second agent may also be an ergot alkaloid or a vinca alkaloid, such as Hydergine™ (Sandoz Pharmaceutical Corp., now Novartis, Basel, Switzerland) and nicergolin; or it may be a nootropic, such as piracetam, oxiracetam, pramiracetam, and aniracetam; which have cholinergic and dopaminergic properties as well as effects on protein processing. B.Saletu, et al., “Nicergoline in senile dementia of Alzheimer type and multi-infarct dementia: a double-blind, placebo-controlled, clinical and EEG/ERP mapping study.” Psychopharmacology 117, 385-95 (1995). In yet another embodiment, the second agent may be a carbamate derivative of physostigmine, such as eptastigrnine, which is an inhibitor of acetylcholinesterase. A.Norberg, et al., “Cholinesterase inhibitors in the treatment of Alzheimer's disease: a comparison of tolerability and pharmacology.” Drug Saf 19, 465-80 (1998).

Cognitive Enhancers—NMDA Receptor Antagonists

Excessive excitation by neurotransmitters can cause the degeneration and death of neurons. It is believed that this degeneration is in part mediated by the excitotoxic actions of the excitatory amino acids glutamate and aspartate at the N-methyl-D-Aspartate (NMDA) receptor. An increased level of one or more glutamate-related compounds is associated with many neurodegenerative disorders and neurodegeneration associated with long term disease states such as Huntington's disease, Alzheimer's disease, amyotrophic lateral sclerosis (ALS, which is also known as motor neuron disease), Parkinson's disease and acquired immunodeficiency (AIDS). Excitatory amino acid receptor antagonists that block NMDA receptors are recognized for usefulness in the treatment of disorders. NMDA receptors are intimately involved in the phenomenon of excitotoxicity, which may be a critical determinant of outcome of several neurological disorders. Disorders known to be responsive to blockade of the NMDA receptor include acute cerebral ischemia (stroke or cerebral trauma, for example), muscular spasm, convulsive disorders, neuropathic pain and anxiety, and may be a significant causal factor in chronic neurodegenerative disorders such as Parkinson's disease, amyotrophic lateral sclerosis (ALS), Alzheimer's disease and Huntington's disease. Compounds that effect the greatest protection of neurons from NMDA receptor-mediated injury, e.g., that injury resulting from stimulation of the NMDA receptor by glutamate or other excitatory amino acids or structurally similar compounds may be used in one embodiment of the invention.

Some examples of NMDA receptor antagonists are known and commercially available. Memantine (Ebixa™ or Axura™ or Namenda™ recently available in the U.S. from Merz Pharmaceuticals, Frankfurt am Main, Germany), which operates by yet another mechanism, appears to prevent or reduce the brain damage caused by Alzheimer's disease by blocking NMDA receptors in the brain. See U.S. Pat. No. 5,614,560. Memantine (1-amino-3,5-dimethyl adamantine) reduces neuronal damage by blocking NMDA receptor-operated channel activation by excitatory amino acids (such as glutamate-related compounds) at concentrations that are readily obtainable in human subjects taking the drug (Wesemann, et al., J Neural Transmission (Supp.) 16, 143 (1980)). Others include ethopropazine (Paridol™), diphenhydramine (Benadryl™), dizocilpine (Neurogaurd”), and amantadine (Symmetrel™).

Several drugs have NMDA antagonist activity without causing hypofunction because of activity at some other type of receptor. In the brains of healthy lab animals, such NMDA antagonists do not cause the vacuoles and other toxic side effects that are caused by NMDA antagonists such as PCP and MK-801, because of activity at the additional neuronal receptors. Such drugs, and the receptors they interact with in addition to NMDA receptors, include the following: Ibogaine, which also suppresses excitatory activity at sigma receptors and which may also be active at serotonin receptors, and Eliprodil, which also increases inhibitory activity at sigma receptors; Certain anti-cholinergic drugs such as procyclidine, trihexyphenydyl, and biperiden, which also suppress excitatory activity at muscarinic acetylcholine receptors; and Certain quinoxalinediones, including NBQX, ACEA 1021, and ACEA 1031, which are discussed below, and which suppress activity at non-NMDA receptors (ie., kainic acid receptors and AMPA receptors), in addition to NMDA receptors. As described in Example 11, NBQX blocks non-NMDA receptors so strongly that it acts as a safener agent when co-administered with MK-801. Accordingly, these and other quinoxalinediones are of great interest to pharmaceutical companies, and offer strong promise as inherently safened NMDA antagonists. Low-toxicity NMDA antagonists offer good candidates both for treating Alzheimer's disease, and for additional developmental research to identify analogs having adjusted balances in their dual or multiple receptor binding affinities. For example, the anti-parkinsonian agents procyclidine, trihexyphenydyl, and biperiden all have affinities for muscarinic receptors that are several times higher than for NMDA receptors. Further examples of NMDA receptor antagonists include those in U.S. Pat. No. 4,906,779, which discloses disubstituted guanidines, e.g., N,N′-di-m-tolyl guanidine, N,N′-di-o-ethylphenyl guanidine, N,N′-di-m-ethylphenyl guanidine, and N,N′-di-o-iodophenyl-guanidine; U.S. Pat. No. 5,498,610, which discloses 5-(1-hydroxy-2-piperidino)-propyl-2(1H,3H)-indolone analogs. A muscarinic agonist may also be used in this invention. A class of styryl amidine derivatives, which are antagonists of the human NMDA receptor, are selective for those containing the NR2B subunit, and may, in some embodiments, be used in the invention. U.S. Published Application No. 2003/0,119,871. Suitable NMDA receptor antagonists also include ipenoxazone, which is available from Nippon Chemiphar Co. Ltd.

The term “agonist,” as used herein, refers to a molecule which, when interacting with a biologically active molecule, causes a change (e.g., enhancement) in the biologically active molecule, or which positively modulates the activity of the biologically active molecule. An agonist interacts with a receptor and initiates a physiological or a pharmacological response characteristic of that receptor. As known in the art, agonists include, but are not limited to proteins, nucleic acids, carbohydrates, lipids or any other molecules which bind or interact with biologically active molecules. The terms “antagonist” or “inhibitor,” as used herein, refer to a molecule which, when interacting with a biologically active molecule, blocks or negatively modulates the biological activity of the biologically active molecule. Antagonists oppose the receptor-associated responses normally induced by other bioactive agents (i.e., agonists). Antagonists and inhibitors include, but are not limited to, proteins, nucleic acids, carbohydrates, lipids or any other molecules that bind or interact with biologically active molecules. Inhibitors and antagonists may effect the biology of entire cells, organs, or organisms (e.g., an inhibitor that slows or prevents neuronal degeneration and death).

Estrogens

Estrogen plays a powerful, pleiotropic role in many neurodegenerative conditions including Alzheimer's disease. Women have been shown to have increased risk, earlier onset, and more rapid progression of Alzheimer's Disease than men, although not gender-specific morbidity. Postmenopausal loss of estrogens leads to generally reversible decreases in memory that respond to estrogen replacement therapy; Besides mechanisms of blocking neurotoxicity directly, estrogen acts at various levels of plasticity: axon sprouting, synaptogenesis, and promoting synaptic transmission (electrophysiologically and biochemically). These effects may be ascribed to either receptor-dependent mechanisms, primarily transcriptional, including direct effects of ER in transcription and indirect effects through other transcription factors like CREB and Akt, as well as their retrograde transport or receptor-independent (rapid) mechanisms involving activational effects of second messenger systems, coexisting neurotransmission, or coordinated activation of both, as well as oxidative effects of the estrogen molecule. Estrogen replacement decreases the risk of Alzheimer's disease in postmenopausal women, delays the age of onset, and perhaps slows the decline. Estrogenic agent include estrogen, lasofoxifene, droloxifene, tamoxifen, and raloxifene (Evista™, Eli Lilly, Indianapolis, Ind.).

Also useful with the present invention are compositions or therapeutic combinations that further comprise hormone replacement agents and compositions. Useful hormone agents and compositions include androgens, estrogens, progestins, their pharmaceutically acceptable salts and derivatives. Combinations of these agents and compositions also are useful.

Examples of estrogens include, but are not limited to, androgen and estrogen combinations such as the combination of esterified estrogens (sodium estrone sulfate and sodium equilin sulfate) and methyltestosterone available from Solvay Pharmaceuticals, Inc., Marietta, Ga., as Estratest™; the blend of nine synthetic estrogenic substances including sodium estrone sulfate, sodium equilin sulfate, sodium 1 7α-dihydroequilin sulfate, sodium 17α-estradiol sulfate, sodium 17α-dihydroequilin sulfate, sodium 17α-dihydroequilenin sulfate, sodium 17β-dihydroequilenin sulfate, sodium equilenin sulfate and sodium 17β-estradiol sulfate; available from Duramed Pharmaceuticals, Inc., Cincinnati, Ohio, as Cenestin™; ethinyl estradiol, available by Schering Plough Corporation, Kenilworth, N.J., as Estinyl™; esterified estrogen combinations such as sodium estrone sulfate and sodium equilin sulfate; available from Solvay as Estratab™ and from Monarch Pharmaceuticals, Bristol, Term., as Menest™; estropipate, available from Pharmacia & Upjohn, Peapack, N.J., as Ogemm and from Women First Health Care, Inc., San Diego, Calif., as Ortho-Est™; and conjugated estrogens (17α-dihydroequilin, 17α-estradiol, and 17β-dihydroequilin), available from Wyeth, Philadelphia, Pa., as Premarin™. Another estrogen example is disclosed in U.S. Pat. No. 6,610,706.

Progestins and estrogens may also be administered as combinations including estradiol and norethindrone, available from Pharmacia & Upjohn, Peapack, N.J., as Activella™; levonorgestrel and ethinyl estradiol, from Wyeth as Alesse™, from Watson Laboratories, Inc., Corona, Calif., as Levora™ and Trivora™, Monarch Pharmaceuticals, as Nordette™, and from Wyethas Triphasil™; ethynodiol diacetate and ethinyl estradiol™; available from G. D. Searle & Co., as Demulen™ and Watson™ as Zovia™; desogestrel and ethinyl estradiol™, from Organon as Desogen™ and Mircette™, and from Ortho-McNeil Pharmaceutical, Raritan, N.J., as OrthoCept™; norethindrone and ethinyl estradiol; available from Parke-Davis, Morris Plains, N.J., under the tradenames Estrostep™ and Femhrtm, from Watson as Microgestin™, Necon™, and Tri-Norinyl™, from Ortho-McNeil as Modicon™ and Ortho-Novem™, and from Warner Chilcott Laboratories, Rockaway, N.J., under the tradename Ovcon™; the combination of norgestrel and ethinyl estradiol; available from Wyeth under the tradenames Ovral™ and Lo/Ovral™, and from Watson under the tradenames Ogestrel™ and Low-Ogestrel™; the combination of norethindrone, ethinyl estradiol, and mestranol, from Watson as Brevicon™ and Norinyl™, the combination of 17β-estradiol and micronized norgestimate, from Ortho-McNeil under the tradename Ortho-Prefest™; the combination of norgestimate and ethinyl estradiol; available from Ortho-McNeil under the tradenames Ortho Cyclemm and Ortho Tri-Ccyclen™; and the combination of conjugated estrogens (sodium estrone sulfate and sodium equilin sulfate) and medroxyprogesterone acetate, from Wyeth under the tradenames Premphase™ and Prempro™.

Examples of progestins include norethindrone; available from ESI Lederle, Inc., Philadelphia, Pa., as Aygestin™, from Ortho-McNeil under the tradename Micronor™, and from Watson as NOR-QD™; norgestrel; available from Wyeth as Ovrette™; micronized progesterone, from Solvay as Prometrium™; and medroxyprogesterone acetate; available from Pharmacia & Upjohn under the tradename Provera™.

Non-Steroidal Anti-Inflammatory Drugs

Nonsteroidal anti-inflammatory drugs (“NSAIDs”) appear to be associated with a lower likelihood of developing Alzheimer's disease. Anti-inflammatory drugs are believed to interfere with aspects of the microglial, astrocytic, and cytokine responses that occur in Alzheimer's disease. NSAIDs, including ibuprofen, naproxen, sulindac, and indomethacin, have been shown to be selective Aβ42-lowering agents. A subset of NSAIDs lower amyloidogenic Aβ42 independently of cyclooxygenase activity. S.Weggen, et al., “A subset of NSAIDs lower amyloidogenic Aβ42 independently of cyclooxygenase activity.” Nature 414, 212-16 (2001). Although the mechanisms by which these NSAIDs lower Aβ42 have not been established, the effect is independent of cyclooxygenase inhibition, which is the primary anti-inflammatory target of these compounds. NSAIDs do not appear to change the total level of Aβ produced but shift cleavage from Aβ42 to a less toxic shorter 38-amino acid Aβ peptide (Aβ38), which suggests that they interact with γ-secretase. One class of developmental compounds are inhibitors of PDE4, which act as anti-inflammatory drug in mice. These anti-inflamatory agent, e.g., rolipram, appear to block the microglial inflammatory response, and may have toxic side effects, but newer analogs without such properties are in development. Wilcock, et al., “Intracranially Administered Anti-Abeta Antibodies Reduce Beta-Amyloid Deposition by Mechanisms Both Independent of and Associated with Microglial Activation. J. Neurosci. 23(9), 3745-51 (2003). For example, Memory Pharmaceutical's MEM 1414 is a PDE4 inhibitor currently in testing for Alzheimer's disease.

Suitable anti-inflammatory agents include COX-2 inhibitors (such as Vioxx™ and Celebrex™), cytokine inhibitors (such as thalidomide disclosed in WO 95/04533 and dexanabinol) complement inhibitors, leukotriene receptor antagonists and combinations thereof. Examples include acetic acid aerivatives sulindac (Clinoril™, Merck & Co., Inc., Rahway, N.J.), indomethacin (Indocin™, Merck & Co., Inc., Rahway, N.J.); etodolac (Lodine™, Wyeth, Madison, N.J.), nabumetone (Relafen™, GlaxoSmithKline, Middlesex, England), tolmetin sodium (Tolectin™, McNeil Pharmaceuticals, Spring House, Pa.); anthranilic acid derivatives: meclofenamate sodium (Meclomen™, Pfizer, New York, N.Y.), mefenamic acid (Ponstel™, Pfizer, New York, N.Y.); enolic acid derivatives: piroxicam (Feldene™, Pfizer, New York, N.Y.), mobic (meloxicam); phenylacetic acid derivatives: arthrotec (diclofenac/misoprostol), Voltaremm (diclofenac); propionic acid derivatives: naproxen sodium (Anaprox™, Naprosyn™, Hoffmann-La Roche Inc. (Roche), Nutley, N.J.), flurbiprofen (Ansaid™, Upjohn, now Pfizer, New York, N.Y.), oxaprozin (Daypro™, G.D Searle, now Pfizer, New York, N.Y.); ibuprofen (Motrin™, Upjohn, now Pfizer, New York, N.Y.), fenoprofen calcium (Nalfomm, Dista, Ranbaxy, Princeton, N.J.), ketoprofen (Oruvail™ or Orudis™, Wyeth, Madison, N.J.), ketorolac tromethamine (Toradol™, Syntex Laboratories, Hoffiann-La Roche Inc. (Roche), Nutley, N.J.); salicylic acid derivative: diflunisal (Dolobid™, Merck & Co., Inc., Rahway, N.J.); and COX-2 selective inhibitors: Bextra™ (valdecoxib), Celebrex™ (celecoxib, Pfizer, New York, N.Y.), and ViOxx™ (rofecoxib, Merck & Co., Inc., Rahway, N.J.). Flurbiprofen is currently the subject of clinical trials with Alzheimer's patients by Myriad Genetics.

Maas BiolAB (Albuquerque, N. Mex.) is developing cyclosporin as an anti-inflammatory neuroprotection agent. EP 813,420 B 1. Cyclosporins, a class of drugs best known as immunosuppressants, were discovered to have a new use as the most effective neuroprotectants across the spectrum of neurological disease models when they cross the blood-brain barrier. Cyclosporins protect the brain's mitochondria and prevent neuron death due to traumatic brain and spinal cord injury, stroke, Alzheimer's, Parkinson's, Huntington's diseases and amyotrophic lateral sclerosis (ALS) animal models.

Anti-Oxidants

As a lipid rich organ, the CNS is particularly susceptible to effects of lipid peroxidation in modulating cellular signaling pathways, cell dysfunction, and cell death in the nervous system. In Alzheimer's disease, emerging evidence provides strong support for a role for oxidative stress in neurodegeneration, as multiple indices of oxidative stress have been observed, including protein oxidation, decreased polyunsaturated fatty acids, mitochondrial and nuclear DNA damage.

Free radicals (e.g., superoxide radicals) are used by phagocytes to kill bacteria and to oxidatively destroy foreign matter. Ordinarily excess superoxide is quenched by superoxide dismutase, however if oxidative stress causes the overproduction of radicals, or if the production of the superoxide exceeds the capacity of superoxide dismutase, then unintended oxidative damage may occur. B. Halliwell, Acta Neurol. Scand. 126, 23-33 (1989). In Alzheimer's subjects, protein oxidation, DNA oxidation, and lipid peroxidation are greater than in age-matched controls. S. S. Pitchumoni, et al., N. Engl. J. Med. 46(12), 1566-72 (1998).

An “antioxidant” is any substance capable of protecting against the damages of oxidative stress caused by reactive oxygen species such as free radicals. Antioxidants are generally desined so that they may be oxidized over other materials. In addition to superoxide dismutase, catalase and glutathione peroxidase react with hydrogen peroxide and convert it to water and diatomic oxygen. Other antioxidants include vitamin E (α-tocopherol), vitamin C (ascorbic acid), vitamin A (retinoic acid), co-enzyme Q, and selegiline.

Vitamin E and its derivitatives, e.g., α-tocopherol, quenches a free radical by donating a hydrogen atom thereby producing a tocopheroxy radical, which scavenges yet another peroxyl radical to produce α-tocopherol quinone, a stable compound. Unlike many other antioxidants, vitamin E is lipophilic and therefore soluble in the central nervous system and able to localize in a cell membrane thus preventing lipid peroxidation. Suitable Vitamin E derivatives include but are not limited to α-tocopherol, β-tocopherol, α-tocopheraol, γ-tocopheral, δ-tocopherol, ζ₁-tocopherol, ζ₂-tocopherol and η-tocopherol, and pharmaceutically acceptable ester derivatives thereof, e.g., the corresponding acetate, succinate and nicotinate forms. Additional antioxidants include citric acid in its various forms, including its administration as a combination of potassium citrate monohydrate and citric acid monohydrate. Additionally or alternatively, agents that supplement the chain-breaking antioxidant property of Vitamin E can be co-adminstered, e.g., ubiquinol, seleno-amino acids and sulfhydryl compounds (e.g., glutathione, sulfhydryl proteins, cysteine and methionine).

Also suitable as second or futher agents are butylated hydroxytoluene, butylated hydroxyanisole, propyl gallate, dodecylgallate, tert-butylhydroquinone, dihydrolipoic acid, prostaglandin B₁ oligomers (also known as polymeric 15-keto prostaglandin B or PGBₓ), 2-aminomethyl-4-tert-butyl-6-iodophenol, 2-aminomethyl-4-tert-butyl-6-propionylphenol, 2,6-di-tert-butyl-4-[2′-thenoyl]phenol, N,N′-diphenyl-p-phenylenediamine, ethoxyquin, probucol, ebselen, 5-[[3,5-bis(1,1-dimethylethyl)-4-hydroxyphen-yl]methylene]-3-(dimethylamino)-4-thiazolidinone, 5-[[3,5-bis(1,1-dimethylethyl)-4-hydroxyphenyl]meth-ylene]-3-(methylamino)-4-thiazolidinone (LY269415), D-myoinositol-1.2.6-trisphosphate, nordihydroguaiaretic acid, deferoxamine mesylate, tirilazad mesylate, derivatives of tirilazad in which the steroid portion of the chemical structure has been replaced with the tetramethyl chroman portion of d-α-tocopherol, trimetazidine, N,N′-dimethylthiourea, 2-(2-hydroxy-4-methylphenyl)aminothiazolehydrochloride, 2-L-oxothiazolidine, and combinations of these compouns or combinations with any other agents disclosed herein.

Additional antioxidants and free radical trapping substances that can be co-administered in accordance with the invention include plant (e.g., vegetable) active ingredients. This category, includes dimethyl sulfoxide, parthenolide, lycopene, daidzin, genistein, quercetin, morin, curcumin, apigenin, sesamol, chlorogenic acid, fisetin, ellagic acid, quillaia saponin, capsaicin, ginsenoside, silymarin, kaempferol, ginkgetin, bilobetin, isoginkgetin, isorhamnetin, herbimycin, rutin, bromelain, levendustin A, and erbstatin.

Glutamate and Aβ together have been shown to stimulate the production of free radicals in cultured neurons, but this process is retarded by the addition of catalase or α-tocopherol and agents that increase catalase activity. H. Hara, et al., Brain Res. 510, 335-38 (1990). Vitamin E has been shown to slow cognitive decline in Alzheimer's disease and in rat models. Patients with Alzheimer's disease treated with vitamin E showed a decreased rate of functional decline. Although it is not clear what causal relation oxidation has to Alzheimer's disease etiology, e.g., whether it is a secondary effect of the stress caused by synaptic or neuronal loss, antioxidant therapies have shown limited but promising efficacy in treating Alzheimer's disease. Vitamin E lacks negative medication interaction and may be used in combination with other Alzheimer's disease therapies. C.Behl, et al., “Vitamin E protects nerve cells from beta-amyloid protein toxicity.” Biochem. Biophys. Res. Commun. 186, 944-50 (1992); M.Sano, et al., “Rationale and design of a multicenter study of selegiline and a-tocopherol in the treatment of Alzheimer disease using novel clinical outcomes.” Alzheimer Dis. Assoc. Disord. 10, 13240 (1996); H.Kappus, et al., “Tolerance and safety of vitamin E: a toxicological position report.” Free Radic. Biol. Med 13, 55-74 (1992)).

Selegiline inhibits monoamine oxidase, which may convert certain protoxins into toxins. L. S. Schneider, J. Clin. Psychiatry 57, 30-36 (1996). Selegiline and other monoamine oxidase type B inhibitors may protect neurons from oxidative damage while not interfering with the action of type A inhibitors, which metabolize serotonin and norepinephrine. Selegiline also inhibits oxidative deamination of dopamine, which prevents the formation of free radicals and subsequent neuronal damage. M. Sano, et al., Alzheimer Dis. Assoc. Disord. 10, 132-140 (1996). Selegiline, through its anti-oxidative and neuroprotective properties may slow progression of Alzheimer's disease. Selegiline effect on catecholamine metabolism may also contribute to the efficacy of selegiline in delaying the progression of Alzheimer's disease in patients with moderate impairment.

Other antioxidants include free radical scavengers (Egb-761, yuyu Industrial, CP1-21, dexanabinol and iron chelators, which prevent iron from reacting to form hydroxyl radicals. Desferrioxamine prevents radical damage in vivo, and clinical trial shows that it may slow the progression of Alzheimer's disease. Yet another example is HCT-1026 (NO-flurbiprofen), which is a nitric oxide-donating derivative of flurbiprofen presently being developed in human clinical trials by NicOx SA (Sophia Antipolis, France). The chronic use of certain NSAIDS may result in gastrointestinal ulcers and impaired kidney function. Nitric oxide is believed to prevent or reverse such side-effects, thus making HCT-1026 particularly noteworthy.

Peroxisome Proliferator-Activated Receptor (PPAR) Agonists

Also useful in the present invention are compositions or therapeutic combinations that further comprise at least one (one or more) activators for peroxisome proliferator-activated receptors (“PPAR”). The activators act as agonists for the peroxisome proliferator-activated receptors. Three subtypes of PPAR have been identified, and these are designated as peroxisome proliferator-activated receptor alpha (“PPARα”), peroxisome proliferator-activated receptor gamma (“PPARγ”) and peroxisome proliferator-activated receptor delta (“PPARδ,” which is also known as “PPARβ” or “NUC1”).

Exposure of mammalian cells to PPAR agonists, particularly PPARα or PPARδ agonists, modulates, e.g., decreases the production or release of Aβ, particularly Aβ42, from the cells. See, U.S. Patent Application Publication No. 2003/0125338, which describes administrations of peroxisome proliferator-activated receptors for the treatment of amyloidosis and conditions and diseases associated therewidth. The peroxisome proliferator-activated receptors (PPARα, PPARδ, PPARβ, and PPARγ) are a subfamily of the nuclear receptor gene family, Desvergne, et al., Endocrine Rev. 20, 649-88 (1999)). PPARs are usually activated by fatty acids and similar derivatives. PPARδ has been identified as being useful in increasing high density lipoprotein (HDL) levels in humans. See, e.g., WO 97/28149, which describes PPAR agonists that are useful for raising high density lipoprotein (HDL) plasma levels in mammals. PPARα activator compounds are useful for, among other things, lowering triglycerides, moderately lowering LDL levels and increasing HDL levels. Useful examples of PPARα activators include fibrates.

In contrast to PPARα, the function of PPARδ is not well understood. Although PPARδ is ubiquitously expressed the brain, adipose tissue and skin have higher levels of relative mRNA expression (J. M. Peters, et al., Mol. Cell. Biol. 20, 5119-28 (2000)). The expression profile of PPARδ suggests that it may be involved in brain functions. G. Xing, et al., Biochem. Biophys. Res. Commun. 217, 1015-25 (1995). Furthermore, PPARδ may be implicated in reverse cholesterol transport, W.R. Oliver, et al., Proc. Nat'l Acad. Sci USA 98, 5306-11 (2001). Examples of PPARδ agonists include valproic Acid (Lampen, et al., Tox. Appl. Pharmacol. 160, 238-49 (1999)), GW501516 (W.R. Oliver, et al., Proc. Nat'lAcad. Sci. USA 98, 5306-11 (2001)), L-165041, L-165461, L-783483, and L-796449 (Berger, et al., J. Biol. Chem. 274, 6718-25 (1999)).

Routine experimentation may be performed to determine if a composition affects the release of Aβ from at least one cell in vivo. A suitable assay involves SM-4 cells, which are stably transfected with Swedish mutant amyloid Precursor Protein, and then treated with a PPARα or PPARδ agonist, such as pirinixic acid, or derivative thereof. After treatment, the media is collected and assayed for Aβ₄₀ or Aβ₄₂. A statistically significant decrease (p<0.05) in Aβ₄₀ or Aβ₄₂ concentration in the media compared to appropriate control(s) indicates that the treatment inhibited or prevented Aβ₄₀ or Aβ₄₂ production or release from the cells. If a compound decreases Aβ₄₂ production or release by a statistically significant amount relative to control (absence of the compound or presence of vehicle) it is considered to be an Aβ₄₂-modulating agent according to the invention.

An exemplary PPAR agonist is pirinixic acid, which has been shown to induce a decrease in Aβ₄₂ production or release from SM-4 cells in a concentration-dependent manner. Pirinixic acid has been identified as a hypolipidemic agent, see, U.S. Pat. No. 3,814,761, which characterized it and related compounds as anti-lipidemic agents. Although it might be tempting to view the activity of pirinixic acid on Aβ42 production or release as being directly related to its hypolipidemic role, particularly in view of the clinical correlation between hypercholesterolemia and Alzheimer's disease. Wolozin, Proc. Nat'l Acad. Sci USA 98, 5371-73 (2001)). Fibrates are known to act as cholesterol-lowering agents but they generally are not known to reduce Aβ42 production or release. For example, it has been reported that when SM-4 cells were treated with clofibrate and the culture media was collected in order to assay Aβ42 levels, clofibrate was found to increase Aβ42 extracellular levels at a concentration range of 50-500 μM. Similar results were found with 5,8,11,14-eicosatetraynoic acid (“ETYA”) at 20-50 μM concentrations. The fact that three PPARα agonists (all of which are cholesterol lowering agents) have disparate effects on Aβ42 production or release from SM-4 cells implies that some PPARα agonists affect Aβ42 production or release via a mechanism that is not strictly concomitant with their role as cholesterol lowering agents. See, U.S. Patent Application Publication No. 2003/0013699, which describes novel heterocycles designed to prevent, treat, or ameliorate symptoms of Alzheimer's Disease, regulating production or levels of amyloid-β peptides in the bloodstream or brain.

Non-limiting examples of suitable fibric acid derivatives (“fibrates”) include clofibrate (such as ethyl 2-(p-chlorophenoxy)-2-methylpropionate, for example Atromid-S™ capsules, which are commercially available from Wyeth, Madison, N.J.); gemfibrozil (such as 5-(2,5-dimethylphenoxy)-2,2-dimethylpentanoic acid, for example, Lopid™ tablets, which are commercially available from Pfizer, New York, N.Y.); ciprofibrate (C.A.S. Registry No. 52214-84-3, see, U.S. Pat. No. 3,948,973, which describes the synthesis of such halocyclopropyl substitutedphenoxyalkanoic acids and esters); bezafibrate (C.A.S. Registry No. 41859-67-0, see, U.S. Pat. No. 3,781,328, which describes the synthesis of novel phenoxy-alkyl-carboxylic acid compounds and their ability to lower the serum lipid and cholesterol level); clinofibrate (C.A.S. Registry No. 30299-08-2, see, U.S. Pat. No. 3,716,583, which describes the preparation of novel anti-atherosclerosis agents); binifibrate (C.A.S. Registry No. 69047-39-8); lifibrol (C.A.S. Registry No. 96609-16-4); fenofibrate (such as Tricomm micronized fenofibrate (2-[4-(4-chlorobenzoyl)-phenoxy]-2-methylpropanoic acid, 1-methylethyl ester), which is available from Abbott Laboratories, Abbott Park, Ill., or Lipanthyl™ micronized fenofibrate, available from Laboratoire Founier, Chenôve, France).

Other examples of PPARα activators include suitable fluorophenyl compounds as disclosed in U.S. Pat. No. 6,028,109, which describes the use of agnosts of PPARα for the manufacture of a medicament for the treatment of obesity and the methods of treating obesity; certain substituted phenylpropionic compounds as disclosed in WO 00/75103, which describes novel substituted phenylpropionic acid derivatives capable of binding as a ligand to PPARα to thereby activate the receptor and thus show a potent effect of lowering blood lipid; and PPARα activator compounds as disclosed in WO 98/43081, which describes methods and compositions for treating a host having a gastrointestinal disease by administering to the host a composition containing a pharmaceutically effective amount of a modulator of a PPAR. Non-limiting examples of suitable PPARγ activators include derivatives of glitazones or thiazolidinediones, such as, troglitazone (such as Rezulin™ 5[[4-[(3,4-dihydro-6-hydroxy-2,5,7,8-tetramethyl-2H-1-benzopyran-2-yl) methoxy]phenyl]methyl]-2,4-thiazolidinedione) commercially available from Pfizer, New York, N.Y.); rosiglitazone (such as Avandia™ rosiglitazone maleate 5-[[4-[2-(methyl-2-pyridinylamino)ethoxy]phenyl]methyl]-2,4-thiazolidinedione, (Z)-2-butenedioate) available from GlaxoSmithKline, Middlesex, England) and pioglitazone (such as Actos™ pioglitazone hydrochloride (5-[[4-[2-(5-ethyl-2-pyridinyl)ethoxy]phenyl]methyl]-2,4-]-thiazolidine-dione monohydrochloride) commercially available from Takeda Pharmaceuticals, Lincolnshire, Ill.). Other useful thiazolidinediones include ciglitazone, englitazone, darglitazone and BRL 49653, see, WO 98/05331, which relates such compounds for the prevention and treatment of type 2 diabetes and cardiovascular disease; PPARγ activator compounds disclosed in WO 00/76488, which describes methods for delaying or preventing the onset of Type 1 diabetes; and PPARγ activator compounds disclosed in U.S. Pat. No.-5,994,554, which describes a method for determining whether a compound does or does not interact directly with PPARY using radiolabeling.

Other useful PPARγ activator compounds include certain acetylphenols, see, e.g., U.S. Pat. No. 5,859,051, which describes acetylphenol and its analogues for use as antiobesity and antidiabetic compounds; certain quinoline phenyl compounds as disclosed in WO 99/20275, which describes the use of such compounds for mediating the activity of PPAR receptors with such compounds; aryl compounds as disclosed by WO 99/38845, which describes such compounds for use as PPARγ modulators for the treatment for conditions such as type 2 diabetes and obesity; certain 1,4-disubstituted phenyl compounds as disclosed in WO 00/63161, which describes such compounds as highly selective agonists for the PPAR receptor or prodrugs of agonists for the PPARγ receptor, and therefore useful in the treatment of type 2 diabetes; certain aryl compounds as disclosed in WO 01/00579, which describes such compounds as modulators of PPARγ activity which are useful in pharmaceutical compositions and methods for the treatment of conditions such as type 2 diabetes and obesity; benzoic acid compounds as disclosed in WO 01/12612 & WO 01/12187, which describe such compounds as PPAR agonists, in particular PPARγ, and so are useful in the treatment of states of insulin resistance, including type 2 diabetes mellitus; and substituted 4-hydroxy-phenylalconic acid compounds as disclosed in WO 97/31907, which describes such compounds as exhibiting activation, including agonist activity, to PPARδ, thereby enabling them to modulate the blood glucose levels in mammals.

PPARδ compounds are useful for, among other things, lowering triglyceride levels or raising HDL levels. Non-limiting examples of PPARδ activators include suitable thiazole and oxazole derivatives, such as C.A.S. Registry No. 317318-324, see, e.g., WO 01/00603, which describes the use of pamoic acid or one of its derivatives for the preparation of a medicament for the treatment of diseases characterized by deposits of amyloid aggregates; suitable non-β-oxidizable fatty acid analogues); certain fluoro, chloro or thio phenoxy phenylacetic acids; see; e.g., WO 97/28149, which describes such compounds as useful for raising high density lipoprotein (HDL) plasma levels in mammals and for preventing, halting or slowing the progresssion of atherosclerotic cardiovascular diseases and related conditions; see, e.g., U.S. Pat. No. 5,093,365, which describes the ability of such fatty acid analogues to lower the concentration of cholesterol and triglyceride in the blood; and PPARδ compounds as disclosed in WO 99/04815, which describes medicinal compositions with a cholesterol-lowering effect containing, as the active ingredient, compounds having the effect of activating a PPARδ receptor, thereby having an LDL-cholesterol-lowering effect.

Moreover, compounds that have multiple functionality for activating various combinations of PPARα, PPARγ and PPARδ are also useful with the practice of the present invention. Non-limiting examples include certain substituted aryl compounds as disclosed in U.S. Pat. No.-6,248,781, which describes the ability of such compounds in the treatment or prevention of conditions mediated by nuclear receptors, in particular PPAR; WO 00/23416; WO 00/23415; WO 00/23425; WO 00/23445; WO 00/23451; and WO 00/63153, all of which describe compounds that may be utilized in the treatment of conditions mediated by PPARα or PPARγ activator compounds, such as diabetes and obesity. Other non-limiting examples of useful PPARα or PPARγ activator compounds include activator compounds as disclosed in WO 97/25042, which describes the use of a pharmaceutically effective amount of an agonist of PPARα and PPARγ for the treatment or prophylaxis of Syndrome X; activator compounds as disclosed in WO 00/63190, which describes novel compounds that may be utilized in the treatment or prevention of conditions mediated by nuclear receptors, in particular PPAR; activator compounds as disclosed in WO 01/21181, which describes novel drugs efficacious against diseases in association with glycometabolism and lipid metabolibsm by inhibiting or promoting PPARα or PPARγ; biaryl-oxa(thia)zole compounds as disclosed in WO 01/16120, in which modulators of PPARs are useful in the treatment of type 2 diabetes and of cardiovascular diseases; compounds as disclosed in WO 00/63196, which describes compounds that are useful in the treatment of conditions mediated by nuclear receptors, in particular Retinoid X Receptor and PPARs families; and WO 00/63209, which describes a pharmaceutical composition useful in the treatment or prevention of conditions mediated by PPARs; substituted 5-aryl-2,4-thiazolidine diones compounds as disclosed in U.S. Pat. No. 6,008,237, which describes substituted 5-aryl-2,4-thiazolidinediones as potent agonists of PPAR, and are therefore useful in the treatment, control or prevention of diabetes, hyperglycemia, vacular restenosis, and other PPAR mediated diseases; arylthiazolidinedione and aryloxazolidinedione compounds as disclosed in WO 00/78312 and WO 00/78313, which describe substituted 5-aryl-2,4-thiazolidinediones and oxazolidinediones as potent agonists of PPAR, and are therefore useful in the treatment, control or prevention of PPAR α or γ mediated diseases; GW2331 or (2-(4-[difluorophenyl]-1-heptyl ureido)-ethyl]-phenoxy)-2-methylbutyric compounds, see, e.g., WO 98/05331, which describes such compounds for the prevention and treatment of type 2 diabetes and cardiovascular disease with diabetic or pre-diabetic conditions or symptoms by behaving as both a PPARα agonist and a PPARγ agonsit, or activating both PPARα and PPARγ; aryl compounds as disclosed in U.S. Pat. No. 6,166,049, which describes a method comprising the administration of PPARα and PPARδ; oxazole compounds as disclosed in WO 01/17994, which describes chemical modification of a phosphorous-based PPAR agonist; and dithiolane compounds as disclosed in WO 01/25225, and WO 01/25226, which describes methods for synthesizing novel dithiolane derivatives with high affinity for PPARα or PPARγ.

Other useful PPAR activator compounds include substituted benzylthiazolidine-2,4-dione compounds as disclosed in WO 01/14349, WO 01/14350, and WO 01/04351, all of which show how such a compound, as a ligand of human PPAR, enhances the transcriptional activity of the receptor and effects the lowering of blood sugar level and lipid level; mercaptocarboxylic compounds as disclosed in WO 00/50392, which demonstrates how such compounds exhibit excellent antihyperglycemic and PPAR-activating effects; ascofuranone compounds as disclosed in WO 00/53563, which demonstrates how such compounds are usable in preventing or treating diabetes, chronic inflammation, digestive cancers, etc.; carboxylic compounds as disclosed in WO 99/46232, which have and effect of regulating PPARs; compounds as disclosed in WO 99/12534, which describes aromatic compounds that exhibit control effects against PPAR; benzene compounds as disclosed in WO 99/15520, which describes compounds that exhibit control effects against PPAR and therefore useful for the treatment of related diseases; o-anisamide compounds as disclosed in WO 01/21578, which describes the ability of such compounds to serve as PPAR agonists; and PPAR activator compounds as disclosed in WO 01/40192, which describes heterocyclic compounds that have the effects of lowering blood glucose level, lowering blood lipid level, ameliorating insulin resistance and activating PPAR.

Cholesterol-Lowering Agents

Since one aspect of the present invention relates to treating Alzheimer's disease, regulating production of or levels of amyloid β (Aβ) peptides or regulating the amount of ApoE isoform 4 in the bloodstream or brain by treatment with a combination of active ingredients wherein the active ingredients may be administered separately, the invention also relates to combining separate pharmaceutical compositions in kit form. That is, the invention includes a kit wherein two separate units are combined: a pharmaceutical composition comprising at least a compound of any of the Formulae described herein and a separate pharmaceutical composition comprising at least one cholesterol biosynthesis inhibitor or lipid-lowering agent as described above. In one embodiment, the kit may include directions for the administration of the separate components. The kit form is particularly advantageous when the separate components must be administered in different dosage forms (e.g., oral and parenteral) or are administered at different dosage intervals.

In another alternative embodiment, the compositions used in the methods of the present invention can further comprise one or more AcylCoA:Cholesterol O-acyltransferase (“ACAT”) Inhibitors, which can reduce LDL and VLDL levels, coadministered with or in combination with the compound(s) of the Formulae herein discussed above. ACAT is an enzyme responsible for esterifying excess intracellular cholesterol and may reduce the synthesis of VLDL, which is a product of cholesterol esterification, and overproduction of apo B-100-containing lipoproteins.

Non-limiting examples of useful ACAT inhibitors include avasimibe ([[2,4,6-tris(1-methylethyl)phenyl]acetyl]sulfamic acid, 2,6-bis(1-methylethyl)phenyl ester, formerly known as C1-1011), HL-004, lecimibide (DuP-128) and CL-277082 (N-(2,4-difluorophenyl)-N-[[4-(2,2-dimethylpropyl)phenyl]methyl]-N-heptylurea). See P. Chang et al., “Current, New and Future Treatments in Dyslipidaemia and Atherosclerosis”, Drugs 60(1), 55-93 (2000).

There is a complex relationship between Alzheimer's disease, cholesterol homeostasis, and agents used for regulating cholesterol levels in the body. WO 00/28981 discloses the administration of an inhibitor of HMG CoA reductase (3-hydroxy-3-methylglutaryl CoA reductase) to reduce the risk of onset of Alzheimer's disease. The inhibitors used were lovastatin, pravastatin, or a combination thereof. However, a similar correlation was not seen with simvastatin. WO 00/31548 also discloses inhibitors of HMG CoA reductase, particularly statins. Interestingly, simvastatin is a suggested inhibitor, contrasting with the results disclosed in WO 00/28981, which states that the prevalence of Alzheimer's disease in simvastatin-treated subjects was not decreased.

More than half of the total body cholesterol in humans is derived from intrinsic biosynthesis. HMG-CoA (3-hydroxy-3-methylglutaryl-coenzyme A) reductase is the enzyme catalyzing the early rate-limiting step in cholesterol biosynthesis, i.e., conversion of HMG-CoA to mevalonate. Cholesterol and triglycerides circulate in the bloodstream as part of lipoprotein complexes. These complexes may be separated by density ultracentrifugation into high (HDL), intermediate (IDL), low (LDL), and very low (VLDL) density lipoprotein fractions. Triglycerides (TG) and cholesterol synthesized in the liver are incorporated into VLDLs and released into the plasma for delivery to pheripheral tissues. In a series of subsequent steps, VLDLs are transformed into IDLs and cholesterol-rich LDLs. HDLs, containing apolipoprotein A, are hypothesized to participate in the reverse transport of cholesterol from tissues back to the liver. Elevated levels of total cholesterol, i.e., hypercholesterolemia, low LDL-cholesterol (LDL-C), and apolipoprotein B (a membrane transport protein for LDL) promote human atherosclerosis. Similarly, decreased levels of HDL-cholesterol (HDL-C) and its transport complex, apolipoprotein A are associated with the development of atherosclerosis. Cardiovascular morbidity and mortality vary directly with the level of total cholesterol and LDL-C, and inversely with the level of HDL-C. HMG-CoA reductase inhibitors have been shown to reduce total serum cholesterol levels, LDL-C, and apolipoprotein B, most likely by increasing the catabolism of LDL and hepatic extraction of LDL precursors, blocking enzymatic cholesterol synthesis, and simultaneously increasing HD levels. These lipid lowering drugs lower serum cholesterol levels and reduce the incidence of both cardiovascular and cerebrovascular events. See, e.g, U.S. Pat. Nos. 5,831,115; 5,807,834; 5,801,143; 5,798,375; and 5,786,485. Statins, well known for the treatment of prevention of coronary heart disease, block a rate-limiting step in the biosynthesis of cholesterol by HMG-CoA reductase. See U.S. Pat. No. 6,465,516.

While Alzheimer's disease is typically characterized pathologically by the presence of senile plaques and neurofibrillary tangles found at autopsy in the brains of subjects afflicted with the disease, vascular components of the disease have also been noted. These include lesions in the cerebral microcirculation and vascular deposits of Aβ protein, which is also a major constituent of the senile plaques found in Alzheimer's disease.

In addition to a relationship with coronary disease, it is known that there is a relationship between serum cholesterol levels and the incidence and the pathophysiology of Alzheimer's disease. Epidemiological studies show that subjects with elevated cholesterol have an increased risk of Alzheimer's disease. (Notkola et al., “Serum total cholesterol, apolipoprotein E epsilon 4 allele, and Alzheimer's disease,” Neuroepidemiology; 17(1): 14-20 (1998); Jarvik et al., “Interactions of apolipoprotein E genotype, total cholesterol level, age and sex in prediction of Alzheimer's disease: a case-control study,” Neurology 45(6): 1092-6 (1995).) Other studies have established that subjects possessing the apolipoprotein ε4 genotype (“apoE4”) that codes for a variant of apolipoprotein, a cholesterol transport protein, have an increased risk for Alzheimer's disease, as well as for elevated levels of cholesterol and for heart disease. R. Mahley, “Cholesterol transport protein with expanding role in cell biology,” Science 240, 622-30 (1988); Saunders, et al., “Association of apolipoprotein E allele ε4 with late-onset familial and sporadic Alzheimer's disease,” Neurology 43, 1467-72 (1993); Corder, et al., “Gene dose of apolipoprotein E type 4 allele and the risk of Alzheimer's disease in late-onset families,” Science 261:921-923 (1993); Jarvik, et al., “Coronary artery disease, hypertension, ApoE and cholesterol: a link to Alzheimer's disease?” Ann. N.Y. Acad. Sci. 826:128-146 (1997). Both apoE4 and a second putative risk factor for Alzheimer's disease, α-2-macroglobulin, bind to a receptor, the lipoprotein receptor related protein, which is important for cellular uptake of cholesterol. (Narita et al., “Alpha2-macroglobulin complexes with and mediates the endocytosis of beta-amyloid peptide via cell surface low-density lipoprotein receptor-related protein,” J. Neurochem. 69(5):1904-11 (1997); and Blacker et al., “Alpha-2 macroglobulin is genetically associated with Alzheimer's disease,” Nature Genetics 19:357-60 (1998).) Other studies have shown that cholesterol increases the production of Aβ protein, which accumulates in the brains of subjects with Alzheimer's disease and is thought by many researchers to cause the neurodegeneration underlying the disease. D.J.Selkoe, “Cell biology of the beta-amyloid precursor protein and the genetics of Alzheimer's disease,” Cold Spring Harbor Symposia on Quantitative Biology, 61, 587-96 (1996); and Simons, et al., “Cholesterol depletion inhibits the generation of β-amyloid in hippocampal neurons,” Proc. Nat'l. Acad. Sci. USA, 95, 6460-04 (1998).

The apolipoprotein E isoform 4 (ApoE isoform 4) is a major genetic risk factor for Alzheimer's disease. PCT Patent Application No. WO 95/06470 discloses administration of an HMG-CoA reductase inhibitor (statin) to regulate levels of (ApoE isoform 4) in humans to prevent and treat Alzheimer's Disease. A normal cellular function of ApoE is uptake and delivery of lipids. The ApoE isoform correlates with an increased risk for atherosclerosis, increased amyloid plaque deposition and increased risk of Alzheimer's disease. K.Fassbender, et al., “Simvastatin Strongly Reduces Levels of Alzheimer's Disease β-amyloid peptides Aβ42 and Aβ40 in vitro and in vivo”, Proc. Nat'l Acad. Sci. USA 98: 5856-5861 (2001). PCT Patent Application WO 00/28981 discloses at page 3 that patients possessing the ApoE isoform 4 have an increased risk for Alzheimer's disease, as well as elevated levels of cholesterol and increased risk for heart disease.

Levels of cholesterol in the brain are critical for synapse formation and maintenance and recent studies identify cholesterol as a limiting factor in synaptogenesis. Reduced cholesterol may place a limit on plastic processes thus reducing the tendency to develop Alzheimer's disease. An issue for very long axons is the ability to supply sufficient cholesterol for rapid axonal growth, especially in regeneration. Alzheimer's disease brain contains less cholesterol, and this contributes to Alzheimer's disease-related alterations in membrane composition, membrane fluidity, and lipid bilayer structure and dynamics. Statins, as inhibitors of cholesterol synthesis, may reduce the prevalence of Alzheimer's disease. Long term potentiation is inhibited by cholesterol biosynthesis inhibitors and long term potentiation induction is associated with pathway-specific increases in lipid production. For example, axonal growth ceases when cholesterol synthesis is inhibited by pravastatin and could be reactivated by addition of cholesterol to either cell bodies or distal axons.

The term, “HMG CoA reductase inhibitor,” refers to any compound which inhibits the bioconversion of 3-hydroxy-3-methylglutaryl coenzyme A to mevalonic acid catalyzed by the enzyme HMG CoA reductase. The inhibiting effect of any such compounds can be readily determined by those skilled in the art according to standard assays. HMG CoA reductase inhibitors will be known to those skilled in the art. Non-limiting examples of suitable cholesterol biosynthesis inhibitors include competitive inhibitors of HMG CoA reductase, the rate-limiting step in cholesterol biosynthesis, squalene synthase inhibitors, squalene epoxidase inhibitors and mixtures thereof. The HMG CoA reductase inhibitors suitable for use in the invention include, but are not limited to, pravastatin (for example Pravachol™ which is available from Bristol Meyers Squibb) and related compounds, as disclosed in U.S. Pat. No. 4,346,227; and lovastatin and related compounds, as disclosed in U.S. Pat. Nos. 4,231,938 and 4,346,227. In some embodiments, lovastatin and pravastatin are used as HMG CoA reductase inhibitors in the invention. Lovastatin, marketed under the trade name Mevacor™, is a competitive inhibitor of HMG CoA reductase.

Other HMG CoA reductase inhibitors which may be employed in the invention include atorvastatin (Lipitor™, Pfizer, New York, N.Y.) and other 6-[2-(substituted-pyrrol-1-yl)alkyl]pyran-2-ones and derivatives, such as disclosed in U.S. Pat. No. 4,647,576; fluvastatin (Lescol™, Novartis, Basel, Switzerland); fluindostatin (Sandoz XU-62-320); pyrazole analogs of mevalonolactone derivatives as disclosed in PCT application WO 86/03488; rivastatin and other pyridyldihydroxyheptenoic acids as disclosed in European Patent 491226A; Searle's SC 45355 (a 3-substituted pentanedioic acid derivative) dichloroacetate; imidazole analogs of mevalonolactone, as disclosed in PCT application WO 86/07054; 3-carboxy-2-hydroxy-propane-phosphonic acid derivatives, as disclosed in French Patent No. 2,596,393; 2,3-di-substituted pyrrole, furan, and thiophene derivatives, as disclosed in European Patent Application No. 0221025; naphthyl analogs of mevalonolactone, as disclosed in U.S. Pat. No. 4,686,237; octahydronaphthalenes, such as those disclosed in U.S. Pat. No. 4,499,289; keto analogs of mevinolin (lovastatin), as disclosed in European Patent Application No. 0,142,146 A2; as well as other HMG CoA reductase inhibitors.

Other examples of suitable HMG CoA reductase inhibitors include statins such as fluvastatin, simvastatin (for example Zocor™ which is available from Merck & Co.), atorvastatin, cerivastatin, CI-981 and pitavastatin (such as NK-104 of Negma Kowa of Japan), rosuvastatin; HMG CoA synthetase inhibitors, for example L-659,699 ((E,E)-1 1-[3′R-(hydroxymethyl)-4′-oxo-2′R-oxetanyl]-3,5,7R-tri-methyl-2,4-undecadienoic acid); squalene synthesis inhibitors, for example squalestatin 1; and squalene epoxidase inhibitors, for example, NB-598 ((E)-N-ethyl-N-(6,6-dimethyl-2-hepten-4-ynyl)-3-[(3,3′-bithiophen-5-yl)methoxy]benzene-methanamine hydrochloride) and other sterol biosynthesis inhibitors such as DMP-565.

In addition, other compounds useful in inhibiting HMG-CoA reductase suitable for use herein are disclosed in U.S. Pat. Nos. 4,904,646 and 5,091,378. Examples of statins include Advicorm (lovastatin/niacin); cerivastatin (Baycol™, Bayer Corp., withdrawn from U.S. market); Mevacor™ (lovastatin, Merck & Co., Inc., Rahway, N.J.); rivastatin; rosuvastatin; pitavastatin; mevastatin; velostatin; and Zocor™ (simvastatin, Merck & Co., Inc., Rahway, N.J.). Further examples of HMG-CoA reductase inhibitors include pyrazole analogs of a mevalonolactone, indene analogs of mevalonolactone, 3-carboxy-2-hydroxypropanephosphinic acid derivatives, 6-[2-(substituted-pyrrol-1-yl)-alkyl]pyran-2-one, heterocyclic analogs of mevalonolactone including imidazole analogs, naphthyl analogs of mevalonolactone, octahydro-naphthalene derivatives, keto analogs of lovastatin, and 2,3-di-substituted pyrrole, furan, or thiophene compounds.

In addition to their direct effects on lipid/cholesterol biosynthesis and metabolism, statins in combination with substrates of nitric oxide synthase are known to facilitate transport of drugs across the blood brain barrier (“BBB”). Inasmuch as Alzheimer's disease is a disease of the brain, an especially useful pharmaceutical composition is a combination of a statin second agent and a nitric oxide synthase substrate second agent (e.g., L-Arg) as well as a first agent as described herein. A family of enzymes called Nitric Oxide Synthase (“NOS”) form nitric oxide from L-arginine, and the nitric oxide produced is responsible for the endothelium dependent relaxation and activation of soluble guanylate cyclase, neurotransmission in the central and peripheral nervous systems, and activated macrophage cytotoxicity. Nitric Oxide Synthase, occurs in many distinct isoforms which include a constitutive form (cNOS) and an inducible form (iNOS). The constitutive form is present in normal endothelial cells, neurons and some other tissues. Formation of nitric oxide by the constitutive form in endothelial cells is thought to play an important role in normal blood pressure regulation, prevention of endothelial dysfunction such as hyperlipodemia, arteriosclerosis, thrombosis, and restenosis. The inducible form of nitric oxide synthase has been found to be present in activated macrophages and is induced in vascular smooth muscle cells, for example, by various cytokines or microbial products. The conversion of precursor substrates such as L-arginine into nitric oxide is enzymatically catalyzed by NOS and the resulting by-product of the conversion of L-arginine is L-citrulline. L-arginine as used herein includes all biochemical equivalents (i.e. salts, precursors, and its basic form).

In one embodiment, this invention provides a method to enhance delivery of a first agent to brain tissue of an individual comprising introducing the composition into the blood stream of the individual substantially contemporaneously with a blood flow enhancing amount of L-arginine. In another embodiment, this invention provides a method to enhance delivery of a desired composition to brain tissue of an individual comprising introducing the composition into the blood stream of the individual substantially contemporaneously with a blood flow enhancing amount of L-arginine or a blood flow-enhancing amount of a non-ecNOS NO-generating system.

In another alternative embodiment, the compositions used in the methods of the present invention may further comprise one or more Cholesteryl Ester Transfer Protein (“CETP”) Inhibitors coadministered with or in combination with the compound(s) of the Formulae described herein. CETP is responsible for the exchange or transfer of cholesteryl ester carrying HDL and triglycerides in VLDL.

Non-limiting examples of suitable CETP inhibitors are disclosed in PCT Patent Application No. WO 00/38721 and U.S. Pat. No. 6,147,090, which are incorporated herein by reference. Pancreatic cholesteryl ester hydrolase (pCEH) inhibitors such as WAY-121898 also may be coadministered with or in combination with the fibric acid derivative(s) and sterol absorption inhibitor(s) discussed above.

In another alternative embodiment, the compositions used in the methods of the present invention may further comprise probucol or derivatives thereof (such as AGI-1067 and other derivatives disclosed in U.S. Pat. Nos. 6,121,319 and 6,147,250), which may reduce LDL and HDL levels, coadministered with or in combination with the compounds of the Formulae herein. In another alternative embodiment, the compositions used in the methods of the present invention may further comprise one or more low-density lipoprotein (LDL) receptor activators, coadministered with or in combination with a compound of any Formula discussed above. Non-limiting examples of suitable LDL-receptor activators include HOE-402, an imidazolidinylpyrimidine derivative that directly stimulates LDL receptor activity. See, M. Huettinger et al., “Hypolipidemic activity of HOE-402 is Mediated by Stimulation of the LDL Receptor Pathway.” Arterioscler. Thromb. 13, 1005-12 (1993).

In another alternative embodiment, the compositions used in the methods of the present invention can further comprise plant sterols, plant stanols or fatty acid esters of plant stanols, such as sitostanol ester used in Benecol™ margarine, which can reduce cholesterol levels, coadministered with or in combination with a compound of any Formula herein. Generally, a total daily dosage of plant sterols, plant stanols or fatty acid esters of plant stanols can range from about 0.5 to about 20 grams per day in single or 2-4 divided doses.

In another alternative embodiment, the compositions used in the methods of the present invention can further comprise one or more antioxidants, such as probucol, tocopherol, ascorbic acid, β-carotene and selenium, or vitamins such as vitamin B₆ or vitamin B₁₂, coadministered with or in combination with a compound of any Formula herein. Generally, a total daily dosage of antioxidants or vitamins can range from about 0.05 to about 10 grams per day in single or 24 divided doses.

In another alternative embodiment, the compositions used in the methods of the present invention can further comprise monocyte and macrophage inhibitors such as polyunsaturated fatty acids (PUFA), thyroid hormones including throxine analogues such as CGS-26214 (a thyroxine compound with a fluorinated ring), gene therapy and use of recombinant proteins such as recombinant apo E, coadministered with or in combination with a compound of any Formula herein. Generally, a total daily dosage of these agents can range from about 0.01 to about 1000 mg/day in single or 2-4 divided doses.

Fassbender et al. disclose that use of simvastatin and lovastatin, alone or in combination with methyl-β-cyclodextrin, can reduce intracellular and secreted Aβ levels in vitro and that treatment of animals with simvastatin reduces brain and cerebrospinal fluid levels of Aβ in vivo.

U.S. Pat. No. 6,071,899 discloses compounds, which may have a general application in any disorder that involves endothelial dysfunction, such as atherosclerosis, or may have a general application in any disorder that involves lipid peroxidation in conjunction with enzyme activity, including inflammatory conditions of the brain such as Alzheimer's Disease (see col. 5, lines 16-29).

PCT Patent Application WO 99/38498 discloses methods for preventing or treating Alzheimer's disease by administering a plasma-triglyceride level-lowering agent (e.g., fibrates), optionally in combination with a cholesterol level-lowering agent such as statins, bile acid sequestrants or agents that block intestinal cholesterol absorption (e.g., β-sitosterol, SCH 48461 ((3R,4S)-1,4-bis-(4-methoxyphenyl)-3-(3-phenylpropyl)-2-azetidinone), CP-148,623, saponins, neomycin and ACAT inhibitors).

U.S. Pat. Nos. 5,767,115, 5,624,920, 5,688,990, 5,656,624 and 5,688,787, respectively, disclose hydroxy-substituted azetidinone compounds and substituted β-lactam compounds useful for lowering cholesterol or in inhibiting the formation of cholesterol-containing lesions in mammalian arterial walls, but does not disclose treatment of Alzheimer's Disease.

Simvastatin has been used to reduce levels of β-amyloid peptides Aβ₄₂ and Aβ₄₀ in vitro and in vivo, for example, in guinea pigs. Wolozin, B. et al., Arch. Neurol. 57:1439-1443, 2000, describe the analysis of a subject population treated with HMG-CoA reductase inhibitors. The authors reported that the prevalence of Alzheimer's disease was 60-73% lower in these subjects than in subjects taking other medications. In this study, a causal relationship could not be established. Jick, H. et al., The Lancet 356:1627-1631, 2000, also reviewed subject records and found that in individuals 50 years and older, statin administration was associated with a substantially lowered risk of dementia, including Alzheimer's disease and other conditions. Similarly, Acyl-CoA: cholesterol acyltransferase (ACAT) inhibitors have been used to decrease plasma cholesterol in various animal models including rats, guinea pigs and rabbits (Tanaka et al., J. Med. Chem. 41:2390-2410, 1998; Junquero et al, Biochem. Pharmacol. 61:97-108, 2001). Examples of ACAT inhibitors include but are not limited to Glibenclamide, CI-976 (PD128042), NTE-122, Fatty acid Anilides, F12511, Avasimibe, TS-962 (HL-004), N-Chlorosulfonyl isocyanate and derivatives, SR-9223i, Pyripyropenes, PD-132301, PD-132301-2, DUP-128, YM-17E, BW447A, Alzheimer's disease 6591, CL-277,082, Melinamide, Hydroxyphenyl Urea derivatives, R-106578, Indoline derivatives with amide or urea moiety, 57-118, 58-035, CI-999, CI-1011, N-alkyl-N-[(fluorophenoxy)benzyl]-N′-arylureas and derivatives, SKF-99085, EAB309, N-alkyl-N-(heteraryl-substituted benzyl)-N′-arylureas and derivatives, F-1394, N-alkyl-N-biphenyllylmethyl-N′-aryl ureas and derivatives, CL 277,082, CL 283,546, CL 283,796, CP-113,818, CP-105,191, Polyacetylene analogs-panaxynol, panaxydol, panaxydiol and panaxytriol, T-2591, 4,4-bis(trifluoromethyl)imidazolines and derivatives, FR145237, FR1 86054, FR129169, Naringenin, Ulimoidol, 23-hydroxyursolic acid, 27-trans-p-coumaroyloxyursolic acid, 27-cis-p-coumaroyloxyursolic acid, Triterpenes and derivatives, N-(4,5-diphenylthiazol-2-yl)-N′-aryl or alkyl (thio)ureas and derivatives, N-(4,5-diphenylthiazol-2-yl)alkanamide-s and derivatives, RP73163, RP64477, Diaryl-substituted heterocyclic ureas and derivatives, Heterocyclic amides and derivatives, Cyclic sulfides derived from hetero-Diels-Alder reaction of thioaldehydes with 1,3-dienes, E5324, Tetrazole amide derivatives of (+/−)-2-dodecyl-alpha-phenyl-N-(2,4,6-trimethoxyphenyl)-2H-tetrazole-5-acetamide, Epicochlioquinone A, acyclic(diphenylethyl) diphenylacetamides, 2-(1,3-dioxan-2-yl)-4,5-diphenyl-1H-imidazoles and derivatives, N-(2,2-dimethyl-2,3-dihydrobenzofuran-7-yl)amide derivatives, FCE 27677, GER1-BPO02-A, TMP-153, amides of 1,2-diarylethylamines and derivatives, F-1394, N-(4-oxochroman-8-yl)amide derivatives, terpendoles, short chain ceramide and dihydroceramide, FY-087, 447C88, cyclandelate, 3-quinolylurea derivatives, N-phenyl-6,11-dihydrodibenz[b,e]oxepin-11-carboxamides and related derivatives, Gypsetin, AS-183, AS-186, 2,6-disubstituted-3-imidazolylbenzopyrane derivatives, Lateritin, 2-(alkylthio)-4,5-diphenyl-1H-imidazoles derivatives, glisoprenins, acaterin, U-73482, purpactins, and chlorpromazine.

Amyloid Inhibitors (Anti-Amyloid Therapeutic Approaches)

Other important targets for therapeutic intervention are the mechanisms which convert APP into Aβ. In particular, down-regulation of the β and gamma-secretases and up-regulation of alpha-secretase (which cleaves within the Aβ peptide) would inhibit the production of Aβ peptide. β-secretase or BACE has been identified by several groups and is an aspartyl protease enzyme. However the molecular identity of the gamma-site APP processing enzyme, gamma-secretase, still remains to be determined. It is clear that presenilins are required for γ-secretase cleavage of APP. Aβ secretion is almost completely blocked in neurons lacking presenilin 1. There are a number of possibilities for the function of presenilins: (1) They could be required for intracellular trafficking and sorting of APP to the γ-secretase compartment, or (2) They could serve as a co-factor for γ-secretase cleavage. (C. Haass, et. al., Science 286, 916-19 (1999); MS Wolfe, et al., Biochemistry 38, 4720-27 (1999); T. Bayer, et al., Brain Pathology 11; 1-11 (2001); B. De Strooper, et al., Nature 391, 387-91 (1998)). See also, WO 2003/103652 and WO 2003/103653.

γ-Secretase cleaves at the C-temminus of Aβ and is primarily responsible for generating the pathogenic 42-amino acid form of Aβ, Aβ42, which forms insoluble toxic fibrils and accumulates in senile plaques. M. Hutton, et al., Essays Biochem. 33, 117-31 (1998); R.L. Nussbaum and C.E. Ellis, N. Engl. J Med 348(14), 1356-64 (2003); T. Iwatsubo, et al., Neuron 13, 45-53 (1994), W. P. Esler and M. S. Wolfe, Science 293, 1449-54 (2001). Although the normal function of APP is unknown, it is predominantly expressed in the brain and is suspected of participating in cell adhesion, synaptic growth, and neural repair.

Amyloid-β forms a continuum of aggregation species: monomeric amyloid-β, soluble oligomeric amyloid-β, insoluble protofibrils, amyloid-β, diffuse amyloid, compact amyloid, and neuritic or senile amyloid, the latter two being the pathologic and diagnostic hallmarks of Alzheimer's disease. Independent of fibril or plaque formation, however, amyloid-β may alter membrane potential and firing, synaptic transmission, synaptic plasticity, and learning. Amyloid-β, especially amyloid-β 1-42, has been shown to be neurotoxic. Accordingly, amyloid-β itself represents a significant drug target. Recent evidence suggests that plaques per se are less toxic than oligomers or protofibrils. These oligomeric forms of Aβ could be responsible for the first stage of the disease when neuronal cell death is initiated. See also, WO 03/050073; WO 03/047576; WO 03/045378; WO 03/043987; WO 03/043975; WO 03/043618; U.S. Pat. No. 6,569,851; WO 03/040096; U.S. Pat. No. 6,552,013; WO 03/037325; WO 03/030886; WO 03/029169; EP 1,298,436; WO 03/027068; U.S. Pat. No. 6,528,505; WO 03/020370; U.S. Pat. No. 6,509,331; WO 03/006453; WO 03/006423; WO 03/006021; WO 03/006013; U.S. Pat. No. 6,509,331; U.S. Pat. No. 6,486,350; WO 03/002122; U.S. Pat. No. 6,476,263; WO 03/000261; WO 02/100856; WO 02/100820; WO 02/100818; WO 02/100410; WO 02/100399; WO 02/098849; WO 02/094768; U.S. Pat. No. 6,476,263; WO 02/076440; U.S. Pat. No. 2002/16320 Al; U.S. Pat. No. 6,329,163; WO 00/202,520; WO 00/202,518; WO 00/202,512; WO 00/202,506; WO 00/202,505; U.S. Pat. No. 6,284,221; U.S. Pat. No. 6,221,645; WO 00/175,165; WO 00/170,672; U.S. Pat. No. 6,262,302; U.S. Pat. No. 6,191,166; U.S. Pat. No. 6,262,302; U.S. Pat. No. 6,153,652; WO 96/40885; U.S. Pat. No. 5,942,400; U.S. Pat. No. 5,744,346; and WO 98/21589.

Dense microspheres or spherons may be turned into plaques when they are burst in vitro or when injected into experimental animals. P. Averback, J Alzheimer's Disease 1, 1-34 (1998). The compound NX-D2858 (Nymox Pharmaceutical Corp., Dorval, Quebec, Canada) blocks the transformation of spherons into senile plaques and may stop or slow the progress of Alzheimer's disease. U.S. published application no. 2003-0083298. Another compound that may be used in the pharmaceutical compositions of the invention is Ateroid™ (Hunter-Fleming,) and related mucopolysaccharides, such as glycosaminoglycans having an average molecular weight equal to 2,400 Da, which have been described as suitable for the treatment of Alzheimer's disease. EP 1,181,024

Another compound is indole-3-propionic acid (Oxygon™, Mindset), which is described as preventing the cytotoxic effects of amyloid beta protein on cells, as well as blocking amyloid deposition and therefore useful in treating a fibrillogenic disease, such as Alzheimer's disease. U.S. Pat. No. 6,395,768 B1. Suitable amyloid aggregation inhibitors also include reumacon available from Conpharm AB.

Additional examples include a variety of polysaccharide compounds. U.S. Pat. No. 6,607,758, WO 03/013442, US 2002/197692, US 2002/150637, and CA 2,323,090

Yet another approach is exemplified by a new chelating agent. “Targeted pharmacological depletion of serum amyloid P component for treatment of human amyloidosis.” M. B.Pepys, et al., Nature 417(6886), 254-59.

Amyloid-Formation Inhibitors

The present invention pertains to a method for treating or preventing a disease state associated with amyloidosis, the method comprising administering to a subject a therapeutically effective amount of an agent for reducing the concentration of fibrillar or soluble Aβ, such that said disease state associated with amyloidosis is treated or prevented.

In an embodiment, the methods of the invention are based, at least in part, on inhibiting an interaction between an amyloidogenic protein and a constituent of basement membrane to inhibit amyloid deposition. The constituent of basement membrane is a glycoprotein or proteoglycan, e.g., heparan sulfate proteoglycan. A therapeutic agent used in the method of the invention may interfere with binding of a basement membrane constituent to a target binding site on an amyloidogenic protein, thereby inhibiting amyloid deposition. In some aspects, the methods of the invention involve administering to a subject a therapeutic agent which inhibits amyloid deposition. “Inhibition of amyloid deposition” is intended to encompass prevention of amyloid formation, inhibition of further amyloid deposition in a subject with ongoing amyloidosis and reduction of amyloid deposits in a subject with ongoing amyloidosis. Inhibition of amyloid deposition is determined relative to an untreated subject or relative to the treated subject prior to treatment. Amyloid deposition is inhibited by inhibiting an interaction between an amyloidogenic protein and a constituent of basement membrane. “Basement membrane” refers to an extracellular matrix comprising glycoproteins and proteoglycans, including laminin, collagen type IV, fibronectin and heparan sulfate proteoglycan (“HSPG”). In one embodiment, amyloid deposition is inhibited by interfering with an interaction between an amyloidogenic protein and a sulfated glycosaminoglycan such as HSPG. Sulfated glycosaminoglycans are known to be present in all types of amyloids (see Snow, et al. Lab. Invest. 56, 120-23 (1987)) and amyloid deposition and HSPG deposition occur coincidentally in animal models of amyloidosis (see Snow, et al. Lab. Invest. 56, 665-75 (1987)).

The ability of a therapeutic compound of the invention to inhibit an interaction between an amyloidogenic protein and a glycoprotein or proteoglycan constituent of a basement membrane may be assessed by an in vitro binding assay, such as that described in U.S. Pat. No. 5,164,295. Alternatively, the ability of a compound to bind to an amyloidogenic protein or to inhibit the binding of a basement membrane constituent (e.g., HSPG) to an amyloidogenic protein (e.g., AP) may be measured using a mass spectrometry assay where soluble protein, e.g., Aβ, is incubated with the compound. A compound that binds to, e.g., Aβ, will induce a change in the mass spectrum of the protein.

For example, a therapeutic agent of the invention may interact with a binding site for a basement membrane glycoprotein or proteoglycan in an amyloidogenic protein and thereby inhibits the binding of the amyloidogenic protein to the basement membrane constituent. Basement membrane glycoproteins and proteoglycans include laminin, collagen type IV, fibronectin and HSPG. In an embodiment, the therapeutic agent inhibits an interaction between an amyloidogenic protein and HSPG. Consensus binding site motifs for HSPG in amyloidogenic proteins have been described (see, e.g., Cardin and Weintraub, Arteriosclerosis 9, 21-32 (1989)).

Metal Chelators

Zn²⁺ mediates neurodegenerative processes observed in seizure, ischemia, trauma, and Alzheimers disease. Zn2+ is observed in the extracellular plaque and degenerating neurons in Alzheimer's disease, which may contribute to neuronal degeneration in Alzheimer's disease. Oxidative damage in the neocortex associated with Alzheimer's disease may be the result of gradual build up of metal ions like zinc and copper. Copper and zinc have particularly high concentrations in the β-amyloid plaques in the brains of Alzheimer's subjects. Both metals are essential, but normally only small amounts are required and excess metals are excreted. It is hypothesized that β-amyloid converts dissolved oxygen to hydrogen peroxide, which in turn causes cell damage. Metal chelators may be used to diminish the oxidative burden. In APP transgenic mice treated with clioquinol (an antibiotic and bioavailable Cu/Zn chelator), marked reduction in Aβ deposition occurred after several months of treatment. Zinc and other divalent cations appear necessary for Aβ aggregation. Thus, metal chelation may have some therapeutic benefit in the treatment of Alzheimer's disease, either by preventing Aβ aggregation or by disrupting preformed aggregates. In the laboratory, the copper-zinc chelator clioquinol may dissolve amyloid-beta deposits in postmortem brain tissue from Alzheimer's disease subjects. In APP transgenic mice treated with clioquinol (an antibiotic and bioavailable Cu/Zn chelator), marked reduction in Aβ deposition occurred after several months of treatment A new study extends these results to mice genetically prone to overproduce amyloid-β. Clioquinol cut amyloid deposits in half over a nine week period with no adverse effects. The mice treated with clioquinol also exhibited significantly improved scores on a behavioral rating scale. The affinity of clioquinol for Zn is in the nanomolar range, whereas the affinity of AP for Zn²⁺ is in the low micromolar range. Clioquinol had been approved by the FDA as an antibiotic, but was removed from the market about 30 years ago because of side effects involving the loss of Vitamin B-12. The antibiotic clioquinol, also known as 5-chloro-7-iodo-8-hydroxyquinoline or iodochlorhydroxyquin, a known copper/zinc chelator is a reasonably well tolerated drug in humans and is currently in a phase II clinical trial for Alzheimer's disease. T.E. Golde, J. Clin. Invest. 111, 11-18 (2003). Clioquinol chelates copper and zinc in vitro, and reduces Aβ deposition in a mouse model. Moreover, interim results from a randomized, double-blind, placebo-controlled clinical trial in 32 subjects with Alzheimer's disease suggested that this drug slows the rate of cognitive decline in the most severely affected group.

Suitable copper/zinc chelators include clioquinol available from PN Gerolymatos SA. Preliminary positive results have been generated by a human clinical trial in which clioquinol, supplemented with B-12, appears to be helpful in humans with Alzheimer's. Bush, et al., Proc. Nat'l Acad. Sci. USA 99, 7317-19 (1999), U.S. Publshed Patent Application No. 2002/0,025,944. See also, “Treatment with a copper-zinc chelator markedly and rapidly inhibits beta-amyloid accumulation in Alzheimer's disease transgenic mice.” R.A. Chemy, et al., Neuron 30, 665-76 (2001); and “The galvanization of P-amyloid in Alzheimer's disease” A.I. Bush and R.E. Tanzi Proc. Nat'l Acad. Sci. USA 99, 7317-19 (2002).

Phanquinone (4,7-phenanthroline-5,6-dione) has hitherto been used for the treatment of various disorders, such as amoebiasis. However, its use for the treatment or prevention of memory improvement has been suggested. Phanquinone has been marketed by Ciba-Geigy as Entobex™. Phanquinone is also a metal chelator in the same family as clioquinol. According to the present invention the use of phanquinone for the manufacture of a pharmaceutical composition for the treatment or prevention of memory impairment is provided.

Behavioral Management of Alzheimer's Disease

Patients with Alzheimer's disease may also be treated for behavioral disturbances associated with progression of the disease. Use of such treatments is intended to decrease psychotic symptoms such as paranoia, delusions and hallucinations, and associated or independent agitation, screaming, combativeness or violence, and thereby increase the comfort and safety of patients. Anti-psychotics and antidepressants can be used intermittently in patients with defined psychotic symptoms.

Benzodiazepines may be used briefly and judiciously for emergency sedation but otherwise should be avoided because they can produce delirium and tend to further compromise residual cognitive capacities. Lithium (Eskalith), centrally active β-adrenergic blockers, carbamazepine (Tegretol™, Ciba-Geigy Pharmaceuticals, now Novartis, Basel, Switzerland), and valproate (Depakene) have been used empirically in the treatment of affective lability and aggressive outbursts. Resperidone can also be used for psychoses associated with Alzheimer's disease. Olanzabine, sertindole, and quetiapine can also be used. Stilt other examples include trazodone; β blockers, propranolol, metoprolol and pindotol (especially for some agitated patients with dementia). When male patients display intrusive disinhibited sexual behavior, a particular problem in patients with frontal lobe dementias, medroxyprogesterone and related hormonal agents may be employed. Glycosaminoglycan polysulfate (Ateroid™) can also improve depressive symptomatology in old-age dementia. Prog. Neuropsychopharmacol. Biol. Psychiatry 13, 977-81 (1989).

Treatment of apathy is also considered. Dopaminergic agents, such as psychostimulant CD-amphelamine, methylphemidate), amantadine (Symmetrel™, Du Pont Multi-Source Products, Wilmington, Del.), bromocriptine and buproprion are helpful in the treatment of severe apathy. A.E.Wallace, et al., “Double-blind, placebo-controlled trial of methylphenidate in older, depressed, medically ill patients.” Am. J. Psychiatry 152, 929-31 (1995).

SSRIs are often chosen as the initial treatment because of their better side effect profiles. Once-a-day dosing may be appropriate. Examples of SSR1s include fluoxetine (Prozac™, Pulvules, Dista, Eli Lilly, Indianapolis, Ind.), paroxetine, sertraline, bupropion, and venlagaxin. Among the tricyclic and heterocyclic agents, theoretical reasoning and clinical experience suggest avoiding agents with prominent anticholinergic activity (e.g., amitriptyline, imipramine). Among the remaining agents, sample dosing strategies are given here for nortriptyline. MAOIs are also considered for individuals unresponsive to or unable to take other agents (tranylcypromine and phenelzine are examples)

When sleep disturbances occur without other psychiatric symptoms beyond the dementia itself, some clinicians prescribe trazodone or zolpidem. Benzodiazepines (e.g., lorazepam or oxazepam) and chloral hydrate may be used. Triazolam in particular is not recommended for individuals with dementia because of its association with amnesia. Diphenhydramine, which is found in most over-the-counter sleep preparations, is used by some clinicians, but its anticholinergic properties make it suboptimal for the treatment of demented patients.

Some individuals with dementia show disinhibited behavior, including making inappropriate jokes, neglecting personal hygiene, exhibiting undue familiarity with strangers, or disregarding conventional rules of social conduct. Occasionally, they may harm others by striking out. Suicidal behavior may occur, especially in mildly impaired individuals, who are more likely to have insight into their deficits and to be capable of formulating (and carrying out) a plan of action. Anxiety is fairly common, and some patients manifest “catastrophic reactions,” overwhelming emotional responses to relatively minor stressors, such as changes in routine or environment. Depressed mood, with or without neurovegetative changes, is quite common, as are sleep disturbances independent of depression. Delusions can occur, especially those involving themes of persecution (e.g., the belief that misplaced possessions have been stolen). Misidentifications of familiar people as unfamiliar (or vice versa) frequently occur. Hallucinations can occur in all sensory modalities, but visual hallucinations are most common. Some patients exhibit a peak period of agitation (or other behavioral disturbances) during the evening hours, which is sometimes referred to as “sundowning.”

Delirium is frequently superimposed on dementia because the underlying brain disease increases susceptibility to the effects of medications or concurrent general medical conditions. Individuals with dementia may also be especially vulnerable to psychosocial stressors (e.g., going to the hospital, bereavement), which may exacerbate their intellectual deficits and associated problems.

Dementia is sometimes accompanied by motor disturbances, which may include gait difficulties, slurred speech, and a variety of abnormal movements. Other neurological symptoms, such as myoclonus and seizures, may also occur.

When male patients display intrusive disinhibited sexual behavior, a particular problem in patients with frontal lobe dementias, medroxyprogesterone and related hormonal agents are sometimes recommended (H.Kyomen, et al., “The use of estrogen to decrease aggressive physical behavior in elderly men with dementia.” J. Am. Geriatr. Soc. 39, 1110-12 (1991); S.S.Rich, et al., “Leuprolide acetate for exhibitionism in Huntington's disease.” Mov. Disord. 9, 353-57 (1994); P.G.Weiler, et al., “Propranolol for the control of disruptive behavior in senile dementia.” J. Geriatr. Psychiatry Neurol. 1, 226-30 (1988)), but only case series support this recommendation at present.

Glycosaminoglycan polysulfate (e.g., Ateroid™) in old-age dementias: effects upon depressive symptomatology in geriatric patients. Prog Neuropsychopharmacol. Biol. Psychiatry 13, 977-81 (1989)) The literature concerning the treatment of apathy is much sparser. There is minimal evidence that dopaminergic agents, such as psychostimulants (d-amphetamine, methylphenidate), amantadine, bromocriptine, and bupropion, are helpful in the treatment of severe apathy, but promising case reports suggest that efficacy studies are warranted (A. E. Wallace, et al., “Double-blind, placebo-controlled trial of methylphenidate in older, depressed, medically ill patients.” Am. J. Psychiatry 152, 929-31 (1995)). Psychostimulants have also received some support for the treatment of depression in elderly individuals with severe general medical disorders (P.Pickett, et al., “Psychostimulant treatment of geriatric depressive disorders secondary to medical illness.” J. Geriatr. Psychiatry Neurol. 3, 146-51 (1990); L.W.Lazarus, et al., “Methylphenidate and nortriptyline in the treatment of poststroke depression: a retrospective comparison.” Arch. Phys. Med. Rehabil. 75, 403-06 (1994); T.R.Price, et al., “Safety and efficacy of ECT in depressed patients with dementia: a review of clinical experience.” Convulsive Ther. 5, 1-74 (1989)).

SSRIs are often chosen as the initial treatment because of their better side effect profiles. Once-a-day dosing is appropriate. Fluoxetine,. Paroxetine,. Sertraline, bupropion and venlagaxin are examples of SSR1s treatment. Among the tricyclic and heterocyclic agents, theoretical reasoning and clinical experience suggest avoiding agents with prominent anticholinergic activity (e.g., amitriptyline, imipramine). Among the remaining agents, sample dosing strategies are given here for nortriptyline

Among the tricyclic and heterocyclic agents, theoretical reasoning and clinical experience suggest avoiding agents with prominent anticholinergic activity (e.g., amitriptyline, imipramine). Among the remaining agents, sample dosing strategies are given here for nortriptyline, desipramine, and trazodone.

Depression is common in patients with dementia. Patients with depression should be carefully evaluated for suicide potential. Depressed mood may respond to improvements in the living situation or stimulation-oriented treatments, but patients with severe or persistent depressed mood with or without a full complement of neurovegetative signs should be treated with antidepressant medications. Although formal evaluation of the efficacy of antidepressants for demented patients is limited, there is considerable clinical evidence supporting their use. The choice among agents is based on the side effect profile and the characteristics of a given patient. MAOIs are also considered for individuals unresponsive to or unable to take other agents (tranylcypromine and phenelzine are examples).

Treatment of sleep disturbance in dementia is aimed at decreasing the frequency and severity of insomnia, interrupted sleep, and nocturnal confusion in patients with dementia. The goals are to increase patient comfort and to decrease the disruption to families and caregivers. Sleep disorder is common in dementia (Satlin A: Sleep disorders in dementia. Psychiatr. Ann. 24, 186-90 (1994); C.C.Hoch, et al., “Sleep patterns in Alzheimer, depressed, and healthy elderly.” West J Nurs. Res. 10, 239-56 (1988)) and is not always so disruptive that the risk of medication side effects is outweighed by the need for a pharmacologic trial. When sleep disturbances occur without other psychiatric symptoms beyond the dementia itself, some clinicians prescribe trazodone or zolpidem. Benzodiazepines (e.g., lorazepam or oxazepam) and chloral hydrate may in some cases be used. Triazolam in particular is not recommended for individuals with dementia because of its association with amnesia. Diphenhydramine, which is found in most over-the-counter sleep preparations, is used by some clinicians, but its anticholinergic properties make it suboptimal for the treatment of demented patients.

Nutritional Supplements: Vitamin B12, Homocvsteine

In another aspect of the invention, a first agent is co-administered with one or more nutritional supplements to improve the safety or efficacy of treatment. For example, treatment can be optimized by provision of nutrients depleted in the subject.

Accordingly, suitable second agents include the following vitamins, some of which have an antioxidant and/or anti-inflammatory effect: retinol, vitamin A aldehyde (retinal), vitamin A acid (retinoic acid), retinyl acetate, vitamin B₁ (thiamine HCl), thiamine propyl disulfide, thiamine disulfide, thiamine disulfide O,O-diisobutyrate, thiamine disulfide hydrochloride, thiamine disulfide phosphate, thiamine mononitrate, thiamine 1,5-salt, thiamine phosphoric acid ester chloride, thiamine phosphoric acid ester phosphate salt, thiamine triphosphoric acid ester, vitamin B₂ (riboflavin), riboflavin tetrabutyrate, riboflavine 5′-phosphate ester monosodium salt, vitamin B₅ (pantothenic acid), pantothenic acid sodium salt, pantothenic acid calcium salt, vitamin B₆ (pyridoxine HCl), pyridoxal, pyridoxal HCI, pyridoxal 5-phosphate, pyridoxal 5-phosphate calcium salt, pyridoxamine, pyridoxamine dihydrochloride, pyridoxamine phosphate, vitamin B₁₂ (cyanocobalamin), methyl vitamin B₁₂ (co-methylcobalamin), vitamin D₂, vitamin D₃, vitamin D₄, vitamin H (biotin), vitamin K₁ (phytonadione), diacetyl dihydro vitamin K₁, vitamin K₁ oxide, vitamin(s) K₂ (menaquinones), vitamin K₂(35), vitamin K₂(35) dihydrodiacetate, vitamin K₂₍₃₀₎, vitamin K₂₍₃₀₎ dihydrodiacetate, vitamin K₅, vitamin K₅ hydrochloride, N-acetyl vitamin K₅, vitamin K₆, vitamin K₆ dihydrochloride, vitamin K₇, vitamin K₇ hydrochloride, vitamin K-S(II), vitamin L₁, Vitamin L₂, vitamin U, methylmethioninesulfonium bromide (bromide analog of vitamin U), α-carotene, βcarotene, γ-carotene, ω-carotene, γ-carotene (lycopene), phytofluene, L-carnitine (vitamin BT), acetyl-L-carnitine, folic acid (vitamin Bc), folinic acid, folinic acid calcium salt pentahydrate, niacinamide, nicotinic acid (vitamin B₃), nicotinic acid sodium salt sesquihydrate, nicotinic acid monoethanolamine salt, and combinations thereof.

Pyrrolidone or pyrrolidine derivatives for improving memory have been suggested in EP 239500, EP 165919, BE 892942, U.S. Pat. No. 5,102,882, EP 296978, EP 296979. Pyridine derivatives for the treatment of impairment of short-term memory are disclosed in U.S. Pat. No. 4,448,779. Choline derivatives for treating mental decline in the elderly are suggested in EP 201623. Indole or indolin derivatives for the improvement of processes involved in learning are disclosed in EP 241006, JP 6107544, U.S. Pat. No. 5,494,928, WO 97/47598, and U.S. Pat. No. 4,778,812. Pilocardin derivatives for improving memory functions are disclosed in U.S. Pat. No. 4,977,176. Glycine-containing compositions for enhancing cognitive functions are disclosed in U.S. Pat. No. 5,731,349. Peptide derivatives for treating mental decline and improving mental capacity are disclosed in U.S. Pat. No. 5,439,930, RU 2099078, and WO 95/15310. Xanthine derivatives for the treatment of age-related memory impairment are disclosed in WO 94/19349.

Compounds enhancing the stimulus-induced release of neurotransmitters, especially acetylcholine, may also be used to treat memory impairment. Examples are 2-benzyl-2-propyl 2-amino-2-R-acetate derivatives disclosed in EP 293351, 1-(4-chlorophenyl)-2-methyl-2-propyl 2-amino-3-methyl-butanoate disclosed in GB 2205097, polycyclic hetero-aromatic derivatives disclosed in U.S. Pat. No. 5,300,642,5-phenyl-4,4-dimethyl-3-oxo or hydroxy-pentylamine derivatives disclosed in EP 322391, 1-oxa-8-azaspiro(4.5)decane derivatives disclosed in EP 491562, derivatives of azacyclic and azabicyclic hydroxylamine disclosed in WO 94/00448, halogenated aromatic derivatives disclosed in EP 627400, derivatives of acyclic and cyclic amides disclosed in WO 95/29909, carbamoyloxypropylamine or carbamoyloxyethylamine derivatives disclosed in WO 96/08468.

Compounds that modulate the function of the kainate receptor may be used to improve memory, e.g, alkyl carboxy amino acids, such as (2S,4R)-4-methyl glutamic acid. WO 96/25387.

Hypothalamic hypophysiotropic hormones, such as somatostatin and growth-hormone releasing factor, may improve learning abilities. EP 326381.

Uronic acids improve cerebral efficiency in general, such as improvement of memory. DE 2555010.

Improvement of memory occurs upon administering spiro(N′-methyl-4′-piperidyl)-N-ethyl-succinimide, a parasympathicomimetic substance also having cholinomimetic, analgetic and sedative activity. U.S. Pat. No. 4,481,206.

WO 98/33498 discloses the use of breflate or analogous compounds thereof for the treatment of a mammal suffering from a cognitive dysfunction. Breflate or analogous compounds thereof enhance the long-term potential of nerve cells. Suitable monoamine reuptake inhibitors include NS-2330. Suitable nootropic agents include oxiracetam available from ISF Societa Per Azioni, pramiracetam available from Warner Lambert Co., idebenone available from Takeda Chemical Inds. Ltd., anapsos available from ASAC Pharmaceuticals International, nebracetam available from Boehringer Ingelheim Corp., JTP-2942 available from Japan Tobacco Inc., fasoracetam available from Nippon Shinyaku Co. Ltd., bacosides available from Central Drug Research Institute, alzene available from Bar-IIan University, KA-672 available from Dr. Willmar Schwabe GmbH & Co., alaptid available from VUFB, IQ-200, ALE-26015 available from Allelix Pharm-Eco LP and combinations thereof.

A useful dopamine receptor agonist is speramine. Useful AMPA receptor ligands include CX-516, CX-691 available from Cortex Pharmaceuticals Inc. and combinations thereof. Suitable calcium channel blockers include tamolarizine available from Nippon Chemiphar Co., Ltd., nimodipine available from Bayer AG, PD-1 76078 available from Elan Pharmaceuticals, Inc., and combinations thereof. Suitable apoptosis inhibitors include acetyl-L-carnitine, CEP-1347 available from Cephalon, Inc., TCH-346 available from Novartis AG and combinations thereof. A useful caspase inhibitor is pralnacasan. Suitable monoamine oxidase inhibitors include moclobemide available from Roche Holding AG, selegiline, rasagiline available from Teva Pharmaceutical Inds. Ltd., SL-25.1188, Ro-41-1049 available from Roche Holding AG, and combinations thereof. A useful 5-HTla receptor agonist is AP-159 available from Asahi Kasei Corp.; a suitable NGF stimulator is xaliprodene available from Sanofi-Synthelabo. Suitable neuroprotective agents include citicholine, GS-1590 available from Leo Pharmaceutical Products Ltd. A/S, CPI-1189 available from Centaur Pharmaceuticals Inc., SR-57667 available from Sanofi-Synthelabo and combinations thereof. Suitable H3 histamine receptor antagonists include GT-2016 and GT-2331 (both available from Gliatech, Inc.) and combinations thereof.

Useful prolylendopeptidase inhibitors include ONO-1603 available from Ono Pharmaceutical Co. Ltd., Z-321 available from Zeria Pharmaceutical Co. Ltd. and combinations thereof. A useful calcium modulator includes neurocalc available from Apollo Biopharmaceuticals, Inc. A suitable corticortropin releasing factor receptor antagonist includes NBI-113 available from Neurocrine Biosciences, Inc. A useful GABA modulator includes NGD 97-1 available from Neurogen Corp. A suitable sigma receptor ligand is igmesine available from Pfizer Inc. A useful imidazoline/alpha adrenergic receptor antagonist is efaroxan available from Reckitt & Colman PLC. A suitable vasoactive intestinal peptide receptor agonist is stearyl-NIe-VIP. A useful benzodiazepine inverse agonist is S-8510 available from Shionogi & Co. Ltd. A suitable cannabinoid receptor agonist is dronabinol available from Unimed Pharmaceuticals Inc. Useful thyrotropin releasing hormone receptor agonists include taltireline available from Tanabe Seiyaku Co. Ltd. and protirelin available from Takeda Chemical Inds., Inc. A suitable 5-HT3 antagonist is GYKI-46903. A useful topoisomerase II inhibitor is iododoxorubicin available from Pharmacia & Upjohn AB. A suitable steroid receptor agonist is GL-701 available from Leland Stanford Junior University. A useful corticosteroid receptor antagonist is anticort. A suitable nitric oxide modulator is GL-701. A suitable RAGE inhibitor is ALT-711 available from Alteon Inc. RAGE is a multiligand receptor of the immunoglobulin superfamily that is implicated in homeostasis and chronic disease. Bucciarelli, et al., Cell Mol Life Sci. 59(7), 1117-28 (2002).

The invention also relates to a pharmaceutical composition comprising at least one compound selected from the group consisting of D-phosphoserine and L-phosphoserine and a second therapeutic agent selected from the group consisting of antipsychotics, antidepressants, psychostimulants, and Alzheimer's disease therapeutics. In these pharmaceutical compositions, the second therapeutic agent is an antipsychotic selected from the group consisting of typical antipsychotics, atypical antipsychotics, and depot antipsychotics. Examples of second therapeutic agents include Chlorpromazine, Thioridazine, Mesoridazine, Fluphenazine, Perphenazine, Trifluoperazine, Thiothixene, Haloperidol (Haldol™, McNeil Pharmaceuticals, Spring House, Pa.), Loxapine, Molindone (Mobamm, Du Pont Multi-Source Products, Wilmington, Del.), Clozapine, Risperidone, Olanzapine, Quetiapine, Haloperidol decanoate, Fluphenazine decanoate, Fluphenazine enanthate, Amitriptyline, Amoxapine, Bupropion, Bupropion SR, Clomipramine, Desipramine, Doxepin, Fluoxetine, Fluvoxamine, Imipramine, Maprotiline, Mirtazapine, Nefazodone, Nortriptyline, Paroxetine, Phenelzine, Protriptyline, Sertraline, Tranylcypromine, Trazodone, Trimipramine, Venlafaxine, Velafaxine XR, Dextroamphetamine, Methamphetamine, Methylphenidate, Pemoline, Donepezil, Tacrine™, Acetoplenazine, Chlorprothixene, Droperidol, Pimozide, Butaperazine, Carphenazine Remoxipride, Piperacetazine, Sulpiride, and Ziprasidone.

In another alternative embodiment, the compositions used in the methods to treat or prevent a neuropsychiatric disorder characterized by attenuated NMDA neurotransmission. The neuropsychiatric disorder may be Alzheimer's disease, Down's syndrome, depression, benign forgetfulness, cerebral amyloid angiopathy, vascular dementia, hemorrhagic stroke, Mild Cognitive Impairment (“MCI”), or close head injury.

In another alternative embodiment, the compositions used in the methods of the present invention can further comprise one or more bile acid sequestrants (insoluble anion exchange resins), coadministered with or in combination with a compound of any Formula herein.

Bile acid sequestrants bind bile acids in the intestine, interrupting the enterohepatic circulation of bile acids and causing an increase in the faecal excretion of steroids. Use of bile acid sequestrants is desirable because of their non-systemic mode of action. Bile acid sequestrants can lower intrahepatic cholesterol and promote the synthesis of apo B/E (LDL) receptors which bind LDL from plasma to further reduce cholesterol levels in the blood.

Non-limiting examples of suitable bile acid sequestrants include cholestyramine (a styrene-divinylbenzene copolymer containing quaternary ammonium cationic groups capable of binding bile acids, such as Questranm cholestyramine which are available from Bristol-Myers Squibb), colestipol (a copolymer of diethylenetriamine and 1-chloro-2,3-epoxypropane, such as Colestid™ tablets which are available from Pharmacia), colesevelam hydrochloride (such as WelChol™ Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl) alkylamines and poliglusam, insoluble quaternized polystyrenes, saponins and mixtures thereof. Other useful bile acid sequestrants are disclosed in PCT Patent Applications Nos. WO 97/11345 and WO 98/57652, and U.S. Pat. Nos. 3,692,895 and 5,703,188 which are incorporated herein by reference. Suitable inorganic cholesterol sequestrants include bismuth salicylate plus montmorillonite clay, aluminum hydroxide and calcium carbonate antacids.

In an alternative embodiment, the compositions used in the methods of the present invention can further comprise one or more ileal bile acid transport (“IBAT”) inhibitors (or apical sodium co-dependent bile acid transport (“ASBT”) inhibitors) coadministered with or in combination with a compound of any Formula herein. The IBAT inhibitors can inhibit bile acid transport to reduce LDL cholesterol levels. Non-limiting examples of suitable IBAT inhibitors include benzothiepines such as therapeutic compounds comprising a 2,3,4,5-tetrahydro-1-benzothie-pine 1,1-dioxide structure such as are disclosed in PCT Patent Application WO 00/38727 which is incorporated herein by reference.

In another alternative embodiment, the compositions used in the methods of the present invention can further comprise nicotinic acid (niacin) or derivatives thereof coadministered with or in combination with a compound of any Formula herein.

As used herein, “nicotinic acid derivative” means a compound comprising a pyridine-3-carboxylate structure or a pyrazine-2-carboxylate structure, including acid forms, salts, esters, zwitterions and tautomers, where available. Examples of nicotinic acid derivatives include niceritrol, nicofuranose and acipimox (5-methylpyrazine-2-carboxylic acid 4-oxide). Nicotinic acid and its derivatives inhibit hepatic production of VLDL and its metabolite LDL and increases HDL and apo A-I levels. An example of a suitable nicotinic acid product is Niaspamm (niacin extended-release tablets) which are available from Kos.

The compositions, therapeutic combinations or methods of the present invention can further comprise one or more obesity control medications. Useful obesity control medications include, but are not limited to, drugs that reduce energy intake or suppress appetite, drugs that increase energy expenditure and nutrient-partitioning agents. Suitable obesity control medications include, but are not limited to, noradrenergic agents (such as diethylpropion, mazindol, phenylpropanolamine, phentermine, phendimetrazine, phendamine tartrate, methamphetamine, phendimetrazine and tartrate); serotonergic agents (such as sibutramine, fenfluramine, dexfenfluramine, fluoxetine, fluvoxamine and paroxtine); thermogenic agents (such as ephedrine, caffeine, theophylline, and selective β3-adrenergic agonists); an alpha-blocking agent; a kainite or AMPA receptor antagonist; a leptin-lipolysis stimulated receptor; a phosphodiesterase enzyme inhibitor; a compound having nucleotide sequences of the mahogany gene; a fibroblast growth factor-10 polypeptide; a monoamine oxidase inhibitor (such as befloxatone, moclobemide, brofaromine, phenoxathine, esuprone, befol, toloxatone, pirlindol, amiflamine, sercloremine, bazinaprine, lazabemide, milacemide and caroxazone); a compound for increasing lipid metabolism (such as evodiamine compounds); and a lipase inhibitor (such as orlistat). Generally, a total dosage of the above-described obesity control medications can range from 1 to 3,000 mg/day, desirably from about 1 to 1,000 mg/day and more desirably from about 1 to 200 mg/day in single or 2-4 divided doses.

The compositions, therapeutic combinations or methods of the present invention can further comprise one or more blood modifiers. Useful blood modifiers include but are not limited to anti-coagulants (argatroban, bivalirudin, dalteparin sodium, desirudin, dicumarol, lyapolate sodium, nafamostat mesylate, phenprocoumon, tinzaparin sodium, warfarin sodium); antithrombotic (anagrelide hydrochloride, bivalirudin, cilostazol, dalteparin sodium, danaparoid sodium, dazoxiben hydrochloride, efegatran sulfate, enoxaparin sodium, fluretofen, ifetroban, ifetroban sodium, lamifiban, lotrafiban hydrochloride, napsagatran, orbofiban acetate, roxifiban acetate, sibrafiban, tinzaparin sodium, trifenagrel, abciximab, zolimomab aritox); fibrinogen receptor antagonists (roxifiban acetate, fradafiban, orbofiban, lotrafiban hydrochloride, tirofiban, xemilofiban, monoclonal antibody 7E3, sibrafiban); platelet inhibitors (cilostazol, clopidogrel bisulfate, epoprostenol, epoprostenol sodium, ticlopidine hydrochloride, aspirin, ibuprofen, naproxen, sulindae, idomethacin, mefenamate, droxicam, diclofenac, sulfinpyrazone, piroxicam, dipyridamole); platelet aggregation inhibitors (acadesine, beraprost, beraprost sodium, ciprostene calcium, itazigrel, lifarizine, lotrafiban hydrochloride, orbofiban acetate, oxagrelate, fradafiban, orbofiban, tirofiban, xemilofiban); hemorrheologic agents (pentoxifylline); lipoprotein associated coagulation inhibitor; Factor VIIa inhibitors (4H-3 1-benzoxazin-4-ones, 4H-3,1-benzoxazin-4-thiones, quinazolin-4-ones, quinazolin-4-thiones, benzothiazin-4-ones, imidazolyl-boronic acid-derived peptide analogues TFPI-derived peptides, naphthalene-2-sulfonic acid {1-[3-(aminoiminomethyl)-benzyl]-2-oxo-pyrrol-idin-3-(S)-yl} amide trifluoroacetate, dibenzofuran-2-sulfonic acid {1-[3-(aminomethyl)-benzyl]-5-oxo-pyrrolidin-3-yl}-amide, tolulene-4-sulfonic acid {1-[3-(aminoiminomethyl)-benzyl]-2-oxo-pyrrolidi-n-3-(S)-yl}-amide trifluoroacetate, 3,4-dihydro-1H-isoquinoline-2-sulfonic acid {1-[3-(aminoiminomethyl)-benzyl]-2-oxo-pyrrolin-3-(S)-yl}-amide trifluoroacetate); Factor Xa inhibitors (disubstituted pyrazolines, disubstituted triazolines, substituted n-[(aminoiminomethyl)phenyl] propylamides, substituted N-[(amino-methyl)phenyl] propylamides, tissue factor pathway inhibitor (TFPI), low molecular weight heparins, heparinoids, benzimidazolines, benzoxazolinones, benzopiperazinones, indanones, dibasic (amidinoaryl) propanoic acid derivatives, amidinophenyl-pyrrolidines, amidinophenylpyrrolines, amidinophenyl-isoxazolidines, amidinoindoles, amidinoazoles, bis-arlysulfonyl-aminobenzamide derivatives, peptidic Factor Xa inhibitors).

The compositions, therapeutic combinations or methods of the present invention can further comprise one or more cardiovascular agents. Useful cardiovascular agents include but are not limited to calcium channel blockers (clentiazem maleate, amlodipine besylate, isradipine (DynaCirc™, Reliant Pharmaceuticals, Liberty Corner, N.J.), nimodipine, felodipine (Plendil™, Merck & Co., Inc., Rahway, N.J.), nilvadipine, nifedipine, teludipine hydrochloride, diltiazem hydrochloride ((Cardizen™ or Cardizem SR™, Aventis, Strasbourg, France), belfosdil, verapamil hydrochloride (Calan™ or Calan SR™, G.D. Searle LLC, Skokie, Ill.), fostedil); adrenergic blockers (fenspiride hydrochloride, labetalol hydrochloride, proroxan, alfuzosin hydrochloride, acebutolol, acebutolol hydrochloride, alprenolol hydrochloride, atenolol, bunolol hydrochloride, carteolol hydrochloride, celiprolol hydrochloride, cetamolol hydrochloride, cicloprolol hydrochloride, dexpropranolol hydrochloride, diacetolol hydrochloride, dilevalol hydrochloride, esmolol hydrochloride, exaprolol hydrochloride, flestolol sulfate, labetalol hydrochloride, levobetaxolol hydrochloride, levobunolol hydrochloride, metalol hydrochloride, metoprolol, metoprolol tartrate, nadolol, pamatolol sulfate, penbutolol sulfate, practolol, propranolol hydrochloride (Inderal™, Wyeth, Madison, N.J.), sotalol hydrochloride, timolol, timolol maleate, tiprenolol hydrochloride, tolamolol, bisoprolol, bisoprolol fumarate, nebivolol); adrenergic stimulants; angiotensin converting enzyme (ACE) inhibitors (benazepril hydrochloride, benazeprilat, captopril (Capoten™, Bristol-Myers Squibb Co., New York, N.Y.), delapril hydrochloride, fosinopril sodium, libenzapril, moexipril hydrochloride, pentopril, perindopril, quinapril hydrochloride, quinaprilat, ramipril (Altace™, Hoechst Marion Roussel, Inc., now Aventis, Strasbourg, France), spirapril hydrochloride, spiraprilat, teprotide, enalapril maleate (Vasotec™, Merck & Co., Inc., Rahway, N.J.), lisinopril (Zestril™, Stuart, AstraZenica, Wilmington, Del.), zofenopril calcium, perindopril erbumine); antihypertensive agents (althiazide, benzthiazide, captopril, carvedilol, chlorothiazide sodium, clonidine hydrochloride (Catapres™, Boehringer Ingelheim, Ridgefield, Conn.), cyclothiazide, delapril hydrochloride, dilevalol hydrochloride, doxazosin mesylate, fosinopril sodium, guanfacine hydrochloride (Tenex™, Robins, ESP Pharmaceuticals, Flanders, N.J.), methyldopa, metoprolol succinate, moexipril hydrochloride, monatepil maleate, pelanserin hydrochloride, phenoxybenzamine hydrochloride, prazosin hydrochloride, primidolol, quinapril hydrochloride, quinaprilat, ramipril, terazosin hydrochloride, candesartan, candesartan cilexetil, telmisartan, amiodipine besylate, amlodipine maleate (Norvasc™, Pfizer, New York N.Y.), bevantolol hydrochloride); angiotensin II receptor antagonists (candesartan, irbesartan, losartan potassium, candesartan cilexetil, telmisartan); anti-anginal agents (amlodipine besylate, amlodipine maleate, betaxolol hydrochloride, bevantolol hydrochloride, butoprozine hydrochloride, carvedilol, cinepazet maleate, metoprolol succinate, molsidomine, monatepil maleate, primidolol, ranolazine hydrochoride, tosifen, verapamil hydrochloride); coronary vasodilators (fostedil, azaclorzine hydrochloride, chromonar hydrochloride, clonitrate, diltiazem hydrochloride, dipyridamole, droprenilamine, erythrityl tetranitrate, isosorbide dinitrate, isosorbide mononitrate, lidoflazine, mioflazine hydrochloride, mixidine, molsidomine, nicorandil, nifedipine (Procardia™, Pfizer, New York, N.Y.), nisoldipine, nitroglycerine, oxprenolol hydrochloride, pentrinitrol, perhexiline maleate, prenylamine, propatyl nitrate, terodiline hydrochloride, tolamolol, verapamil); diuretics (the combination product of hydrochlorothiazide and spironolactone and the combination product of hydrochlorothiazide and triamterene).

Blood-Brain Barrier

Nitric oxide is a vasodilator of the peripheral vasculature in normal tissue of the body. Increasing generation of nitric oxide by nitric oxide synthase causes vasodilation without loss of blood pressure. The blood-pressure-independent increase in blood flow through brain tissue increases cerebral bioavailability of blood-born compositions. This increase in nitric oxide may be stimulated by administering L-arginine. As nitric oxide is increased, cerebral blood flow is consequently increased, and drugs in the blood stream are carried along with the increased flow into brain tissue. Therefore, L-arginine may be used in the pharmaceutical compositions of the invention to enhance delivery of agents to brain tissue after introducing a pharmaceutical composition into the blood stream of the subject substantially contemporaneously with a blood flow enhancing amount of L-arginine. WO 00/56328

Agents of the invention that exert their physiological effect in vivo in the brain may be more useful if they gain access to target cells in the brain. Non-limiting examples of brain cells are neurons, glial cells (astrocytes, oligodendrocytes, microglia), cerebrovascular cells (muscle cells, endothelial cells), and cells that comprise the meninges. The blood brain barrier (“BBB”) typically restricts access to brain cells by acting as a physical and functional blockade that separates the brain parenchyma from the systemic circulation (see, e.g., Pardridge, et al., J. Neurovirol. 5(6), 556-69 (1999); Rubin, et al., Rev. Neurosci. 22, 11-28 (1999)). Circulating molecules are normally able to gain access to brain cells via one of two processes: lipid-mediated transport through the BBB by free diffusion, or active (or catalyzed) transport.

The agents of the invention may be formulated to improve distribution in vivo, for example as powdered or liquid tablet or solution for oral administration or as a nasal spray, nose drops, a gel or ointment, through a tube or catheter, by syringe, by packtail, by pledget, or by submucosal infusion. For example, the blood-brain barrier (BBB) excludes many highly hydrophilic agents. To ensure that the more hydrophilic therapeutic agents of the invention cross the BBB, they may be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs (“targeting moieties” or “targeting groups” or “transporting vectors”), thus providing targeted drug delivery (see, e.g., V.V. Ranade J. Clin. Pharmacol. 29, 685 (1989)). Likewise, the agents may be linked to targeting groups that facilitate penetration of the blood brain barrier. In one embodiment, the method of the present invention employs a naturally occurring polyamine linked to an agent that is a small molecule and is useful for inhibiting Aβ deposition.

To facilitate transport of agents of the invention across the BBB, they may be coupled to a BBB transport vector (for review of BBB transport vectors and mechanisms, see, Bickel, et al., Adv. Drug Delivery Reviews 46, 247-79 (2001)). Exemplary transport vectors include cationized albumin or the OX26 monoclonal antibody to the transferrin receptor; these proteins undergo absorptive-mediated and receptor-mediated transcytosis through the BBB, respectively. Natural cell metabolites that may be used as targeting groups, include, inter alia, putrescine, spermidine, spermine, or DHA. Other exemplary targeting moieties include folate or biotin (see, e.g., U.S. Pat. No. 5,416,016); mannosides (Umezawa, et al., Biochem. Biophys. Res. Commun. 153, 1038 (1988)); antibodies (P.G. Bloeman, et al., FEBS Lett. 357, 140 (1995); M. Owais, et al., Antimicrob. Agents Chemother. 39, 180 (1995)); surfactant protein A receptor (Briscoe, et al., Am. J. Physiol. 1233, 134 (1995)); gp120 (Schreier, et al., J. Biol. Chem. 269, 9090 (1994)); see also, K. Keinanen and M.L. Laukkanen, FEBS Lett. 346, 123 (1994); J.J. Killion and I.J. Fidler, Immunomethods 4, 273 (1994).

Examples of other BBB transport vectors that target receptor-mediated transport systems into the brain include factors such as insulin, insulin-like growth factors (“IGF-I,” and “IGF-II”), angiotensin II, atrial and brain natriuretic peptide (“ANP,” and “BNP”), interleukin I (“IL-1”) and transferrin. Monoclonal antibodies to the receptors that bind these factors may also be used as BBB transport vectors. BBB transport vectors targeting mechanisms for absorptive-mediated transcytosis include cationic moieties such as cationized LDL, albumin or horseradish peroxidase coupled with polylysine, cationized albumin or cationized immunoglobulins. Small basic oligopeptides such as the dynorphin analogue E-2078 and the ACTH analogue ebiratide may also cross the brain via absorptive-mediated transcytosis and are potential transport vectors.

Other BBB transport vectors target systems for transporting nutrients into the brain. Examples of such BBB transport vectors include hexose moieties, e.g., glucose and monocarboxylic acids, e.g., lactic acid and neutral amino acids, e.g., phenylalanine and amines, e.g., choline and basic amino acids, e.g., arginine, nucleosides, e.g., adenosine and purine bases, e.g., adenine, and thyroid hormone, e.g., triiodothyridine. Antibodies to the extracellular domain of nutrient transporters may also be used as transport vectors. Other possible vectors include angiotensin II and ANP, which may be involved in regulating BBB permeability.

In some cases, the bond linking the therapeutic agent to the transport vector may be cleaved following transport into the brain in order to liberate the biologically active agent. Exemplary linkers include disulfide bonds, ester-based linkages, thioether linkages, amide bonds, acid-labile linkages, and Schiff base linkages. Avidin/biotin linkers, in which avidin is covalently coupled to the BBB drug transport vector, may also be used. Avidin itself may be a drug transport vector.

Transcytosis, including receptor-mediated transport of compositions across the blood brain barrier, may also be suitable for the agents of the invention. Transferrin receptor-mediated delivery is disclosed in U.S. Pat. Nos. 5,672,683; 5,383,988; 5,527,527; 5,977,307; and 6,015,555. Transferrin-mediated transport is also known. P.M. Friden, et al., Pharmacol. Exp. Ther. 278, 1491-98 (1996); H.J. Lee, J. Pharmacol. Exp. Ther. 292, 1048-52 (2000). EGF receptor-mediated delivery is disclosed in Y. Deguchi, et al., Bioconjug. Chem. 10, 32-37 (1999), and transcytosis is described in A. Cerletti, et al., J. Drug Target. 8, 435-46 (2000). Insulin fragments have also been used as carriers for delivery across the blood brain barrier. M. Fukuta, et al., Pharm. Res. 11. 1681-88 (1994). Delivery of agents via a conjugate of neutral avidin and cationized human albumin has also been described. Y.S. Kang, et al., Pharm. Res. 1, 1257-64 (1994).

Other modifications in order to enhance penetration of the agents of the invention across the blood brain barrier may be accomplished using methods and derivatives known in the art. For example, U.S. Pat. No. 6,024,977 discloses covalent polar lipid conjugates for targeting to brain and central nervous system. U.S. Pat. No. 5,017,566 discloses cyclodextrin derivatives comprising inclusion complexes of lipoidal forms of dihydropyridine redox targeting moieties. U.S. Pat. No. 5,023,252 discloses the use of pharmaceutical compositions comprising a neurologically active drug and a compound for facilitating transport of the drug across the blood-brain barrier including a macrocyclic ester, diester, amide, diamide, amidine, diamidine, thioester, dithioester, thioamide, ketone or lactone. U.S. Pat. No. 5,024,998 discloses parenteral solutions of aqueous-insoluble drugs with cyclodextrin derivatives. U.S. Pat. No. 5,039,794 discloses the use of a metastatic tumor-derived egress factor for facilitating the transport of compounds across the blood-brain barrier. U.S. Pat. No. 5,112,863 discloses the use of N-acyl amino acid derivatives as antipsychotic drugs for delivery across the blood-brain barrier. U.S. Pat. No. 5,124,146 discloses a method for delivery of therapeutic agents across the blood-brain barrier at sites of increase permeability associated with brain lesions. U.S. Pat. No. 5,153,179 discloses acylated glycerol and derivatives for use in a medicament for improved penetration of cell membranes. U.S. Pat. No. 5,177,064 discloses the use of lipoidal phosphonate derivatives of nucleoside antiviral agents for delivery across the blood-brain barrier. U.S. Pat. No. 5,254,342 discloses receptor-mediated transcytosis of the blood-brain barrier using the transferrin receptor in combination with pharmaceutical compounds that enhance or accelerate this process. U.S. Pat. No. 5,258,402 discloses treatment of epilepsy with imidate derivatives of anticonvulsive sulfamate. U.S. Pat. No. 5,270,312 discloses substituted piperazines as central nervous system agents. U.S. Pat. No. 5,284,876 discloses fatty acid conjugates of dopamine drugs. U.S. Pat. No. 5,389,623 discloses the use of lipid dihydropyridine derivatives of anti-inflammatory steroids or steroid sex hormones for delivery across the blood-brain barrier. U.S. Pat. No. 5,405,834 discloses prodrug derivatives of thyrotropin releasing hormone. U.S. Pat. No. 5,413,996 discloses acyloxyalkyl phosphonate conjugates of neurologically-active drugs for anionic sequestration of such drugs in brain tissue. U.S. Pat. No. 5,434,137 discloses methods for the selective opening of abnormal brain tissue capillaries using bradykinin infused into the carotid artery. U.S. Pat. No. 5,442,043 discloses a peptide conjugate between a peptide having a biological activity and incapable of crossing the blood-brain barrier and a peptide which exhibits no biological activity and is capable of passing the blood-brain barrier by receptor-mediated endocytosis. U.S. Pat. No. 5,466,683 discloses water soluble analogues of an anticonvulsant for the treatment of epilepsy. U.S. Pat. No. 5,525,727 discloses compositions for differential uptake and retention in brain tissue comprising a conjugate of a narcotic analgesic and agonists and antagonists thereof with a lipid form of dihydropyridine that forms a redox salt upon uptake across the blood-brain barrier that prevents partitioning back to the systemic circulation.

Still further examples of modifications that enhance penetration of the blood brain barrier are described in International (PCT) Application Publication Number WO 85/02342, which discloses a drug composition comprising a glycerolipid or derivative thereof. PCT Publication Number WO 089/11299 discloses a chemical conjugate of an antibody with an enzyme which is delivered specifically to a brain lesion site for activating a separately-administered neurologically-active prodrug. PCT Publication Number WO 91/04014 discloses methods for delivering therapeutic and diagnostic agents across the blood-brain barrier by encapsulating the drugs in liposomes targeted to brain tissue using transport-specific receptor ligands or antibodies. PCT Publication Number WO 91/04745 discloses transport across the blood-brain barrier using cell adhesion molecules and fragments thereof to increase the permeability of tight junctions in vascular endothelium. PCT Publication Number WO 91/14438 discloses the use of a modified, chimeric monoclonal antibody for facilitating transport of substances across the blood-brain barrier. PCT Publication Number WO 94/01131 discloses lipidized proteins, including antibodies. PCT Publication Number WO 94/03424 discloses the use of amino acid derivatives as drug conjugates for facilitating transport across the blood-brain barrier. PCT Publication Number WO 94/06450 discloses conjugates of neurologically-active drugs with a dihydropyridine-type redox targeting moiety and comprising an amino acid linkage and an aliphatic residue. PCT Publication Number WO 94/02178 discloses antibody-targeted liposomes for delivery across the blood-brain barrier. PCT Publication Number WO 95/07092 discloses the use of drug-growth factor conjugates for delivering drugs across the blood-brain barrier. PCT Publication Number WO 96/00537 discloses polymeric microspheres as injectable drug-delivery vehicles for delivering bioactive agents to sites within the central nervous system. PCT Publication Number WO 96/04001 discloses omega-3-fatty acid conjugates of neurologically-active drugs for brain tissue delivery. PCT WO 96/22303 discloses fatty acid and glycerolipid conjugates of neurologically-active drugs for brain tissue delivery.

In general, it is well within the ordinary skill in the art to prepare an ester, amide or hydrazide derivative of an agent of the invention, for example, from the corresponding carboxylic acid and a suitable reagent. For instance, a carboxylic acid-containing compound, or a reactive equivalent thereof, may be reacted with a hydroxyl-containing compound, or a reactive equivalent thereof, so as to provide the corresponding ester. See, e.g., “Comprehensive Organic Transformations,” 2^{nd} Ed., by R.C. Larock, VCH Publishers John Wiley & Sons, Ltd. (199989); “March's Advanced Organic Chemistry,” 5^{th} Ed., by M.B. Smith and J. March, John Wiley & Sons, Ltd. (2000).

The compound may also act from the periphery, causing a change in the equilibrium of the amyloid protein concentration in the two compartments i.e. (systemic vs central). In this case a compound may not be required to penetrate the brain to induce or decrease the concentration of Aβ in the brain (a “sink” effect)

Prodrugs

The present invention is also related to prodrugs of the agents of the Formulae disclosed herein. Prodrugs are agents which are converted in vivo to active forms (see, e.g., R.B. Silverman, 1992, “The Organic Chemistry of Drug Design and Drug Action,” Academic Press, Chp. 8). Prodrugs can be used to alter the biodistribution (e.g., to allow agents which would not typically enter the reactive site of the protease) or the pharmacokinetics for a particular agent. For example, a carboxylic acid group, can be esterified, e.g., with a methyl group or an ethyl group to yield an ester. When the ester is administered to a subject, the ester is cleaved, enzymatically or non-enzymatically, reductively, oxidatively, or hydrolytically, to reveal the anionic group. An anionic group can be esterified with moieties (e.g., acyloxymethyl esters) which are cleaved to reveal an intermediate agent which subsequently decomposes to yield the active agent. The prodrug moieties may be metabolized in vivo by esterases or by other mechanisms to carboxylic acids.

Examples of prodrugs and their uses are well known in the art (see, e.g., Berge, et al., “Pharmaceutical Salts”, J. Pharm. Sci. 66, 1-19 (1977)). The prodrugs can be prepared in situ during the final isolation and purification of the agents, or by separately reacting the purified agent in its free acid form with a suitable derivatizing agent. Carboxylic acids can be converted into esters via treatment with an alcohol in the presence of a catalyst.

Examples of cleavable carboxylic acid prodrug moieties include substituted and unsubstituted, branched or unbranched lower alkyl ester moieties, (e.g., ethyl esters, propyl esters, butyl esters, pentyl esters, cyclopentyl esters, hexyl esters, cyclohexyl esters), lower alkenyl esters, dilower alkyl-amino lower-alkyl esters (e.g., dimethylaminoethyl ester), acylamino lower alkyl esters, acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, dilower alkyl amides, and hydroxy amides.

Pharmaceutical Prevarations

In another embodiment, the present invention relates to pharmaceutical compositions comprising agents according to any of the Formulae herein for the treatment of an amyloid-β related disease, as well as methods of manufacturing such pharmaceutical compositions.

In general, the agents of the present invention may be prepared by the methods illustrated in the general reaction schemes as, for example, in the patents and patent applications refered to herein, or by modifications thereof, using readily available starting materials, reagents and conventional synthesis procedures. In these reactions, it is also possible to make use of variants which are in themselves known, but are not mentioned here. Functional and structural equivalents of the agents described herein and which have the same general properties, wherein one or more simple variations of substituents are made which do not adversely affect the essential nature or the utility of the agent.

The agents of the invention may be supplied in a solution with an appropriate solvent or in a solvent-free form (e.g., lyophilized). In another aspect of the invention, the agents and buffers necessary for carrying out the methods of the invention may be packaged as a kit. The kit may be commercially used according to the methods described herein and may include instructions for use in a method of the invention. Additional kit components may include acids, bases, buffering agents, inorganic salts, solvents, antioxidants, preservatives, or metal chelators. The additional kit components are present as pure compositions, or as aqueous or organic solutions that incorporate one or more additional kit components. Any or all of the kit components optionally further comprise buffers.

The therapeutic agent may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

To administer the therapeutic agent by other than parenteral administration, it may be necessary to coat the agent with, or co-administer the agent with, a material to prevent its inactivation. For example, the therapeutic agent may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., J. Neuroimmunol. 7, 27 (1984)).

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and ftngi.

Suitable pharmaceutically acceptable carriers include, without limitation, any non-immunogenic pharmaceutical adjuvants suitable for oral, parenteral, nasal, mucosal, transdermal, intravascular (IV), intraarterial (IA), intramuscular (IM), and subcutaneous (SC) administration routes, such as phosphate buffer saline (PBS).

The vehicle can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents are included, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic agent in the required amount in an appropriate solvent with one or a combination of ingredients enumerated above, as required, followed by filtered sterilization. Generally, dispersions are prepared by incorporating the therapeutic agent into a sterile vehicle which contains a basic dispersion medium and the required other ingredients from those enumerated above. In the case of sterile powders for the preparation of sterile injectable solutions, the methods of preparation are vacuum drying and freeze-drying which yields a powder of the active ingredient (i.e., the therapeutic agent) plus any additional desired ingredient from a previously sterile-filtered solution thereof.

The therapeutic agent can be orally administered, for example, with an inert diluent or an assimilable edible carrier. The therapeutic agent and other ingredients may also be enclosed in a hard or soft shell gelatin capsule, compressed into tablets, or incorporated directly into the subject's diet. For oral therapeutic administration, the therapeutic agent may be incorporated with excipients and used in the form of ingestible tablets, buccal tablets, troches, capsules, elixirs, suspensions, syrups, wafers, and the like. The percentage of the therapeutic agent in the compositions and preparations may, of course, be varied. The amount of the therapeutic agent in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic agent calculated to produce the desired therapeutic effect in association with the required pharmaceutical vehicle. The specification for the dosage unit forms of the invention are dictated by and directly dependent on (a) the unique characteristics of the therapeutic agent and the particular therapeutic effect to be achieved, and (b) the limitations inherent in the art of compounding such a therapeutic agent for the treatment of amyloid deposition in subjects.

The present invention therefore includes pharmaceutical formulations comprising the agents of the Formulae described herein, including pharmaceutically acceptable salts thereof, in pharmaceutically acceptable carriers for aerosol, oral and parenteral administration. Also, the present invention includes such agents, or salts thereof, which have been lyophilized and which may be reconstituted to form pharmaceutically acceptable formulations for administration, as by intravenous, intramuscular, or subcutaneous injection. Administration may also be intradermal or transdermal.

In accordance with the present invention, an agent of the Formulae described herein, and pharmaceutically acceptable salts thereof, may be administered orally or through inhalation as a solid, or may be administered intramuscularly or intravenously as a solution, suspension or emulsion. Alternatively, the agents or salts may also be administered by inhalation, intravenously or intramuscularly as a liposomal suspension.

Pharmaceutical formulations are also provided which are suitable for administration as an aerosol, by inhalation. These formulations comprise a solution or suspension of the desired agent of any Formula herein, or a salt thereof, or a plurality of solid particles of the agent or salt. The desired formulation may be placed in a small chamber and nebulized. Nebulization may be accomplished by compressed air or by ultrasonic energy to form a plurality of liquid droplets or solid particles comprising the agents or salts. The liquid droplets or solid particles should have a particle size in the range of about 0.5 to about 5 microns. The solid particles can be obtained by processing the solid agent of any Formula described herein, or a salt thereof, in any appropriate manner known in the art, such as by micronization. The size of the solid particles or droplets will be, for example, from about 1 to about 2 microns. In this respect, commercial nebulizers are available to achieve this purpose.

A pharmaceutical formulation suitable for administration as an aerosol may be in the form of a liquid, the formulation will comprise a water-soluble agent of any Formula described herein, or a salt thereof, in a carrier which comprises water. A surfactant may be present which lowers the surface tension of the formulation sufficiently to result in the formation of droplets within the desired size range when subjected to nebulization.

Peroral compositions also include liquid solutions, emulsions, suspensions, and the like. The pharmaceutically acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol. and water. For a suspension, typical suspending agents include methyl cellulose, sodium carboxymethyl cellulose, tragacanth, and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Pharmaceutical compositions may also be coated by conventional methods, typically with pH or time-dependent coatings, such that the subject agent is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, waxes, and shellac.

Other compositions useful for attaining systemic delivery of the subject agents include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

The compositions of this invention can also be administered topically to a subject, e.g., by the direct laying on or spreading of the composition on the epidermal or epithelial tissue of the subject, or transdermally via a “patch”. Such compositions include, for example, lotions, creams, solutions, gels and solids. These topical compositions may comprise an effective amount, usually at least about 0.1%, or evan from about 1% to about 5%, of an agent of the invention. Suitable carriers for topical administration typically remain in place on the skin as a continuous film, and resist being removed by perspiration or immersion in water. Generally, the carrier is organic in nature and capable of having dispersed or dissolved therein the therapeutic agent. The carrier may include pharmaceutically acceptable emolients, emulsifiers, thickening agents, solvents and the like.

Active agents are administered at a therapeutically effective dosage sufficient to inhibit amyloid deposition in a subject. A “therapeutically effective” dosage inhibits amyloid deposition by, for example, at least about 20%, or by at least about 40%, or even by at least about 60%, or by at least about 80% relative to untreated subjects. In the case of an Alzheimer's subject, a “therapeutically effective” dosage stabilizes cognitive function or prevents a further decrease in cognitive function (i.e., preventing, slowing, or stopping disease progression). The present invention accordingly provides therapeutic drugs. By “therapeutic” or “drug” is meant an agent having a beneficial ameliorative or prophylactic effect on a specific disease or condition in a living human or non-human animal.

Furthermore, active agents are administered at a therapeutically effective dosage sufficient to decrease deposition in a subject of amyloid protein, e.g., Aβ40 or Aβ42. A therapeutically effective dosage inhibits amyloid deposition by, for example, at least about 15%, or by at least about 40%, or even by at least 60%, or at least by about 80% relative to untreated subjects.

In another embodiment, active agents are administered at a therapeutically effective dosage sufficient to increase or enhance amyloid protein, e.g., Aβ40 or Aβ42, in the.blood of a subject A therapeutically effective dosage increases the concentration by, for example, at least about 15%, or by at least about 40%, or even by at least 60%, or at least by about 80% relative to untreated subjects.

In yet another embodiment, active agents are administered at a therapeutically effective dosage sufficient to improve ADAS-cog test scores by, e.g., at least about 1 point, at least about 2 points, at least about 3 points, at least about 4 points, at least about 5 points, at least about 10 points, at least about 12 points, at least about 15 points, or at least about 20 points relative to untreated subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50, and usually a larger therapeutic index are more efficacious. While agents that exhibit toxic side effects may be used, care should be taken to design a delivery system that targets such agents to the site of affected tissue in order to minimize potential damage to uninfected cells and, thereby, reduce side effects.

It is understood that appropriate doses depend upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses depend upon the potency with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

The ability of an agent to inhibit amyloid deposition can be evaluated in an animal model system that may be predictive of efficacy in inhibiting amyloid deposition in human diseases, such as a transgenic mouse expressing human APP or other relevant animal models where Aβ deposition is seen. Likewise, the ability of an agent to prevent or reduce cognitive impairment in a model system may be indicative of efficacy in humans. Alternatively, the ability of an agent can be evaluated by examining the ability of the agent to inhibit amyloid fibril formation in vitro, e.g., using a fibrillogenesis assay such as that described herein, including a ThT, CD, or EM assay. Also the binding of an agent to amyloid fibrils may be measured using a MS assay as described herein.

Pharmaceutically Acceptable Salts

Certain embodiments of the present agents can contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of agents of the present invention. These salts can be prepared in situ during the final isolation and purification of the agents of the invention, or by separately reacting a purified agent of the invention in its free base form with a suitable organic or inorganic acid, and isolating the salt thus formed.

Representative salts include the hydrohalide (including hydrobromide and hydrochloride), sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, 2-hydroxyethanesulfonate, and laurylsulphonate salts and the like. See, e.g., Berge et al., “Pharmaceutical Salts”, J. Pharm. Sci. 66, 1-19 (1977).

In other cases, the agents of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of agents of the present invention.

These salts can likewise be prepared in situ during the final isolation and purification of the agents, or by separately reacting the purified agent in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Those skilled in the art will recognize, or be able to ascertain using no more than routine experimentation, many equivalents to the specific embodiments and methods described herein. Such equivalents are intended to be encompassed by the scope of the following claims. All patents, patent applications, and literature references cited herein are hereby expressly incorporated by reference in their entirety. This invention is further illustrated by the following examples which should not be construed as limiting.

“Pharmaceutically acceptable salts” also includes, for example, derivatives of agents modified by making acid or base salts thereof, as described further below and elsewhere in the present application. Examples of pharmaceutically acceptable salts include mineral or organic acid salts of basic residues such as amines; and alkali or organic salts of acidic residues such as carboxylic acids. Pharmaceutically acceptable salts include the conventional non-toxic salts or the quaternary ammonium salts of the parent agent formed, for example, from non-toxic inorganic or organic acids. Such conventional non-toxic salts include those derived from inorganic acids such as hydrochloric, hydrobromic, sulfuric, sulfamic, phosphoric, and nitric acid; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic acid. Pharmaceutically acceptable salts may be synthesized from the parent agent which contains a basic or acidic moiety by conventional chemical methods. Generally, such salts may be prepared by reacting the free acid or base forms of these agents with a stoichiometric amount of the appropriate base or acid in water or in an organic solvent, or in a mixture of the two.

It is to be understood that wherever values and ranges are provided herein, e.g., in ages of subject populations, dosages, and blood levels, all values and ranges encompassed by these values and ranges, are meant to be encompassed within the scope of the present invention. Morover, all values in between these values and ranges may also be the upper or lower limits of a range.

### EXAMPLES

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. The contents of all references, issued patents, and published patent applications cited throughout this application are hereby incorporated by reference. The invention is further illustrated by the following example, which should not be construed as further limiting.

Binding and Antifibrillozenic Assays

Test compounds were purchased from commercial sources or synthesized and screened by mass spectroscopy (“MS”) assays. The MS assay gives data on the ability of compounds to bind to an amyloid.

In the mass spectroscopy (“MS”) assay, samples were prepared as aqueous solutions containing 20% ethanol, 200 μM of a test compound and 20 pM of solubilized AP40. The pH value of each sample was adjusted to 7.4 (0.2) by addition of 0.1% aqueous sodium hydroxide. The solutions were then analyzed by electrospray ionization mass spectroscopy using a Waters ZQ 4000 mass spectrometer. Samples were introduced by direct infusion at a flow-rate of 25 μL/min within 2 hr. after sample preparation. The source temperature was kept at 70° C. and the cone voltage was 20 V for all the analysis. Data were processed using Masslynx 3.5 software. The MS assay gives data on the ability of compounds to bind to soluble Aβ, whereas the ThT, EM and CD assays give data on inhibition of fibrillogenesis. The results from the assay for binding to Aβ are summarized in Table 2. “+++” indicates strong binding; “++” indicates moderate binding; “+” indicates weak binding; “−” indicates no detectable binding; and entries left blank were not determined.

An ultraviolet absorption assay is also available, and this assay gives an indication of the ability of test compounds to bind to (fibrillar) Aβ. The experiments were carried out in a blinded fashion. Test compound at 20 M μ was incubated with 50 μM Aβ(1-40) fibers for 1 h at 37° C. in Tris buffered saline (20 mM Tris, 150 mM NaCl, pH 7.4 containing 0.01 sodium azide). Following incubation, the solution was centrifuged for 20 min at 21,000 g to sediment the Aβ (1-40) fibers along with any bound test compound. The amount of test compound remaining in the supernatant was determined by reading the absorbance. The fraction of test compound bound was then calculated by comparing the amount remaining in the supernatants of incubations with Aβ to the amount remaining in control incubations which do not contain Aβ fibers. Thioflavin T and Congo Red, both of which are known to bind to AD fibers, are included in each assay run as positive controls. Before assaying, test compounds were diluted to 40 μM, which is twice the concentration in the final test, and then scanned using the Hewlett Packard 8453 UV/VIS spectrophotometer to determine if the absorbance was sufficient for detection.

Observed Synergistic Effects of Combination Therapy in Human Patients

In this example, mild and moderate patients have been treated with an alkanesulfonic acid, namely 3-amino-1-propanesulfonic acid, in combination with other therapeutic compounds used to diminish symptoms characteristic of Alzheimer's disease (e.g., loss of cognitive functions). The examples comprise the use of an alkanesulfonic acid in combination with cognitive enhancers such as acetylcholine esterase inhibitors (“AChEi”). The effect of these combination therapies on the change in ADAS-cog score in the patients was determined.

Patients were treated with the test alkanesulfonic acid for a period of 9 months. A group of patients received the test compound alone while another group were treated with the test compound in combination with an AChEi, namely donepezil.

Effect on ADAS-Cog. Upon entering the study, Alzheimer's patients were categorized as being “mild” or “moderate” according to their MMSE (“Mini Mental State Examination”) score. B.W.Rover, et al., “Mini-mental state exam in clinical practice.” Hospital Practice 22(1 A), 99 et seq. (1987). (According to this examination a MMSE score in the range of 19 to 26 was considered “mild,” and a score in the range 13 to 18 was considered “moderate”). Then, the change in mental function of these patients was analyzed by using their ADAS-Cog scores, which were recorded periodically over a nine month period. During this time, certain of the patients received the test alkanesulfonic acid compound, and others received the same alkanesulfonic acid concomitantly with donepezil, an acetylcholinesterase inhibitor. The average change in ADAS-Cog score for each group of patients was compared to the standard reported change in similar Alzheimer's disease patients who have been treated with donepezil alone. The reference and experimental data are tabulated below, according to the particular therapeutic regimen and the patient population:

| AD Patient | | Change in ADAS-Cog |
|---|---|---|
| Group(s) | Treatment | over 9 months |
| Mild | AchEi alone | +5.0 reference |
| Moderate | | +2.5 reference |
| Mild + Moderate | Test compound alone | −0.5 observed |
| combined | | |
| Mild + Moderate | Test compound + AchEi | −3.0 observed |
| combined | | |

A positive change in ADAS-Cog score reflects a deterioration of cognitive function of a “mild AD” patient; stabilization is seen by a change of ±1; and a negative change shows an improvement in cognitive function. The medical literature predicts that patients with Alzheimer's disease who do not receive any treatment will, on average, have a change in ADAS-cog score over nine months varying from +2.5 (for “mild” patients) to +5 (for “moderate” patients).

As the results indicate, patients who were treated with the test compound alone had a stable ADAS-Cog score (−0.5 on average, both the mild and moderate groups are considered together for convenience), and therefore the test compound appears to have limited a further decrease in the cognitive function of patients in this group over the test period.

The quantitative effects for each therapeutic drug regimen when administered separately are known. An additive effect for the combined mild plus moderate patient group would therefore be in the range of (+5.0 to +2.5)+(−0.5), which calculates to a deterioration on the ADAS-Cog scale of +4.5 to +2.0 points. Surprisingly, the opposite effect is observed. The effect of concomitant administration of the test alkanesulfonic acid compound with the acetylcholinesterase inhibitor led to an improvement of cognitive function (−3.0), whereas the predicted result is a decline. These results show an example of the benefit of having combination therapy for Alzheimer's disease patients.

The alkanesulfonic acid used in the study is known to have an effect on the concentration of Aβ in the brain. We also determined the effect of test compound on the change in Aβ CSF levels of patients with mild to moderate Alzheimer's disease compared to that seen in patients treated with a triple combination of test compound with AChEi and statin.

Patients treated with alkanesulfonic acid test compound in the presence or absence of AChEi and statin were evaluated for their Aβ CSF concentration at time 0 and 3 months following the initiation of treatment with the alkanesulfonic acid. The change in AP₄₂ CSF concentration was compared to the respective placebo group.

Patients treated with the test compound had a decrease of 34% in AβCSF concentration. The test compound was previously shown to decrease the levels of both soluble and insoluble AP₄₂ in the brain of transgenic mice. Based on the mice studies the test compound is hypothesized to favor the clearance of Aβ from the brain and CSF prior to its deposition. The decrease in Aβ. CSF concentration seen in patients treated with the test compound is greater than that seen in the placebo group, where patients showed a non-significant increase of 15% in their Aβ CSF concentration. This result demonstrates a difference of 49% between the two groups. Patients treated with the triple therapy (test compound and AchEi and statin) showed a decrease of 31% while patients on AChEi and statin showed an increase of 45% in their Aβ42 CSF levels. This triple therapy showed a greater effect on the change (−76%) of Aβ₄₂ CSF concentration when compared to the appropriate controls.

In sum, when compared to the respective placebo group, the combination of test compound with AchEi and statin showed a much greater effect on the Aβ CSF concentration than test compound alone.

Methodology. CSF was obtained from patients before and after treatment with the test compound at daily doses of 100 mg, 200 mg, or 300 mg. CSF was fractionated by FPLC following treatment with formic acid, and then the Aβ containing fraction was lyophilised. The amount of Aβ peptide was measured using an ELISA assay (Biosource). The test alkanesulfonic acid-containing composition was found to reduce the CSF level of Aβ when patients were treated with 200 or 300 mg daily doses. A majority of patients on placebo and on 100 mg daily doses showed stable Aβ CSF levels over a 3-month period, whereas the greatest reduction of Aβoccurred in patients receiving 200 or 300 mg daily doses. The presence of a drug in the cerebrospinal fluid suggests that the drug crosses the blood brain barrier to penetrate the brain. The presence of the alkanesulfonic acid in the CSF was determined in patients who had received treatment for three months. In these patients, CSF was collected five hours following dosing, and levels of alkanesulfonic acid were determined by LC-MS/MS. The test alkanesulfonic acid was found to be present in CSF of patients in a dose-dependent manner, e.g., patients receiving 200 or 300 mg daily dosing had a greater concentration than that seen in patients with 100 mg daily dosing.

Cognitive function with combination therapy—alkanesulfonic acidplus an acetyl cholenesterase inhibitor. Mild to moderate Alzheimer's disease patients who had been on cognition enhancers (Aricept™ or Exelon™) were co-medicated with daily doses of test drug (300 mg alkanesulfonic acid) for six months. At the time the experiments were undertaken the peer-reviewed medical literature indicated that patients who had been on ACHE inhibitors for more than twelve months would be expected to show a decline in cognitive functions of at least two to three points on the ADAS-Cog scale over a six month period. In order to determine whether the drug could potentiate or even stabilize the benefits of ACHE inhibitors, patients were treated with both ACHE and the test drug for a period of six months. Although the expectation was that the cognitive functions of patients receiving only AChE inhibitors would inevitably decline, the study shows that patients receiving co-medication had stable or improved ADAS-Cog, as illustrated in Graph A of the Figures, attached hereto. These results show that the test drug given concomitantly with ACHE inhibitors is able to maintain and even improve the cognitive function of patients.

1. A method of concomitant therapeutic treatment of a subject, comprising administering to a subject in need thereof an effective amount of a first agent and a second agent, wherein said first agent treats an amyloid-P disease, neurodegeneration, or cellular toxicity; and said second agent is a therapeutic drug or nutritive supplement.

2. A method of concomitant therapeutic treatment of a subject, comprising administering to a subject in need thereof an effective amount of a first agent and a second agent, wherein said first agent prevents, or slows, or stops progression of an amyloid-β disease; and said second agent is a therapeutic drug or nutritive supplement.

3. A method of concomitant therapeutic treatment of a subject, comprising administering to a subject in need thereof an effective amount of a pharmaceutical composition for treating an amyloid-β disease, said pharmaceutical composition comprising a first agent and a second agent in a pharmaceutically acceptable carrier, wherein said first agent treats an amyloid-β disease; and said second agent is a therapeutic drug or nutritive supplement.

4. A method of concomitant therapeutic treatment of a subject, comprising administering to a subject in need thereof an effective amount of a pharmaceutical composition, wherein said pharmaceutical composition comprising a first agent and a second agent in a pharmaceutically acceptable carrier, and wherein said pharmaceutical composition prevents, or slows, or stops progression of an amyloid-β disease in said subject.

5. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or inhibits amyloid-β fibril formation, neurodegeneration, or cellular toxicity; and said second agent is a therapeutic drug or nutritive supplement, such that activities of daily living otherwise impaired by said amyloid-β disease are improved or stabilized.

6. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or inhibits amyloid-β fibril formation, neurodegeneration, or cellular toxicity; and said second agent is a therapeutic drug or nutritive supplement, such that said pharmaceutical composition inhibits an interaction between an amyloidogenic protein and a glycoprotein or proteoglycan constituent of a basement membrane to thereby prevent or inhibit amyloid deposition.

7. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or inhibits amyloid-β fibril formation, neurodegeneration, or cellular toxicity; and said second agent is a therapeutic drug or nutritive supplement, such that the concentration of amyloid-β or tau in the CSF of said subject changes versus an untreated subject.

8. A method of concomitant therapeutic treatment of a subject, comprising administering to a subject in need thereof an effective amount of a pharmaceutical composition for treating or preventing an amyloid-β disease, said pharmaceutical composition comprising a first agent and at least two second agents in a pharmaceutically acceptable carrier, wherein said first agent prevents or inhibits amyloid-β fibril formation, neurodegeneration, or cellular toxicity; and each of said second agent is a therapeutic drug or nutritive supplement.

9. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent binds amyloid-β; and said second agent is a therapeutic drug or nutritive supplement; such that amyloid-β fibril formation, neurodegeneration, or cellular toxicity in said subject is prevented or inhibited.

10. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent binds amyloid-β; and said second agent is a therapeutic drug or nutritive supplement; such that cognitive function is stabilized or further deterioration in cognitive function is prevented, slowed, or stopped in said subject.

11. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent binds amyloid-β; and said second agent is a therapeutic drug or nutritive supplement; such that activities of daily living otherwise impaired by said amyloid-β disease are improved or stabilized in said subject.

12. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent binds amyloid-β; and said second agent is a therapeutic drug or nutritive supplement; such that said pharmaceutical composition inhibits an interaction between an amyloidogenic protein and a glycoprotein or proteoglycan constituent of a basement membrane to thereby prevent or inhibit amyloid deposition in said subject.

13. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent binds amyloid-β; and said second agent is a therapeutic drug or nutritive supplement; such that the concentration of amyloid-β or tau in the CSF of said subject changes versus an untreated subject.

14. A method of concomitant therapeutic treatment of a subject, comprising administering to a subject in need thereof an effective amount of a pharmaceutical composition for treating or preventing an amyloid-β disease, said pharmaceutical composition comprising a first agent and at least two second agents in a pharmaceutically acceptable carrier, wherein said first agent binds amyloid-β; and said second agent is a therapeutic drug or nutritive supplement; such that amyloid-β fibril formation, neurodegeneration, or cellular toxicity in said subject is prevented or inhibited.

15. The method of any of claim 1, wherein said first agent prevents or inhibits β-amyloid fibril formation.

16. The method of any of claim 1, wherein said first agent prevents β-amyloid peptide, in its soluble, oligomeric form or in its fibrillar form, from binding or adhering to a cell surface and causing cell damage or toxicity.

17. The method of any of claim 1, wherein said first agent blocks amyloid-induced cellular toxicity or microglial activation.

18. The method of claim 1, wherein said first agent blocks amyloid-induced neurotoxicity.

19. The method of claim 1, wherein said first agent reduces the rate or amount of βamyloid aggregation, fibril formation, or deposition.

20. The method of claim 1, wherein said first agent slows the rate of amyloid-β fibril formation or deposition.

21. The method of claim 1, wherein said first agent lessens the degree of amyloid-β deposition.

22. The method of claim 1, wherein said first agent inhibits, reduces, or prevents amyloid-β fibril formation.

23. The method of any of claim 1, wherein said first agent inhibits amyloid-β induced inflammation.

24. The method of any of claim 1, wherein said first agent enhances the clearance of amyloid-β from the brain.

25. The method of any of claim 1, wherein said first agent alters the equilibrium of amyloid-β between the CSF or brain and the plasma and decreases the amount of amyloid-β in the brain versus the equilibrium distribution in an untreated subject.

26. The method of claim 1, wherein said first agent reverses deposition of amyloid in a subject having amyloid deposits.

27. The method of claim 1, wherein said first agent favors deposition of amyloid in a subject having amyloid deposits.

28. The method of any of claim 1, wherein said first agent favors plaque clearance or slows deposition in a subject having amyloid deposits.

29. The method of claim 1, wherein said first agent decreases the amyloid-β concentration in the brain of a subject versus an untreated subject.

30. The method of claim 1, wherein said first agent penetrates into the brain.

31. The method of claim 1, wherein said first agent maintains soluble amyloid in a non-fibrillar form.

32. The method of claim 1, wherein said first agent increases the rate of clearance of soluble amyloid from the brain of a subject versus an untreated subject.

33. The method claim 1, wherein said first agent inhibits or reduces an interaction between amyloid-β and a cell surface constituent.

34. The method of claim 33 wherein said cell surface constituent is a glycosaminoglycan or proteoglycan constituent of a basement membrane.

35. The method of claim 10 wherein said amyloid-β is a peptide having 39-43 amino-acids.

36. The method of claim 10 wherein said amyloid-β is an amyloidogenic peptide produced from βAPP.

37. The method of claim 1, wherein said amyloid-β disease is Mild Cognitive Impairment or Mild-to-Moderate Cognitive Impairment.

38. The method of claim 1, wherein said amyloid-β disease is vascular dementia.

39. The method of claim 1, wherein said amyloid-β disease is Alzheimer's disease.

40. The method of claim 39, wherein said Alzheimer's disease is sporadic (non-hereditary) Alzheimer's disease.

41. The method of claim 39, wherein said Alzheimer's disease is familial (hereditary) Alzheimer's disease.

42. The method of claim 1, wherein said amyloid-β disease is cerebral amyloid angiopathy or hereditary cerebral hemorrhage.

43. The method of claim 1, wherein said amyloid-β disease is senile dementia.

44. The method of claim 1, wherein said amyloid-β disease is Down's syndrome, inclusion body myositis, or age-related macular degeneration.

45. The method of claim 3, wherein said pharmaceutical composition is therapeutically or prophylactically administered to a subject.

46. The method of claim 3, wherein said pharmaceutical composition is orally administered to a subject.

47. The method of claim 1, wherein said first agent and said second agent are simultaneously administered to a subject.

48. The method of claim 1, wherein said first agent is packaged in a separate container for sale or delivery to consumers from the container in which said second agent is packaged.

49. The method of claim 1, wherein said second or subsequent agent is packaged in a separate container for sale or delivery to consumers from the container in which said first agent is packaged.

50. The method of claim 1, wherein said first agent and said second agent act on different targets.

51. The method of claim 1, wherein said first agent and said second agent modulate different biological processes in the pathogenesis of Alzheimer's disease.

52. The method of claim 1, wherein said first agent and said second agent have different binding affinities or specificities for peptides, proteins, or enzymes involved in the pathogenesis of Alzheimer's disease.

53. The method of claim 1, wherein said first agent and said second agent when simultaneously present in a subject act synergistically to reduce, inhibit, or ameliorate the symptoms or pathogenesis of Alzheimer's disease.

54. The method of claim 1, wherein said subject is a human.

55. The method of claim 1, wherein said subject is a human over 40 years old.

56. The method of claim 1, wherein said subject is a human over 50 years old.

57. The method of claim 1, wherein said subject is a human over 60 years old.

58. The method of claim 1, wherein said subject is a human over 70 years old.

59. The method of claim 1, wherein said subject is a female human.

60. The method of claim 1, wherein said subject is a postmenopausal female human.

61. The method of claim 60, wherein said subject is on hormone replacement therapy.

62. The method of claim 1, wherein said subject is a male human.

63. The method of claim 1, wherein said subject has Alzheimer's disease or a genetic predisposition for developing Alzheimer's disease.

64. The method of claim 1, wherein said subject has vascular dementia.

65. The method of claim 1, wherein said subject has senile dementia.

66. The method of claim 1, wherein said subject has Mild Cognitive Impairment.

67. The method of claim 1, wherein said subject has a genomic mutation in the APP gene.

68. The method of claim 1, wherein said subject has a genomic mutation in the ApoE gene.

69. The method of claim 1, wherein said subject has a genomic mutation in a presenilin gene.

70. The method of claim 1, wherein said subject has familial, sporadic, or idiopathic Alzheimer's disease or cerebral amyloid angiopathy.

71. The method of claim 1, wherein said subject has amyloid deposits.

72. The method of claim 1, wherein said subject's brain has amyloid-β amyloid deposits.

73. A pharmaceutical composition comprising a first agent in a first pharmaceutically acceptable carrier and a second agent in a second pharmaceutically acceptable carrier, wherein said first pharmaceutically acceptable carrier is different from said second pharmaceutically acceptable carrier.

74. A kit comprising a first pharmaceutical composition comprising a first agent in a pharmaceutically acceptable carrier and a second pharmaceutical composition comprising a second agent in a pharmaceutically acceptable carrier, wherein said first pharmaceutical composition is different from said second pharmaceutical composition.

75. A pharmaceutical composition for the treatment of an amyloid-β disease comprising a first agent and a second agent in a pharmaceutically acceptable carrier, wherein said first agent prevents or inhibits amyloid-β fibril formation, neurodegeneration, or cellular toxicity; and said second agent is a therapeutic drug or nutritive supplement.

76. A pharmaceutical composition for the treatment of an amyloid-β disease comprising a first agent and a second agent in a pharmaceutically acceptable carrier, wherein said first agent prevents or inhibits amyloid-β fibril formation, neurodegeneration, or cellular toxicity; and said second agent is a therapeutic drug or nutritive supplement, such that cognitive function is stabilized or further deterioration in cognitive function is prevented, slowed, or stopped.

77. A pharmaceutical composition for the treatment of an amyloid-β disease comprising a first agent and a second agent in a pharmaceutically acceptable carrier, wherein said first agent prevents or inhibits amyloid-β fibril formation, neurodegeneration, or cellular toxicity; and said second agent is a therapeutic drug or nutritive supplement, such that activities of daily living otherwise impaired by an amyloid-β disease are improved or stabilized.

78. A pharmaceutical composition for the treatment of an amyloid-β disease comprising a first agent and a second agent in a pharmaceutically acceptable carrier, wherein said first agent prevents or inhibits amyloid-β fibril formation, neurodegeneration, or cellular toxicity; and said second agent is a therapeutic drug or nutritive supplement, such that said pharmaceutical composition inhibits an interaction between an amyloidogenic protein and a glycoprotein or proteoglycan constituent of a basement membrane to thereby prevent or inhibit amyloid deposition.

79. A pharmaceutical composition for the treatment of an amyloid-β disease comprising a first agent and a second agent in a pharmaceutically acceptable carrier, wherein said first agent prevents or inhibits amyloid-β fibril formation, neurodegeneration, or cellular toxicity; and said second agent is a therapeutic drug or nutritive supplement, such that the concentration of amyloid-β or tau in the CSF of said subject changes versus an untreated subject.

80. A pharmaceutical composition for the treatment of an amyloid-β disease comprising a first agent and at least two second agents in a pharmaceutically acceptable carrier, wherein said first agent prevents or inhibits amyloid-β fibril formation, neurodegeneration, or cellular toxicity; and each of said second agent is a therapeutic drug or nutritive supplement.

81. A pharmaceutical composition for the treatment of an amyloid-β disease comprising a first agent and a second agent in a pharmaceutically acceptable carrier, wherein said first agent binds amyloid-β; and said second agent is a therapeutic drug or nutritive supplement; such that amyloid-β fibril formation, neurodegeneration, or cellular toxicity in said subject is prevented or inhibited.

82. A pharmaceutical composition for the treatment of an amyloid-β disease comprising a first agent and a second agent in a pharmaceutically acceptable carrier, wherein said first agent binds amyloid-β; and said second agent is a therapeutic drug or nutritive supplement; such that cognitive function is stabilized or further deterioration in cognitive function is prevented, slowed, or stopped in said subject.

83. A pharmaceutical composition for the treatment of an amyloid-β disease comprising a first agent and a second agent in a pharmaceutically acceptable carrier, wherein said first agent binds amyloid-β; and said second agent is a therapeutic drug or nutritive supplement; such that activities of daily living otherwise impaired by said amyloid-β disease are improved or stabilized in said subject.

84. A pharmaceutical composition for the treatment of an amyloid-β disease comprising a first agent and a second agent in a pharmaceutically acceptable carrier, wherein said first agent binds amyloid-β; and said second agent is a therapeutic drug or nutritive supplement; such that said pharmaceutical composition inhibits an interaction between an amyloidogenic protein and a glycoprotein or proteoglycan constituent of a basement membrane to thereby prevent or inhibit amyloid deposition in said subject.

85. A pharmaceutical composition for the treatment of an amyloid-β disease comprising a first agent and a second agent in a pharmaceutically acceptable carrier, wherein said first agent binds amyloid-β; and said second agent is a therapeutic drug or nutritive supplement; such that the concentration of amyloid-β or tau in the CSF of said subject changes versus an untreated subject.

86. A pharmaceutical composition for the treatment of an amyloid-β disease comprising a first agent and at least two second agents in a pharmaceutically acceptable carrier, wherein said first agent binds amyloid-β; and said second agent is a therapeutic drug or nutritive supplement; such that amyloid-β fibril formation, neurodegeneration, or cellular toxicity in said subject is prevented or inhibited.

87. The pharmaceutical composition of claim 73, wherein said first agent prevents or inhibits β-amyloid fibril formation.

88. The pharmaceutical composition of claim 73, wherein said first agent prevents β-amyloid peptide, in its soluble, oligomeric form or in its fibrillar form, from binding or adhering to a cell surface and causing cell damage or toxicity.

89. The pharmaceutical composition of claim 73 wherein said first agent blocks amyloid-induced cellular toxicity or microglial activation.

90. The pharmaceutical composition of claim 73, wherein said first agent blocks amyloid-induced neurotoxicity.

91. The pharmaceutical composition of claim 73, wherein said first agent reduces the rate or amount of β-amyloid aggregation, fibril formation, or deposition.

92. The pharmaceutical composition of claim 73, wherein said first agent slows the rate of amyloid-β fibril formation or deposition.

93. The pharmaceutical composition of claim 73, wherein said first agent lessens the degree of amyloid-β deposition.

94. The pharmaceutical composition of claim 73, wherein said first agent inhibits, reduces, or prevents amyloid-β fibril formation.

95. The pharmaceutical composition of claim 73, wherein said first agent inhibits amyloid-β induced inflammation.

96. The pharmaceutical composition of claim 73, wherein said first agent enhances the clearance of amyloid-β from the brain.

97. The pharmaceutical composition of claim 73, wherein said first agent alters the equilibrium of amyloid-β between the brain and the plasma and decreases the amount of amyloid-β in the brain versus the equilibrium distribution in an untreated subject.

98. The pharmaceutical composition of claim 73, wherein said first agent reverses or favors deposition of amyloid in a subject having amyloid deposits.

99. The pharmaceutical composition of claim 73, wherein said first agent favors plaque clearance or slows deposition in a subject having amyloid deposits.

100. The pharmaceutical composition of claim 73, wherein said first agent decreases the amyloid-β concentration in the brain of a subject versus an untreated subject.

101. The pharmaceutical composition of claim 73, wherein said first agent penetrates into the brain.

102. The pharmaceutical composition of claim 73, wherein said first agent maintains soluble amyloid in a non-fibrillar form.

103. The pharmaceutical composition of claim 73, wherein said first agent increases the rate of clearance of soluble amyloid from the CSF or brain of a subject versus an untreated subject.

104. The pharmaceutical composition of claim 73, wherein said first agent inhibits or reduces an interacting between amyloid-β and a cell surface constituent.

105. The pharmaceutical composition of claim 104, wherein said cell surface constituent is a glycosaminoglycan or proteoglycan constituent of a basement membrane.

106. The pharmaceutical composition of claim 73, wherein said first agent and said second agent are packaged in separate containers for sale or delivery to the consumer.

107. The pharmaceutical composition of claim 75, wherein said first agent and said second agent are dissolved in a liquid pharmaceutically acceptable carrier.

108. The pharmaceutical composition of claim 73, wherein said first agent and said second agent are present as a homogenous mixture in a capsule or pill.

109. The pharmaceutical composition of claim 73, wherein said pharmaceutical composition further comprises a pharmaceutically acceptable acid, base, buffering agent, inorganic salt, solvent, or preservative.

110. The pharmaceutical composition of claim 73, further comprising a compound that increases the cerebral bioavailability of either said first agent or said second agent.

111. The use of a first agent and a second agent in the preparation of a pharmaceutical composition for the treatment or prevention of an amyloid-β disease comprising a first agent and a second agent in a pharmaceutically acceptable carrier, wherein said first agent prevents or inhibits amyloid-β fibril formation, neurodegeneration, or cellular toxicity; and said second agent is a therapeutic drug or nutritive supplement.

112. The pharmaceutical composition of claim 81, wherein said amyloid-β is a peptide having 39-43 amino-acids.

113. The pharmaceutical composition of claim 81, wherein said amyloid-β is an amyloidogenic peptide produced from βAPP.

114. The pharmaceutical composition of claim 75, wherein said amyloid-β disease is Mild Cognitive Impairment or Mild-to-Moderate Cognitive Impairment.

115. The pharmaceutical composition of claim 75, wherein said amyloid-β disease is vascular dementia.

116. The pharmaceutical composition of claim 75, wherein said amyloid-β disease is Alzheimer's disease.

117. The pharmaceutical composition of claim 116, wherein said Alzheimer's disease is sporadic (non-hereditary) Alzheimer's disease.

118. The pharmaceutical composition of claim 116, wherein said Alzheimer's disease is familial (hereditary) Alzheimer's disease.

119. The pharmaceutical composition of claim 75, wherein said amyloid-β disease is cerebral amyloid angiopathy or hereditary cerebral hemorrhage.

120. The pharmaceutical composition of claim 75, wherein said amyloid-β disease is senile dementia.

121. The pharmaceutical composition of claim 75, wherein said amyloid-β disease is Down's syndrome, Mild Cognitive Impairment, inclusion body myositis, or age-related macular degeneration.

122. The method of claim 1, wherein said first agent is a substituted or unsubstituted alkanesulfonic acid, substituted or unsubstituted alkanesulfuric acid, substituted or unsubstituted alkylthiosulfonic acid, substituted or unsubstituted alkylthiosulfuric acid, or an ester or amide thereof, including pharmaceutically acceptable salts thereof.

123. The method of claim 1, wherein said first agent is a substituted or unsubstituted alkanesulfonic acid, or an ester or amide thereof, including pharmaceutically acceptable salts thereof.

124. The method of claim 1, wherein said first agent is a substituted or unsubstituted lower alkanesulfonic acid, or an ester or amide thereof, including pharmaceutically acceptable salts thereof.

125. The method of claim 1, wherein said first agent is a (substituted- or unsubstituted-amino)-substituted alkanesulfonic acid, or an ester or amide thereof, including pharmaceutically acceptable salts thereof.

126. The method of claim 1, wherein said first agent is a (substituted- or unsubstituted-amino)-substituted lower alkanesulfonic acid, or an ester or amide thereof, including pharmaceutically acceptable salts thereof.

127. The method of claim 122, wherein said substituted or unsubstituted alkanesulfonic acid is a substituted or unsubstituted straight-chain alkanesulfonic acid, substituted or unsubstituted cycloalkanesulfonic acid, substituted or unsubstituted branched-chain alkanesulfonic acid.

128. The method of claim 126, wherein said amino substituent has the formula —NR^{a}R^{b}, wherein R^{a} and R^{b} are each independently hydrogen, an alkyl group, an aryl group, or a heterocyclyl group, or R^{a} and R^{b}, taken together with the nitrogen atom to which they are attached, form a heterocyclic moiety having from 3 to 8 atoms in the ring.

129. The method of claim 128, wherein said heterocyclic moiety is a piperidinyl or pyrrolidinyl group.

130. The method of claim 125, wherein said amino substituent is an alkylamino or dialkylamino group.

131. The method of claim 125, wherein said alkanesulfonic acid is an alkyl group substituted with at least a group of the formula —SO₃H or —SO₃⁻X⁺, where X⁺ is a cationic group at physiologic pH.

132. The method of claim 131, wherein said cationic group is a hydrogen atom or a sodium atom.

133. The method of claim 131, wherein said cationic group is an amino group.

134. The method of claim 123, wherein said alkanesulfonic acid is substituted with a straight or branched alkyl or cycloalkyl group, or a group of the formula —NH₂, —SO₃H, —OSO₃H, —CN, —NO₂, —F, —Cl, —Br, —I, —CH₂OCH₃, —OCH₃, —SH, —SCH₃, —OH, or —CO₂H.

135. The method of claim 123, wherein said alkanesulfonic acid is substituted with substituent selected from the group consisting of halogeno, trifluoromethyl, nitro, cyano, C₁-C₆ alkyl, C₂-C₆ alkenyl, C₂-C₆ alkynyl, C₁-C₆ alkylcarbonyloxy, arylcarbonyloxy, C₁-C₆ alkoxycarbonyloxy, aryloxycarbonyloxy, C₁-C₆ alkylcarbonyl, C₁-C₆ alkoxycarbonyl, C₁-C₆ alkoxy, C₁-C₆ alkylthio, arylthio, heterocyclyl, aralkyl, and aryl groups.

136. The method of claim 1, wherein said first agent is a compound or mixture of compounds having the following structure 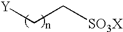
where Y is —NR^{a}R^{b} or —SO₃⁻X⁺, wherein n is an integer from 1 to 5, and X⁺ is hydrogen or a cationic group.

137. The method of claim 1, wherein said first agent is a compound or mixture of compounds having one of the following structures 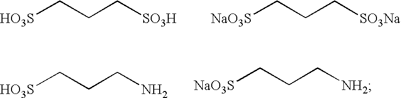
and pharmaceutically acceptable salts thereof.

138. The method of claim 1, wherein said first agent is 3-amino-1-propanesulfonic acid and pharmaceutically acceptable salts thereof.

139. The method of claim 1, wherein said second agent is curative of Alzheimer's disease or palliative of the symptoms thereof.

140. The method of claim 1, wherein said second agent is therapeutic drug that is useful in the treatment of Alzheimer's disease or a condition associated with Alzheimer's disease.

141. The method of claim 1, wherein said second agent is neuroprotective or neurotrophic.

142. The method of claim 1, wherein said second agent alters the biodistribution of amyloidogenic peptides between the periphery and the central nervous system.

143. The method of claim 1, wherein said second agent alters both the biodistribution and the equilibrium amount of the aggregation forms of amyloid-β from monomeric amyloid-β, soluble oligomeric amyloid-β, insoluble protofibrils, diffuse amyloid, compact amyloid, and neuritic amyloid, versus an untreated subject.

144. The method of claim 1, wherein said second agent alters the equilibrium amounts of the aggregation forms of amyloid-β, said forms including monomeric amyloid-β, soluble oligomeric amyloid-β, insoluble protofibrils, diffuse amyloid, compact amyloid, and neuritic amyloid, versus an untreated subject.

145. The method of claim 1, wherein said second agent alters reduces the neurotoxicity of oligomers or protofibrils of amyloid-β.

146. The method of claim 1, wherein said second agent enhances cognitive function or memory.

147. The method of claim 1, wherein said second agent potentiates cholinergic neurotransmission.

148. The method of claim 1, wherein said second agent inhibits acetylcholinesterase or potentiates choline acetyltransferase.

149. The method of claim 1, wherein said second agent is a cholinesterase inhibitor.

150. The method of claim 1, wherein said second agent is an acetylcholinesterase inhibitor.

151. The method of claim 1, wherein said second agent is a butyrylcholinesterase inhibitor.

152. The method of claim 1, wherein said second agent is phenserine.

153. The method of claim 1, wherein said second agent is tacrine (Cognex™, 1,2,3,4-tetrahydro-9-acridinamine), donepezil (Aricept™, 2,3-dihydro-5,6-dimethoxy-2-((1-(phenylmethyl)-4-piperidinyl)methyl)-1H-inden-1-one), rivastigmine (Exelon™, ethylmethylcarbamic acid 3-((1S)-1-(dimethylamino)ethyl)phenyl ester), or galanthamine (Reminyl™, (4aS,6R,8aS)-4a,5,9,10,11,12-hexahydro-3-methoxy-11-methyl-6H-benzofuro(3a,3,2-ef)(2)benzazepin-6-ol).

154. The method of claim 1, wherein said second agent is a steroidal sex hormone.

155. The method of claim 1, wherein said second agent is estrogen with or without progestins.

156. The method of claim 1, wherein said second agent is a substituted indole.

157. The method of claim 1, wherein said second agent is 3,3′-disubstituted-1,3-dihydro-2H-pyrrolo[2,3-b]heterocyclic-2-one.

158. The method of claim 1, wherein said agent is NO-flurbiprofen.

159. The method of claim 1, wherein said agent is flurbiprofen.

160. The method of claim 1, wherein said second agent stimulates neurons to release acetylcholine.

161. The method of claim 1, wherein said second agent is indole-3-propionic acid.

162. The method of claim 1, wherein said second agent is a muscarinic acetylcholine receptor agonist.

163. The method of claim 162, wherein said second agent is xanomeline.

164. The method of claim 1, wherein said second agent is an ergot alkaloid or a vinca alkaloids.

165. The method of claim 1, wherein said agent is hydrolysed in vivo to produce a compound with anticholinesterase activity.

166. The method of claim 1, wherein said second agent is a carbamate derivative of physostigmine.

167. The method of claim 1, wherein said second agent is a NMDA receptor antagonist.

168. The method of claim 1, wherein said second agent is memantine (Ebixa™ or Axura™, 3,5-dimethyl-1-adamantanamine).

169. The method of claim 1, wherein further comprising a neuroprotective agent that protects against NMDA agonist damage.

170. The method of claim 1, wherein said second agent inhibits the biosynthesis of amyloid-β.

171. The method of claim 1, wherein said second agent is a protease inhibitor that inhibits the biosynthesis of amyloid-β.

172. The method of claim 1, wherein said second agent is a β- or γ-secretase inhibitor.

173. The method of claim 1, wherein said second agent is an agonist of α-secretase.

174. The method of claim 1, wherein said second agent is a metal chelating compound.

175. The method of claim 1, wherein said second agent forms a stable chelate with a divalent metal ion.

176. The method of claim 1, wherein said second agent is a copper or zinc chelatoring compound.

177. The method of claim 1, wherein said second agent is a β-amino acid.

178. The method of claim 1, wherein said second agent is clioquinol.

179. The method of claim 1, wherein said second agent decrease the interaction of copper or zinc with amyloid-β peptides.

180. The method of claim 1, wherein said second agent is a cholinesterase inhibitor that inhibits the translation or processing of APP mRNA.

181. The method of claim 1, wherein said second agent is phenserine.

182. The method of claim 1, wherein said second agent is wortmannin.

183. The method of claim 1, wherein said second agent is leteprinim (Neotrofin™ or AIT-082, 4-((3-(1,6-dihydro-6-oxo-9H-purin-9-yl)-1-oxopropyl)amino) benzoic acid).

184. The method of claim 1, wherein said second agent prevents oligomerization or fibrillogenesis of Aβ or enhanes its clearance from the brain

185. The method of claim 1, wherein said second agent is an anti-fibrillogenic small molecule compound.

186. The method of claim 1, wherein said second agent is mixture of glycosaminoglycans having an average molecular weight equal to 2,400 Da.

187. The method of claim 1, wherein said second agent is a mucopolysaccharide (e.g., Ateroid™).

188. The method of claim 1, wherein said second agent is a THT analog.

189. The method of claim 1, wherein said second agent is an anti-inflammatory drug.

190. The method of claim 1, wherein said second agent is a nonsteroidal anti-inflammatory drug.

191. The method of claim 1, wherein said second agent is an inhibitor of cyclooxygenase.

192. The method of claim 1, wherein said second agent is ibuprofen, indomethacin, or sulindac sulphide.

193. The method of claim 1, wherein said second agent is a nonsteroidal anti-imflammatory drug that inhibit the biosynthesis of amyloid-β.

194. The method of claim 1, wherein said second agent is an antioxidant.

195. The method claim 1, wherein said second agent is capable of protecting against oxidative damage caused by reactive oxygen species.

196. The method of claim 1, wherein said second agent is melatonin.

197. The method of claim 1, wherein said second agent is curcumin.

198. The method of claim 1, wherein said second agent is vitamin E (α-tocopherol), vitamin C (ascorbic acid), vitamin B 12, vitamin A (retinoic acid), or co-enzyme Q.

199. The method of claim 1, wherein said second agent is selegiline.

200. The method of claim 1, wherein said second agent is homocysteine.

201. The method of claim 1, wherein said second agent is an iron chelate or an iron chelating ligand.

202. The method of claim 1, wherein said second agent is desferrioxamine.

203. The method of claim 1, wherein said second agent is a kinase/phosphatase inhibitor.

204. The method of claim 1, wherein said second agent inhibits the hyperphosphorylation of tau.

205. The method of claim 1, wherein said second agent inhibits GSK-3.

206. The method of claim 1, wherein said second agent is lithium.

207. The method of claim 1, wherein said second agent inhibits phosphorylation of poly(O) ataxin.

208. The method of claim 1, wherein said second agent inhibits Akt kinase.

209. The method of claim 1, wherein said second agent is an antihypercholesterolemic drug.

210. The method of claim 1, wherein said second agent is a statin.

211. The method of claim 1, wherein said second agent is an inhibitor of squalene oxide synthetase (HMG-COA reductase).

212. The method of claim 211, wherein said second agent is avorstatin, or another statin.

213. The method of claim 140, wherein said condition associated with Alzheimer's disease is a symptom characteristic of Alzheimer's disease.

214. The method of claim 140, wherein said condition associated with Alzheimer's disease is hypothyroidism.

215. The method of claim 140, wherein said condition associated with Alzheimer's disease is cerebrovascular or cardiovascular disease.

216. The method of claim 140, wherein said condition associated with Alzheimer's disease is memory loss, anxiety, or a behavioral dysfunction.

217. The method of claim 216, wherein said behavioral dysfunction is apathy, aggression, or incontinence.

218. The method of claim 140, wherein said condition associated with Alzheimer's disease is a psychological condition.

219. The method of claim 140, wherein said condition associated with Alzheimer's disease is a neurological condition.

220. The method of the claim 219, wherein said neurological condition is Huntington's disease, amyotrophic lateral sclerosis, acquired immunodeficiency, Parkinson's disease, aphasia, apraxia, agnosia, Pick disease, dementia with Lewy bodies, altered muscle tone, seizures, sensory loss, visual field deficits, incoordination, gait disturbance, transient ischemic attack or stroke, transient alertness, attention deficit, frequent falls, syncope, neuroleptic sensitivity, normal pressure hydrocephalus, subdural hematoma, brain tumor, posttraumatic brain injury, or posthypoxic damage.

221. The method of claim 218, wherein said psychological condition is depression, delusions, illusions, hallucinations, sexual disorders, weight loss, psychosis, a sleep disturbance such as insomnia, behavioral disinhibition, poor insight, suicidal ideation, depressed mood or irritability, anhedonia, social withdrawal, or excessive guilt.

222. The method of claim 1, wherein said therapeutic drug is a psychotropic medication.

223. The method of claim 1, wherein said therapeutic drug is an antidepressant.

224. The method of claim 1, wherein said therapeutic drug is a selective serotonin reuptake inhibitor.

225. The method of claim 1, wherein said therapeutic drug is an atypical antidepressant.

226. The method of claim 1, wherein said therapeutic drug is an antipsychotic.

227. The method of claim 1, wherein said therapeutic drug is an appetite stimulants.

228. The method of claim 1, wherein said therapeutic drug is a drug used to treat a condition associated with Alzheimer's disease.

229. The method of claim 1, wherein said nutritive supplement is a precursor of acetylcholine.

230. The method of claim 1, wherein said nutritive supplement is lecithin or choline.

231. The method of claim 1, wherein said nutritive supplement is Ginkgo biloba.

232. The method of claim 1, wherein said nutritive supplement is acetyl-L-carnitine.

233. The method of claim 1, wherein said nutritive supplement is idebenone.

234. The method of claim 1, wherein said nutritive supplement is propentofylline or a xanthine derivative.

235. The pharmaceutical composition of claim 73, wherein said first agent is a substituted or unsubstituted alkanesulfonic acid, substituted or unsubstituted alkanesulfuric acid, substituted or unsubstituted alkylthiosulfonic acid, substituted or unsubstituted alkylthiosulfuric acid, or an ester or amide thereof, including pharmaceutically acceptable salts thereof.

236. The pharmaceutical composition of claim 73, wherein said first agent is a substituted or unsubstituted alkanesulfonic acid, or an ester or amide thereof, including pharmaceutically acceptable salts thereof.

237. The pharmaceutical composition of claim 73, wherein said first agent is a substituted or unsubstituted lower alkanesulfonic acid, or an ester or amide thereof, including pharmaceutically acceptable salts thereof.

238. The pharmaceutical composition of claim 73, wherein said first agent is a (substituted- or unsubstituted-amino)-substituted alkanesulfonic acid, or an ester or amide thereof, including pharmaceutically acceptable salts thereof.

239. The pharmaceutical composition of claim 73, wherein said first agent is a (substituted- or unsubstituted-amino)-substituted lower alkanesulfonic acid, or an ester or amide thereof, including pharmaceutically acceptable salts thereof.

240. The pharmaceutical composition of 235, wherein said substituted or unsubstituted alkanesulfonic acid is a substituted or unsubstituted straight-chain alkanesulfonic acid, substituted or unsubstituted cycloalkanesulfonic acid, substituted or unsubstituted branched-chain alkanesulfonic acid.

241. The pharmaceutical composition of claim 239, wherein said amino substituent is has the formula —NR^{a}R^{b}, wherein R^{a} and R^{b} are each independently hydrogen, an alkyl group, an aryl group, or a heterocyclyl group, or R^{a} and R^{b}, taken together with the nitrogen atom to which they are attached, form a heterocyclic moiety having from 3 to 8 atoms in the ring.

242. The pharmaceutical composition of claim 241, wherein said heterocyclic moiety is a piperidinyl or pyrrolidinyl group.

243. The pharmaceutical composition of claim 241, wherein said amino substituent is an alkylamino or dialkylamino group.

244. The pharmaceutical composition of claim 238, wherein said alkanesulfonic acid is an alkyl group substituted with at least a group of the formula —SO₃H or —SO₃⁻X⁺, where X⁺ is a cationic group at physiologic pH.

245. The pharmaceutical composition of claim 244, wherein said cationic group is a hydrogen atom or a sodium atom.

246. The pharmaceutical composition of claim 244, wherein said cationic group is an amino group.

247. The pharmaceutical composition of claim 236, wherein said alkanesulfonic acid is substituted with a straight or branched alkyl or cycloalkyl group, or a group of the formula —NH₂, —SO₃H, —OSO₃H, —CN, —NO₂, —F, —Cl, —Br, —I, —CH₂OCH₃, —OCH₃, —SH, —SCH₃, —OH, or —CO₂H.

248. The pharmaceutical composition of claim 236, wherein said alkanesulfonic acid is substituted with substituent selected from the group consisting of halogeno, trifluoromethyl, nitro, cyano, C₁-C₆ alkyl, C₂-C₆ alkenyl, C₂-C₆ alkynyl, C₁-C₆ alkylcarbonyloxy, arylcarbonyloxy, C₁-C₆ alkoxycarbonyloxy, aryloxycarbonyloxy, C₁-C₆ alkylcarbonyl, C₁-C₆ alkoxycarbonyl, C₁-C₆ alkoxy, C₁-C₆ alkylthio, arylthio, heterocyclyl, aralkyl, and aryl groups.

249. The pharmaceutical composition of claim 73, wherein said first agent is a compound or mixture of compounds having the following structure 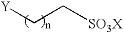
where Y is —NR^{a}R^{b} or —SO₃⁻X⁺, wherein n is an integer from 1 to 5, and X⁺ is hydrogen or a cationic group.

250. The pharmaceutical composition of claim 73, wherein said first agent is a compound or mixture of compounds having one of the following structures 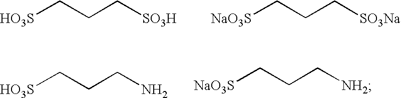
and pharmaceutically acceptable salts thereof.

251. The pharmaceutical composition of claim 73, wherein said first agent is 3-amino-1-propanesulfonic acid and pharmaceutically acceptable salts thereof.

252. The pharmaceutical composition of claim 73, wherein said second agent is curative of Alzheimer's disease or palliative of the symptoms thereof.

253. The pharmaceutical composition of claim 73, wherein said second agent is therapeutic drug that is useful in the treatment of Alzheimer's disease or a condition associated with Alzheimer's disease.

254. The pharmaceutical composition of claim 73, wherein said second agent is neuroprotective or neurotrophic.

255. The pharmaceutical composition of claim 73, wherein said second agent alters the biodistribution of amyloidogenic peptides between the periphery and the central nervous system.

256. The pharmaceutical composition of claim 73, wherein said second agent alters both the biodistribution and the equilibrium amount of the aggregation forms of amyloid-β from monomeric amyloid-P, soluble oligomeric amyloid-β, insoluble protofibrils, diffuse amyloid, compact amyloid, and neuritic amyloid, versus an untreated subject.

257. The pharmaceutical composition of claim 73, wherein said second agent alters the equilibrium amounts of the aggregation forms of amyloid-β, said forms including monomeric amyloid-β, soluble oligomeric amyloid-β, insoluble protofibrils, diffuse amyloid, compact amyloid, and neuritic amyloid, versus an untreated subject.

258. The pharmaceutical composition of claim 73, wherein said second agent alters reduces the neurotoxicity of oligomers or protofibrils of amyloid-β.

259. The pharmaceutical composition of claim 73, wherein said second agent enhances cognitive function or memory.

260. The pharmaceutical composition of claim 73, wherein said second agent potentiates cholinergic neurotransmission.

261. The pharmaceutical composition of claim 73, wherein said second agent inhibits acetylcholinesterase or potentiated choline acetyltransferase.

262. The pharmaceutical composition of claim 73, wherein said second agent is a cholinesterase inhibitor.

263. The pharmaceutical composition of claim 73, wherein said second agent is an acetylcholinesterase inhibitor.

264. The pharmaceutical composition of claim 73, wherein said second agent is a butyrylcholinesterase inhibitor.

265. The pharmaceutical composition of claim 73, wherein said second agent is phenserine.

266. The pharmaceutical composition of claim 73, wherein said second agent is tacrine (Cognex™, 1,2,3,4-tetrahydro-9-acridinamine), donepezil (Aricept™, 2,3-dihydro-5,6-dimethoxy-2-((1-(phenylmethyl)-4-piperidinyl)methyl)-1H-inden-1-one), rivastigmine (Exelon™, ethylmethylcarbamic acid 3-((1S)-1-(dimethylamino)ethyl)phenyl ester), or galanthamine (Reminyl™, (4aS,6R,8aS)-4a,5,9,10,11,12-hexahydro-3-methoxy-11-methyl-6H-benzofuro(3a,3,2-ef)(2)benzazepin-6-ol).

267. The pharmaceutical composition of claim 73, wherein said second agent is a steroidal sex hormone.

268. The pharmaceutical composition of claim 73, wherein said second agent is estrogen with or without progestins.

269. The pharmaceutical composition of claim 73, wherein said second agent is a substituted indole.

270. The pharmaceutical composition of claims 73, wherein said second agent is 3,3′-disubstituted-1,3-dihydro-2H-pyrrolo[2,3-b]heterocyclic-2-one.

271. The pharmaceutical composition of claim 73, wherein said second agent stimulates neurons to release acetylcholine.

272. The pharmaceutical composition of claim 73, wherein said second agent is indole-3-propionic acid.

273. The pharmaceutical composition of claim 73, wherein said second agent is a muscarinic acetylcholine receptor agonist.

274. The pharmaceutical composition of the claim 273, wherein said second agent is xanomeline.

275. The pharmaceutical composition of claim 73, wherein said second agent is an ergot alkaloid or a vinca alkaloids.

276. The pharmaceutical composition of claim 73, wherein said agent is hydrolysed in vivo to produce a compound with anticholinesterase activity.

277. The pharmaceutical composition of claim 73, wherein said second agent is a carbamate derivative of physostigmine.

278. The pharmaceutical composition of claim 73, wherein said second agent is a NMDA receptor antagonist.

279. The pharmaceutical composition of claim 73, wherein said second agent is memantine (Ebixa™ or Axura™, 3,5-dimethyl-1-adamantanamine).

280. The pharmaceutical composition of claim 73, wherein comprising a neuroprotective agent that protects against NMDA agonist damage.

281. The pharmaceutical composition of claim 73, wherein said second agent inhibits the biosynthesis of amyloid-β.

282. The pharmaceutical composition of claim 73, wherein said second agent is a protease inhibitor that inhibits the biosynthesis of amyloid-β.

283. The pharmaceutical composition of claim 73, wherein said second agent is a βor γ-secretase inhibitor.

284. The pharmaceutical composition of claim 73, wherein said second agent is an agonist of α-secretase.

285. The pharmaceutical composition of claim 73, wherein said second agent is a metal chelating compound.

286. The pharmaceutical composition of claim 73, wherein said second agent forms a stable chelate with a divalent metal ion.

287. The pharmaceutical composition of claim 73, wherein said second agent is a copper or zinc chelator.

288. The pharmaceutical composition of claim 73, wherein said second agent is a β-amino acid.

289. The pharmaceutical composition of claim 73, wherein said second agent is clioquinol.

290. The pharmaceutical composition of claim 73, wherein said second agent decrease the interaction of copper or zinc with amyloid-β peptides.

291. The pharmaceutical composition of claim 73, wherein said second agent is a cholinesterase inhibitor that inhibits the translation or processing of APP mRNA.

292. The pharmaceutical composition of claim 73, wherein said second agent is phenserine.

293. The pharmaceutical composition of claim 73, whereinsaid second agent is wortmannin.

294. The pharmaceutical composition of claim 73, wherein said second agent is leteprinim (Neotrofin™ or AIT-082, 4-((3-(1,6-dihydro-6-oxo-9H-purin-9-yl)-1-oxopropyl)amino) benzoic acid).

295. The pharmaceutical composition of claim 73, wherein said second agent prevents oligomerization or fibrillogenesis of Aβ or enhanes its clearance from the brain.

296. The pharmaceutical composition of claim 73, wherein said second agent is an anti-fibrillogenic small molecule compound.

297. The pharmaceutical composition of claim 73, wherein said second agent is mixture of glycosaminoglycans having an average molecular weight equal to 2,400 Da.

298. The pharmaceutical composition of claim 73, wherein said second agent is a mucopolysaccharide (e.g., Ateroid™).

299. The pharmaceutical composition of claim 73, wherein said second agent is a THT analog.

300. The pharmaceutical composition of claim 73, wherein said second agent is an anti-inflammatory drug.

301. The pharmaceutical composition of claim 73, wherein said second agent is a nonsteroidal anti-inflammatory drug.

302. The pharmaceutical composition of claim 73, wherein said second agent is an inhibitor of cyclooxygenase.

303. The pharmaceutical composition of claim 73, wherein said second agent is ibuprofen, indomethacin, or sulindac sulphide.

304. The pharmaceutical composition of claim 73, wherein said second agent is a nonsteroidal anti-imflammatory drug that inhibit the biosynthesis of amyloid-β.

305. The pharmaceutical composition of claim 73, wherein said second agent is an antioxidant.

306. The pharmaceutical composition of claim 73, wherein said second agent is capable of protecting against oxidative damage caused by reactive oxygen species.

307. The pharmaceutical composition of claim 73, wherein said second agent is melatonin.

308. The pharmaceutical composition of claim 73, wherein said second agent is curcumin.

309. The pharmaceutical composition of claim 73, wherein said second agent is vitamin E (α-tocopherol), vitamin C (ascorbic acid), vitamin B12, vitamin A (retinoic acid), or co-enzyme Q.

310. The pharmaceutical composition of claim 73, wherein said second agent is selegiline.

311. The pharmaceutical composition of claim 73, wherein said second agent is homocysteine.

312. The pharmaceutical composition of claim 73, wherein said second agent is an iron chelate or an iron chelating ligand.

313. The pharmaceutical composition of claim 73, wherein said second agent is desferrioxamine.

314. The pharmaceutical composition of claim 73, wherein said second agent is a kinase/phosphatase inhibitor.

315. The pharmaceutical composition of claim 73, wherein said second agent inhibits the hyperphosphorylation of tau.

316. The pharmaceutical composition of claim 73, wherein said second agent inhibits GSK-3.

317. The pharmaceutical composition of claim 73, wherein said second agent is lithium.

318. The pharmaceutical composition of claim 73, wherein said second agent inhibits phosphorylation of poly(Q) ataxin.

319. The pharmaceutical composition of claim 73, wherein said second agent inhibits Akt kinase.

320. The pharmaceutical composition of any of the foregoing claim 73, wherein said second agent is an antihypercholesterolemic drug.

321. The pharmaceutical composition of claim 73, wherein said second agent is a statin.

322. The pharmaceutical composition of claim 73, wherein said second agent is an inhibitor of squalene oxide synthetase (HMG-COA reductase).

323. The pharmaceutical composition of claim 73, wherein said second agent is avorstatin, or another statin.

324. The pharmaceutical composition of claim 73, wherein said second agent is an agonist of α-secretase.

325. The pharmaceutical composition of claim 253, wherein said condition associated with Alzheimer's disease is a symptom characteristic of Alzheimer's disease.

326. The pharmaceutical composition of claim 253 wherein said condition associated with Alzheimer's disease is hypothyroidism.

327. The pharmaceutical composition of claim 253, wherein said condition associated with Alzheimer's disease is cerebrovascular or cardiovascular disease.

328. The pharmaceutical composition of claim 253 wherein said condition associated with Alzheimer's disease is memory loss, anxiety, or a behavioral dysfunction.

329. The pharmaceutical composition of claim 328, wherein said behavioral dysfunction is apathy, aggression, or incontinence.

330. The pharmaceutical composition of claims 253, wherein said condition associated with Alzheimer's disease is a psychological condition.

331. The pharmaceutical composition of claim 253, wherein said condition associated with Alzheimer's disease is a neurological condition.

332. The pharmaceutical composition of claim 331, wherein said neurological condition is Huntington's disease, amyotrophic lateral sclerosis, acquired immunodeficiency, Parkinson's disease, aphasia, apraxia, agnosia, Pick disease, dementia with Lewy bodies, altered muscle tone, seizures, sensory loss, visual field deficits, incoordination, gait disturbance, transient ischemic attack or stroke, transient alertness, attention deficit, frequent falls, syncope, neuroleptic sensitivity, normal pressure hydrocephalus, subdural hematoma, brain tumor, posttraumatic brain injury, or posthypoxic damage.

333. The pharmaceutical composition of claim 330, wherein said psychological condition is depression, delusions, illusions, hallucinations, sexual disorders, weight loss, psychosis, a sleep disturbance such as insomnia, behavioral disinhibition, poor insight, suicidal ideation, depressed mood or irritability, anhedonia, social withdrawal, or excessive guilt.

334. The pharmaceutical composition of claim 75, wherein said therapeutic drug is a psychotropic medication.

335. The pharmaceutical composition of claim 75, wherein said therapeutic drug is an antidepressant.

336. The pharmaceutical composition of claim 75 wherein said therapeutic drug is a selective serotonin reuptake inhibitor.

337. The pharmaceutical composition of claim 75, wherein said therapeutic drug is an atypical antidepressant.

338. The pharmaceutical composition of claim 75, wherein said therapeutic drug is an antipsychotic.

339. The pharmaceutical composition of claim 75, wherein said therapeutic drug is an appetite stimulants.

340. The pharmaceutical composition of claim 75, wherein said therapeutic drug is a drug used to treat a condition associated with Alzheimer's disease.

341. The pharmaceutical composition of claim 75, wherein said nutritive supplement is a precursor of acetylcholine.

342. The pharmaceutical composition of claim 75, wherein said nutritive supplement is lecithin or choline.

343. The pharmaceutical composition of claim 75, wherein said nutritive supplement is Ginkgo biloba.

344. The pharmaceutical composition of claim 75, wherein said nutritive supplement is acetyl-L-carnitine.

345. The pharmaceutical composition of claim 75, wherein said nutritive supplement is idebenone.

346. The pharmaceutical composition of claim 75, wherein said nutritive supplement is propentofylline or a xanthine derivative.

347. A method of concomitant therapeutic treatment of a subject, comprising administering to a subject in need thereof an effective amount of a first agent for treating or preventing an amyloid-β disease and a second agent, wherein said first agent prevents or inhibits amyloid-β fibril formation, neurodegeneration, or cellular toxicity; and said second agent is a therapeutic drug or nutritive supplement.

348. A method of concomitant therapeutic treatment of a subject, comprising administering to a subject in need thereof an effective amount of a first agent for treating or preventing an amyloid-β disease and a second agent, wherein said first agent prevents or inhibits amyloid-β fibril formation, neurodegeneration, or cellular toxicity; and said second agent is a therapeutic drug or nutritive supplement, such that cognitive function is stabilized or further deterioration in cognitive function is prevented, slowed, or stopped.

349. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or inhibits amyloid-β fibril formation, neurodegeneration, or cellular toxicity; and said second agent is a therapeutic drug or nutritive supplement.

350. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or inhibits amyloid-β fibril formation, neurodegeneration, or cellular toxicity; and said second agent is a therapeutic drug or nutritive supplement, such that cognitive function is stabilized or further deterioration in cognitive function is prevented, slowed, or stopped.

351. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or treats amyloid-β related disease; and said second agent is a therapeutic drug or nutritive supplement, such that activities of daily living otherwise impaired by said amyloid-β disease are improved or stabilized.

352. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or treats amyloid-β related disease; and said second agent is a therapeutic drug or nutritive supplement, such that said pharmaceutical composition inhibits an interaction between an amyloidogenic protein and a glycoprotein or proteoglycan constituent of a basement membrane to thereby prevent or inhibit amyloid deposition.

353. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or treats amyloid-β related disease; and said second agent is a therapeutic drug or nutritive supplement, such that the concentration of amyloid-β or tau in the CSF of said subject changes versus an untreated subject.

354. A method of concomitant therapeutic treatment of a subject, comprising administering to a subject in need thereof an effective amount of a pharmaceutical composition for treating or preventing an amyloid-β disease, said pharmaceutical composition comprising a first agent and at least two second agents in a pharmaceutically acceptable carrier, wherein said first agent prevents or treats amyloid-β related disease; and each of said second agent is a therapeutic drug or nutritive supplement.

355. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or treats amyloid-β related disease; and said second agent is a therapeutic drug or nutritive supplement; such that amyloid-β fibril formation, neurodegeneration, or cellular toxicity in said subject is prevented or inhibited.

356. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or treats amyloid-β related disease; and said second agent is a therapeutic drug or nutritive supplement; such that cognitive function is stabilized or further deterioration in cognitive function is prevented, slowed, or stopped in said subject.

357. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or treats amyloid-β related disease; and said second agent is a therapeutic drug or nutritive supplement; such that activities of daily living otherwise impaired by said amyloid-β disease are improved or stabilized in said subject.

358. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or treats amyloid-β related disease; and said second agent is a therapeutic drug or nutritive supplement; such that said pharmaceutical composition inhibits an interaction between an amyloidogenic protein and a glycoprotein or proteoglycan constituent of a basement membrane to thereby prevent or inhibit amyloid deposition in said subject.

359. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or treats amyloid-β related disease; and said second agent is a therapeutic drug or nutritive supplement; such that the concentration of amyloid-β or tau in the CSF of said subject changes versus an untreated subject.

360. A method of concomitant therapeutic treatment of a subject, comprising administering to a subject in need thereof an effective amount of a pharmaceutical composition for treating or preventing an amyloid-β disease, said pharmaceutical composition comprising a first agent and at least two second agents in a pharmaceutically acceptable carrier, wherein said first agent prevents or treats amyloid-β related disease; and said second agent is a therapeutic drug or nutritive supplement; such that amyloid-β fibril formation, neurodegeneration, or cellular toxicity in said subject is prevented or inhibited.

361. A pharmaceutical composition for the treatment of an amyloid-β disease comprising a first agent and a second agent in a pharmaceutically acceptable carrier, wherein said first agent prevents or treats amyloid-β related disease; and said second agent is a therapeutic drug or nutritive supplement.

362. A pharmaceutical composition for the treatment of an amyloid-P disease comprising a first agent and a second agent in a pharmaceutically acceptable carrier, wherein said first agent prevents or treats amyloid-β related disease; and said second agent is a therapeutic drug or nutritive supplement, such that cognitive function is stabilized or further deterioration in cognitive function is prevented, slowed, or stopped.

363. A pharmaceutical composition for the treatment of an amyloid-β disease comprising a first agent and a second agent in a pharmaceutically acceptable carrier, wherein said first agent prevents or treats amyloid-β related disease; and said second agent is a therapeutic drug or nutritive supplement, such that activities of daily living otherwise impaired by an anyloid-β disease are improved or stabilized.

364. A pharmaceutical composition for the treatment of an amyloid-β disease comprising a first agent and a second agent in a pharmaceutically acceptable carrier, wherein said first agent prevents or treats amyloid-β related disease; and said second agent is a therapeutic drug or nutritive supplement, such that said pharmaceutical composition inhibits an interaction between an amyloidogenic protein and a glycoprotein or proteoglycan constituent of a basement membrane to thereby prevent or inhibit amyloid deposition.

365. A pharmaceutical composition for the treatment of an amyloid-β disease comprising a first agent and a second agent in a pharmaceutically acceptable carrier, wherein said first agent prevents or treats amyloid-β related disease; and said second agent is a therapeutic drug or nutritive supplement, such that the concentration of amyloid-β or tau in the CSF of said subject changes versus an untreated subject.

366. A pharmaceutical composition for the treatment of an amyloid-β disease comprising a first agent and at least two second agents in a pharmaceutically acceptable carrier, wherein said first agent prevents or treats amyloid-β related disease; and each of said second agent is a therapeutic drug or nutritive supplement.

367. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or treats amyloid-β related disease; and said second agent is a therapeutic drug or nutritive supplement, such that the level of amyloid-β in the CSF of the subject is decreased versus an untreated subject.

368. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or treats amyloid-β related disease; and said second agent is a therapeutic drug or nutritive supplement, such that the level of amyloid-β in the CSF or the plasma of the subject is decreased versus an untreated subject.

369. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent prevents or treats amyloid-β related disease; and said second agent is a therapeutic drug or nutritive supplement, such that the level of amyloid-β in the plasma of the subject is modulated versus an untreated subject.

370. A pharmaceutical composition for the treatment of an amyloid-β disease comprising a first agent and a second agent in a pharmaceutically acceptable carrier, wherein said first agent prevents or treats amyloid-β related disease; and said second agent is a therapeutic drug or nutritive supplement, such that the level of amyloid-β in the CSF or the plasma of the subject is decreased or modulated versus an untreated subject.

371. A method of preventing or treating an amyloid-β related disease in a subject, said method comprising administering to a subject in need thereof an effective amount of a first agent that prevents or treats amyloid-β related disease, and a second agent that is a therapeutic drug or nutritive supplement.

372. The method of claim 371, wherein said first agent prevents or inhibits amyloid-β fibril formation, neurodegeneration, or cellular toxicity.

373. A method for preventing or treating Alzheimer's disease in a subject, said method comprising administering to a subject in need thereof an effective amount of a first agent that prevents or treats Alzheimer's disease, and a second agent that is a therapeutic drug or nutritive supplement.

374. The method of claim 373, wherein said first agent prevents or inhibits amyloid-β fibril formation, neurodegeneration, or cellular toxicity.

375. The method of claim 373, wherein said first agent is 3-amino-1-propanesulfonic acid or a pharmaceutically acceptable salt thereof.

376. The method of claim 371, wherein said amyloid-β related disease is Alzheimer's disease.

377. A method of preventing or treating Alzheimer's disease comprising concomitantly administering to a subject in need thereof an effective amount of a first agent that is efficacious in preventing or treating Alzheimer's disease in said subject and a second agent, wherein said first agent comprises 3-amino-1-propanesulfonic acid or a pharmaceutically acceptable salt thereof.

378. A method of preventing or treating Mild Cognitive Impairment comprising concomitantly administering to a subject in need thereof an effective amount of a first agent that is efficacious in preventing or treating Mild Cognitive Impairment in said subject and a second agent, wherein said first agent comprises 3-amino-1-propanesulfonic acid or a pharmaceutically acceptable salt thereof.

379. A method of preventing or treating Alzheimer's disease comprising concomitantly administering to a subject in need thereof an effective amount of a first agent that is efficacious in preventing or treating Alzheimer's disease in said subject and a second agent, wherein said first agent is 3-amino-1-propanesulfonic acid.

380. A method of preventing or treating Mild Cognitive Impairment comprising concomitantly administering to a subject in need thereof an effective amount of a first agent that is efficacious in preventing or treating Mild Cognitive Impairment in said subject and a second agent, wherein said first agent is 3-amino-1-propanesulfonic acid.

381. The method of claim 377, wherein said second agent is a cholinesterase inhibitor.

382. The method of claim 377, wherein said second agent is a statin.

383. The method of claim 377, wherein said second agent is memantine.

384. A pharmaceutical composition for preventing or treating Alzheimer's disease comprising an effective amount of a first agent that is efficacious in preventing or treating Alzheimer's disease in said subject and a second agent, wherein said first agent comprises 3-amino-1-propanesulfonic acid or a pharmaceutically acceptable salt thereof.

385. A pharmaceutical composition for preventing or treating Mild Cognitive Impairment comprising an effective amount of a first agent that is efficacious in preventing or treating Mild Cognitive Impairment in said subject and a second agent, wherein said first agent comprises 3-amino-1-propanesulfonic acid or a pharmaceutically acceptable salt thereof.

386. A pharmaceutical composition for preventing or treating Alzheimer's disease comprising an effective amount of a first agent that is efficacious in preventing or treating Alzheimer's disease in said subject and a second agent, wherein said first agent is 3-amino-1-propanesulfonic acid.

387. A pharmaceutical composition for preventing or treating Mild Cognitive Impairment comprising an effective amount of a first agent that is efficacious in preventing or treating Mild Cognitive Impairment in said subject and a second agent, wherein said first agent is 3-amino-1-propanesulfonic acid.

388. The pharmaceutical composition of claim 384, wherein said second agent is a cholinesterase inhibitor.

389. The pharmaceutical composition of claim 384, wherein said second agent is a statin.

390. The pharmaceutical composition of claim 384, wherein said second agent is memantine.

391. A method of concomitant therapeutic treatment of a subject, comprising administering to a subject in need thereof an effective amount of a pharmaceutical composition for treating or preventing an amyloid-β disease, said pharmaceutical composition comprising a first agent and a second agent in a pharmaceutically acceptable carrier, wherein said first agent modulates amyloid-β levels in the plasma or CSF; and said second agent is a therapeutic drug or nutritive supplement.
